SEQUENCE LISTING (984 sequences)

SEQ ID NO: 1 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 2 - protein, 453 aa, Artificial Sequence
  His-tagged protein
GALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTGGGSGGGSHHHHHHHHHH

SEQ ID NO: 3 - protein, 674 aa, Artificial Sequence
  Fc-tagged protein
GALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTGGGSGGGSPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 124 aa, Mus musculus
AVQLQESGPDLVKPSQSLSLTCTVTGYSITSNYNWHWIRQFPGNKLEWMGYIHYSDDTNYNPSLKSRISITRDPSKNQFFLQLNSVTTEDTATYYCARWSYYYGGPYYYSMDYWGQGTSVTVSS

SEQ ID NO: 5 - protein, 124 aa, Mus musculus
DVQLQESGPDLVKPSQSLSLTCTVTGYSITSGYNWHWIRQFPGNKLEWMGYIHYSGGTNYNPSLKSRISITRDASKNQFFLQLNSVTTEDTATYYCGRWSYYYGSPYYYSMDYWGQGTSVTVSS

SEQ ID NO: 6 - protein, 120 aa, Mus musculus
DVQLQESGPGLVKPSQSLSLTCSVTGFSITSGYYWNWIRQFPGNKLEWMGYISYDGSNNYNPSLKNRISITRDTSKNQFFLNLNSVTTEDTATYYCARDYYGYPAWFTYWGQGTLVTVSA

SEQ ID NO: 7 - protein, 119 aa, Mus musculus
DVQLQESGPGLVKPSQSLSLTCSVTGYSITDGYFWNWFRQFPGNKLEWMGFLNYDGSNNYNPSLKNRLSITRDTSENQFFLRLSSVTTEDTATYYCARRKGWLYAMDYWGQGTSVTVSS

SEQ ID NO: 8 - protein, 119 aa, Mus musculus
DVQLQESGPGLVKPSQSLSLTCSVTGYSITSAYFWNWFRQFPGNKLEWLGFINYDGSNNYNPSLKNRISITRDTSKNQFFLRLSSVTTEDTATYYCARRKGWLYAMDYWGQGTSVTVSS

SEQ ID NO: 9 - protein, 119 aa, Mus musculus
DVQLQESGPGLVKPSQSLSLTCSVTGYSITSAYFWNWFRQFPGNKLEWMGYINFDGSSNYNPSLKNRISITRDTSKNQFFLRLSSVTSEDTATYFCARRKGWLYAMDYWGQGTSITVSS

SEQ ID NO: 10 - protein, 119 aa, Mus musculus
DVQLQESGPGLVKPSQSLSLTCSVTGYSITSAYFWNWFRQFPGNRLEWLGFINYDGTNTYNPSLKNRISITRDTSKNQFFLRLSSVTTEDTATYYCARRKGWLYAMDYWGQGTSVTVSS

SEQ ID NO: 11 - protein, 119 aa, Mus musculus
DVQLQESGPGLVKPSQSLSLTCSVTGYSITSAYFWNWFRQFPGNRLEWMGFINYDGTNTYNPSLKNRISITRDTSKNQFFLRLSSVTTEDTATYYCARRKGWLYAMDYWGQGTSVTVSS

SEQ ID NO: 12 - protein, 119 aa, Mus musculus
DVQLQESGPGLVKPSQSLSLTCSVTGYSITSDSFWNWIRQVPGNKLEWMGFISYDGSNNYNPSLKNRISITRDTSKNQFFLKLNSVTTEDTATYYCARRKGWLYAMDYWGQGTSVTVSS

SEQ ID NO: 13 - protein, 119 aa, Mus musculus
DVQLQESGPGLVKPSQSLSLTCSVTGYSITSDYFWNWIRQFPGNKLEWMGFISYDGSNNYNPSLKNRISITRDTSKNQFFLKLNSVTSEDTATYFCARRKGWLYAMDYWGQGTSVTVSS

SEQ ID NO: 14 - protein, 119 aa, Mus musculus
DVQLQESGPGLVKPSQSLSLTCSVTGYSITSDYFWNWIRQFPGNKLEWMGFISYDGSNYCNPSLKNRISITRDTSKNQFFLKLNSVTTEDTATYYCARRKGWLYAMDYWGQGTSVTVSS

SEQ ID NO: 15 - protein, 119 aa, Mus musculus
DVQLQESGPGLVKPSQSLSLTCSVTGYSITSGYFWNWFRQFPGDKLEWMGYINYDGSNNYNPSLKNRVSISRDTSKNQFFLRLSSVTAEDTATYYCARRKGWLYAMDYWGQGTSVTVSS

SEQ ID NO: 16 - protein, 119 aa, Mus musculus
DVQLQESGPGLVKPSQSLSLTCSVTGYSITSGYFWNWFRQFPGNKLEWMGFINYDGSDNYNPSLKNRISITRDTSENQFFLRLSSVTTEDTATYYCARRKGWLYAMDSWGQGTSVTVSS

SEQ ID NO: 17 - protein, 119 aa, Mus musculus
DVQLQESGPGLVKPSQSLSLTCSVTGYSITSGYFWNWFRQFPGNKLEWMGFINYDGSNNYNPSLKNRISITRDTSKNQFFLRLSSVTSEDTATYYCARRKGWLYAMDYWGQGTSVTVSS

SEQ ID NO: 18 - protein, 119 aa, Mus musculus
DVQLQESGPGLVKPSQSLSLTCSVTGYSITSGYFWNWFRQFPGNKLEWMGFINYDGSSNYNPSLKNRISITRDTSENQFFLRLSSVTTEDTATYYCARRKGWLYAMDYWGQGTSVTVSS

SEQ ID NO: 19 - protein, 119 aa, Mus musculus
DVQLQESGPGLVKPSQSLSLTCSVTGYSITSGYFWNWFRQFPGNKLEWMGFLNYDGSNNYNPSLKNRISITRDTSENQFFLRFNSVTTEDTATYYCARRKGWLYAMDYWGQGTSVTVSS

SEQ ID NO: 20 - protein, 119 aa, Mus musculus; identical to SEQ ID NO: 19

SEQ ID NO: 21 - protein, 119 aa, Mus musculus
DVQLQESGPGLVKPSQSLSLTCSVTGYSITSGYFWNWFRQFPGNKLEWMGFLNYDGSNNYNPSLKNRISITRDTSENQFFLRLSSVTTEDTATYFCARRKGWLYAMDYWGQGTSVTVSS

SEQ ID NO: 22 - protein, 119 aa, Mus musculus
DVQLQESGPGLVKPSQSLSLTCSVTGYSITSGYFWNWFRQFPGNKLEWMGFLNYDGSNNYNPSLKNRISITRDTSENQFFLRLSSVTTEDTATYYCARRKGWLYAMDYWGQGTSVTVSS

SEQ ID NO: 23 - protein, 119 aa, Mus musculus
DVQLQESGPGLVKPSQSLSLTCSVTGYSITSGYFWNWFRQFPGNKLEWMGFLNYDGSNNYNPSLKNRISITRDTSKNQFFLRLSSVTTEDTATYYCARRKGWLYAMDYWGQGTSVTVSS

SEQ ID NO: 24 - protein, 119 aa, Mus musculus
DVQLQESGPGLVKPSQSLSLTCSVTGYSITSGYFWNWFRQFPGNKLEWMGFLNYDGSSNYNPSLKNRISITRDTSENQFFLRLSSVTTEDTATYYCARRKGWLYAMDYWGQGTSVTVSS

SEQ ID NO: 25 - protein, 119 aa, Mus musculus
DVQLQESGPGLVKPSQSLSLTCSVTGYSITSGYFWNWIRQFPGNKLEWMGFINYDGSNNYNPSLKNRISITRDTSKNQFFLKLNSVTTEDTATYYCARRKGWLYAMDYWGQGTSVTVSS

SEQ ID NO: 26 - protein, 116 aa, Mus musculus
DVQLQESGPGLVKPSQSLSLTCSVTGYSITSGYYWNWIRQFPGNKLEWMGYISYDGSNNYNPSLKNRISITRDTSKNQFFLKLNSVTTEDTATYYCAREGDYGSYWGQGTLVTVSA

SEQ ID NO: 27 - protein, 119 aa, Mus musculus
DVQLQESGPGLVKPSQSLSLTCSVTGYSITSGYYWNWIRQFPGNKLEWMGYISYDGSNNYNPSLKNRISITRDTSKNQFFLKLTSVTTEDTATYYCTRRKGWLYAMDYWGQGTSVTVSS

SEQ ID NO: 28 - protein, 121 aa, Mus musculus
DVQLQESGPGLVKPSQSLSLTCTVTGYSITSDYAWNWIRQFPGNKLEWMGYISYSGSTNYNPSLKSRISITRDTSKNQFFLQLNSVTTEDTATYYCASGYYGIHYYAMDYWGQGTSVTVSS

SEQ ID NO: 29 - protein, 119 aa, Mus musculus
DVQLQESGPGLVKSSQSLSLTCSVTGYSITSGYFWNWIRQFPGNKLEWMGFINYDGSSNYNPSLKNRISITRDTSKNQFFLKLNSVTTEDTATYYCARRKGWLYAMDYWGQGTSVTVSS

SEQ ID NO: 30 - protein, 125 aa, Mus musculus
EIHLQQTGPELVKPGASVKISCKASGYSFTAYIMLWVKQSHGKSLEWIGNINPYYGSTSYNLKFKGKATLTVDQSSSTAYMQLNSLTSDDSAVYYCARGPPYYYGSSRDYALDYWGQGTSVTVSS

SEQ ID NO: 31 - protein, 115 aa, Mus musculus
EIQLQQTGPELVKPGASVKISCKASGYSFTDYIMLWVKQSHGKSLEWIGNINPYYDTTAYNLKFKGKATLTVDKSSSTAYMLLNSLTSEDSAVYYCATYDGFAYWGQGTLVTVSA

SEQ ID NO: 32 - protein, 115 aa, Mus musculus
EIQLQQTGPELVKPGASVKISCKASGYSFTDYIMLWVKQSHGKSLEWIGNINPYYGTTSYNLKFKGKATLTVDKSSSTAYMQLNSLTSEDSAVYYCATYDGFAYWGQGTLVTVSA

SEQ ID NO: 33 - protein, 115 aa, Mus musculus
EIQLQQTGPELVKPGASVKISCKASGYSFTDYIMLWVKQSHGKSLEWIGNINPYYYTTSYNLKFKGKATLTVDKSSSTAYMQLNSLTSEDSAVYYCATYDGFAYWGQGTLVTVSA

SEQ ID NO: 34 - protein, 118 aa, Mus musculus
EVHLQQSGAELVRPGALVKLSCKASGFNIKDYYMHWVKQRPEQGLEWIGWIDPENGNTIYDPKFQGKASITADTSSNTAYLQLSSLTSEDTAVYYCTIGVRRDYFDYWGQGTTLTVSS

SEQ ID NO: 35 - protein, 117 aa, Mus musculus
EVKLEESGGGLVQPGGSMRLSCVASGFTFSNYWMNWVRQSPEKGLEWVAEIRLKFNNYTTHYAESVKGRFTISRDDSKSSVYLQMNNLRTEDTGIYYCIKFGGMDYWGPGTSVTVSS

SEQ ID NO: 36 - protein, 119 aa, Mus musculus
EVKLVESGGGLVKPGGSLKLSCAASGFAFNSYDMSWVRQTPEKRLEWVATISDGGSFTYYPDSVKGRFTISRDNARNTLYLHMSSLRSEDTALYYCARHQITTLRMDYWGQGTSVTVSS

SEQ ID NO: 37 - protein, 119 aa, Mus musculus
EVKLVESGGGLVKPGGSLKLSCAASGFAFSSYDMSWVRQTPEKRLEWVAAISDGGSYTYYPDSVKGRFTISRDNAGNTLYLQMSSLRSEDTALYYCARHRVTTVRMDYWGQGTSVTVSS

SEQ ID NO: 38 - protein, 119 aa, Mus musculus
EVKLVESGGGLVKPGGSLKLSCAASGFAFSSYDMSWVRQTPEKRLEWVATISDGGSYTYYPDSVKGRFTISRDNAGKTLYLQMSSLRPEDTALYYCARHRVTTVRMDYWGQGTSVTVSS

SEQ ID NO: 39 - protein, 119 aa, Mus musculus
EVKLVESGGGLVKPGGTLKLSCAASGFAFSDYDMSWVRQTPEKRLEWVATISDGGSYTYYPDSVKGRFTISRDNARNTLYLQMSSLRSEDTALYYCARHLITTLRMDYWGQGASVTVSS

SEQ ID NO: 40 - protein, 119 aa, Mus musculus
EVKLVESGGGLVKPGGTLKLSCAASGFAFSDYDMSWVRQTPEKRLEWVATISDGGSYTYYPDSVKGRFTVSRDNARNTLYLQMSSLRSEDTALYYCARHLITTLRMDYWGQGASVTVSS

SEQ ID NO: 41 - protein, 119 aa, Mus musculus
EVKLVESGGGLVKPGGTLKLSCAASGFAFSDYDMSWVRQTPEKRLEWVATISNDGSYTYYPDSVKGRFTISRDNARNTLYLQMSSLRSEDTALYYCARHLITTLRMDYWGQGASVTVSS

SEQ ID NO: 42 - protein, 118 aa, Mus musculus
EVMLVASGGGLVKPGGSLKLSCAASGFTFSSYTVSWVRQTPEKRLEWVASIISGGGDIYYPDSVRGRFTISRDNARNNLYLQMSSLRSEDTALYYCARWYSAGYFDVWGAGTTVTVSS

SEQ ID NO: 43 - protein, 117 aa, Mus musculus
EVMLVASGGGLVKPGGSLKLSCAASGFTFSSYTVSWVRQTPEKRLEWVATISGGGVTYYPDSVRGRFTISRDNAKNNLYLQMRSLRSEDTALYYCARWYSVGYFDVWGAGTTVTVSS

SEQ ID NO: 44 - protein, 117 aa, Mus musculus
EVMLVASGGGLVKPGGSLKLSCAASGFTFSSYTVSWVRQTPEKRLEWVATITGGGDTYYPDSVRGRFTISRDNAKNNLYLQMSSLRSEDTALYYCARWYSVGYFDVWGAGTTVTVSS

SEQ ID NO: 45 - protein, 118 aa, Mus musculus
EVMLVASGGGLVKPGGSLKLSCAASGFTFSSYTVSWVRQTPEKRLEWVATITSGGGDTYYPDSVRGRFTISRDNAKNNLYLEMSSLRSEDTALYYCARWYSAGYFDVWGAGTTVTVSS

SEQ ID NO: 46 - protein, 117 aa, Mus musculus
EVMLVASGGGLVKPGGSLNLSCAASGFTFSSYTVSWVRQTPEKRLEWVATISGGGVTYYPDTVRGRFTISRDNAKNNLYLQMRSLRSEDTALYYCARWYSVGYFDVWGAGTTVTVSS

SEQ ID NO: 47 - protein, 118 aa, Mus musculus
EVMLVESGGGLVKPGGSLKLSCAASGFTFSSYAMSWVRQTPEKRLEWVATIFFGSSVTYFPASVKGRFTISRDNANNTLFLQLSSLRSEDTAMYYCARQGTSPWFAYWGQGTLVTVSA

SEQ ID NO: 48 - protein, 118 aa, Mus musculus
EVMLVESGGGLVKPGGSLKLSCAASGFTFSSYTMSWVRQTPEKRLEWVATIISGGGNTYYPDSVKGRFTISRDNAKNNLYLQMSSLRSEDTALYYCARWYSAGYFDVWGAGTTVTVSS

SEQ ID NO: 49 - protein, 118 aa, Mus musculus
EVMLVESGGGLVKPGGSLKLSCAASGFTFSSYTMSWVRQTPEKRLEWVATISGGVGDTYYPDSVKGRFTISRDNAKNNLYLQMSSLRSEDTALYYCARQGGFPWFAYWGQGTLVTVSA

SEQ ID NO: 50 - protein, 117 aa, Mus musculus
EVQLQPSGAELVKPGASVKLSCTASGFNIEDTYIHWMKQRPEQGLEWIGRIDPANGNTISDPKFQGKATITADTSSNTAYLQLSSLTSEDTAVYYCARDGYYAPGYWGQGTTLTVSS

SEQ ID NO: 51 - protein, 117 aa, Mus musculus
EVQLQPSGAELVKPGASVKLSCTASGFNIEDTYIHWMKQRPEQGLEWIGRIDPANGNTISDPKFRGKATITADTSSNTAYLQLSSLTSEDTAVYYCARDGYYAPGYWGQGTTLTVSS

SEQ ID NO: 52 - protein, 117 aa, Mus musculus
EVQLQPSGSELVKPGASVKLSCTASGFNIEDTYIHWMKQRPEQGLEWIGRIDPANGYTTYDPKFQDKATITADTSSNTAYLQLSSLTSEDTAVYYCARDGYYAPGYWGQGTTLTVSS

SEQ ID NO: 53 - protein, 121 aa, Mus musculus
EVQLQQFGAELVKPGASMKISCKASGYTFTDYNMAWVKQSHGKSLEWIGDINPNYDSTSYNQKFKGKATLTVDKSSSTAYMELRSLTSEDAALYYCARSGFDGYYYAMDYWGQGTSVTVSS

SEQ ID NO: 54 - protein, 121 aa, Mus musculus
EVQLQQFGAELVKPGASVKISCKASDYTFTDYNMDWVKQSHGKSLEWIGDINPNNGGTIYNLKFKGKATLTVDKSSSTAYMELRSLTSEDTAVYYCARRHYGNYFYAMDYWGQGTSVTVSS

SEQ ID NO: 55 - protein, 121 aa, Mus musculus
EVQLQQFGAELVKPGASVKISCKASGYTFTDYNIDWVKQSHGKSLEWIGDINPNGDFTTYNLKFKGKATLTVDKSSSTAYMELRSLTSEDTAVYYCARRHYGNYFYAMDYWGQGTSVTVSS

SEQ ID NO: 56 - protein, 121 aa, Mus musculus
EVQLQQFGAELVKPGASVKISCKASGYTFTDYNMDWVKQNHGKSLEWIGDINPNNGGTIYNLKFKGKATLTVDKSSSTAYMELRSLTSEDTAVYYCARRHYGNYFYAMDYWGQGTSVTVSS

SEQ ID NO: 57 - protein, 121 aa, Mus musculus
EVQLQQFGAELVKPGASVKISCKASGYTFTDYNMDWVKQSHGKSLEWIGDINPNDDSTKYNLKFKGKATLTVDKSSSTAYMELRSLTSEDTAVYYCARRHYGNYFYAMDYWGQGTSVTVSS

SEQ ID NO: 58 - protein, 121 aa, Mus musculus
EVQLQQFGAELVKPGASVKISCKASGYTFTDYNMDWVKQSHGKSLEWIGDINPNDDSTSYNLKFKGKATLTVDKSSSTAYMELRSLTSEDTAVYYCARRHYGNYFYAMDYWGQGTSVTVSS

SEQ ID NO: 59 - protein, 121 aa, Mus musculus
EVQLQQFGAELVKPGASVKISCKASGYTFTDYNMDWVKQSHGKSLEWIGDINPNNGGTIYNLKFKGKATLTVDKSSSTAYMELRSLTSEDTAVYYCARRHYGNYFYAMDYWGQGTSVTVSS

SEQ ID NO: 60 - protein, 121 aa, Mus musculus
EVQLQQFGAELVKPGASVKISCKASGYTFTDYNMDWVKQSHGKSLEWIGDINPNNGGTIYNQKFKGKATLTVDKSSSTAYMELRSLTSEDTAVYYCARRHYGNYFYAMDYWGQGTSVTVSS

SEQ ID NO: 61 - protein, 121 aa, Mus musculus
EVQLQQFGAELVKPGASVKISCKASGYTFTDYNMDWVKQSHGKSLEWIGDINPNNGGTLYNLKFKGKATLTVDKSSSTAYMELRSLTSEDTAVYYCARRHYGNYFYAMDYWGQGTSVTVSS

SEQ ID NO: 62 - protein, 121 aa, Mus musculus
EVQLQQFGAELVKPGASVKISCKASGYTFTDYNMDWVKQSHGKSLEWIGDINPNYDSTNYNLKFKGKATLTVDKSSSTAYMELRSLTSEDTAVYYCARRHYGNYFYAMDYWGQGTSVTVSS

SEQ ID NO: 63 - protein, 121 aa, Mus musculus
EVQLQQFGAELVKPGASVKISCKASGYTFTDYNMDWVKQSHGKSLEWIGDISPNYESTNYSLKFKGKATLTVDKSSSTAYMELRSLTSEDTAVYYCARRHYGNYFYAMDYWGQGTSVTVSS

SEQ ID NO: 64 - protein, 121 aa, Mus musculus
EVQLQQFGAELVKPGASVKISCKASGYTFTDYNVDWVKQSHGKSLEWIGDINPNDDSTSYNLKFKGKATLTVDKSSSTAYMELRSLTSEDTAVYYCARRHYGNYFYAMDYWGQGTSVTVSS

SEQ ID NO: 65 - protein, 119 aa, Mus musculus
EVQLQQFGTELVKPGASVKISCKASGYTFTDYNINWVKQSHGRSLEWIGDINPNYDSTSYNQNFKGKATLTVDKSSSTSYMELRSLTSEDTAVYYCARGGFYGNFFDYWGQGTTLTVSS

SEQ ID NO: 66 - protein, 119 aa, Mus musculus
EVQLQQFGTELVKPGASVKISCKASGYTFTDYNINWVKQSHGRSLEWIGDINPNYDSTSYNQNFKGKATLTVDKSSSTSYMELRSLTSEDTAVYYGARGGFYGNFFDYWGQGTTLTVSS

SEQ ID NO: 67 - protein, 118 aa, Mus musculus
EVQLQQSGADLVRPGALVKLSCKTSGFNIKDYYMHWVKQRPEQGLEWIGWIDPENGNTIYDPKFQGKASITADTSSSTAYLQLSSLTSEDTAVYYCSIGVRRDYFAYWGQGTTLTVSS

SEQ ID NO: 68 - protein, 118 aa, Mus musculus
EVQLQQSGAEFVRPGALVKLSCKTSGFNIKDYYMHWVKQRPEQGLEWIGWIDPENGNTIYDPKFQGKASITADTSSNTAYLQLSSLTSEDTAVYYCTIGVRRDYFDYWGQGTTLTVSS

SEQ ID NO: 69 - protein, 117 aa, Mus musculus
EVQLQQSGAELVKPGASVKLSCTASGFNIEDTYIHWMKQRPEQGLEWIGRIDPANGNPIYDPKFQGKATITADSSSNTAYLQLSSLTSEDTAVYYCARDGYYAPGYWGQGTTLTVSS

SEQ ID NO: 70 - protein, 117 aa, Mus musculus
EVQLQQSGAELVKPGASVKLSCTASGFNIEDTYIHWMKQRPEQGLEWIGRIDPANGNPKSDPRFQGKATITADSSSNTAYLQFSSLTSEDTAVYYCARDGYYAPGYWGQGTTLTVSS

SEQ ID NO: 71 - protein, 117 aa, Mus musculus
EVQLQQSGAELVKPGASVKLSCTASGFNIEDTYIHWMKQRPEQGLEWIGRIDPANGNRKYDPRFQGKATITADSSSNTAYLQLSSLTSEDTAVYYCARDGYYAPGYWGQGTTLTVSS

SEQ ID NO: 72 - protein, 117 aa, Mus musculus
EVQLQQSGAELVKPGASVKLSCTASGFNIEDTYIHWMKQRPEQGLEWIGRIDPANGNTISDPRFRGKATITADTSSNTAYLQFSSLTSEDTAVYYCARDGYYAPGYWGQGTTLTVSS

SEQ ID NO: 73 - protein, 114 aa, Mus musculus
EVQLQQSGAELVKPGASVKLSCTASGFNIEDTYMHWVKERPEQGLEWIGRNDPANGNTEYDPKFQGKATITSDTSSNTAYLQLSTLTSEDTAVYYCASLWVDYWGQGTSVTVSS

SEQ ID NO: 74 - protein, 117 aa, Mus musculus
EVQLQQSGAELVKPGASVKLSCTASGFNIKDTYIHWMKQRPEQGLEWIGRIDPANANTQYDPKFQGKATITADTSSNTAYLQLSSLTSEDTAVYYCAHDGYYAPGYWGQGTTLTVSS

SEQ ID NO: 75 - protein, 117 aa, Mus musculus
EVQLQQSGAELVKPGASVKLSCTASGFNIKDTYIHWMKQRPEQGLEWIGRIDPANGNTIYDPKFQGKATITADTSSNTAYLQLSSLTSEDTAVYYCAHDGYYAPGYWGQGTTLTVSS

SEQ ID NO: 76 - protein, 117 aa, Mus musculus
EVQLQQSGAELVKPGASVKLSCTASGFNIKDTYIHWMKQRPEQGLEWIGRIDPANGNTKYDPKFQGKATITADTSSNTAYLQLSSLTSEDTAVYYCAHDGYYAPGYWGQGTTLTVSS

SEQ ID NO: 77 - protein, 117 aa, Mus musculus
EVQLQQSGAELVKPGASVKLSCTASGFNIKDTYIHWMKQRPIQGLEWIGRIDPANGNSHYDPKFQGKATITADTSSNTAYLQLSSLTSEDTAVYYCAHDGYYAPGYWGQGTTLTVSS

SEQ ID NO: 78 - protein, 117 aa, Mus musculus
EVQLQQSGAELVKPGASVKLSCTASGFNIKDTYIHWMMQRPEQGLEWIGRIDPANGNTIYDPKFQGKATITADTSSNTAYLHLSSLTSEDTAVYYCAHDGYYAPGYWGQGTTLTVSS

SEQ ID NO: 79 - protein, 117 aa, Mus musculus
EVQLQQSGAELVKPGASVKLSCTASGFNIKDTYIHWMMQRPEQGLEWIGRIDPANGNTIYDPKFQGKATITADTSSNTAYLQLSSLTSEDTAVYYCAHDGYYAPGNWGQGTTLTVSS

SEQ ID NO: 80 - protein, 117 aa, Mus musculus
EVQLQQSGAELVKPGASVKLSCTASGFNIKDTYIHWMMQRPEQGLEWIGRIDPANGNTIYDPKFQGKATITADTSSNTAYLQLSSLTSEDTAVYYCAHDGYYAPGYWGQGTTLTVSS

SEQ ID NO: 81 - protein, 117 aa, Mus musculus
EVQLQQSGAELVKPGASVKLSCTASGFNIKDTYIHWMMQRPEQGLEWIGRIDPANGNTNYDPKFQGKATITADTSSNTAYLQLSSLTSEDTAVYYCAHDGYYAPGYWGQGTTLTVSS

SEQ ID NO: 82 - protein, 117 aa, Mus musculus
EVQLQQSGAELVKPGASVKLSCTASGFNIKDTYIHWVKQRPEQGLEWIGRIDPANGNRIYDPKFQGKATITADSSSNTAYLQLSSLTSEDTAVYFCARDGYYAPGYWGQGTTLTVSS

SEQ ID NO: 83 - protein, 117 aa, Mus musculus
EVQLQQSGAELVKPGASVKLSCTASGFNIKDTYIHWVKQRPEQGLEWIGRIDPANGNRIYDPKFQGKATITADSSSNTAYLQLSSLTSEDTAVYYCARDGYYAPGYWGQGTTLTVSS

SEQ ID NO: 84 - protein, 117 aa, Mus musculus
EVQLQQSGAELVKPGASVKLSCTASGFNIKDTYIHWVKQRPEQGLEWIGRIDPANGNRKYDPKFQAKATITADSSSNTAYLQLSSLTSEDTAVYYCARDGYYAPGYWGQGTTLTVSS

SEQ ID NO: 85 - protein, 117 aa, Mus musculus
EVQLQQSGAELVKPGASVKLSCTASGFNIKDTYIHWVKQRPEQGLEWIGRIDPANGNRKYDPKFQGKATITADSLSNTAYLQLSSLTSEDTAVYYCARDGYYAPGHWGQGTTLTVSS

SEQ ID NO: 86 - protein, 117 aa, Mus musculus
EVQLQQSGAELVKPGASVKLSCTASGFNIKDTYIHWVKQRPEQGLEWIGRIDPANGNRKYDPKFQGKATITADSSSNTAYLQLSSLASEDTAVYYCARDGYSAPGYWGQGTTLTVSS

SEQ ID NO: 87 - protein, 117 aa, Mus musculus
EVQLQQSGAELVKPGASVKLSCTASGFNIKDTYIHWVKQRPEQGLEWIGRIDPANGNRKYDPKFQGKATITADSSSNTAYLQLSSLTSEDTAVYYCARDGYYAPGYWGQGTTLTVSS

SEQ ID NO: 88 - protein, 117 aa, Mus musculus
EVQLQQSGAELVKPGASVKLSCTASGFNIKDTYIHWVKQRPEQGLEWIGRIDPANGNRKYDPKFQGTATITADSSSNTAYLQLSSLTSEDTAVYYCARDGYYAPGYWGQGTTLTVSS

SEQ ID NO: 89 - protein, 117 aa, Mus musculus
EVQLQQSGAELVKPGASVKLSCTASGFNIKDTYMHWVKQRPEQGLEWIGRIDPANGNRKYDPKFQGKATITADSSSNTAYLQLSSLTSEDTAVYYCARDGYYAPGYWGQGTTLTVSS

SEQ ID NO: 90 - protein, 117 aa, Mus musculus
EVQLQQSGAELVKPGASVKLSCTASGFNIKDTYMHWVKQRPEQGLEWIGRIDPANGNRKYDPKFQGKATITADSSSNTAYLQLSSLTSEDTAVYYWARDGYYAPGYWGQGTTLTVSS

SEQ ID NO: 91 - protein, 117 aa, Mus musculus
EVQLQQSGAELVKPGASVKLSCTASGFNIKDTYMHWVKQRPEQGLEWIGRIDPANGNTGYDPKFQGKATITADTSSNTAYLQLSSLTSEDTAVYYCAHDGYYAPGYWGQGTTLTVSS

SEQ ID NO: 92 - protein, 117 aa, Mus musculus
EVQLQQSGAELVKPGASVKLSCTASGFNIKDTYMHWVKQRPEQGLEWIGRIDPANGNTHYDPKFQVKATITADTSSNTAYLQLSSLTSEDTAVYYCTHDGYYAPGYWGQGTTLTVSS

SEQ ID NO: 93 - protein, 117 aa, Mus musculus
EVQLQQSGAELVKPGASVKLSCTASGFNIKDTYMHWVKQRPEQGLEWIGRIDPANGNTIYDPKFQAKATITADTSSNTAYLQLSSLTSEDTAVYYCAHDGYYAPGYWGQGTTLTVSS

SEQ ID NO: 94 - protein, 117 aa, Mus musculus
EVQLQQSGAELVKPGASVKLSCTASGFNIKDTYMHWVKQRPEQGLEWIGRIDPANGNTIYDPNFQGKATITADTSSNTAYLQLSSLTSEDTAVYYCAHDGYYAPGYWGQGTTLTVSS

SEQ ID NO: 95 - protein, 117 aa, Mus musculus
EVQLQQSGAELVKPGASVKLSCTASGFNIKDTYMHWVKQRPEQGLEWIGRIDPANGNTNYDPKFQGKATITTDTSSNTAYLQLSSLTSEDTAVYYCAHDGYYAPGFWGQGTTLTVSS

SEQ ID NO: 96 - protein, 117 aa, Mus musculus
EVQLQQSGAELVKPGASVKLSCTASGFNIKDTYMNWVKQRPEQGLEWIGRIDPANGNTKYDPKFQGKATITADTSSNTAYLQLSSLTSEDTAVYYCAPFSYGTLAYWGQGTTLTVSS

SEQ ID NO: 97 - protein, 117 aa, Mus musculus
EVQLQQSGAELVKPGASVKLSCTASGLNIKDTYIHWVKQRPEQGLEWIGRIDPANGNRKYDPKFQGKATITADSSSNTAYLQLSSLTSEDTAVYYCARDGYYAPGYWGQGTTLTVSS

SEQ ID NO: 98 - protein, 117 aa, Mus musculus
EVQLQQSGAELVKPGASVKLSCTASGVNIKDTYIHWVKQRPEQGLEWIGRIDPANGNRKSDPKFQGKATITADSSSNTAYLQLSSLTSEDTAVYYCARDGYYAPGYWGQGTTLTVSS

SEQ ID NO: 99 - protein, 117 aa, Mus musculus
EVQLQQSGAELVKPGASVKLSCTASGVNIKDTYIHWVKQRPEQGLEWIGRIDPANGNRKYDPKFQGKATITADSSSNTAYLQLSSLTSEDTAVYYCARDGYYAPGYWGQGTTLTVSS

SEQ ID NO: 100 - protein, 114 aa, Mus musculus
EVQLQQSGAELVKPGASVKLSCTVSGFNIKDTYMHWVKQRPEQGLEWIGMIDPANFNTKYDPKFQGKATVTADTSSNTAYLQLSSLTSEDTAVYYCASLWVDYWGQGTSVTVSS

SEQ ID NO: 101 - protein, 117 aa, Mus musculus
EVQLQQSGAELVKPGASVKVSCTASGFNIKDTYIHWVKQRPEQGLEWIGRIDPANGNRKYDPKFQGKATITADSLSNTAYLQLSSLTSEDTAVYYCARDGYYAPGHWGQGTTLSVSS

SEQ ID NO: 102 - protein, 118 aa, Mus musculus
EVQLQQSGAELVRPGALVKLSCKASGCNIKDYYMHWVKQRPEQGLEWIGWIDPENGNTIYDPKFQAKASITADTSSNTAYLQFSSLTSEDTAVYYCTIGVRRDYFAYWGQGTTLTVSS

SEQ ID NO: 103 - protein, 118 aa, Mus musculus
EVQLQQSGAELVRPGALVKLSCKASGCNIKDYYMHWVKQRPEQGLEWIGWIDPENGNTIYDSKFQAKASITADTSSNTAYLQFSSLTSEDTAVYYCAIGVRRDYFAYWGQGTTLTVSS

SEQ ID NO: 104 - protein, 118 aa, Mus musculus
EVQLQQSGAELVRPGALVKLSCKASGFNIKDFYMHWVKQRPEQGLEWIGWIDPENGNTIYDPKFQGKASITADTSSNTAYLQLSSLTSEDTAVYYCAIGVRRDYFGYWGQGTTLTVSS

SEQ ID NO: 105 - protein, 118 aa, Mus musculus
EVQLQQSGAELVRPGALVKLSCKASGFNIKDYYMHWVKQRPEQGLEWIGWIDPENGNTIYDPKFQGKARITADTSSNTAYLQLSSLTSEDTAVYYCAIGVRRDYFAYWGQGTTLTVSS

SEQ ID NO: 106 - protein, 118 aa, Mus musculus
EVQLQQSGAELVRPGALVKLSCKASGFNIKDYYMHWVKQRPEQGLEWIGWIDPENGNTIYDPKFQGKASITADTSSNTAYLQLSSLTSEDTAVYYCAIGVRRDYFAYWGQGTTLTVSS

SEQ ID NO: 107 - protein, 118 aa, Mus musculus
EVQLQQSGAELVRPGALVKLSCKASGFNIKDYYMHWVKQRPEQGLEWIGWIDPENGNTIYDPKFQGKASITADTSSNTAYLQLSSLTSEDTAVYYCAIGVRRDYFDYWGQGTTLTVSS

SEQ ID NO: 108 - protein, 118 aa, Mus musculus
EVQLQQSGAELVRPGALVKLSCKTSGFNIKDYYMHWVKQRPEQGLEWIGWIDPENGNTIYDPKFQDKAGITADTSSNTAYLQLSSLTSEDSAVYYCAIGVRRDYFAYWGQGTTLTVSS

SEQ ID NO: 109 - protein, 118 aa, Mus musculus
EVQLQQSGAELVRPGALVKLSCKTSGFNIKDYYMHWVKQRPEQGLEWIGWIDPENGNTIYDPKFQGKASITADTSSNTAYLQLNSLTSEDTAVYYCAIGVRRDYFDYWGQGTTLTVSS

SEQ ID NO: 110 - protein, 118 aa, Mus musculus
EVQLQQSGAELVRPGALVKLSCKTSGFNIKDYYMHWVKQRPEQGLEWIGWIDPENGNTIYDPKFQGKASITADTSSNTAYLQLSSLTSEDTAVYYCAIGVRRDYFDYWGQGTTLTVSS

SEQ ID NO: 111 - protein, 122 aa, Mus musculus
EVQLQQSGPELLKPGASVKISCKTSGYTFTEYTLHWVKQSHGKSLEWIGVINPNSGGTTYNQKFKDKATLTVDKSSSSAYMELRSLTSEDSAVYYCTRPHSLLRLRREFDYWGQGTTLTVSS

SEQ ID NO: 112 - protein, 118 aa, Mus musculus
EVQLQQSGPELVKPGASMKISCKVSGFSFTGYTMNWVKQSHGKNLEWIGLINTYNGGTFYNQKFKAKATLTVDKSSSTAHMELLSLTSEDSAVYYCARSPGRYSMDYWGQGTSVTVSS

SEQ ID NO: 113 - protein, 122 aa, Mus musculus
EVQLQQSGPELVKPGASMQISCKTSGYTFTEYTVHWVKQSHGKSLEWIGVINPSTGGTTYNQKFKGKATLTVDQSSSTAYMELRSLTSEDSAVYYCARPHSLLRLRREFDYWGQGTTLTVSS

SEQ ID NO: 114 - protein, 122 aa, Mus musculus
EVQLQQSGPELVKPGASVKISCKTSGYTFAEYTMHWVKQSHGKSLEWIGVINPNNGGTTYNQQFKGKATLTVDKSSSTAYMELRSLTSEDSAVYYCTRPHSLLRLRREFDYWGQGTTLTVSS

SEQ ID NO: 115 - protein, 118 aa, Mus musculus
EVQLQQSGPELVKPGASVKISCKTSGYTFTEYPMHWVKQSHGKSLEWIGGINPNNGGTTYNQKFKGKATLTVDKSSSTAYMELRSLTSEDSAVYYCALYGNYWYFDVWGAGTTVTVSS

SEQ ID NO: 116 - protein, 122 aa, Mus musculus
EVQLQQSGPELVKPGASVKISCKTSGYTFTEYTIHWVKQSHGKSLEWIGVINPNSGGTTYNQKFKGKATLTVDHSSSTAYMELRSLTSEDSAVYYCARPHSLLRLRREFDYWGQGTTLTVSS

SEQ ID NO: 117 - protein, 122 aa, Mus musculus
EVQLQQSGPELVKPGASVKISCKTSGYTFTEYTIHWVKQSHGKSLEWIGVINPNSGGTTYNQKFKGKATLTVDQSSSTAYMELRSLTSEDSAVYYCARPHSLLRLRREFDYWGQGTTLTVSS

SEQ ID NO: 118 - protein, 122 aa, Mus musculus
EVQLQQSGPELVKPGASVKISCKTSGYTFTEYTMHLVKQSHGKSLEWIGVINPNNGGTTYNQKFKGKATLTVDKSSSTAYMELRSLTSEDSAVYYCARPHSLLRLRREFDYWGQGTTLTVSS

SEQ ID NO: 119 - protein, 122 aa, Mus musculus
EVQLQQSGPELVKPGASVKISCKTSGYTFTEYTMHWVKQSHGKSLEWIGAVNPNTGGTTYNQKFKDKATLTIDKSSSTAYMELRSLTSEDSAVYYCARPHSLLRLRREFDYWGQGTTLTVSS

SEQ ID NO: 120 - protein, 122 aa, Mus musculus
EVQLQQSGPELVKPGASVKISCKTSGYTFTEYTMHWVKQSHGKSLEWIGGINPNNGGTSYNQKFKGKATLTVDKSSSTAYMELRSLTSEDSAVYYCARPHSLLRLRREFDYWGQGTTLTVSS

SEQ ID NO: 121 - protein, 122 aa, Mus musculus
EVQLQQSGPELVKPGASVKISCKTSGYTFTEYTMHWVKQSHGKSLEWIGVINPNNGGTTYNQKFKGKATLTVDKSSSTAYMELRSLTSEDSAVYYCARPHSLLRLRREFDYWGQGTTLTVSS

SEQ ID NO: 122 - protein, 122 aa, Mus musculus
EVQLQQSGPELVKPGASVKISCKTSGYTFTEYTMHWVKQSHGKSLEWIGVINPNNGGTTYNQKFKGKATLTVDKSSSTAYMELRSLTSEDSAVYYCTRPHSLLRLRREFDYWGQGTTLTVSS

SEQ ID NO: 123 - protein, 122 aa, Mus musculus
EVQLQQSGPELVKPGASVKISCKTSGYTFTEYTMHWVKQSHGKSLEWIGVINPNSGGTTYNQKFKDKATLTVDKSSSTAYMELRSLTSEDSAVYYCTRPHSLLRLRREFDYWGQGTTLTVSS

SEQ ID NO: 124 - protein, 122 aa, Mus musculus
EVQLQQSGPELVKPGASVKISCKTSGYTFTEYTMHWVKQSHGKSLEWIGVINPNSGGTTYNQKFKGKATLTVDKSSSTAYMELRSLTSEDSAVYYCARPHSLLRLRREFDYWGQGTTLTVSS

SEQ ID NO: 125 - protein, 122 aa, Mus musculus
EVQLQQSGPELVKPGASVKISCKTSGYTFTEYTMHWVKQSHGKSLEWIGVINPNSGGTTYNQKFKGKATLTVDQSSSTAYMELLSLTSEDSAVYYCARPHSLLRLRREFDYWGQGTTLTVSS

SEQ ID NO: 126 - protein, 122 aa, Mus musculus
EVQLQQSGPELVKPGASVKISCKTSGYTFTEYTMHWVKQSHGKSLEWIGVINPNSGGTTYNQKFKGKATLTVDQSSSTAYMELRSLTSEDSAVYYCARPHSLLRLRREFDYWGQGTTLTVSS

SEQ ID NO: 127 - protein, 122 aa, Mus musculus
EVQLQQSGPELVKPGASVKISCKTSGYTFTEYTMHWVKQSHGKSLEWIGVINPNSGGTTYNQKFKGKATLTVDQSSSTAYMELRSLTYEDSAVYYCARPHSLLRLRREFDYWGQGTTLTVSS

SEQ ID NO: 128 - protein, 122 aa, Mus musculus
EVQLQQSGPELVKPGASVKISCKTSGYTFTEYTMHWVKQSHGKSLEWIGVINPNSGGTTYNQKFKGKATLTVDQSSTTAYMELRSLTSEDSAVYYCARPHSLLRLRREFDYWGQGTTLTVSS

SEQ ID NO: 129 - protein, 122 aa, Mus musculus
EVQLQQSGPELVKPGASVKISCKTSGYTFTEYTMHWVKQSHGKSLEWIGVINPNTGGTTYNQKFKGKATLTVDKSSTTAYMELRSLTSEDSAVYYCARPHSLLRLRREFDYWGQGTTLTVSS

SEQ ID NO: 130 - protein, 122 aa, Mus musculus
EVQLQQSGPELVKPGASVKISCKTSGYTFTEYTMHWVKQSHGKSLEWIGVINPNTGGTTYNQKFKGKATLTVDQSSTTAYMELRSLTSEDSAVYYCARPHSLLRLRREFDYWGQGTTLTVSS

SEQ ID NO: 131 - protein, 122 aa, Mus musculus
EVQLQQSGPELVKPGASVKISCKTSGYTFTEYTMHWVKQSQGKSLEWIGAVNLNTGGTTYNQKFKDKATLTIDKSSSTAYMELRSLTSEDSAVYYCARPHSLLRLRREFDYWGQGTTLTVSS

SEQ ID NO: 132 - protein, 118 aa, Mus musculus
EVQLQQSGPELVKPGASVKMSCKASGYTFTSYVIHWVKQKPGQGLEWIGYINPYHDGTKYNEKFKGKATLTSDKSSSTAYMELSSLTSEDSAVYYCARSRTVIGFDYWGQGTTLTVSS

SEQ ID NO: 133 - protein, 118 aa, Mus musculus
EVQLQQSGPELVKPGASVKMSCKASGYTFTSYVMHWVKQKPGQGLEWIGYINPYHDGTKYNEKFIGKATLTSDKSSSTAYMELSSLTSEDSAVYYCARSRTVIGFDYWGQGTTLTVSS

SEQ ID NO: 134 - protein, 118 aa, Mus musculus
EVQLQQSGPELVKPGASVKMSCKASGYTFTSYVMHWVKQKPGQGLEWIGYINPYHDGTKYNEKFKGKATLTSDKSSSTAYMELSSLTSEDSAVYYCARSRTVIGFDYWGQGTTLTVSS

SEQ ID NO: 135 - protein, 116 aa, Mus musculus
EVQLQQSGPELVKPGASVKMSCKASGYTFTSYVMHWVKQKPGQGLEWIGYINPYNDATKNNEKFKVKATLTSDKSSNTAYMELSSLTSEDSAVYYCARGVRLLDYWGQGTTLTVSS

SEQ ID NO: 136 - protein, 118 aa, Mus musculus
EVQLQQSGPELVKPGASVKMSCKASGYTFTSYVMHWVKQKPGQGLEWIGYINPYTDGTKYNEKFKGKATLTSDKSSSTAYMELSSLTSEDSAVYYCAKSRTVIGFDYWGQGTTLTVSS

SEQ ID NO: 137 - protein, 118 aa, Mus musculus
EVQLQQSGPELVKPGASVKMSCKASGYTFTSYVMHWVRQKPGQGLEWIGYINPYNDGTKYNEKFKGKATLTSDKSSSTANMELSSLTSEDSAVYYCARSRTVVGFDYWGQGTTLTVSS

SEQ ID NO: 138 - protein, 121 aa, Mus musculus
EVQLQQSGPELVKPGASVKMSCKASGYTFTTYVMHWVKQKPGQGLEWICYINPYNDGSNYNEKFKGKATLTSDKSSTTAYMELSSLTSEDSAVYYCARWVYYDFGGSMDYWGQGTSVTVSS

SEQ ID NO: 139 - protein, 121 aa, Mus musculus
EVQLQQSGPELVKPGASVKMSCKASGYTFTTYVMHWVKQKPGQGLEWIGYINPYNDGSNYNEKFKGKATLTSDKSSTTAYMELSSLTSEDSAVYYCARWVYYDFGGSMDYWGQGTSVTVSS

SEQ ID NO: 140 - protein, 122 aa, Mus musculus
EVQLQQSGPELVKPGTSVKISCKTSGYTFTEYTMHWVKQSHGKSLEWIGAVNPNTGGTTYNQKFKDKATLTIDKSSSTAYMQLRSLTSEDSAVYYCARPHSLLRLRREFDYWGQGTTLTVSS

SEQ ID NO: 141 - protein, 118 aa, Mus musculus
EVQLQQSGPELVKPGTSVKMSCKASGYTFTSYVMHWVKQKPGQGLEWIGYINPYHDGTQYNEKFKGKATLTSDKSSSTAYMELSSLTSEDSAVYYCARSRTVIGFDYWGQGTTLTVSS

SEQ ID NO: 142 - protein, 122 aa, Mus musculus
EVQLQQSGPELVKSGASVKISCKTSGYTFTEYTMHWVKQSHGKSLEWIGVINPNSGGTTYNQKFKGKATLTVDQSSSTAYMELRSLTSEESAVYYCTRPHSLLRLRREFDYWGQGTTLTVSS

SEQ ID NO: 143 - protein, 122 aa, Mus musculus
EVQLQQSGPELVTPGASVNISCKTSGYTFTEYTMHWVRQSHGKSLEWIGVINPNSGGTTYNQKFKDKATLTVDKSSTTAFMELRSLTSEDSAVYYCARPHSLLRLRREFDYWGQGTTLTVSS

SEQ ID NO: 144 - protein, 122 aa, Mus musculus
EVQLQQYGPELVKPGASVKISCKTSGYTFTEYTMHWVRQSHGKSLEWIGVINPNNGGTTYNQKFKGKATLTVDKSSTTAFMELRSLTSEDSAVYYCARPHSLLRLRREFDYWGQGTTLTVSS

SEQ ID NO: 145 - protein, 117 aa, Mus musculus
EVQLVESGGDLAKPGGSLKLSCAASGFTLSRYGMSWVRQTPDKRLEWVATSSGGGIYTYYPDSVKGRFTISRDNAKNTLYLQMSSLKSEDTAMYYCARHDGVPMDYWGQGTSVTVSS

SEQ ID NO: 146 - protein, 118 aa, Mus musculus
EVQLVESGGDLVKPGGSLKLSCAASGFTFSSYGMSWVRQTPDKRLEWVATIFSGDSNTYYLDSVKGRFIISRDNAKNTLYLQMSSLKSEDTAMYYCSRQGHRSWFAYWGQGTLVTVSA

SEQ ID NO: 147 - protein, 117 aa, Mus musculus
EVQLVESGGDLVKPGGSLKLSCAASGFTFSTYGMSWVRQTPDKRLEWVATISGGGIYTYYPDSVKGRFTISSDNAKNTLYLQMSSLKSEDTAMYYCVRMITTYFDHWGQGTTLTVSS

SEQ ID NO: 148 - protein, 118 aa, Mus musculus
EVQLVESGGDLVKPGGSLKVSCAASGFTFSSYGMSWVRQTPDKRLEWVATIFSGDSYTYYSDSVKGRFTISRDNAKNTLYLQMSSLKSEDTAMYYCARQGHRSWFAYWGQGTLVTVSA

SEQ ID NO: 149 - protein, 121 aa, Mus musculus
EVQLVESGGGLVKPGGSLKLSCAASGFTFSSYAMSWVRQTPEKRLEWVATISSGGSYTYYPDSVKGRFTISRDIAENTLFLQMSSLRSEDTAMYYCARHEDTTYYYAMDYWGQGTSVTVSS

SEQ ID NO: 150 - protein, 124 aa, Mus musculus
EVQLVESGGGLVLPKGSLKLSCAASGFTFNTYAMNWVRQAPGKGLEWVARIRIKSNNYATSYADSVKDRFTISRDDSQSMLYLQMNNLKTEDTAVYYCVRQAYGNSYFFAMDYWGQGTSVTVSS

SEQ ID NO: 151 - protein, 114 aa, Mus musculus
EVQLVETGGGLVQPKGSLKLSCAASGFTFNTNAMNWVRQAPGKGLEWVARIRSKSNNYATNYVDSVKDRFTISRDDSQSILYLQMNNLKTEDTAMYYCTAMDYWGQGTSVTVSS

SEQ ID NO: 152 - protein, 119 aa, Mus musculus
HVQLQQSGPELVRPGASVKLSCKASGYIFITYWMTWVKQRPGQGLEWIGQIFPASGSTTYNEMFEGKATLTVDTSSSTAYMQLSSLTSEDSAVYYCASYDGYYSWFAYWGQGTLVTVSA

SEQ ID NO: 153 - protein, 119 aa, Mus musculus
QGQMQQSGAELVKPGASVKLSCKTSGFTFSHSYISWLKQKPGQSLEWIAWIYAGTGGTTYNQKFTGKAQLTVDTSSSTAYMQFSSLTTEDSAIYYCARHETYGSSPDYWGQGTTLTVSS

SEQ ID NO: 154 - protein, 120 aa, Mus musculus
QGQMQQSGAELVKPGASVKLSCKTSGFTFSSFYISWLKQKPGQSLEWIAWIDAGTGGTSYNQKFTGKAQLTVDTFSSTAYMQFSSLTTEDSAIYYCARHVGRLRGYFDVWGAGTTVTVSS

SEQ ID NO: 155 - protein, 118 aa, Mus musculus
QGQMQQSGAELVKPGASVKLSCKTSGFTFSSNYISWLKQKPGQSLEWIAWIYAGTGSTGYNQKFTGKAQLTVDTFSSTAYMQFSSLTTEDSAIYYCASHDGHDAMDYWGQGTSVTVSS

SEQ ID NO: 156 - protein, 120 aa, Mus musculus
QGQMQQSGAELVKPGASVKLSCKTSGFTFSSNYISWLNQKPGQSLEWIAWIDAGTGGSSYNQKFTGKAQLTVDTSSSTAYMQFSSLTTEDSAIYYCARHVGRLRGYFDVWGAGTRVTVSS

SEQ ID NO: 157 - protein, 118 aa, Mus musculus
QGQMQQSGAELVKPGASVKLSCKTSGFTFSSRYISWLKQKPGQSLEWIAWIYAGTGGTGYNQKFTGKAQLTVDTSSSTAYMQFSSLTTEDSAMYYCASHDGHDAMDYWGQGTSVTVSS

SEQ ID NO: 158 - protein, 118 aa, Mus musculus
QGQMQQSGAELVKPGASVKLSCKTSGFTFSSRYISWLKQYPGQSLEWIAWIYAGTGGSGYNQKFTGKAQLTVDTSSSTAYMQFSSLTTEDSAMYYCASHDGHDAMDYWGQGTSVTVSS

SEQ ID NO: 159 - protein, 119 aa, Mus musculus
QGQMQQSGAELVKPGASVKLSCKTSGFTFSSSYISWLKQKPGQSLEWIAWIYAGTGDTSYNQKFTGKAQLTVDTSSSTAYMQFSSLTTEDSAIYYCASPTGINWYFDVWGAGTTVTVSS

SEQ ID NO: 160 - protein, 119 aa, Mus musculus
QGQMQQSGAELVKPGASVKLSCKTSGFTFSSSYISWLKQKPGQSLEWIAWIYAGTGDTSYNQKFTGKAQLTVDTSSSTAYMQFSSLTTEDSAIYYCASPTGVNWYFDVWGAGTTVTVSS

SEQ ID NO: 161 - protein, 119 aa, Mus musculus
QGQMQQSGAELVKPGASVKLSCKTSGFTFSSSYISWLKQKPGQSLEWIAWIYAGTGGTSYNQKFTGKAQLTVDTSSSTAYMQFSSLTTEDSAIYYCARPNGYGYGMDYWGQGTSVTVSS

SEQ ID NO: 162 - protein, 119 aa, Mus musculus
QGQMQQSGAELVKPGASVKLSCKTSGFTFSSSYISWLKQKPGQSLEWIAWIYAGTGGTSYNQKFTGKAQLTVDTSSSTAYMQFSSLTTEDSAIYYCARQDSYGRGPDCWGQGTTLTVSS

SEQ ID NO: 163 - protein, 119 aa, Mus musculus
QGQMQQSGAELVKPGASVKLSCKTSGFTFSSSYISWLKQKPGQSLEWIAWIYAGTGGTSYNQKFTGKAQLTVDTSSSTAYMQFSSLTTEDSAIYYCARQDSYGRSPDYWGQGTTLTVSS

SEQ ID NO: 164 - protein, 119 aa, Mus musculus
QGQMQQSGAELVKPGASVKLSCKTSGFTFSSSYISWLKQKPGQSLEWIAWIYAGTGGTTYNQKFTGKAQLTVDTSSSTAYMQFSSLTTEDSAIYYCARHETYGSGPDYWGQGTTLTVSS

SEQ ID NO: 165 - protein, 119 aa, Mus musculus
QGQMQQSGAELVKPGASVKLSCKTSGFTFSSSYISWLKQKPGQSLEWIAWIYAGTGGTTYNQKFTGKAQLTVDTSSSTAYMQFSSLTTEDSAIYYCARHETYGSSPDYWGQGTTLTVSS

SEQ ID NO: 166 - protein, 119 aa, Mus musculus
QGQMQQSGAELVKPGASVKLSCKTSGFTFSSSYISWLKQKPGQSLEWIAWIYAGTGGTTYNQKFTGKAQLTVDTSSSTAYMQFSSLTTEDSAIYYCARQDSYGRSPDYWGQGTTLTVSS

SEQ ID NO: 167 - protein, 119 aa, Mus musculus
QGQMQQSGAELVKPGASVKLSCKTSGFTFSSSYISWLKQKPGQSLEWIAWIYAGTGGTTYNQKFTGRAQLTVDTSSSTAYMQFSSLTTEDSAIYYCARQDSYGRSPDYWGQGTTLTVSS

SEQ ID NO: 168 - protein, 118 aa, Mus musculus
QGQMQQSGAELVKPGASVKLSCRTSGFTFSSRYISWLKQKPGQSLEWIAWIYAGTGGTSYNQKFTGKAQLTVDTSSSTAYMQFSSLTTEDSAIYYCASHDGLDAMDYWGQGTSVTVSS

SEQ ID NO: 169 - protein, 120 aa, Mus musculus
QGQMQQSGAELVKPGASVRLSCKTSGFTFSSFYISWLKQKPGQSLEWIAWIYAGPGYTSYNQKFTGKAQLTVDTSSSTAYMQFSSLTTEDSAIYYCARHVGRQRGYFDVWGAGTTVTVSS

SEQ ID NO: 170 - protein, 119 aa, Mus musculus
QGQMQQSGAELVKSGASVKLSCKTSGFTFSSSYISWLKQKPGQSLEWIAWIYAGTGGTTYNEKFTGKAQLTVDTSSSTAYMQFSSLTTEDSAIYYCGRHNGYGIGMDYWGQGTSVTVSS

SEQ ID NO: 171 - protein, 120 aa, Mus musculus
QGQMQQSGGELVKPGASVKLSCKTSGLTFSSSYISWLKQKPGQSLEWIAWIDAGTGGTSYNQKFTGKAQLTVDTSSSTAYMQFSSLTTEDSAIYYCARHVGRLRGYFDVWGAGTTVTVSS

SEQ ID NO: 172 - protein, 118 aa, Mus musculus
QGQMQQSGPELVKPGASVKLSCKTSGFTFSSNYISWLKQKPGQSLEWIAWIYAGTGGTGYNQKFTGKAQLTVDTSSSTAYMQFSSLTTEDSAIYYCASHDGHDAMDYWGQGTSVTVSS

SEQ ID NO: 173 - protein, 118 aa, Mus musculus
QIQLQQSGAELVRPGVSVKISCKGSGYTFTDYPIHWVKQSHTTSLEWIGVIITYYGDATYNQKFKGKATMTVDKSSSTAYMELARLTSDDSAIYYCASHRGKGYFDVWGAGTTVTVSS

SEQ ID NO: 174 - protein, 116 aa, Mus musculus
QIQLQQSGPELVKPGASVKISCKASGYTFTDYFINWVKQKPGQGLEWIGWIYPGNINTNYNENFKGKATLTVDTSSSTAYMQLSSLTSEDTAVYFCAGGNYALDYWGQGTSVTVSS

SEQ ID NO: 175 - protein, 116 aa, Mus musculus
QIQLQQSGPELVKPGASVKISCKASGYTFTDYYINWVKQKPGQGLEWIGCIYPGNDNSNYNEKFKGKATLTVDTSSSTAYMQLSSLTSEDTAVYFCAGGNYAMDYWGQGTSVTVSS

SEQ ID NO: 176 - protein, 116 aa, Mus musculus
QIQLQQSGPELVNPGASVMISCKASGYTFTAYYMNWVKQKPGQGLEWIGWIYPGNDNTNYNEKFKGKATLTVDTSSSTAFMQLSSLTSEDTAVYFCAGGNYAMDYWGLGTSVTVSS

SEQ ID NO: 177 - protein, 119 aa, Mus musculus
QIQLVQSGPELKKPGETVKISCKASGYTFTNSGMNWVKQAPGKGLKWMGWINTYTGEPTYVDDFKGRFAFSLETSASTAYLQINNLKNEDTATYFCAPTIQVRRLFDYWGQGTTLTVSS

SEQ ID NO: 178 - protein, 119 aa, Mus musculus
QIQLVQSGPELKKPGETVKISCKASGYTFTNSGMNWVKQAPGKGLKWMGWINTYTGEPTYVDDFKGRFAFSLETSASTAYLQINNLKNEDTATYFCAPTIQVRRLVDYWGQGTTLTVSS

SEQ ID NO: 179 - protein, 118 aa, Mus musculus
QIQLVQSGPELKKPGETVKISCKASGYTFTNYGMNWVKQAPGKGLKWMGWINTNTGEPTYAEEFKGRFAFSLETSASTAYLQINNLKNEDTATYFCARRGYYGRGDYWGQGTTLTVSS

SEQ ID NO: 180 - protein, 118 aa, Mus musculus
QVQLKESGPGLVAPSQSLSITCTVSGFSLTSYDISWIRQPPGKGLEWLGVIWTGGGTNYNSAFMSRLSISKDNSKSQVFLKMNSLQTDDTAIYYCVRWGDDYDWFAYWGQGTLVTVSA

SEQ ID NO: 181 - protein, 119 aa, Mus musculus
QVQLKQSGPSLVQPAQSLSITCTVSGFSLTTYGVHWLRQSPGKGLEWLGVIWRGGGTDYNAPFMSRLSITKDDSKSQLFFKMNSLHADDTAIYYCAIYSDHDTYALDYWGQGTSVTVSS

SEQ ID NO: 182 - protein, 118 aa, Mus musculus
QVQLQHSGAELVRPGVSVKISCKGSGYTFTDYAMHWVKQSHAKSLEWIGVISNYNGDVTYNQKFKGKATMTVDKSSSTAYMELARLTSEDSAIYYCARRHGNRAMDYWGQGTSVTVSS

SEQ ID NO: 183 - protein, 120 aa, Mus musculus
QVQLQQPGAELVKPGASVKLSCKASGYSFTSYYMYWVKQRPGHGLEWIGEINPSNGGTYFNVKFQSKATLTVDKSSTTAYMQLSSLTSEDSAVYFCTREGNYGYSWYAYWGQGTLVTVSA

SEQ ID NO: 184 - protein, 115 aa, Mus musculus
QVQLQQPGAELVKPGASVKLSCKASGYTFTSYWMHWVKQRPGQGLEWIGEIDPSDSYTNYNQKFKGKATLTVDKSSSTAYMQLSSLTSEDSAVYYCAGGIHFDYWGQGTTLTVSS

SEQ ID NO: 185 - protein, 120 aa, Mus musculus
QVQLQQPGAELVKPGASVKMSCKASGYTFTSYNLHWLKLTPGQGLEWIGIIYPGKDDTSYNQKFKGKATLTADTSSSTAYMQLSSLTSEDSAVYYCARVYYGNYGGFDYWGQGTLVTVSA

SEQ ID NO: 186 - protein, 120 aa, Mus musculus
QVQLQQPGSELVRPGASVKLSCKASGYTFTSYWMYWVKQRPGQGLEWIGNIYPGSGSVNYDEKFKSKATLTVDTSSSTAYMQLSSLTSEDSAVYYCTRGGIGVRSPFDVWGAGTTVTVSS

SEQ ID NO: 187 - protein, 115 aa, Mus musculus
QVQLQQPGSVLVRPGASVKLSCKASGYTFTSSWMHWAKQRPGQGLEWIGEIHPNSGNTNYNEKFKGKATLTIDTSSSTAYVDLSSLASEDSAVYYCVRLGLFDFWGQGTTLTVSS

SEQ ID NO: 188 - protein, 113 aa, Mus musculus
QVQLQQSGAELAKPGASVKMSCKASGYTFTNYWMHWIKQRPGQGLEWIGYISPRTGNTEYNQKFKDKATLTADKSSSIVYMQLSSLTSEDSAVYYCTRGEAYWGQGTLVTVSA

SEQ ID NO: 189 - protein, 113 aa, Mus musculus
QVQLQQSGAELAKPGASVKMSCKASGYTFTNYWMHWVKQRPGQGLEWIGYISPRTGNTEYNQKFKDKATLTADKSSSIVYIQLSSLTSEDSAVYYCTRGEAYWGQGTLVTVSA

SEQ ID NO: 190 - protein, 113 aa, Mus musculus
QVQLQQSGAELAKPGASVKMSCKASGYTFTNYWMHWVKQRPGQGLEWIGYISPRTGNTEYNQKFKDKATLTADKSSSIVYMQLSSLTSEDSALYYCTRGEAYWGQGTLVTVSA

SEQ ID NO: 191 - protein, 113 aa, Mus musculus
QVQLQQSGAELAKPGASVKMSCKASGYTFTNYWMHWVKQRPGQGLEWIGYISPRTGNTEYNQKFKDKATLTADKSSSIVYMQLSSLTSEDSAVYYCARGEAYWGQGTLVTVSA

SEQ ID NO: 192 - protein, 113 aa, Mus musculus
QVQLQQSGAELAKPGASVKMSCKASGYTFTNYWMHWVKQRPGQGLEWIGYISPRTGNTEYNQKFKDKATLTADKSSSIVYMQLSSLTSEDSAVYYCTRGEAYWGQGTLVTVSA

SEQ ID NO: 193 - protein, 113 aa, Mus musculus; identical to SEQ ID NO: 192

SEQ ID NO: 194 - protein, 113 aa, Mus musculus
QVQLQQSGAELAKPGASVKMSCKASGYTFTNYWMHWVKQRPGQGLEWIGYISPRTGNTEYNQKFKGKATLTADKSSSIVYMHLSSLTSEDSAVYYCARGEAYWGQGTLVTVSA

SEQ ID NO: 195 - protein, 113 aa, Mus musculus; identical to SEQ ID NO: 194

SEQ ID NO: 196 - protein, 113 aa, Mus musculus
QVQLQQSGAELAKPGASVKMSCKASGYTFTNYWMHWVKQRPGQGLEWIGYISPRTGNTEYNQKFRDKATLTADKSSSIVYMQLSSLTSEDSAVYYCTRGEAYWGQGTLVTVSA

SEQ ID NO: 197 - protein, 113 aa, Mus musculus
QVQLQQSGAELAKPGASVKMSCKASGYTFTNYWMHWVKQRPGQGLEWIGYISPRTGNTNYNQRFKDKATLTADKSSSIVYMQLSSLTSEDSAVYYCARGEAYWGQGTLVTVSA

SEQ ID NO: 198 - protein, 113 aa, Mus musculus
QVQLQQSGAELAKPGASVKMSCKASGYTFTNYWMHWVKQRPGQGLEWIGYISPRTTYTEYNQKFKDKATLTADKSSSTAFLQLSSLTSEDSAVYYCTRGEAYWGQGTLVTVSA

SEQ ID NO: 199 - protein, 120 aa, Mus musculus
QVQLQQSGAELAKPGASVKMSCKASGYTFTTYWMHWVKQRPGQGLEWIGYINPSTGYTYYNQKFKDKATLTADKSSSTAYMQLSSLTSEDSAVYFCASYYGTSQAWFAYWGQGTLVTVSA

SEQ ID NO: 200 - protein, 120 aa, Mus musculus
QVQLQQSGAELARPGASVKLSCKASGYTFTSYWMQWIKQRPGQGLEWIGAIYPGDGDTRYTQKFKGKATLTADKSSSTAYMQLSSLASEDSAVYYCARVEADDLWYFDVWGAGTTVTVSS

SEQ ID NO: 201 - protein, 120 aa, Mus musculus
QVQLQQSGAELSKPGASVKMSCKASGYTFTTYWMHWVKQRPGQGLEWIGYINPSTGYTYYNQKFKDKATLTADKSSSTAYMQLSSLTSEDSAVYFCASYYGTSQAWFAYWGQGTLVTVSA

SEQ ID NO: 202 - protein, 120 aa, Mus musculus
QVQLQQSGAELVRPGTSVKVSCKASGYAFTNYLIEWVKQRPGQGLEWIGVINPGSGGPNYNEKFKGKATLTADKSSSTAYMQLSSLTSDDSAVYFCASDNYGNYDVLDYWGQGTSVTVSS

SEQ ID NO: 203 - protein, 113 aa, Mus musculus
QVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCDREDAYWGQGTLVTVSA

SEQ ID NO: 204 - protein, 121 aa, Mus musculus
QVQLQQSGPDLVRPGVSVKISCKGSGYTFTDYALHWVKQSHAKSLEWIGVINTYSSNTYYNQNFEGKATMTVDKSSNTAYMELARLTSEDSAIYYCARRRGSHRDAYFDYWGQGTTLTVSS

SEQ ID NO: 205 - protein, 121 aa, Mus musculus
QVQLQQSGPDLVRPGVSVKISCKGSGYTFTDYALHWVKQSHAKSLEWIGVINTYSSNTYYNQNFEGKATMTVDKSSSTAYMELARLTSEDSAIYYCARRRGSHRDAYFDYWGQGTTLTVSS

SEQ ID NO: 206 - protein, 117 aa, Mus musculus
QVQLQQSGPELVKPGASVKMSCKASGYTFTDYVMSWVKQRTGQGLEWIGEIYPGGNRTYYSEKFKGKATLTADKSSNTAYMQVSSLTAEDSAVYFCARFNDWAMDHWGQGTSVTVSS

SEQ ID NO: 207 - protein, 117 aa, Mus musculus
QVQLQQSGPELVKPGASVKMSCKASGYTFTDYVMSWVKQRTGQGLEWIGEIYPGGNSTYYNEKFKGKATLTADKSSNTAYMQLSSLTSEDSAVYFCARFNDWSMDYWGQGTSVTVSS

SEQ ID NO: 208 - protein, 117 aa, Mus musculus
QVQLQQSGPELVKPGASVKMSCKASGYTFTDYVMSWVTQRTGQGLEWIGEIYPGGSSIHYNEKFKDKATLTADKSSNTAYMQLSSLTSEDSAVYFCARFNDWAMDYWGQGTSVTVSS

SEQ ID NO: 209 - protein, 117 aa, Mus musculus
QVQLQQSGPELVKPGASVKMSCKASGYTFTDYVMSWVTQRTGQGLEWIGEIYPGGSSIYYNEKFKDKATLTADKSSNTAYMQLSSLTSEDSAVYFCARFNDWAMDYWGQGTSVTVSS

SEQ ID NO: 210 - protein, 115 aa, Mus musculus
QVQLQQSGPELVKPGASVRISCKASGYTFTSYFIHWVKQRPGQGLEWIGWIYPGNVNTNYNERFKGKATLTADKSSSTAYMQLSSLTSEDSAVYFCARDYAMDYWGQGTSVTVSS

SEQ ID NO: 211 - protein, 115 aa, Mus musculus
QVQLQQSGPELVKPGASVRISCKASGYTFTTYFIHWVKQRPGQGLEWIGWIYPGNVNVNYNEKFKGKATLTADKSSSTAYMHLSSLTSEDSAVYFCSRDYAMDYWGQGTSVTVSS

SEQ ID NO: 212 - protein, 115 aa, Mus musculus
QVQLQQSGPELVKPGASVRISCKASGYTFTTYYIHWVKQRPGQGLEWIGWIYPGNVNTNYNEKFKGKATLTADKSSSTAYMQLSSLTSEDSAVYFCTRDYAMDYWGQGTSVTVSS

SEQ ID NO: 213 - protein, 115 aa, Mus musculus
QVQLQQSGPELVKPGASVRISCKASGYTFTTYYIHWVKQRPGQGLEWIGWIYPGNVNVNYNEKFKGKATLTADKSSSTAYMQLSSLTSEDSAVYFCTRDYAMDYWGQGTSVTVSS

SEQ ID NO: 214 - protein, 115 aa, Mus musculus
QVQLQQSGPELVKPGASVRISCKASGYTFTTYYIHWVKQRPGQGLEWIGWIYPGNVNVNYNERFKGKATLTADKSSSTAYMQLSSLTSEDSAVYFCTRDYAMDYWGQGTSVTVSS

SEQ ID NO: 215 - protein, 115 aa, Mus musculus
QVQLQQSGPELVKPGASVRISCTASGYTFTTYYIHWVKQRPGQGLEWIGWIYPGNVNTNYNEKFKGKATLTADKSSSTAYMQLSSLTSEDSAVYFCARDYAMDYWGQGTSVTVSS

SEQ ID NO: 216 - protein, 115 aa, Mus musculus
QVQLQQSGPELVKPWASVRISCKASGYTFTTYYIHWVKQRPGQGLEWIGWIYPGNVNVNYNERFKGKATLTADKPSSTAYMQLSSLTSEDSAVYFCSRDYAMDYWGQGTSVTVSS

SEQ ID NO: 217 - protein, 121 aa, Mus musculus
QVQLQQSGPELVRPGVSVKISCKGFGYTFTDYAMHWVKQSHAKSLEWIGVINTYSGNTYYNQKFKDKATMTVDKSSSTAYMELARLTSEDSAIYYCARRRGSHRDAYFDYWGQGTPLTVSS

SEQ ID NO: 218 - protein, 120 aa, Mus musculus
QVQLQQSGPELVRPGVSVKISCKGSGYTFTDYAIHWVKQSHAKSLEWIGVINTHSDNTNYFQRFKGKATMTVDKSSSTAYMELARLTSEDSAIYYCARPNYDGHWYFDVWGAGTTVTVSS

SEQ ID NO: 219 - protein, 121 aa, Mus musculus
QVQLQQSGPELVRPGVSVKISCKGSGYTFTDYAIHWVKQSHAKSLEWIGVINTYSGNTNYNQNFKVKATMTVDKSSNTAYMELARLTSEDSAIYYCARRRGIHRDAYFDYWGQGTTLTVSS

SEQ ID NO: 220 - protein, 122 aa, Mus musculus
QVQLQQSGPELVRPGVSVKISCKGSGYTFTDYAMHWVKQSHAKSLEWIGIINSYSGNTNYNQKFKGKATMTVDKSSSTAYMELARLTSEDSAIYYCARWAITTGVYYAMDYWGQGTSVTVSS

SEQ ID NO: 221 - protein, 121 aa, Mus musculus
QVQLQQSGPELVRPGVSVKISCKGSGYTFTDYAMHWVKQSHAKSLEWIGVINTYSANTNYNQKFKGKATMTVDKSSSTAYLELARLTSEDSAIYYCARRRGSHRDAYFDYWGQGTTLTVSS

SEQ ID NO: 222 - protein, 121 aa, Mus musculus
QVQLQQSGPELVRPGVSVKISCKGSGYTFTDYAMHWVKQSHAKSLEWIGVINTYSGNTNYNQNFKVKATMTVDKSSSTAYMELARLTSEDSAIYYCARRRGSHRDAYFDYWGQGTTLTVSS

SEQ ID NO: 223 - protein, 121 aa, Mus musculus
QVQLQQSGPELVRPGVSVKISCKGSGYTFTDYAMHWVKQSHVKSLEWIGVINTYSGNTNYNQKFKGKATMTVDKSSSTAYMELARLTSEDSAIYYCARRRGSHQDAYFDYWGQGTTLTVSS

SEQ ID NO: 224 - protein, 121 aa, Mus musculus
QVQLQQSGPELVRPGVSVKISCKGSGYTFTDYAVHWMKQSHAKSLEWIGVINTYSGNTYYNLHFKDKATMTVDKSSSTAYMELATLTSEDSAIYYCARRRGSHRDAYFDYWGQGTTLTVSS

SEQ ID NO: 225 - protein, 121 aa, Mus musculus
QVQLQQSGPELVRPGVSVKISCKGSGYTFTDYAVHWVKQSHAKSLEWIGVINTYSGNTYYNLHFKGKATMTVDKSSSTAYMELARLTSEDSAIYYCARRRGSHRDAYFDYWGQGTTLTVSS

SEQ ID NO: 226 - protein, 121 aa, Mus musculus
QVQLQQSGPELVRPGVSVKISCKGSGYTFTDYAVHWVKQSHAKSLEWIGVINTYSGNTYYNLHFKGKATMTVDKSSSTAYMELATLTSEDSAIYYCARRRGSHRDAYFDYWGQGTTLTVSS

SEQ ID NO: 227 - protein, 121 aa, Mus musculus
QVQLQQSGPELVRPGVSVKISCKGSGYTFTDYAVHWVKQSHAKSLEWIGVINTYSGNTYYNLNFTGKATMTVDKSSSTAYMELARLTSEDSAIYYCARRRGSHRDAYFDYWGQGTTLTVSS

SEQ ID NO: 228 - protein, 121 aa, Mus musculus
QVQLQQSGPELVSPGVSVKISCKGSGYTFTDYAVHWVKQSHAKSLEWIGVINTYSGNTYFNLHFKGKATMTVDKSSSTAYMELASLTSEDSAIYYCARRRGSHRDAYFDYWGQGTTLTVSS

SEQ ID NO: 229 - protein, 117 aa, Mus musculus
QVQLQQSGPEVVKPGASVKMSCKASGYTFTDYVMTWVKQRTGQGLEWIGEIYPGGNRTYYKEKFKGKATLTADKSSNTAYMQLSSLTSEDSAVYFGARFNDWAMDYWGQGTSVTVSS

SEQ ID NO: 230 - protein, 118 aa, Mus musculus
QVQLQQSGTELVRPGASVTLSCKTSGYTFTDYEMHWVKQTPVHGLEWIGGIDPETGATAYNQKFKGKATLTADKSSSTAYMELRSLTSEDSAVYYCTARQLGLPFAYWGQGTLVTVSA

SEQ ID NO: 231 - protein, 122 aa, Mus musculus
QVTLKESGPGILQPSQTLSLTCSFSGFSLNTSNLGVGWIRQPSGKGLEWLAHIWWDDDKRYNPALKSRLTISKDSSSNQVFLKIASVDTADTATYYCARKNHYYGHWYFDVWGAGTTVTVSS

SEQ ID NO: 232 - protein, 122 aa, Mus musculus
QVTLKESGPGILQPSQTLSLTCSFSGFSLNTSNMGVGWIRQPSGKGLEWLAHIWWDDDKRYNPALKSRLTISKDSSSNQVFLKIASVDTADTATYYCARKNHYYGHWYFDVWGAGTTVTVSS

SEQ ID NO: 233 - protein, 122 aa, Mus musculus
QVTLKESGPGILQPSQTLSLTCSFSGFSLSTPGLGVGWIRQPSGKGLEWLAHIWWDDDKRYNPALKSRLTISKDTSSNQVFLKIASVDTADTATYYCARKNHYYGHWYFDVWGAGTTVTVSS

SEQ ID NO: 234 - protein, 122 aa, Mus musculus
QVTLKESGPGILQPSQTLSLTCSFSGFSLSTPNMGVGWIRQPSGKGLEWLAHIWWDDDKRQNAALKSRLTISKETSSNQVFLKIASVDTADTATYYCARKIHYYGHWYFDVWGAGTTVTVSS

SEQ ID NO: 235 - protein, 122 aa, Mus musculus
QVTLKESGPGILQPSQTLSLTCSFSGFSLSTPSMGVGWIRQPSGKGLEWLAHIWWDDDKRSDPALKSRLTISKDTSSNQVFLKIASVDTADTGTYYCARKNHYSGHWFFDVWGAGTTVTVSS

SEQ ID NO: 236 - protein, 122 aa, Mus musculus
QVTLKESGPGILQPSQTLSLTCSFSGFSLSTSGMGVGWIRQPSGKGLEWLAHIWWDDDKRYNPALKSRLTISKDSSSNQVFLKIASVDTADTATYYCARKNHYYGHWYFDVWGAGTTVTVSS

SEQ ID NO: 237 - protein, 122 aa, Mus musculus
QVTLKESGPGILQPSQTLSLTCSFSGFSLSTSNLGVGWIRQPSGKGLEWLAHIWWDDDKRYNPDLESRLTISKDTSSNQVFLKIASVDTADTATYYCARKSHYYGHWYFDVWGAGTTVTVSS

SEQ ID NO: 238 - protein, 122 aa, Mus musculus
QVTLKESGPGILQPSQTLSLTCSFSGFSLSTSNMGVGWIRQPSGKGLEWLAHIWWDDDKRYHPALKSRLTISKDTSSNQVFLKIASVDTADTATYYCARKSHYYGHWYFDFWGAGTTVTVSS

SEQ ID NO: 239 - protein, 122 aa, Mus musculus
QVTLKESGPGILQPSQTLSLTCSFSGFSLSTSNMGVGWIRQPSGKGLEWLAHIWWDDDKRYNPALKSRLTISKDSSNNQVFLKIASVDTSDTAIYYCGRKSHYYGHWYFDVWGAGTTVTVSS

SEQ ID NO: 240 - protein, 122 aa, Mus musculus
QVTLKESGPGILQPSQTLSLTCSFSGFSLSTSNMGVGWIRQPSGKGLEWLAHIWWDDDKRYNPALKSRLTISKDTSSNQVFLKIASVDTADSATYYCARKNHYYGHWYFDFWGAGTTVTVSS

SEQ ID NO: 241 - protein, 122 aa, Mus musculus
QVTLKESGPGILQPSQTLSLTCSFSGFSLSTSNMGVGWIRQPSGKGLEWLAHIWWDDDKRYNPALKSRLTISKDTSSNQVFLKIASVDTADTATYYCARKNHYYGHWYFDFWGAGTTVTVSS

SEQ ID NO: 242 - protein, 122 aa, Mus musculus
QVTLKESGPGILQPSQTLSLTCSFSGFSLSTSNMGVGWIRQPSGKGLEWLAHIWWDDDKRYNPDLKSRLTISKDTSSNQVFLKIASVDTADTATYYCARKNHYYGHWYFDVWGAGTTVTVSS

SEQ ID NO: 243 - protein, 122 aa, Mus musculus
QVTLKESGPGILQPSQTLSLTCSFSGFSLSTSNMGVGWIRQPSGKGLEWLAHIWWDDDKRYNPDLKSRLTISKDTSSNQVFLKIASVDTADTATYYCARKSHYYGHWYFDVWGAGTTVTVSS

SEQ ID NO: 244 - protein, 122 aa, Mus musculus
QVTLKESGPGILQPSQTLSLTCSFSGFSLSTSNMGVGWIRQPSGKGLEWLAHIWWDDDKRYNPDLKSRLTISKDTSSNQVFLKIASVDTADTATYYCGRKSHYYGHWYFDVWGAGTTVTVSS

SEQ ID NO: 245 - protein, 122 aa, Mus musculus
QVTLKESGPGILQPSQTLSLTCSFSGFSLSTSNMGVGWIRQPSGKGLEWLAHIWWDDDTRYKPALKSRLTISKDTSSNQVFLKIASVDTADTATYFCARKNHYYGHWYFDVWGAGTTVTVSS

SEQ ID NO: 246 - protein, 117 aa, Mus musculus
QVTLKESGPGILQPSQTLSLTCSFSGFSLSTYGIGVGWIRQPSGKGLEWLAHIWWNDNKYYNTALKSRLTISKDTSNNQVFLKIASVDTADTATYYCARIDYWFAYWGQGTLVTVSA

SEQ ID NO: 247 - protein, 107 aa, Mus musculus
DIKMTQSPSSMYASLGERVTITCKASQDINSYLSWFQQKTGKSPKTLIYRANRLVDGVPSRFSGSGSGQDYSLTISSLEYEDMGIYYCLQYDEFPPTFGGGTKLEIK

SEQ ID NO: 248 - protein, 107 aa, Mus musculus
DIKMTQSPSSMYASLGERVTITCKASQDINTYLSWFQQKPGKSPKTLIYRANILVDGVPSRFSGSGSGQDYSLTISSLEYEDMGIYYCLQYDEFPPTFGGGTKLEIK

SEQ ID NO: 249 - protein, 111 aa, Mus musculus
DIVLTQSPASLAVSLGQRVTISCKASQSVDYDGDSYMNWYQQKPGQPPKVLIYAASNLESGIPARFSGSGSGTDFTLNIHPVEEEDAATYYCQQSNEDPRTFGGGTKLEIK

SEQ ID NO: 250 - protein, 107 aa, Mus musculus
DIQMTQSTSSLSASLGDRVTISCRASQDISNYLNWYQQKPDGTVKLLIYYTSRLHSGVPSRFSGSGSGTDYSLTISSLEQEDIATYFCQQGNALPWTFGGGTKLEIK

SEQ ID NO: 251 - protein, 107 aa, Mus musculus
DIQMTQTTSSLSASLGDRVTISCRASQDISNYLNWYQQKPDGTVKLLIYYTSRLHSGVPSRFSGSGSGTAYSLTISNLEEEDIATYFCQQGKSLPWTFGGGTKLEIK

SEQ ID NO: 252 - protein, 107 aa, Mus musculus
DIQMTQPTSSLSASLGDRVTISCRASQDISNYLNWYQQKPDGTVKLLIYYTSRLHSGVPSRFSGSGSGTDYSLTISNLDQEDIATYFCQQGNTLPWTFGGGTKLEIK

SEQ ID NO: 253 - protein, 107 aa, Mus musculus
DIQMTQPTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIK

SEQ ID NO: 254 - protein, 107 aa, Mus musculus
DIQMTQTTSSLSASLGDRVTISCRASQDINNYLNWYQQKPDGIVKLLIYYTSRLNSGVPSRFSGSGSGTDYSLTISNLEQEDIATYFCQQGNTLPWTFGGGTKLEIK

SEQ ID NO: 255 - protein, 107 aa, Mus musculus
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIK

SEQ ID NO: 256 - protein, 107 aa, Mus musculus
DIQMTQTTSSLSASLGDRVTISCRASQDISNYLNWYQQKPDGTVKLLIYYTSRLHSGVPSRFSGSGSGTAYSLTISNLEEADIATYFCQQGKSLPWTFGGGTKLEIK

SEQ ID NO: 257 - protein, 107 aa, Mus musculus
DIQMTQTTSSLSASLGDRVTISCRASQDISNYLNWYQQKPDGIVKLLIYYTSRLNSGVPSRFSGSGSGTDYSLTISNLEQEDIATYFCQQGNTLPWTFGGGTKLEIK

SEQ ID NO: 258 - protein, 107 aa, Mus musculus
DIQMTQTTSSLSASLGDRVTISCRASQDISNFLNWFQQKPDGTVKLLIYYTSRLHSGVPSRFSGSGSGTDYSLTISNLEQEDIASYFCQQGKTLPWTFGGGTKLEIK

SEQ ID NO: 259 - protein, 107 aa, Mus musculus
DIQMTQTTSSLSASLGDRVTISCRASQDISNYLNWYQQKPDGTVKLLIYYTSRLHSGVPSRFSGSGSGTDYSLTISNLEQEDLATYFCQQGNTLPWTFGGGTKLEIK

SEQ ID NO: 260 - protein, 107 aa, Mus musculus
DIQMTQPTSSLSASLGDRVTISCRASQDINNYLNWYQQKPDGTVKLLIYYTSRLHSGVPSRFSGSGSGTDYSLTISNLEQEDIATYFCQQGNTLPWTFGGGTKLEIK

SEQ ID NO: 261 - protein, 107 aa, Mus musculus
DIQMTQTTSSLSASLGDRVTISCRASHDIHNYLNWYQQKPDGTVKLLIYYTSRLHSGVPSRFSGSGSGTDYSLTISNLEQEDIATYFCQQGNTLPWTFGGGTKLEIK

SEQ ID NO: 262 - protein, 108 aa, Mus musculus
EIVLTQSPTTMAASPGEKITITCSASSSISSNYLHWYQQKPGFSPKLLIYRTSNLASGVPARFSGSGSGTSYSLTIGTMEAEDVATYYCQQGSSIPRTFGSGTKLEIK

SEQ ID NO: 263 - protein, 107 aa, Mus musculus
DIQMTQSTSSLSASLGDRVTISCRASQDISNYLNWYQQKPDGTVKLLIYYTSRLHSGVPSRFSGSGSGTDYSLTISSLEQEDIATYFCQQGIALPWTFGGGTKLEIK

SEQ ID NO: 264 - protein, 111 aa, Mus musculus
DIVLTQSPASLAVSLGQRATISCRASESVDSYGNSFMHWYQQKPGQPPKLLIYIASNLESGVPARFSGSGSRTDFTLTIDPVEADDAATYYCQQSNEDPRTFGGGTKLEIK

SEQ ID NO: 265 - protein, 107 aa, Mus musculus
SIVMTQTPKFLLVSAGDRVTITCRASQSVTNDVAWYQQKPGQSPKLLIYYASNRYTGVPDRFTGSGYGTDFTFTISTVQAEDLAVYFCQQDYNSPYTFGGGTKLEIK

SEQ ID NO: 266 - protein, 107 aa, Mus musculus
DIQMTQTTSSLSASLGDRVTISCRASQDISNYLNWYQQKPDGIVKLLIYYTSRLNSGVPSRFSGSGSGTDYSLTISSLEQEDIATYFCQQGNTLPWTFGGGTKLESK

SEQ ID NO: 267 - protein, 107 aa, Mus musculus
DIQMTQSTSSLSASLGDRVSISCRASQDISNYLNWYQQKPDGTVKLLIYYTSRLHSGVPSRFSGSGSGTDYSLTISSLEQEDVATYFCQQGNALPWTFGGGTKLEIK

SEQ ID NO: 268 - protein, 107 aa, Mus musculus
DIQMTQTTSSLSASLGDRVTISCRTSQDISNYLNWYQQKPDGVVKLLIYYTSRLNSGVPSRFSGSGSGTDYSLTISNLEQEDIATYFCQQGNTLPWTFGGGTKLEIK

SEQ ID NO: 269 - protein, 107 aa, Mus musculus
DIVMTQSHKFMSTSVGDRVSITCKASQDVGTAVAWYQQKPGQSPKLLIYWASTRHTGVPDRFTGSGSGTDFTLTISNVQSEDLADYFCQQYSSYPYTFGGGTKLEIK

SEQ ID NO: 270 - protein, 107 aa, Mus musculus
DIQMTQTTSSLSASLGDRVTISCRASQDISNYLNWYQQKPDGTVKLLIYYTSRLHSGVPSRFSGSGSGTDYSLTISNLEQEDIATYFCQQGNALPWTFGGGTKLEIK

SEQ ID NO: 271 - protein, 107 aa, Mus musculus
DIVMTQSHKFMSTSVGDRVSITCKASQDVNTAVAWYQQRPGQSPKLLIYSASYRFIGVPDRFTGSGSGTDFTFTISTVQAEDLAVYYCQQHYSTPFTFGSGTKLEIK

SEQ ID NO: 272 - protein, 112 aa, Mus musculus
DVLMTQTPLSLPVSLGDHASISCRSSQSIVHTNGNTYLEWYLQKPGQSPKLLIYKVSNRFSGVPDRFSGSGSGTDFTLKISRVEAEDLGLYYCFQGSHVPYTFGGGTKLEIK

SEQ ID NO: 273 - protein, 112 aa, Mus musculus
DVVMTQTPLSLPVSLGDQASISCRSSQSLVHSNGNTYLHWYLQKPGQSPNLLIYKVSNRFSGVPDRFSGSGSGTDFTLKISRVEAEDLGVYFCSQSTHGPWTFGGGTKLEIK

SEQ ID NO: 274 - protein, 112 aa, Mus musculus
DVVMTQTPLSLPVSLGDQASISCRSSQSLVHSYGNTYLHWHLQKPGQSPKLLIYKVSNRFSGVPDRFSGSGSGTDFTLKISRVEAEDLGVYFCSQSTHGPWTFGGGTKLEIK

SEQ ID NO: 275 - protein, 112 aa, Mus musculus
DVVMTQTPLSLPVSLGDQASISCRSSQSLVHSNGNTYLHWHLQKPGQSPKLLIYKVSNRFSGVPDRFSGSGSGTDFTLKISRVEAEDLGVYFCSQSTHGPWTFGGGTKLEIK

SEQ ID NO: 276 - protein, 108 aa, Mus musculus
EIVLTQSPTPMAASPGEKITITCSASSSISSNYLHWYRQKPGFSPKLLIYRTSNLASGVPPRFSGSGSGTSYSLTIGTMEAEDVATYYCQQGSSLPRTFGSGTKLEIK

SEQ ID NO: 277 - protein, 106 aa, Mus musculus
QIVLTQSPAIMSASPGEKVTITCSASSSVNYMHWFQQKPGTSPKLWIYSTSNLASGVPARFSGSGSGTSYSLTISRMEAEDAATYYCQQRSTYPRTFGGGTKLEIK

SEQ ID NO: 278 - protein, 107 aa, Mus musculus
DILMTQSPSSMSVSLGDTVSITCHASQGISSNIGWLQRKPGKSFKGLIYHGTNLEDGLPSRFSGSGSGADYSLTISSLESEDFADYYCVQYAQFPYTFGGGTKLELK

SEQ ID NO: 279 - protein, 107 aa, Mus musculus
DILMTQSPSSMSVSLGDTVSITCHASQGISSNIGWLQQKPGKSFKGLIYHGTNLEDGLPSRFSGSGSGADYSLTISSLESEDFADYYCVQYAQFPYTFGGGTKLEIK

SEQ ID NO: 280 - protein, 107 aa, Mus musculus
DILMTQSPSSMSVSLGDTVSITCHASQGISSNIGWLQQKPGKSFKGLIYHGTNLEDGLPSRFSGSGSGADYSLTISSLESEDFADYYCVQYAQFPYTFGGGTKLEIL

SEQ ID NO: 281 - protein, 107 aa, Mus musculus
DILMTQSPSSMSVSLGDSVSITCHASQGISSNIGWLQQKAGKSFKGLIYHGTNLEDGVPSRFSGSGSGADYSLTISSLESEDFADYYCVQYAQFPYTFGGGTKVEIK

SEQ ID NO: 282 - protein, 107 aa, Mus musculus
DILMTQSPSSMSVSLGDTVTITCHASQGISSNIGWLQQKPGKSFKGLIYHGTNLEDGVPSKFGGSGSGADYSLTISSLESEDFADYYCVQYAQFPYTFGGGTKLEIK

SEQ ID NO: 283 - protein, 107 aa, Mus musculus
DILMTQSPSSMSVSLGDSVSITCHASQGISSNIGWLQQKAGKSFKGLIYHGTNLEDGVPSRFSGSGSGADYSLTISSLESEDFADYYCVQYAQFPYTFGGGTTLEIK

SEQ ID NO: 284 - protein, 107 aa, Mus musculus
DILMTQSPSSMSVSLGDSVSITCHASQGISSNMGWLQQKAGKSFKGLIYHGTNLEDGVPSRFSGSGSGADYSLTISSLESEDFADYYCVQYAQFPYTFGGGTKLEIK

SEQ ID NO: 285 - protein, 107 aa, Mus musculus
DILMTQSPSSMSVSLGDSVSITCHASQGISSNMGWLQQKTGKSFKGLIYHGTNLEDGVPSRFSGSGSGADYSLTISSLESEDFADYYCVQYAQFPYTFGGGTKLEIK

SEQ ID NO: 286 - protein, 110 aa, Mus musculus
DIVLTQSPASLTVSLGQRATISCRASKSVSTSAYSYMHWYQQKPGQPPKLLIYLASNLESGVPARFSGSGSGTDFTLNIHPVEEEDAATYYCQHSRELLTFGSGTKLEIK

SEQ ID NO: 287 - protein, 110 aa, Mus musculus
DIVLTQSPPSLAVSLGQRATISCRASKSVSTSAYSYMHWYQQKPGQPPKLLIYLASNLESGVPARFSGSGSGTDFTLNIHPVEEEDAATYYCQHSRELLTFGSGTKLEIK

SEQ ID NO: 288 - protein, 110 aa, Mus musculus
DIVLTQSPASLTVSLGQRATISCRASKSVSTSAYSYMHWYQQKLGQPPKLLIYLASNLESGVPARFSGSGSGTDFTLNIHPVEEEDAATYYCQHSRELLTFGSGTKLEIK

SEQ ID NO: 289 - protein, 110 aa, Mus musculus
DIVLTQSPASLAVSLGQRATISCRASKSVSTSAYSYMHWYQQKPGQPPKLLIYLASNLESGVPARFSGSGSGTDFTLNIHPVEEEDAATYYCQHSRELLTFGSGTKLEIK

SEQ ID NO: 290 - protein, 110 aa, Mus musculus
DIVLTQSPASLAVSLGQRATISCRASKSVSTSNYSYMHWYQQKPGQPPKLLLYLASNLESGVPARFSGSGSGTDFTLNIHPVEEEDAATYYCQHSRELPTFGGGTKLEIK

SEQ ID NO: 291 - protein, 110 aa, Mus musculus
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELLTFGSGTKLEIK

SEQ ID NO: 292 - protein, 111 aa, Mus musculus
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPYTFGGGTKLEIK

SEQ ID NO: 293 - protein, 107 aa, Mus musculus
DIVMTQFQKFMSTSVGDRVSVTCKASQNVDTNVAWYQQKPGQSPKLLIYSGSYRFSGVPDRFTGSGSGTDFTLTISNVQSEDLAEYFCQQYNTYPYTFGGGTKLEIK

SEQ ID NO: 294 - protein, 107 aa, Mus musculus
DIVMTQSQKFMSTSVGDRVSVTCKASQNVDTNVAWYQQKPGQSPKLLIYSASYRFSGVPDRFTGSGSGTDFTLTISNVQSEDLAEYFCQQYNSYPYTFGGGTKLEIK

SEQ ID NO: 295 - protein, 111 aa, Mus musculus
DIVLTQSPVSLVVSLGQRATISCRANETVDSYGSSFMHWYQQKPGHPPKLLIYRASNLESGIPARFSGSGSRTDFTLTINPVEADDVATYFCQQSNEDPFTFGSGTKLEIK

SEQ ID NO: 296 - protein, 107 aa, Mus musculus
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPLTFGAGTKLELK

SEQ ID NO: 297 - protein, 111 aa, Mus musculus
DIVLTQSPASLAVSLGQRATISCRASESVDSYGNSFMHWYQQKPGQPPKLLVYIASNLESGVPARFSGSGSRTDFTLTIDPVEADDAAAYYCQQSNEDPRTFGGGTKLEIK

SEQ ID NO: 298 - protein, 106 aa, Mus musculus
DIQMTQSSSYLSVSLGGRVTITCKASDHINNWLAWYQQKPGNAPRLLISGATSLETGVPSRFSGSGSGKDYTLSITSLQTEDVATYYCQQYWSTPTFGGGTKLEIK

SEQ ID NO: 299 - protein, 111 aa, Mus musculus
DIVLTQSPASLAVSLGQRATISCRASESVDSFGHSFMHWYQQKPGQPPKLLIYLASNLESGVPARFSGSGSRTDFTLTIDPVEADDGATYYCQQSNEDPRTFGGGTKLEIK

SEQ ID NO: 300 - protein, 111 aa, Mus musculus
DIVLTQSPASLAVSLGQRATISCRASESVDSFGNSFMHWYQQKPGQPPKLLIYIASNLESGVPARFSGSGSRTDFTLTIGPVEADDAATYYCQQSNEDPRTFGGGTKLEII

SEQ ID NO: 301 - protein, 111 aa, Mus musculus
DIVLTQSPASLAVSLGQRATISCRASESVDNFGNSFMHWYQQKPGQPPKLLIYLASNLESGVPARFSGSGSRTDFTLTIDPVEADDAATYYCQQSNEDPRTFGGGTKLEIK

SEQ ID NO: 302 - protein, 111 aa, Mus musculus
DIVLTQSPASLAVSLGQRATISCRASESVDSFGNSFMHWYQQKPGQPPKLLIYLASNLESGVPARFSGSGSRTDFTLTIDPVEADDAATYYCQQSNEDPRTFGGGTKLEIK

SEQ ID NO: 303 - protein, 111 aa, Mus musculus
DIVLTQSPASLAVSLGQRATISCRASESVDSYGNSFMHWYQQKPGQPPKLLIYLASNLESGVPARFSGSGSRTDFTLTIDPVEADDAATYYCQQSNEDPRTFGGGTKLEIK

SEQ ID NO: 304 - protein, 107 aa, Mus musculus
DIVMTQSQKFMSTSVGDRVSVTCKASQNVGTDVAWYQQKPGQSPKLLIYSASYRFSGVPDRFTGSGSGTDFSLAISNVQSEDLAEYFCQQYNSYPYTFGGGAKLEIK

SEQ ID NO: 305 - protein, 107 aa, Mus musculus
DIQMTQSSSYLSVSLGGRVTITCKASDHINNWLGWYQQKPGNAPRLLISGATSLETGVPSRFSGSGSGKDYTLSITSLQTEDVATYYCQQYWSIPYTFGGGTKLEIK

SEQ ID NO: 306 - protein, 111 aa, Mus musculus
DIVLTQSPASLAVSLGQRATISCRASESVDSFGNSFMHWYQQKPGQPPKLLIYIASNLESGVPARFSGSGSRTDFTLTIDPVEADDAATYYCQQSNEDPRTFGGGTRLEIK

SEQ ID NO: 307 - protein, 111 aa, Mus musculus
DIVLTQSPDSLAVSLGQRATISCRASESVDSFGHSFMHWYQQKPGQPPKLLIYLASNLESGVPARFSGSGSRTDFTLTIDPVEADDAATYYCQQSNEDPRTFGGGTKLEIK

SEQ ID NO: 308 - protein, 111 aa, Mus musculus
DIVLTQSPASLAVSLGQRATISCRAGESVDSFGNSFMHWYQQKPGQPPKLLIYLASNLESGVPARFSGSGSRTDFTLTIDPVEADDAAAYYCQQSNEDPRTFGGGTKLEIK

SEQ ID NO: 309 - protein, 111 aa, Mus musculus
DIVLTQSPASLAVSLGQRATISCRASESVDSSGNSFMHWYQQKPGQPPKLLIYRASNLESGIPARFSGSGSRTDFTLTINPVEADDVATYYCQQSNEDPFTFGSGTKLEIK

SEQ ID NO: 310 - protein, 108 aa, Mus musculus
EIVLTQSPTTMAASPGEKITITCSASSSISSNYLNWYQQKPEFSPKLLIYRTSNLASGVPARFSGSGSGTSYSLTIGTMEAEDVATYYCQQGSSLPRTFGSGTKLEIK

SEQ ID NO: 311 - protein, 108 aa, Mus musculus
EIVLTQSPTTMAASPGEKITITCSASSSISSNYLHWYQQKPGFSPKLFIYRTSNLASGVPARFSGSGSGTSYSLTIGTMEAEDVATYYCQQGSSIPRTFGSGTKLEIK

SEQ ID NO: 312 - protein, 108 aa, Mus musculus
EIVLTQSPTTMAASPGEKITITCSASSSISSNYLNWYQQKPEFSPKLLIYRTSNLASGVPARFSGSGSGTSYSLTIGTMEAEDVATYYCQQGSSIPRTFGSGTKLEIK

SEQ ID NO: 313 - protein, 107 aa, Mus musculus
DIVMTQSQKFMSTSVGDRVSVTCKASQNVGTNVAWYQQKPGQSPKLLIYSASYRYSGVPDRFTGSGSGTDFTLAISNVQSEDLAEYFCQQYNSYPYTFGGGTKLEIK

SEQ ID NO: 314 - protein, 106 aa, Mus musculus
DIQMTQSPSSLSASLGGKVTITCKASQDINKYIAWYQHKPVKGPRLLIHYTSTLQPGIPSRFSGSGSGRDYSFSISNLEPEDIATYYCLQYDNLRTFGGGTKLEIK

SEQ ID NO: 315 - protein, 112 aa, Mus musculus
DIVMTQAAPSIPVTPGESVSISCRSSKSLLHSNGNTFLYWFLLRPGQSPHLLIYRMSNLASGVPDRFSGSGSGTAFTLRISRVEAEDVGVYYCMQHLEFPYTFGGGTKLEIK

SEQ ID NO: 316 - protein, 107 aa, Mus musculus
DIVMTQSQKFMSTSVGDRVSVTCKASQNVGTNVAWYQQRPGQSPKLLIYSASYRYSGVPDRFTGSGSGTDFTLTISNVQSEDLAEYFCQQYNNYPYTFGGGTKLEIK

SEQ ID NO: 317 - protein, 107 aa, Mus musculus
DIVMTQSQKFMSTSVGDRVSVTCKASQNVGTDVAWYQQKPGQSPKLLIYSASYRFSGVPDRFTGSGSGTDFTLTISNVQSEDLAEYFCQQYNIYPYTFGGGTKLEIK

SEQ ID NO: 318 - protein, 107 aa, Mus musculus
DIVMTQSQKFMSTSVGDRVSVTCKASQNVGTDVAWYQQKPGQSPKLLIYSASYRFSGVPDRFTGSGSGTDFTLTISNVQSEDLAEYFCQQYNSYPYTFGGGTKLEIK

SEQ ID NO: 319 - protein, 107 aa, Mus musculus
ETTVTQSPASLSMAIGEKVTIRCITSTDIDDDMNWYQQKPGEPPKLLISEGNTLRPGVPSRFSSSGYGTDFVFTIENMLSEDVADYYCLQSDNLPFTFGSGTKLEIK

SEQ ID NO: 320 - protein, 107 aa, Mus musculus
DIVMTQSQKFMSTSVGDRVSVTCKASQNVGTNVAWYQQKPGQSPKLLIYSASYRFSGVPDRFTGSGSGTDFTLTISNVQSEDLAEYFCQQYNSYPYTFGGGTKLEIK

SEQ ID NO: 321 - protein, 107 aa, Mus musculus
DIVMTQSQKFMSTSVGDRVSVTCKASQNVGTDVAWYQQKPGQSPKLLIYSASYRFSGVPDRFTGSGSGTDFTLTISNVQSEDLAEYSCQQYNSYPYTFGGGTKLEIK

SEQ ID NO: 322 - protein, 107 aa, Mus musculus
DIVMTQSQKFMSTSVGDRVSVTCKASQNVGTNVAWYQQKPGQSPKLLIYSASYRYSGVPDRFTGSGSGTDFTLTISNVQSEDLAEYFCQQYNSYPYTFGGGTKLEIK

SEQ ID NO: 323 - protein, 107 aa, Mus musculus
DIVMTQSQKFMSTSVGDRVSVTCKASQNVGTNVAWYQQKSGQSPKLLIYSASYRFSGVPDRFTGSGSGTDFTLTISNVQSEDLAEYFCQQYNSYPYTFGGGTKLEIK

SEQ ID NO: 324 - protein, 107 aa, Mus musculus
DNVMTQSQKFMSTSVGDRVSVTCKASQNVGTNVAWYQQKSGQSPKLLIYSASYRFSGVPDRFTGSGSGTDFTLTISNVQSEDLAEYFCQQYNSYPYTFGGGTKLEIK

SEQ ID NO: 325 - protein, 107 aa, Mus musculus
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIK

SEQ ID NO: 326 - protein, 107 aa, Mus musculus
DIVMTQSQKFMSTSVGDRVSVTCKASQNVGTDVAWYQQKPGQSPKLLIYSASYRFSGVPDRFTGSGSGTDFTLAISNVQSEDLAEYFCQQYNSYPYTFGGGTKLEIK

SEQ ID NO: 327 - protein, 107 aa, Mus musculus
DILMTQSPSSMSVSLGDTVSITCHASQGISSNIGWLQRKPGKSFKGLIYHGTNLEDGLPSRFSGSGSGADYSLTISSLESEDFADYYCVQYAQFPYTFGGGTKLEIK

SEQ ID NO: 328 - protein, 111 aa, Mus musculus
DIVLTQSPASLAVSLGQRATISCKASQSVDYDGDNYMNWYQQKPGQPPKLLIYAASNLESGIPARFSGSGSGTDFTLNIHPVEEEDAATYYCQQSYEDPWTFGGGTKLEIK

SEQ ID NO: 329 - protein, 107 aa, Mus musculus
DIVMTQSQKFMSTSVGDGVSVTCKASQNVDSNVAWYHQKPGQSPKLLIYSASYRFSGVPDRFTGSGSGTDFTLTISNVQSEDLAEYFCQQYNSYPYTFGGGTKLEVK

SEQ ID NO: 330 - protein, 107 aa, Mus musculus
DIVMTQSQKFMSTSVGDRVSVTCKASQNVGTNIAWYQQKPGQSPKLLIYSASYRYSGVPDRFSGSGSGTDFTLAISNVQSEDLAEYFCQQYNTYPYTFGGGTKLEIK

SEQ ID NO: 331 - protein, 107 aa, Mus musculus
DIVMTQSPATLSVTPGDRVSLSCRASQSISDYLHWYQQKSHESPRLLIKYVSQSISGIPSRFSGSGSGSDFTLSINSVEPEDVGVYYCQNGHSFPFTFGSGTKLEIK

SEQ ID NO: 332 - protein, 107 aa, Mus musculus
DIVMTQSQKFMSTSVGDRVSVTCKASQNVGTDVAWYQQKPGQSPKLLIYSASYRYSGVPDRFTGSGSGTDFTLAISNVQSEDLAEYFCQQYNSYPYTFGGGTKLEIK

SEQ ID NO: 333 - protein, 106 aa, Mus musculus
QIVLTQSPALMSASPGEKVTMTCSASSSVSYMYWYQQKPRSSPKPWIYLTSNLASGVPARFSGSGSGTSYSLTISSMEAEDAATYYCQQWSSNPLTFGAGTKLELK

SEQ ID NO: 334 - protein, 107 aa, Mus musculus
DIVMTQSQKFMSTSVGDRVSVTCKASQNVGTNVAWYQQKPGQSPKLLIYSASYRFSGVPDRFAGSGSGTDFTLAISNVQSEDLADYFCQQYNSYPYTFGGGTKLEMK

SEQ ID NO: 335 - protein, 107 aa, Mus musculus
DIVMTQSQKFMSTSVGDRVSVTCKASQNVGTDVAWYQQKPGQSPKLLIYSASYWFSGVPDRFTGSGSGTDFTLAISNVQSEDLAEYFCQQYNSYPYTFGGGTKLEIK

SEQ ID NO: 336 - protein, 111 aa, Mus musculus
DIVLTQSPASLAVSLGQRATISCRASESVESNGNSFMHWYQQKPGQSPKLLIYRASNLESGIPARFSGSGSRTDFTLTIYPVEADDVATYYCQQTDEDPFTFGSGTKLGIK

SEQ ID NO: 337 - protein, 107 aa, Mus musculus
DIVMTQSPATLSVTPGDRVSLSCRASQRISDYLHWYQQKSHESPRLLIKYASQSISGIPSRFSGSGSGSDFTLSINSVEPEDVGMYYCQNGHSFPFTFGSGTKLEIK

SEQ ID NO: 338 - protein, 107 aa, Mus musculus
DIVMTQSQKFMSTSVGDRVSVTCKASQNVGTDVAWYQQKPGQSPKLLIYSASYRFSGVPDRFTGSGSGTDFTLAISNVQSEDLAEYFCQQYNSYPYTFGGGTKVEIK

SEQ ID NO: 339 - protein, 107 aa, Mus musculus
DIVMTQSQKFMSTSVGDRVSVTCKASQNVGTDVAWYQQKPGQSPKLLIYSASYRYNGVPDRFTGSGSGTDFTLAISNVQSEDLAEYFCQQYNSYPYTFGGGTKLEIK

SEQ ID NO: 340 - protein, 107 aa, Mus musculus
DIVMTQSQKFMSTSVGDRVSVTCKASQNVGTDVAWYQQKPGQSPKLLIYSASYRYNGVPDRFTGSGSGTDFTLAISNVQSEDLAEYFCQQYNTYPYTFGGGTKLEIK

SEQ ID NO: 341 - protein, 107 aa, Mus musculus
DVQITQSPSYLAASPGETITINCRASKSISKYLAWYQEKPGKTNKLLIYSGSTLQSGIPSRFSGSGSGTDFTLTISSLEPEDFAMYYCQQHNEYPLTFGAGTKLELK

SEQ ID NO: 342 - protein, 107 aa, Mus musculus
DIVMTQSQKFMSTSVGDRVSVTCKASQNVGTNVAWYQQKPGQSPKLLIYSASYRFSGVPDRFTGSGSGTDFTLAISNVQSEDLAEYFCQQYNSYPYTFGGGTQLEIK

SEQ ID NO: 343 - protein, 107 aa, Mus musculus
SIVMTQTPKFLLVSAGDRVTITCKASQSVSNDVAWYQQKPGQSPKLLIYYASNRYTGVPDRFTGSGYGTDFTFTISTVQAEDLAVYFCQQDYSSPWTFGGGTKLEIK

SEQ ID NO: 344 - protein, 107 aa, Mus musculus
DIVMTQSQKFMSTSVGDRVSVTCKASQNVDTNVAWYQQKPGQSPKLLIYSASYRFSGVPDRFTGSGSGTDFTLTISNVQSEDLAEYFCQQYNIYPYTFGGGTKLEIK

SEQ ID NO: 345 - protein, 107 aa, Mus musculus
DIVMTQSQKFMSTSVGDRVSVTCKASQNVDTNVAWYQQKPGQSPKLLIYSASYRYSGVPDRFTGSGSGTDFTLTISNVQSEDLAEYFCQQYNSYPYTFGGGTKLEIK

SEQ ID NO: 346 - protein, 112 aa, Mus musculus
DVVMTQTPLSLPVSLGDQASISCRSSQSLVHSNGNTYLHWHLQKPGQSPKLLIYKVSNRFSGVPDRFSGSGSGTDFTLKISRVEAEDLGVYFCSQSTHAPWTFGGGTKLEIK

SEQ ID NO: 347 - protein, 107 aa, Mus musculus
DIVMTQSQEFMSTSVGDRVSVTCKASQNVGTNVAWYQQKPGQSPKLLIYSASYRFSGVPDRFTGSGSGTDFTLTISNVQSEDLAEYFCQQYNNYPYTFGGGTKLEIK

SEQ ID NO: 348 - protein, 107 aa, Mus musculus
DIVMTQSQKFMSTSVGDRVSVTCKASQNVGTNVAWYQQKPGQSPELLIYSASYRYSGVPDRFTGSGSGTDFTLTISNVQSEDLAEYFCQQYNSYPFTFGGGTKLEIK

SEQ ID NO: 349 - protein, 108 aa, Mus musculus
EIVLTQSPTTMATSPGEKITITCSASSSIISNYLHWYQQKPGFSPKLLIYRTSNLASGVPARFSGSGSGTSYSLTIGTMETEDVATYYCQQGSSLPRTFGSGTKLEIK

SEQ ID NO: 350 - protein, 107 aa, Mus musculus
DIVMTQSQKFMSTSVGDRVSVTCKASQNVGTDVAWYQQKPGQSPKLLIYSASYRFSGVPDRFTGSGSGTDFSLAISNVQSEDLAEYFCQQYNSYPYTFGGGTKLEIK

SEQ ID NO: 351 - protein, 107 aa, Mus musculus
DIVMTQSQKFMSTSVGDRVSVTCKASQNVGTDVAWYQQKPGQSPKLLIYSASYRFSGVPDRFTGSGSGTDFTLAISNVQSEDLAEYFCQQYNSYPYTFGGGAKLEIK

SEQ ID NO: 352 - protein, 107 aa, Mus musculus
DIVMTQSQKFMSTSVGDRVSVTCKASQNVGTNVAWYQQKPGQSPKLLIYSASYRFSGVPDRFTGSGSGTDFSLAISNVQSEDLAEYFCQQYNSYPYTFGGGTKLEIK

SEQ ID NO: 353 - protein, 107 aa, Mus musculus
DIVMTQSQKFMSTSVGDRVSVTCKASQNVGTNVAWYQQKPGQSPKLLIYSASYRFSGVPDRFTGSGSGTDFTLAISNVQSEDLAEYFCQQYNSYPYTFGGGTKLEIK

SEQ ID NO: 354 - protein, 107 aa, Mus musculus
SIVMTQTPKFLLVSAGDRFTITCKASQNVDNYVAWYQQKPGQSPKLLIYYASNRYTGVPDRFTGSGYGTDFTFTITTVQAEDLAVYFCQQDYSSPYTFGGGTKLEIK

SEQ ID NO: 355 - protein, 112 aa, Mus musculus
DIVMTQAAPSVPVTPGESVSISCRSNKSLLHSNGNTFLYWFLQRPGQSPQVLIYRMSNLASGVPDRFSGSGSGTAFTLRISRVESEDVGVYYCMQHLEYPYTFGGGTKLEIK

SEQ ID NO: 356 - protein, 108 aa, Mus musculus
EIVLTQSPTTMAASPGEKITITCSASSSISSNYLNWYQQKPEFSPKLLIYRTSYLASGVPARFSGSGSGTSYSLTIGTMEAEDVATYYCQQGSSIPRTFGSGTKLEIK

SEQ ID NO: 357 - protein, 108 aa, Mus musculus
EIVLTQSPTTMAASPGEKITITCSATSIISSNYLHWYQQKPGFSPKLLIYRTSNLASGVPARFSGSGSGTSYSLTIGTMEAEDVATYYCQQGSSIPRTFGSGTKLDIK

SEQ ID NO: 358 - protein, 108 aa, Mus musculus
EIVLTQSPTTMAASPGEKITIICNASSSISSNYFNWYQQKPEFSPKLLIYRTSNLASGVPARFSGSGSGTSYSLTIGTMEAEDVATYYCQQGSSLPRTFGSGTKLEIK

SEQ ID NO: 359 - protein, 108 aa, Mus musculus
EIVLTQSPTTMAASPGEKITITCSANSNISSNYLNWYQQKPVFSPKLLIYRTSNLASGVPTRFSGSGSGTSYSLTIGTMEAEDVATYYCQQGSSIPRTFGSGTKLEIK

SEQ ID NO: 360 - protein, 107 aa, Mus musculus
DIKMTQSPSSMYASLGERVTITCKASQDIKSYLSWYQQKPWKSPKTLIYYATSLADGVPSRFSGSGSGQDYSLTISSLESDDTATYYCLQHGESPFTFGSGTKLEIK

SEQ ID NO: 361 - protein, 111 aa, Mus musculus
DIVLTQSPASLAVSLGQRATISCRASKSVSSSGYNYLHWYQQKPGQPPKLLIYLASNLESGVPARFSGSGSGTDFTLNIHPVEEEDAATYYCQHSGELPLTFGAGTKLELK

SEQ ID NO: 362 - protein, 108 aa, Mus musculus
DIVMTQSPATLSVTPGDRVSLSCRASQSISDYLHWYQQKSHESPRLLIKYASQSISGIPSRFSGSGSGSDFTLSINSVEPEDVGVYYCQNGHSFPPFTFGSGTKLEIK

SEQ ID NO: 363 - protein, 112 aa, Mus musculus
DVVMTQTPLSLPVSLGDQASISCRSSQSLVHSDGNTYLHWYLQKPGQSPKLLIYKVSNRFSGVPDRFSGSGSGTDFTLMISRVEAEDLGVYFCSQSTHVPWTFGGGTKLEIK

SEQ ID NO: 364 - protein, 108 aa, Mus musculus
EIVLTQSPTTMAASPGEKITITCSASSSINSNYLNWYQQKPEFSPKLLIYRTSNLASGVPTRFSGSGSGTSYSLTIGTMEAEDVATYYCQQGSSIPRTFGSGTKLEIK

SEQ ID NO: 365 - protein, 108 aa, Mus musculus
EIVLTQSPTTMAASPGEKITITCSASSSISSNYLNWYQQKPEFSPKLLIYRTSNLASGVPGRFSGSGSGTSYSLTIGTMEAEDVATYYCQQGSSIPRTFGSGTKLEIK

SEQ ID NO: 366 - protein, 106 aa, Mus musculus
QIVLTQSPALMAASPGEKVTITCSVSSSISYMYWYQQKSETSPKPWIYGTSNLASGVPVRFSGSGSGTSYSLTISSMEAEDAATYYCQQWSSYPLTFGAGTKLELK

SEQ ID NO: 367 - protein, 108 aa, Mus musculus
EIVLTQSPTTMAASPGEKITITCSASSSISSNYLNWYQQKPVFSPKLLIYRTSNLASGVPTRFSGSGSGTSYSLTIGTMEAEDVATYYCQQGSSIPRTFGSGTKLEIK

SEQ ID NO: 368 - protein, 108 aa, Mus musculus
EIVLTQSPTTVAASPGEKITITCSASSSISSNHLNWYQQKPGFSPKLLIYRTSNLASGVPARFSGSGSGTSYSLTIGTMEAEDVATYYCQQGSSIPRTFGSGTKLEIK

SEQ ID NO: 369 - protein, 111 aa, Mus musculus
DIVLTQSPASLAVSLGQRATISCKASQSVDYNGYSYLNWYQQKPGQPPKLLIYPASNLESGIPARFSGSGSGTDFTLNIHPVEEEDAATYYCQQSKEDPYTFGGGTKLEIK

SEQ ID NO: 370 - protein, 106 aa, Mus musculus
QIVLTQSPALMSASPGEKVTMTCSASSSVSYMYWYQQKPRSSPKPWIYLTSNLASGVPARFSGSGSGTSYSLTISSMEAEDAATYYCQQWSSNPLTFGTGTKLELK

SEQ ID NO: 371 - protein, 106 aa, Mus musculus
QIVLTQSPAIMSASPGEKVTMTCSASSSVSYMHWYQQKSGTSPKRWIYDTSKLASGVPARFSGSGSGTSYSLTISSMEAEDAATYYCQQWSSNPLTFGAGTKLELK

SEQ ID NO: 372 - protein, 106 aa, Mus musculus
QIVLTQSPALMSASPGEKVTMTCSASSSVSYMYWYQQKPRSSPKPWIYLTSNLASGVPARFSGSGSGTSYSLTISTMEAEDAATYYCQQWSSNPLTFGAGTKLELK

SEQ ID NO: 373 - protein, 106 aa, Mus musculus
QIVLTQSPALMSASPGEKVTMTCSATSSVSYMYWYQQKPRSSPKPWIYLTSNLASGVPARFSGSGSGTSYSLTISSMEAEDAATYYCQQWSSNPLTFGAGTKLELK

SEQ ID NO: 374 - protein, 106 aa, Mus musculus
QIVLTQSPALMSASPGEKVTMTCSASSSVSYMYWYQQKPRSSPKPWIYLTSNLASGVPARFSGSGSGTSYSLTISSMEAEDAATYYCHQWSSNPLTFGAGTKLELK

SEQ ID NO: 375 - protein, 106 aa, Mus musculus
QIVLTQSPALMSASPGEKVTMTCSASSSVSYMYWYQQKPRSSPKPWIYLTSNLASGVPARFNGSGSGTSYSLTISSMEAEDAATYYCQQWSSNPLTFGAGTKLELK

SEQ ID NO: 376 - protein, 106 aa, Mus musculus
QIVLTQSPALMSVSPGEKVTMTCSASSSVSYMYWYQQKPRSSPKPWIYLTSNLASGVPARFSGSGSGTSYSLTISSMEAEDAATYYCQQWSSNPLTFGAGTKLELK

SEQ ID NO: 377 - protein, 106 aa, Mus musculus
QIVLTQSPALMSASPGEKVTMTCSASSSVSYIYWYQQKPRSSPKPWIYLTSNLASGVPARFSGSGSGTSYSLTISSMEAEDAATYYCQQWSSNPLTFGAGTKLELK

SEQ ID NO: 378 - protein, 106 aa, Mus musculus
QIVLTQSPALMSASPGEKVTMTCSASSSVSYMYWYQQKPRSSPKPWIYLTSSLASGVPARFSGSGSGTSFSLTISNMEAEDAATYYCHQWSSNPLTFGAGTKLELK

SEQ ID NO: 379 - protein, 106 aa, Mus musculus
QIVLTQSPALMSASPGEKVTMTCSASSSVSYMYWYQQKPRSSPKPWIYLTSNLASGVPARFSGSGSGTSYSLTISSMEAEDAATYYCQQWSSNPLTFGAGTKLELN

SEQ ID NO: 380 - protein, 106 aa, Mus musculus
QIVLSQSPAILSASPGEKVTMTCRASSSVSYMHWYQQKPGSSPKPWIYATSNLASGVPARFSGSGSGTSYSLTISRVEAEDAATYYCQQWSSNPLTFGAGTKLELK

SEQ ID NO: 381 - protein, 106 aa, Mus musculus
QIVLTQSPALMSASPGEKVTMTCSASSSVSYIYWYQQKPGSSPKPWIYLTSNLASGVPPRFSGSGSGTSYSLTISSMEAEDAASYYCQQWSSNPLTFGAGTKLELK

SEQ ID NO: 382 - protein, 107 aa, Mus musculus
DIVMTQSPATLSVTPGDRVSLSCRASQSISDYLHWYQQKSHESPRLLIKYASQSISGIPSRFSGSGSGTDFTLSINSVEPEDVGVYYCQSGHSFPLTFGAGTKLEVK

SEQ ID NO: 383 - protein, 107 aa, Mus musculus
DIQMTQSPASLSASVGETVTITCRASENIYSYLAWHKQKQGKSPQFLVYNAKTLAEGVPSRFSGSGSGTQFSLKINGLQPEDFGSYYCQHHYVIPPTFGGGTKLEIK

SEQ ID NO: 384 - protein, 107 aa, Mus musculus
DIVMTQSPATLSVTPGDRVSLSCRASQNISDYLHWYQQRSHESPRLLIKYASLSISGIPSRFSGSGSGSDFTLSINSVEPEDVGLYYCQNGHSFPLTFGAGTKLELK

SEQ ID NO: 385 - protein, 107 aa, Mus musculus
DIVMTQSPATLSVTPGDRVSLSCRASQNISDYLHWYQQRSHESPRLLIKYASLSISGIPSRFSGSGSGSDFTLSINSVEPEDVGVYYCQNAHSFPLTFGAGTKLELK

SEQ ID NO: 386 - protein, 106 aa, Mus musculus
QNVLTQSPALMSASPGEKVTMTCSASSSVSYMYWYQQKPRSSPKPWIYLTSNLASGVPARFSGSGSGTSYSLTISSMEAEDAATYYCQQWSSNPLTFGAGTKLELK

SEQ ID NO: 387 - protein, 106 aa, Mus musculus
QIVLTQSPALMSASPGEKVTMTCSASSSVSYIYWYQQKPRSSPKPLIYLTSNLASGVPARFSGSGSGTSYSLTISSMEAEDAASYYCQQWSSNPLTFGAGTKLELK

SEQ ID NO: 388 - protein, 107 aa, Mus musculus
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 389 - protein, 106 aa, Mus musculus
QIVLTQSPALMSASPGEKVTMTCSASSSVSYMYWYQQKPRSSPKPWIYLTSNLASGVPARFSGSGSGTSYSLTISNMEAEDAATYYCQQWSSNPLTFGAGTKLELK

SEQ ID NO: 390 - protein, 108 aa, Mus musculus
DIQMTQSPAFLSASVGESVTITCRASENIYSYLAWYQQKQGKSPQLLVYNAKTLAEGVPSRFSGSGSGTHFSLKINSLQPEDFGGYYCQHHYGIPPWTFGGGTKLEIK

SEQ ID NO: 391 - protein, 111 aa, Mus musculus
DIVLTQSPASLAVSLGQRATFSCRASKSVSTSGYSYMHWYQQKPGQPPKLLIYLASNLESGVPARFSGSGSGTDFTLNIHPVEEEDAAAYYCQHSRELPYTFGGGTKLEIK

SEQ ID NO: 392 - protein, 111 aa, Mus musculus
DIVLTQSPASLVVSLGQRATISCRASKSVSTSGYSYMHWYQQKPGQPPKLLIYLASNLESGVPARFSGSGSGTDFTLNIHPVEEEDAATYYCQHSRELPSTFGGGTKLEIK

SEQ ID NO: 393 - protein, 107 aa, Mus musculus
DIQMTQSPASLSASVGETVTITCRASENIYSYLVWYQQKQGKSPQLLVYYAKTLAEGVPSRFSGSGSGTQFSLKINSMQPEDFGSYYCQHHYGTPLTFGVGTKVELK

SEQ ID NO: 394 - protein, 108 aa, Mus musculus
QIVLTQSPAIMSASPGEKVTLTCSASSSVSSSHMYWYQQKPGSSPKLWIYSTSNLASGVPARFSGSGSGTSYSLTISSMEAEDAASYFCHQWSSYPYTFGGGTKLEIK

SEQ ID NO: 395 - protein, 112 aa, Mus musculus
DVVMTQTPLSLPVSLGDQASISCSSSQSLVYSNGNTYLHWYLQKPGQSPKLLIYKVSNRFPGVPDRFSGSGSGTDFTLKISRVEAEDLGVYFCSQSTHVPYTFGGGTKLEIK

SEQ ID NO: 396 - protein, 106 aa, Mus musculus
QIVLTQSPAIMSASPGEKVTISCSASSSVSYMYWYQQKPGSSPKPWIYRTSNLASGVPARFSGSGSGTSYSLTISSMEAEDAATYYCQQYHSFPRTFGGGTKLEIK

SEQ ID NO: 397 - protein, 107 aa, Mus musculus
DIQMTQSSSYLSVSLGGRVTITCKASDHINNWLAWYQQKPGNAPRLLISGATSLETGVPSRFSGSGSGKDYTLSITSLQIEDVATYSCQQYWSIPYTFGGGTKLEIK

SEQ ID NO: 398 - protein, 107 aa, Mus musculus
DIVMTQSHKFMSTSVGDRVNITCKASQDVNTAVAWYQQKPGQSPKLLFYSTSYRYTGVPDRFTGSGSGTDFTFTISSVQAEDLAVYYCQQHYSSPWTFGGGTKLEIK

SEQ ID NO: 399 - protein, 107 aa, Mus musculus
DIVLTQSPAALSVTPGDSVSLSCRASQSISSTLHWYQQKSHESPRLLIRYASQSISGIPSRFSGSGSGTDFTLSINSVETEDFGMYFCHQSYSWPHTFGAGTKLELK

SEQ ID NO: 400 - protein, 107 aa, Mus musculus
DIVMTQSHKFMSTSVGDRVSITCRASQDVSTAVAWYQQKPGQSPKLLIYSASYRYTGVPDRFTVSGSGTDFTFTISSVQAEDLAVYYCQQHYSTPWTFGGGTKLEIK

SEQ ID NO: 401 - protein, 107 aa, Mus musculus
DIVLTQSPAALSVTPGDSVSLSCRASQSISNTLHWYQQKSHESPRLLIRYASQSISGIPSRFSGSGSGTDFTLSINSVETEDFGMYFCQQSNDWPHTFGAGTKLELK

SEQ ID NO: 402 - protein, 107 aa, Mus musculus
DIVLTQSPAALSVTPGDSVSLSCRASQSISNTLHWYQQKSHESPRLLIRYASQSISGIPSRFSGSGSGTDFTLSINSVETEDFGMYFCQQSYDWPHTFGAGTKLELK

SEQ ID NO: 403 - protein, 107 aa, Mus musculus
DIVLTQSPAALSVTPGDSVSLSCRASQSISNTLHWYQQKSHESPRLLIRYASQSISGIPSRFSGSGSGTDFTLSINSVETEDFGMYFCQQSYNWPHTFGAGTKLELK

SEQ ID NO: 404 - protein, 107 aa, Mus musculus
DIQMTQSPASLSGSVGETVTITCRASENIYSFLAWYQQKQGKSPQLLIYNARTLAGGVPSRFSGSRSGTQFSLRINSLQPEDFGTYFCQHHYGPPFTFGGGTNLEIK

SEQ ID NO: 405 - protein, 107 aa, Mus musculus
DIQMTQSPASLSASVGETVTITCRASENIYSFLAWYQQKQGKSPQLLIYNARALAEGVTSRFSGSGSGTQFSLRINSLQPEDFGTYFCQHHYGPPFTFGGGTNLEIK

SEQ ID NO: 406 - protein, 107 aa, Mus musculus
DIQMTQSSSYLSVSLGGRVTITCKASDHISNWLAWYQQKPGNAPRLLISGATSLETGVPSRFSGSGSGKDYTLSITSLQTEDVATYYCQQYWSLPYTFGGGTKLEIK

SEQ ID NO: 407 - protein, 107 aa, Mus musculus
DIQMTQSSSYLSVSLGGRVTITCKASDHINNWLAWYQQKPGNAPRLLISGATSLETGVPSRFSGSGSGKDYTLSITSLQTEDVATYFCQQYWTIPYTFGGGTKLEIK

SEQ ID NO: 408 - protein, 107 aa, Mus musculus
DIQMTQSSSYLSVSLGGRVTITCKASDHINNWLAWYQQKPGNAPRLLISGATSLETGVPSRFSGSGSGKDYTLSITSLQTEDVATYYCQQYWSIPYTFGGGTKLEIK

SEQ ID NO: 409 - protein, 107 aa, Mus musculus
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 410 - protein, 107 aa, Mus musculus
DIVLTQSPATLSVTPGDRVSLSCRASQSINNYLHWYQQKSHESPRLLIKYASHSISGIPSRFSGSGSGTDFTLSINSVETEDFGVYFCQQSNSWPLTFGAGTKLELK

SEQ ID NO: 411 - protein, 107 aa, Mus musculus
DIVMTQSHKFMSTSVGDRVSITCKASQDVSTAVAWYQQKPGQSPKLLIYSASYRYTGVPDHFTVSGSGTDFTFTISSVQAEDLAVYYCQQHYSTPWTFGGGTKLEIK

SEQ ID NO: 412 - protein, 107 aa, Mus musculus
DIQMTQSSSYLSVSLGGRVTITCKASDHINNWLAWYQQKPGNAPRLLISGATSLEPGVPSRFSGSGSGKDYTLTITSLQTEDVATYYCQQYWSLPYMFGGGTKLEIK

SEQ ID NO: 413 - protein, 107 aa, Mus musculus
DIVMTQSHKFMSTSVGDRVSITCKASQDVTTAVAWYQQKPGQSPKLLIYSASYRYTGVPDRFTVSGSGTDFTLTISSVQAEDLAVYYCQQHYSAPWTFGGGTKLKIT

SEQ ID NO: 414 - protein, 107 aa, Mus musculus
DIVLTQSPAALSVTPGDSVSLSCRASQSISNTLHWYQQKSHESPRLLIRYASQSISGIPSRFSGSGSGTDFTLSINSVETEDFGMYFCQQSNSWPHTFGAGTKLELK

SEQ ID NO: 415 - protein, 110 aa, Mus musculus
DIVLTQSPASLAVSLGQRATISCRASKSVSTSGYSYMHWYQQKPGQAPKLLIYLASNLESGVPARFSGSGSGTDFTLNIQPVEEEDAATYYCQHSRELYTFGGGTKLEIK

SEQ ID NO: 416 - protein, 107 aa, Mus musculus
DIVLTQSPATLSVTPGDRVSLSCRASQSISNYLHWYQQKSRESPRLLIKYASQSISGIPSRFSGSGSGTDFTLSINSVETEDFGMYFCQQSNSWPLTFGAGTKLELK

SEQ ID NO: 417 - protein, 107 aa, Mus musculus
DIVLTQSPATLSVTPGDRVSLSCRASQSISNYLHWYQQKSHESPRLLIKYASQSISGIPSRFSGSGSGTDFTLSINSVETEDFGMYFCQQSNSWPLTFGAGTKLELK

SEQ ID NO: 418 - protein, 107 aa, Mus musculus
DIVLTQSPATLSVTPGDRVSLSCRASQSISNYLHWYQQKSHESPRLLIKYASQSISGIPSRFSGSGSGTDFTLSISSVETEDFGMYFCQQSNSWPLTFGAGTKLELK

SEQ ID NO: 419 - protein, 106 aa, Mus musculus
QIVLSQSPAILSASPGEKVTMTCRASSSVSYMHWYQQKPGSSPKPWIFATSNLAYGVPDRFSGSGSGTSYSLTISRMEAEDAATYYCQQWSSTPPTFGGGTKLEIK

SEQ ID NO: 420 - protein, 111 aa, Mus musculus
DIVLTQSPASLAVSLGQRATISCRASESVDSYGNSFMHWYQQKPGQPPKLLIYRASNLESGIPARFSGSGSRTDFTLTINPVEADDVATYYCQQSNEDPYTFGGGTKLEIK

SEQ ID NO: 421 - protein, 107 aa, Mus musculus
SIVMTQTPKFLLVSAGDRVTITCKASQSVSNDVAWYQQKPGQSPKLLIYYASNRYTGVPDRFTGSGYGTDFTFTISTVQAEDLAVYFCQQDYSSPPTFGGGTKLEIK

SEQ ID NO: 422 - protein, 113 aa, Mus musculus
DIVMSQSPSSLAVSVGEKVTMSCKSSLSLLYSSNQKNYLAWYQQKPGQSPKLLIYWASTRESGVPDRFTGSGSGTGFTLTISSVKAEDLAVYYCQQYYSYPLTFGAGTKLELK

SEQ ID NO: 423 - protein, 106 aa, Mus musculus
DIVMTQSHKFMSTSVGDRVSITCKASQDVGTAVAWYQQKPGQSPKLLIYRASTRHTGVPDRFTGSGSGTDFILTISNVQSEDLADYFCQQYSTYYTFGGGTKLEIK

SEQ ID NO: 424 - protein, 107 aa, Mus musculus
DIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPWTFGGGTKLEIK

SEQ ID NO: 425 - protein, 112 aa, Mus musculus
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPFTFGSGTKLEIK

SEQ ID NO: 426 - protein, 111 aa, Mus musculus
DIVLTQSPASLAVSQGQRATISCRASESVDSYGYSFMHWYQQKPGQPPKLLIYLASNLESGVPARFSGSGSRTDFTLTIDPVEADDAATYYCQQNNEDPFTFGSGTKLEIK

SEQ ID NO: 427 - protein, 111 aa, Mus musculus
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVWTFGGGTKLEIK

SEQ ID NO: 428 - protein, 107 aa, Mus musculus
DIQMTQSSSYLSVSLGGRVTITCKASDHINNWLAWYQQKPGNAPRLLISGATSLETGIPSRFSGSGSGKDYSLSITSLQIEDVATYSCQQYWSIPYTFGGGTKLEIK

SEQ ID NO: 429 - protein, 107 aa, Mus musculus
DIQMTQSPASLAASVGETVTITCRTSENIYYSLAWFQQKQGKSPQLLIYNANSLEDGVPSRFSASGSGTQFSMKIKSMQPEDTATYFCKQSYDVPYTFGGGTKLEIK

SEQ ID NO: 430 - protein, 107 aa, Mus musculus
DIQMTQSPASLAASVGETVTITCRTSENIYYSLAWFQQKQGKSPQLLIYNANSLEDGVPSRFSGSGSGTQFSMKIKSMQPEDTATYFCKQSYDVPYTFGGGTKLEIK

SEQ ID NO: 431 - protein, 107 aa, Mus musculus
DIQMTQSPASLAASVGETVTITCRTSENIYYSLAWFQLKQGKSPQLLIYNANTLEDGVPSRFSASGSGTQFSMKIKSMQPEDTATYFCKQSYDVPYTFGGGTKLEIK

SEQ ID NO: 432 - protein, 107 aa, Mus musculus
DIQMTQSPASLAASVGETVTITCRTSENIYYSLAWFQLKQGKSPQLLIYNANSLEDGVPSRFSGSGSGTQFSMKIKSMQPEDSATYFCKQSYDVPYTFGGGTKLEIK

SEQ ID NO: 433 - protein, 107 aa, Mus musculus
DIQMTQSPASLAASVGETVTITCRTSENIYYSLAWFQQKQGKSPQLLIYNAKSLEDGVPSRFSASGSGTQFSMKIKSMQPEDTATYFCKQSYDVPYTFGGGTKLEIK

SEQ ID NO: 434 - protein, 107 aa, Mus musculus
DIQMTQSPASLAASVGETVTITCRTSENIYYSLAWFQQKQGKSPQLLIYNANSLEDGVPSRFSGSGSGTQFSMKIKSMQPEDTATYFCKQAYDVPYTFGGGTKLEIK

SEQ ID NO: 435 - protein, 107 aa, Mus musculus
DIQMTQSPASLAASVGETVTITCRASENIYYTLAWFQQKQGNSPQLLIYNANSLEDGVPSRFSGSGSGTQYSMKINSMQPEDTATYFCKQAYDVPYTFGGGTKLEIK

SEQ ID NO: 436 - protein, 107 aa, Mus musculus
DIQMTQSPSSLSASLGERVSLTCRASQEISGYLTWLQQKPDGTIKRLIYAASTLDSGVPKRFSGSRSESDYSLTISSLESEDFADYYCLQSASYPPTFGGGTKLEIK

SEQ ID NO: 437 - protein, 112 aa, Mus musculus
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPYTFGGGTKLEIK

SEQ ID NO: 438 - protein, 107 aa, Mus musculus
DIVMTQSPATLSVTPGDRVSLSCRASQSISDYLHWYQQKSHESPRLLIKYASQSISGIPSRFSGSGSGSDFTLSINSVEPEDVGVYYCQNGHSFPFTFGSGTKLEIK

SEQ ID NO: 439 - protein, 107 aa, Mus musculus
DIVMTQSPATLSVTPGDRVSLSCRASQSISDYLHWYQQKSHESPRLLIKYASQSISGIPSRFSGSGSGSDFTLSINSVEPEDVGVYYCQNGHSFPFTFGSGTRLEIK

SEQ ID NO: 440 - protein, 107 aa, Mus musculus
DIVMTQSPATLSVTLGDRVSLSCRASQSISDYLHWYQQKSHESPRLLIKYASQSISGIPSRFSGSGSGSDFTLSINSVEPEDVGVYYCQNGHSFPFTFGSGTKLEIK

SEQ ID NO: 441 - protein, 112 aa, Mus musculus
DVVMTQTPLSLPVSLGDQASISCRSRQSLVHSNGITYLHWYLQKPGQSPKLLIDKVFNRFSGVPDRFSGSGSGTDFTLKISRVEAEDLGVYFCSQSTHVPWTFGGGTKLEIK

SEQ ID NO: 442 - protein, 112 aa, Mus musculus
DVVMTQTPLSLPVSLGDQASISCRSRQSLVHSNGITYLHWYLQKPGQSPKLLIYKVSNRFSGVPDRFSGSGSGTDFTLKISRVEAEDLGVYFCSQSTHVPWTFGGGTKLEIK

SEQ ID NO: 443 - protein, 112 aa, Mus musculus
DVVMTQTPLSLPVSLGDQASISCRSRQSLVHSNGITYLHWYLQKPGQSPKLLIYKVSNRFSGVPDRFSGSGSGTDFTLKISRVEAEDLGVYFCSQSTHFPWTFGGGTKLEIK

SEQ ID NO: 444 - protein, 107 aa, Mus musculus
DIVLTQSPATLSVTPGDRVSLSCRASQSINNYLHWYQQKSHESPRLLIKYASHSISGIPSRFSGSGSGTDFTLSINSVETEDFGMYFCQQSSTWPLTFGAGTKLELK

SEQ ID NO: 445 - protein, 107 aa, Mus musculus
DIVLIQSPATLSVTPGDRVSLSCRASQGINNYLHWYQQKSHESPRLLIKYASHSISGIPSRFSGSGSGTDFTLSINSVETEDFGMYFCQQSNSWPLTFGAGTKLELK

SEQ ID NO: 446 - protein, 107 aa, Mus musculus
DIVLTQSPATLSVTPGDRVSLSCRASQSINNYLHWYQQKSHESPRLLIKYASHSISGIPSRFSGSGSGTDFTLSINSVESEDFGKYFCQQSNSWPLTFGAGTQLELK

SEQ ID NO: 447 - protein, 107 aa, Mus musculus
DIVLIQSPATLSVTPGDRVSLSCRASQSINNYLHWYQQKSHESPRLLIKYASHSISGIPSRFSGSGSGTDFTLSINSVETEDFGMYFCQQSNSWPLTFGAGTKLELK

SEQ ID NO: 448 - protein, 107 aa, Mus musculus
DIVLTQSPATLSVTPGDRVSLSCRASQSINNYLHWYQQKSHESPRLLIKYASHSISGIPSRFSGSGSGTDFTLSINSVETEDFGMYFCQQSNSWPLTFGAGTKLELK

SEQ ID NO: 449 - protein, 107 aa, Mus musculus
DIVLTQSPATLSVTPGDRVSLSCRASQRINSYLHWYQQKSHESPRLLIKYASHSISGIPSRFSGSGSGTDFTLSINSVETEDFGMYFCQQSNSWPLTFGAGTKLELK

SEQ ID NO: 450 - protein, 107 aa, Mus musculus
DIVMTQSPATLSVTPGDRVSLSCRASQSISDYLHWYQQKSHESPRLLIKSASQSISGIPSRFSGSGSGSDFTLSINSVEPEDVGVYYCQNGHSFPFTFGSGTKLEIE

SEQ ID NO: 451 - protein, 111 aa, Mus musculus
DVVMAPTPLTLSVTIGQPASISCKSSQSLLDSDGETYLNWLLQRPGQSPKRLIYLVSKLDSGVPDRFTGSGSGTDFTLKISRVEAEDLGVYYCWEGTHFWPFGGGTKLEIK

SEQ ID NO: 452 - protein, 107 aa, Mus musculus
DIVMTQSPATLSVTPGDRVSLSCRASQSISDYLYWYQQKSHESPRLLIKYASQSISGIPSRFSGSGSGSDFTLSISSVEPEDVGVYYCQNGHSFPFTFGSGTKLEIK

SEQ ID NO: 453 - protein, 108 aa, Mus musculus
ENVLTQSPVIMSASPGEKVTMTCRASSSVSSSYLHWYQQKSGASPKLWIYSTSNLASGVPARFSGSGSGTSYSLTISSVEAEDAATYYCQQYSGYPLTFGSGTKLEIK

SEQ ID NO: 454 - protein, 110 aa, Mus musculus
DIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPTFGGGTKLEIK

SEQ ID NO: 455 - protein, 107 aa, Mus musculus
DIVMTQSPATLSVTPGDRVSLSCRASQSISDYLHWYQQKSHESPRLLIKYASQSISGIPSRFSGSGSGSDFTLSISSVEPEDVGVYYCQNGHSFPFTFGSGTKLEIK

SEQ ID NO: 456 - protein, 107 aa, Mus musculus
DIVMTQSPATLSVTPGDRVSLSCRASQSISDYLHWYQQKSHESPRLLIKYASQSISGIPSRFSGSGSGSDFTLSINNVEPEDVGVYYCQNSHSFPFTFGSGTKLEIK

SEQ ID NO: 457 - protein, 107 aa, Mus musculus
DIVMTQSPATLSVTPGDRVSLSCRASQSISDYLHWYQQKSHESPRLLIKYASQSISGIPSRFSGSGSGSDFTLSINSVDPEDVGMYYCQNSHSFPFTFGSGTKLEIK

SEQ ID NO: 458 - protein, 107 aa, Mus musculus
DIVMTQSPATLSVTPGDSVSLSCRASQTISDYLHWYQQKSHESPRLLIKYASQSISGIPSRFSGSGSGSDFTLSINSVEPEDVGVYYCQNSHSFPFTFGSGTKLEIK

SEQ ID NO: 459 - protein, 107 aa, Mus musculus
DIVMTQSPATLSVTPGDRVSLSCRASQSISDYLHWYQQKSHESPRLLIKYASQSISGIPSRFSGSGSGSDFTLTINSVEPEDIGLYYCQNGHSFPFTFGSGTKLEIK

SEQ ID NO: 460 - protein, 112 aa, Mus musculus
DVVMTQTPLSLPVSLGDQASISCRSRQSLVHSNGITYLHWYLQKPGQSPKLLIFKVSNRFSGVPDRFSGSGSGTDFTLKISRVEAEDLGVYFCSQSTHVPWTFGGGTKLEIK

SEQ ID NO: 461 - protein, 107 aa, Mus musculus
SIVVTQTPKFLLVSAGDRVTITCKASQSVSNDVAWYQQKPGQSPKLLIYYASNRYTGVPDRFTGSGYGTDFTFTISTVQAEDLAVYFCQQDYTSPPTFGGGTKLEIK

SEQ ID NO: 462 - protein, 107 aa, Mus musculus
SIVMTQTPKFLLVSAGDRVTITCKANQFVSNFVAWYQQKPGQSPKLLIYYASNRYTGVPDRFTGSGYGTDFTFTITAVQAEDLAVYFCQQDYSSPYTFGGGTKLEIK

SEQ ID NO: 463 - protein, 107 aa, Mus musculus
SIVMTQTPKFLLVSAGDRVTITCKASQFVSNFVAWYQQKPGQSPKLLIYYASNRYTGVPDRFTGSGYGTDFTFTITTVQAEDLAVYFCQQDYSSPYTFGGGTKLEIK

SEQ ID NO: 464 - protein, 107 aa, Mus musculus
SIVMTQTPKFLLVSAGDRVTITCKASQSVNNDVAWYQQKPGQSPKLLIYYASNRYTGVPDRFTGSGYGTDFTFTISTVQAEDLAVYFCQQDYNSPYAFGGGTKLEIK

SEQ ID NO: 465 - protein, 107 aa, Mus musculus
SIVMTQTPKFLLVSAGDRVTITCKASQSVINDVAWYQQKPGQSPKLLIYYASNRYTGVPDRFTGSGYGTDFTFTISSVQAEDLAVYFCQQDYSSPYTFGGGTKLEIK

SEQ ID NO: 466 - protein, 107 aa, Mus musculus
SIVMTQTPKFLLVSAGDRVTITCKASQSVINDVAWFQQKPGQSPNLLIYYASNRYTGVPARFTGSGYGTDFTFTISTVQAEDLAVYFCQQDYSSPYTFGGGTKLEIK

SEQ ID NO: 467 - protein, 107 aa, Mus musculus
SIVMTQTPKFLLVSAGDRVTITCKASQSVSNYVAWYQQKPGQSPKLLIYYASNRYTGVPDRFTGSGYGTDFTFTITTVQAEDLAVYFCQQDYSSPYTFGGGTKLEIK

SEQ ID NO: 468 - protein, 107 aa, Mus musculus
SIVMTQTPKFLLVSAGDRVTITCKASQSVINDVAWYQQKPGQSPKLLIYYASNRYTGVPDRFTGSGYGTDFTFTISTVQAEDLAVYFCQQDYSSPYTFGGGTKLEIK

SEQ ID NO: 469 - protein, 107 aa, Mus musculus
SVVMTQTPKFLLVSAGDRITITCKASQSVINDVAWYQQKPGQSPKLLIYYASNRYTGVPDRFTGSGYGTDFTFTISTVQAEDLAVYFCQQDYNSPFTFGGGTKLEIK

SEQ ID NO: 470 - protein, 107 aa, Mus musculus
SIVMTQTPKFLLVSTGDRFTITCKASQNVDNYVAWYQQKPGQSPKLLIYYASNRYTGVPDRFTGSGYGTDFTFTITTVQAEDLAVYFCQQDYSSPYTFGGGTKLEIK

SEQ ID NO: 471 - protein, 107 aa, Mus musculus
SLVMTQTPKFLLVSPGDRITITCKASQSVINDVAWYQQKPGQSPKLLIYYASNRNTGVPDRFTGSGYGTDFTLTISTVQAEDLAVYFCQQDYNSPYTFGGGTKLEIK

SEQ ID NO: 472 - protein, 107 aa, Mus musculus
SIVMTQTPKFLLVSAGDRITITCKASQSVINDVAWYQQKPGQSPKLLMYYASNRNTGVPDRFTGSGYGTDFTLTISTVQAEDLAVYFCQQDYNSPYTFGGGTKLEIK

SEQ ID NO: 473 - protein, 107 aa, Mus musculus
DIQMTQSPASLSVSVGETVTITCRASENIYSNLAWYQQKQGKSPQLLVYAATNLADGVPSRFSGSGSGTQYSLKINSLQSEDFGSYYCQHFWGTPFTFGSGTKLEIK

SEQ ID NO: 474 - protein, 106 aa, Mus musculus
DIQMTQSPSSLSASLGGKVTITCKASQDINKYISWYQHKPGKGPRLLIHYTSTLQPGIPSRFSGSGSGRDYSFSISNLEPEDIATYYCLQYDNLWTFGGGTKLEIK

SEQ ID NO: 475 - protein, 107 aa, Mus musculus
SIVMTQTPKFLLVSAGDRVTITCKASQSVINDVAWFQQQPGQSPKLLIYYASNRYTGVPDRFTGSGYGTDFTFTISTVQAEDLAVYFCQQDYSSPYTFGGGTKLEIK

SEQ ID NO: 476 - protein, 107 aa, Mus musculus
SIVMTQTPKFLLVSAGDRVTITCKASQSVSNDVAWYQQKPGQSPKLLIYYASNRYTGVPDRFTGSGYGTDFTFTISTVQAEDLAVYFCQQDYNSPYTFGGGTKLEIK

SEQ ID NO: 477 - protein, 107 aa, Mus musculus
NIVMTQTPRFLLVSAGDRVTITCKASQTVTNDVAWYQQKPGQSPKLLIYYASNRYTGVPDRFTGSGYGTDFTFTISTVQAEDLAVYFCQQDYISPYTFGGGTKLEIK

SEQ ID NO: 478 - protein, 107 aa, Mus musculus
DIQMTQTTSSLSASLGDRVTINCSASQGIINYLNWYQQKPDGTVKLLIYYTSSLHSGVPSRFSGSGSGTDYSLTISNLEPEDIASYYCQQYSKLPYTFGGGTKVEIK

SEQ ID NO: 479 - protein, 9 aa, Mus musculus
GYSITSNYN

SEQ ID NO: 480 - protein, 7 aa, Mus musculus
IHYSDDT

SEQ ID NO: 481 - protein, 17 aa, Mus musculus
ARWSYYYGGPYYYSMDY

SEQ ID NO: 482 - protein, 9 aa, Mus musculus
GYSITSGYN

SEQ ID NO: 483 - protein, 7 aa, Mus musculus
IHYSGGT

SEQ ID NO: 484 - protein, 17 aa, Mus musculus
GRWSYYYGSPYYYSMDY

SEQ ID NO: 485 - protein, 9 aa, Mus musculus
GFSITSGYY

SEQ ID NO: 486 - protein, 7 aa, Mus musculus
ISYDGSN

SEQ ID NO: 487 - protein, 13 aa, Mus musculus
ARDYYGYPAWFTY

SEQ ID NO: 488 - protein, 9 aa, Mus musculus
GYSITDGYF

SEQ ID NO: 489 - protein, 7 aa, Mus musculus
LNYDGSN

SEQ ID NO: 490 - protein, 12 aa, Mus musculus
ARRKGWLYAMDY

SEQ ID NO: 491 - protein, 9 aa, Mus musculus
GYSITSAYF

SEQ ID NO: 492 - protein, 7 aa, Mus musculus
INYDGSN

SEQ ID NO: 493 - protein, 7 aa, Mus musculus
INFDGSS

SEQ ID NO: 494 - protein, 7 aa, Mus musculus
INYDGTN

SEQ ID NO: 495 - protein, 9 aa, Mus musculus
GYSITSDSF

SEQ ID NO: 496 - protein, 9 aa, Mus musculus
GYSITSDYF

SEQ ID NO: 497 - protein, 9 aa, Mus musculus
GYSITSGYF

SEQ ID NO: 498 - protein, 7 aa, Mus musculus
INYDGSD

SEQ ID NO: 499 - protein, 12 aa, Mus musculus
ARRKGWLYAMDS

SEQ ID NO: 500 - protein, 7 aa, Mus musculus
INYDGSS

SEQ ID NO: 501 - protein, 7 aa, Mus musculus
LNYDGSS

SEQ ID NO: 502 - protein, 9 aa, Mus musculus
GYSITSGYY

SEQ ID NO: 503 - protein, 9 aa, Mus musculus
AREGDYGSY

SEQ ID NO: 504 - protein, 12 aa, Mus musculus
TRRKGWLYAMDY

SEQ ID NO: 505 - protein, 9 aa, Mus musculus
GYSITSDYA

SEQ ID NO: 506 - protein, 7 aa, Mus musculus
ISYSGST

SEQ ID NO: 507 - protein, 14 aa, Mus musculus
ASGYYGIHYYAMDY

SEQ ID NO: 508 - protein, 8 aa, Mus musculus
GYSFTAYI

SEQ ID NO: 509 - protein, 8 aa, Mus musculus
INPYYGST

SEQ ID NO: 510 - protein, 18 aa, Mus musculus
ARGPPYYYGSSRDYALDY

SEQ ID NO: 511 - protein, 8 aa, Mus musculus
GYSFTDYI

SEQ ID NO: 512 - protein, 8 aa, Mus musculus
INPYYDTT

SEQ ID NO: 513 - protein, 8 aa, Mus musculus
ATYDGFAY

SEQ ID NO: 514 - protein, 8 aa, Mus musculus
INPYYGTT

SEQ ID NO: 515 - protein, 8 aa, Mus musculus
INPYYYTT

SEQ ID NO: 516 - protein, 8 aa, Mus musculus
GFNIKDYY

SEQ ID NO: 517 - protein, 8 aa, Mus musculus
IDPENGNT

SEQ ID NO: 518 - protein, 11 aa, Mus musculus
TIGVRRDYFDY

SEQ ID NO: 519 - protein, 8 aa, Mus musculus
GFTFSNYW

SEQ ID NO: 520 - protein, 8 aa, Mus musculus
IRLKFNNY

SEQ ID NO: 521 - protein, 8 aa, Mus musculus
IKFGGMDY

SEQ ID NO: 522 - protein, 8 aa, Mus musculus
GFAFNSYD

SEQ ID NO: 523 - protein, 8 aa, Mus musculus
ISDGGSFT

SEQ ID NO: 524 - protein, 12 aa, Mus musculus
ARHQITTLRMDY

SEQ ID NO: 525 - protein, 8 aa, Mus musculus
GFAFSSYD

SEQ ID NO: 526 - protein, 8 aa, Mus musculus
ISDGGSYT

SEQ ID NO: 527 - protein, 12 aa, Mus musculus
ARHRVTTVRMDY

SEQ ID NO: 528 - protein, 8 aa, Mus musculus
GFAFSDYD

SEQ ID NO: 529 - protein, 12 aa, Mus musculus
ARHLITTLRMDY

SEQ ID NO: 530 - protein, 8 aa, Mus musculus
ISNDGSYT

SEQ ID NO: 531 - protein, 8 aa, Mus musculus
GFTFSSYT

SEQ ID NO: 532 - protein, 8 aa, Mus musculus
IISGGGDI

SEQ ID NO: 533 - protein, 11 aa, Mus musculus
ARWYSAGYFDV

SEQ ID NO: 534 - protein, 8 aa, Mus musculus
ISGGGVTY

SEQ ID NO: 535 - protein, 11 aa, Mus musculus
ARWYSVGYFDV

SEQ ID NO: 536 - protein, 8 aa, Mus musculus
ITGGGDTY

SEQ ID NO: 537 - protein, 8 aa, Mus musculus
ITSGGGDT

SEQ ID NO: 538 - protein, 8 aa, Mus musculus
GFTFSSYA

SEQ ID NO: 539 - protein, 8 aa, Mus musculus
IFFGSSVT

SEQ ID NO: 540 - protein, 11 aa, Mus musculus
ARQGTSPWFAY

SEQ ID NO: 541 - protein, 8 aa, Mus musculus
IISGGGNT

SEQ ID NO: 542 - protein, 8 aa, Mus musculus
ISGGVGDT

SEQ ID NO: 543 - protein, 11 aa, Mus musculus
ARQGGFPWFAY

SEQ ID NO: 544 - protein, 8 aa, Mus musculus
GFNIEDTY

SEQ ID NO: 545 - protein, 8 aa, Mus musculus
IDPANGNT

SEQ ID NO: 546 - protein, 10 aa, Mus musculus
ARDGYYAPGY

SEQ ID NO: 547 - protein, 8 aa, Mus musculus
IDPANGYT

SEQ ID NO: 548 - protein, 8 aa, Mus musculus
GYTFTDYN

SEQ ID NO: 549 - protein, 8 aa, Mus musculus
INPNYDST

SEQ ID NO: 550 - protein, 14 aa, Mus musculus
ARSGFDGYYYAMDY

SEQ ID NO: 551 - protein, 8 aa, Mus musculus
DYTFTDYN

SEQ ID NO: 552 - protein, 8 aa, Mus musculus
INPNNGGT

SEQ ID NO: 553 - protein, 14 aa, Mus musculus
ARRHYGNYFYAMDY

SEQ ID NO: 554 - protein, 8 aa, Mus musculus
INPNGDFT

SEQ ID NO: 555 - protein, 8 aa, Mus musculus
INPNDDST

SEQ ID NO: 556 - protein, 8 aa, Mus musculus
ISPNYEST

SEQ ID NO: 557 - protein, 12 aa, Mus musculus
ARGGFYGNFFDY

SEQ ID NO: 558 - protein, 11 aa, Mus musculus
SIGVRRDYFAY

SEQ ID NO: 559 - protein, 8 aa, Mus musculus
IDPANGNP

SEQ ID NO: 560 - protein, 8 aa, Mus musculus
IDPANGNR

SEQ ID NO: 561 - protein, 8 aa, Mus musculus
NDPANGNT

SEQ ID NO: 562 - protein, 7 aa, Mus musculus
ASLWVDY

SEQ ID NO: 563 - protein, 8 aa, Mus musculus
GFNIKDTY

SEQ ID NO: 564 - protein, 8 aa, Mus musculus
IDPANANT

SEQ ID NO: 565 - protein, 10 aa, Mus musculus
AHDGYYAPGY

SEQ ID NO: 566 - protein, 8 aa, Mus musculus
IDPANGNS

SEQ ID NO: 567 - protein, 10 aa, Mus musculus
AHDGYYAPGN

SEQ ID NO: 568 - protein, 10 aa, Mus musculus
ARDGYYAPGH

SEQ ID NO: 569 - protein, 10 aa, Mus musculus
ARDGYSAPGY

SEQ ID NO: 570 - protein, 10 aa, Mus musculus
THDGYYAPGY

SEQ ID NO: 571 - protein, 10 aa, Mus musculus
AHDGYYAPGF

SEQ ID NO: 572 - protein, 10 aa, Mus musculus
APFSYGTLAY

SEQ ID NO: 573 - protein, 8 aa, Mus musculus
GLNIKDTY

SEQ ID NO: 574 - protein, 8 aa, Mus musculus
GVNIKDTY

SEQ ID NO: 575 - protein, 8 aa, Mus musculus
IDPANFNT

SEQ ID NO: 576 - protein, 8 aa, Mus musculus
GCNIKDYY

SEQ ID NO: 577 - protein, 11 aa, Mus musculus
TIGVRRDYFAY

SEQ ID NO: 578 - protein, 11 aa, Mus musculus
AIGVRRDYFAY

SEQ ID NO: 579 - protein, 8 aa, Mus musculus
GFNIKDFY

SEQ ID NO: 580 - protein, 11 aa, Mus musculus
AIGVRRDYFGY

SEQ ID NO: 581 - protein, 11 aa, Mus musculus
AIGVRRDYFDY

SEQ ID NO: 582 - protein, 8 aa, Mus musculus
GYTFTEYT

SEQ ID NO: 583 - protein, 8 aa, Mus musculus
INPNSGGT

SEQ ID NO: 584 - protein, 15 aa, Mus musculus
TRPHSLLRLRREFDY

SEQ ID NO: 585 - protein, 8 aa, Mus musculus
GFSFTGYT

SEQ ID NO: 586 - protein, 8 aa, Mus musculus
INTYNGGT

SEQ ID NO: 587 - protein, 11 aa, Mus musculus
ARSPGRYSMDY

SEQ ID NO: 588 - protein, 8 aa, Mus musculus
INPSTGGT

SEQ ID NO: 589 - protein, 15 aa, Mus musculus
ARPHSLLRLRREFDY

SEQ ID NO: 590 - protein, 8 aa, Mus musculus
GYTFAEYT

SEQ ID NO: 591 - protein, 8 aa, Mus musculus
GYTFTEYP

SEQ ID NO: 592 - protein, 11 aa, Mus musculus
ALYGNYWYFDV

SEQ ID NO: 593 - protein, 9 aa, Mus musculus
GYTFTEYTM

SEQ ID NO: 594 - protein, 8 aa, Mus musculus
VNPNTGGT

SEQ ID NO: 595 - protein, 8 aa, Mus musculus
INPNTGGT

SEQ ID NO: 596 - protein, 8 aa, Mus musculus
VNLNTGGT

SEQ ID NO: 597 - protein, 8 aa, Mus musculus
GYTFTSYV

SEQ ID NO: 598 - protein, 8 aa, Mus musculus
INPYHDGT

SEQ ID NO: 599 - protein, 11 aa, Mus musculus
ARSRTVIGFDY

SEQ ID NO: 600 - protein, 8 aa, Mus musculus
INPYNDAT

SEQ ID NO: 601 - protein, 9 aa, Mus musculus
ARGVRLLDY

SEQ ID NO: 602 - protein, 8 aa, Mus musculus
INPYTDGT

SEQ ID NO: 603 - protein, 11 aa, Mus musculus
AKSRTVIGFDY

SEQ ID NO: 604 - protein, 8 aa, Mus musculus
INPYNDGT

SEQ ID NO: 605 - protein, 11 aa, Mus musculus
ARSRTVVGFDY

SEQ ID NO: 606 - protein, 8 aa, Mus musculus
GYTFTTYV

SEQ ID NO: 607 - protein, 8 aa, Mus musculus
INPYNDGS

SEQ ID NO: 608 - protein, 14 aa, Mus musculus
ARWVYYDFGGSMDY

SEQ ID NO: 609 - protein, 8 aa, Mus musculus
GFTLSRYG

SEQ ID NO: 610 - protein, 8 aa, Mus musculus
SSGGGIYT

SEQ ID NO: 611 - protein, 10 aa, Mus musculus
ARHDGVPMDY

SEQ ID NO: 612 - protein, 8 aa, Mus musculus
GFTFSSYG

SEQ ID NO: 613 - protein, 8 aa, Mus musculus
IFSGDSNT

SEQ ID NO: 614 - protein, 11 aa, Mus musculus
SRQGHRSWFAY

SEQ ID NO: 615 - protein, 8 aa, Mus musculus
GFTFSTYG

SEQ ID NO: 616 - protein, 8 aa, Mus musculus
ISGGGIYT

SEQ ID NO: 617 - protein, 10 aa, Mus musculus
VRMITTYFDH

SEQ ID NO: 618 - protein, 8 aa, Mus musculus
IFSGDSYT

SEQ ID NO: 619 - protein, 11 aa, Mus musculus
ARQGHRSWFAY

SEQ ID NO: 620 - protein, 8 aa, Mus musculus
ISSGGSYT

SEQ ID NO: 621 - protein, 14 aa, Mus musculus
ARHEDTTYYYAMDY

SEQ ID NO: 622 - protein, 8 aa, Mus musculus
GFTFNTYA

SEQ ID NO: 623 - protein, 8 aa, Mus musculus
IRIKSNNY

SEQ ID NO: 624 - protein, 15 aa, Mus musculus
VRQAYGNSYFFAMDY

SEQ ID NO: 625 - protein, 8 aa, Mus musculus
GFTFNTNA

SEQ ID NO: 626 - protein, 8 aa, Mus musculus
IRSKSNNY

SEQ ID NO: 627 - protein, 5 aa, Mus musculus
TAMDY

SEQ ID NO: 628 - protein, 8 aa, Mus musculus
GYIFITYW

SEQ ID NO: 629 - protein, 8 aa, Mus musculus
IFPASGST

SEQ ID NO: 630 - protein, 12 aa, Mus musculus
ASYDGYYSWFAY

SEQ ID NO: 631 - protein, 8 aa, Mus musculus
GFTFSHSY

SEQ ID NO: 632 - protein, 8 aa, Mus musculus
IYAGTGGT

SEQ ID NO: 633 - protein, 12 aa, Mus musculus
ARHETYGSSPDY

SEQ ID NO: 634 - protein, 8 aa, Mus musculus
GFTFSSFY

SEQ ID NO: 635 - protein, 8 aa, Mus musculus
IDAGTGGT

SEQ ID NO: 636 - protein, 13 aa, Mus musculus
ARHVGRLRGYFDV

SEQ ID NO: 637 - protein, 8 aa, Mus musculus
GFTFSSNY

SEQ ID NO: 638 - protein, 8 aa, Mus musculus
IYAGTGST

SEQ ID NO: 639 - protein, 11 aa, Mus musculus
ASHDGHDAMDY

SEQ ID NO: 640 - protein, 8 aa, Mus musculus
IDAGTGGS

SEQ ID NO: 641 - protein, 8 aa, Mus musculus
GFTFSSRY

SEQ ID NO: 642 - protein, 8 aa, Mus musculus
IYAGTGGS

SEQ ID NO: 643 - protein, 8 aa, Mus musculus
GFTFSSSY

SEQ ID NO: 644 - protein, 8 aa, Mus musculus
IYAGTGDT

SEQ ID NO: 645 - protein, 12 aa, Mus musculus
ASPTGINWYFDV

SEQ ID NO: 646 - protein, 12 aa, Mus musculus
ASPTGVNWYFDV

SEQ ID NO: 647 - protein, 12 aa, Mus musculus
ARPNGYGYGMDY

SEQ ID NO: 648 - protein, 12 aa, Mus musculus
ARQDSYGRGPDC

SEQ ID NO: 649 - protein, 12 aa, Mus musculus
ARQDSYGRSPDY

SEQ ID NO: 650 - protein, 12 aa, Mus musculus
ARHETYGSGPDY

SEQ ID NO: 651 - protein, 11 aa, Mus musculus
ASHDGLDAMDY

SEQ ID NO: 652 - protein, 8 aa, Mus musculus
IYAGPGYT

SEQ ID NO: 653 - protein, 13 aa, Mus musculus
ARHVGRQRGYFDV

SEQ ID NO: 654 - protein, 12 aa, Mus musculus
GRHNGYGIGMDY

SEQ ID NO: 655 - protein, 8 aa, Mus musculus
GLTFSSSY

SEQ ID NO: 656 - protein, 8 aa, Mus musculus
GYTFTDYP

SEQ ID NO: 657 - protein, 8 aa, Mus musculus
IITYYGDA

SEQ ID NO: 658 - protein, 11 aa, Mus musculus
ASHRGKGYFDV

SEQ ID NO: 659 - protein, 8 aa, Mus musculus
GYTFTDYF

SEQ ID NO: 660 - protein, 8 aa, Mus musculus
IYPGNINT

SEQ ID NO: 661 - protein, 9 aa, Mus musculus
AGGNYALDY

SEQ ID NO: 662 - protein, 8 aa, Mus musculus
GYTFTDYY

SEQ ID NO: 663 - protein, 8 aa, Mus musculus
IYPGNDNS

SEQ ID NO: 664 - protein, 9 aa, Mus musculus
AGGNYAMDY

SEQ ID NO: 665 - protein, 8 aa, Mus musculus
GYTFTAYY

SEQ ID NO: 666 - protein, 8 aa, Mus musculus
IYPGNDNT

SEQ ID NO: 667 - protein, 8 aa, Mus musculus
GYTFTNSG

SEQ ID NO: 668 - protein, 8 aa, Mus musculus
INTYTGEP

SEQ ID NO: 669 - protein, 12 aa, Mus musculus
APTIQVRRLFDY

SEQ ID NO: 670 - protein, 12 aa, Mus musculus
APTIQVRRLVDY

SEQ ID NO: 671 - protein, 8 aa, Mus musculus
GYTFTNYG

SEQ ID NO: 672 - protein, 8 aa, Mus musculus
INTNTGEP

SEQ ID NO: 673 - protein, 11 aa, Mus musculus
ARRGYYGRGDY

SEQ ID NO: 674 - protein, 8 aa, Mus musculus
GFSLTSYD

SEQ ID NO: 675 - protein, 7 aa, Mus musculus
IWTGGGT

SEQ ID NO: 676 - protein, 12 aa, Mus musculus
VRWGDDYDWFAY

SEQ ID NO: 677 - protein, 8 aa, Mus musculus
GFSLTTYG

SEQ ID NO: 678 - protein, 8 aa, Mus musculus
IWRGGGTD

SEQ ID NO: 679 - protein, 13 aa, Mus musculus
AIYSDHDTYALDY

SEQ ID NO: 680 - protein, 8 aa, Mus musculus
GYTFTDYA

SEQ ID NO: 681 - protein, 8 aa, Mus musculus
ISNYNGDV

SEQ ID NO: 682 - protein, 11 aa, Mus musculus
ARRHGNRAMDY

SEQ ID NO: 683 - protein, 8 aa, Mus musculus
GYSFTSYY

SEQ ID NO: 684 - protein, 8 aa, Mus musculus
INPSNGGT

SEQ ID NO: 685 - protein, 13 aa, Mus musculus
TREGNYGYSWYAY

SEQ ID NO: 686 - protein, 8 aa, Mus musculus
GYTFTSYW

SEQ ID NO: 687 - protein, 8 aa, Mus musculus
IDPSDSYT

SEQ ID NO: 688 - protein, 8 aa, Mus musculus
AGGIHFDY

SEQ ID NO: 689 - protein, 8 aa, Mus musculus
GYTFTSYN

SEQ ID NO: 690 - protein, 8 aa, Mus musculus
IYPGKDDT

SEQ ID NO: 691 - protein, 13 aa, Mus musculus
ARVYYGNYGGFDY

SEQ ID NO: 692 - protein, 8 aa, Mus musculus
IYPGSGSV

SEQ ID NO: 693 - protein, 13 aa, Mus musculus
TRGGIGVRSPFDV

SEQ ID NO: 694 - protein, 8 aa, Mus musculus
GYTFTSSW

SEQ ID NO: 695 - protein, 8 aa, Mus musculus
IHPNSGNT

SEQ ID NO: 696 - protein, 8 aa, Mus musculus
VRLGLFDF

SEQ ID NO: 697 - protein, 8 aa, Mus musculus
GYTFTNYW

SEQ ID NO: 698 - protein, 8 aa, Mus musculus
ISPRTGNT

SEQ ID NO: 699 - protein, 6 aa, Mus musculus
TRGEAY

SEQ ID NO: 700 - protein, 6 aa, Mus musculus
ARGEAY

SEQ ID NO: 701 - protein, 8 aa, Mus musculus
ISPRTTYT

SEQ ID NO: 702 - protein, 8 aa, Mus musculus
GYTFTTYW

SEQ ID NO: 703 - protein, 8 aa, Mus musculus
INPSTGYT

SEQ ID NO: 704 - protein, 13 aa, Mus musculus
ASYYGTSQAWFAY

SEQ ID NO: 705 - protein, 8 aa, Mus musculus
IYPGDGDT

SEQ ID NO: 706 - protein, 13 aa, Mus musculus
ARVEADDLWYFDV

SEQ ID NO: 707 - protein, 8 aa, Mus musculus
GYAFTNYL

SEQ ID NO: 708 - protein, 8 aa, Mus musculus
INPGSGGP

SEQ ID NO: 709 - protein, 13 aa, Mus musculus
ASDNYGNYDVLDY

SEQ ID NO: 710 - protein, 8 aa, Mus musculus
INPGSGGT

SEQ ID NO: 711 - protein, 6 aa, Mus musculus
DREDAY

SEQ ID NO: 712 - protein, 8 aa, Mus musculus
INTYSSNT

SEQ ID NO: 713 - protein, 14 aa, Mus musculus
ARRRGSHRDAYFDY

SEQ ID NO: 714 - protein, 8 aa, Mus musculus
GYTFTDYV

SEQ ID NO: 715 - protein, 8 aa, Mus musculus
IYPGGNRT

SEQ ID NO: 716 - protein, 10 aa, Mus musculus
ARFNDWAMDH

SEQ ID NO: 717 - protein, 8 aa, Mus musculus
IYPGGNST

SEQ ID NO: 718 - protein, 10 aa, Mus musculus
ARFNDWSMDY

SEQ ID NO: 719 - protein, 8 aa, Mus musculus
IYPGGSSI

SEQ ID NO: 720 - protein, 10 aa, Mus musculus
ARFNDWAMDY

SEQ ID NO: 721 - protein, 8 aa, Mus musculus
GYTFTSYF

SEQ ID NO: 722 - protein, 8 aa, Mus musculus
IYPGNVNT

SEQ ID NO: 723 - protein, 8 aa, Mus musculus
ARDYAMDY

SEQ ID NO: 724 - protein, 8 aa, Mus musculus
GYTFTTYF

SEQ ID NO: 725 - protein, 8 aa, Mus musculus
IYPGNVNV

SEQ ID NO: 726 - protein, 8 aa, Mus musculus
SRDYAMDY

SEQ ID NO: 727 - protein, 8 aa, Mus musculus
GYTFTTYY

SEQ ID NO: 728 - protein, 8 aa, Mus musculus
TRDYAMDY

SEQ ID NO: 729 - protein, 8 aa, Mus musculus
INTYSGNT

SEQ ID NO: 730 - protein, 8 aa, Mus musculus
INTHSDNT

SEQ ID NO: 731 - protein, 13 aa, Mus musculus
ARPNYDGHWYFDV

SEQ ID NO: 732 - protein, 14 aa, Mus musculus
ARRRGIHRDAYFDY

SEQ ID NO: 733 - protein, 8 aa, Mus musculus
INSYSGNT

SEQ ID NO: 734 - protein, 15 aa, Mus musculus
ARWAITTGVYYAMDY

SEQ ID NO: 735 - protein, 8 aa, Mus musculus
INTYSANT

SEQ ID NO: 736 - protein, 14 aa, Mus musculus
ARRRGSHQDAYFDY

SEQ ID NO: 737 - protein, 7 aa, Mus musculus
IYPGGNR

SEQ ID NO: 738 - protein, 8 aa, Mus musculus
GYTFTDYE

SEQ ID NO: 739 - protein, 8 aa, Mus musculus
IDPETGAT

SEQ ID NO: 740 - protein, 11 aa, Mus musculus
TARQLGLPFAY

SEQ ID NO: 741 - protein, 9 aa, Mus musculus
GFSLNTSNL

SEQ ID NO: 742 - protein, 8 aa, Mus musculus
AHIWWDDD

SEQ ID NO: 743 - protein, 14 aa, Mus musculus
ARKNHYYGHWYFDV

SEQ ID NO: 744 - protein, 9 aa, Mus musculus
GFSLNTSNM

SEQ ID NO: 745 - protein, 9 aa, Mus musculus
GFSLSTPGL

SEQ ID NO: 746 - protein, 9 aa, Mus musculus
GFSLSTPNM

SEQ ID NO: 747 - protein, 14 aa, Mus musculus
ARKIHYYGHWYFDV

SEQ ID NO: 748 - protein, 9 aa, Mus musculus
GFSLSTPSM

SEQ ID NO: 749 - protein, 14 aa, Mus musculus
ARKNHYSGHWFFDV

SEQ ID NO: 750 - protein, 9 aa, Mus musculus
GFSLSTSGM

SEQ ID NO: 751 - protein, 9 aa, Mus musculus
GFSLSTSNL

SEQ ID NO: 752 - protein, 14 aa, Mus musculus
ARKSHYYGHWYFDV

SEQ ID NO: 753 - protein, 9 aa, Mus musculus
GFSLSTSNM

SEQ ID NO: 754 - protein, 14 aa, Mus musculus
ARKSHYYGHWYFDF

SEQ ID NO: 755 - protein, 14 aa, Mus musculus
GRKSHYYGHWYFDV

SEQ ID NO: 756 - protein, 14 aa, Mus musculus
ARKNHYYGHWYFDF

SEQ ID NO: 757 - protein, 9 aa, Mus musculus
GFSLSTYGI

SEQ ID NO: 758 - protein, 8 aa, Mus musculus
AHIWWNDN

SEQ ID NO: 759 - protein, 9 aa, Mus musculus
ARIDYWFAY

SEQ ID NO: 760 - protein, 6 aa, Mus musculus
QDIKSY

SEQ ID NO: 761 - protein, 3 aa, Mus musculus
YAT

SEQ ID NO: 762 - protein, 9 aa, Mus musculus
LQHGESPFT

SEQ ID NO: 763 - protein, 6 aa, Mus musculus
QDINSY

SEQ ID NO: 764 - protein, 3 aa, Mus musculus
RAN

SEQ ID NO: 765 - protein, 9 aa, Mus musculus
LQYDEFPPT

SEQ ID NO: 766 - protein, 6 aa, Mus musculus
QDINTY

SEQ ID NO: 767 - protein, 6 aa, Mus musculus
QGISSN

SEQ ID NO: 768 - protein, 3 aa, Mus musculus
HGT

SEQ ID NO: 769 - protein, 9 aa, Mus musculus
VQYAQFPYT

SEQ ID NO: 770 - protein, 6 aa, Mus musculus
QDINNY

SEQ ID NO: 771 - protein, 3 aa, Mus musculus
YTS

SEQ ID NO: 772 - protein, 9 aa, Mus musculus
QQGNTLPWT

SEQ ID NO: 773 - protein, 6 aa, Mus musculus
QDISNY

SEQ ID NO: 774 - protein, 6 aa, Mus musculus
ENIYSY

SEQ ID NO: 775 - protein, 3 aa, Mus musculus
NAK

SEQ ID NO: 776 - protein, 10 aa, Mus musculus
QHHYGIPPWT

SEQ ID NO: 777 - protein, 6 aa, Mus musculus
ENIYYT

SEQ ID NO: 778 - protein, 3 aa, Mus musculus
NAN

SEQ ID NO: 779 - protein, 9 aa, Mus musculus
KQAYDVPYT

SEQ ID NO: 780 - protein, 6 aa, Mus musculus
ENIYYS

SEQ ID NO: 781 - protein, 9 aa, Mus musculus
KQSYDVPYT

SEQ ID NO: 782 - protein, 6 aa, Mus musculus
ENIYSF

SEQ ID NO: 783 - protein, 3 aa, Mus musculus
NAR

SEQ ID NO: 784 - protein, 9 aa, Mus musculus
QHHYGPPFT

SEQ ID NO: 785 - protein, 9 aa, Mus musculus
QHHYVIPPT

SEQ ID NO: 786 - protein, 3 aa, Mus musculus
YAK

SEQ ID NO: 787 - protein, 9 aa, Mus musculus
QHHYGTPLT

SEQ ID NO: 788 - protein, 6 aa, Mus musculus
GNIHNY

SEQ ID NO: 789 - protein, 9 aa, Mus musculus
QHFWSTPWT

SEQ ID NO: 790 - protein, 6 aa, Mus musculus
ENIYSN

SEQ ID NO: 791 - protein, 3 aa, Mus musculus
AAT

SEQ ID NO: 792 - protein, 9 aa, Mus musculus
QHFWGTPFT

SEQ ID NO: 793 - protein, 6 aa, Mus musculus
QEISGY

SEQ ID NO: 794 - protein, 3 aa, Mus musculus
AAS

SEQ ID NO: 795 - protein, 9 aa, Mus musculus
LQSASYPPT

SEQ ID NO: 796 - protein, 6 aa, Mus musculus
QDINKY

SEQ ID NO: 797 - protein, 8 aa, Mus musculus
LQYDNLRT

SEQ ID NO: 798 - protein, 8 aa, Mus musculus
LQYDNLWT

SEQ ID NO: 799 - protein, 6 aa, Mus musculus
DHINNW

SEQ ID NO: 800 - protein, 3 aa, Mus musculus
GAT

SEQ ID NO: 801 - protein, 9 aa, Mus musculus
QQYWSLPYM

SEQ ID NO: 802 - protein, 9 aa, Mus musculus
QQYWSIPYT

SEQ ID NO: 803 - protein, 9 aa, Mus musculus
QQYWTIPYT

SEQ ID NO: 804 - protein, 8 aa, Mus musculus
QQYWSTPT

SEQ ID NO: 805 - protein, 6 aa, Mus musculus
DHISNW

SEQ ID NO: 806 - protein, 9 aa, Mus musculus
QQYWSLPYT

SEQ ID NO: 807 - protein, 9 aa, Mus musculus
QQGNALPWT

SEQ ID NO: 808 - protein, 9 aa, Mus musculus
QQGIALPWT

SEQ ID NO: 809 - protein, 6 aa, Mus musculus
QGIINY

SEQ ID NO: 810 - protein, 9 aa, Mus musculus
QQYSKLPYT

SEQ ID NO: 811 - protein, 6 aa, Mus musculus
HDIHNY

SEQ ID NO: 812 - protein, 6 aa, Mus musculus
QDISNF

SEQ ID NO: 813 - protein, 9 aa, Mus musculus
QQGKTLPWT

SEQ ID NO: 814 - protein, 9 aa, Mus musculus
QQGKSLPWT

SEQ ID NO: 815 - protein, 6 aa, Mus musculus
QGISNY

SEQ ID NO: 816 - protein, 9 aa, Mus musculus
QQYSKLPLT

SEQ ID NO: 817 - protein, 6 aa, Mus musculus
QGINNY

SEQ ID NO: 818 - protein, 3 aa, Mus musculus
YAS

SEQ ID NO: 819 - protein, 9 aa, Mus musculus
QQSNSWPLT

SEQ ID NO: 820 - protein, 6 aa, Mus musculus
QSINNY

SEQ ID NO: 821 - protein, 6 aa, Mus musculus
QSISNT

SEQ ID NO: 822 - protein, 9 aa, Mus musculus
QQSNDWPHT

SEQ ID NO: 823 - protein, 9 aa, Mus musculus
QQSNSWPHT

SEQ ID NO: 824 - protein, 9 aa, Mus musculus
QQSYDWPHT

SEQ ID NO: 825 - protein, 9 aa, Mus musculus
QQSYNWPHT

SEQ ID NO: 826 - protein, 6 aa, Mus musculus
QSISST

SEQ ID NO: 827 - protein, 9 aa, Mus musculus
HQSYSWPHT

SEQ ID NO: 828 - protein, 10 aa, Mus musculus
KSVSTSGYSY

SEQ ID NO: 829 - protein, 3 aa, Mus musculus
LAS

SEQ ID NO: 830 - protein, 9 aa, Mus musculus
QHSRELPYT

SEQ ID NO: 831 - protein, 10 aa, Mus musculus
QSVDYDGDNY

SEQ ID NO: 832 - protein, 9 aa, Mus musculus
QQSYEDPWT

SEQ ID NO: 833 - protein, 10 aa, Mus musculus
QSVDYNGYSY

SEQ ID NO: 834 - protein, 3 aa, Mus musculus
PAS

SEQ ID NO: 835 - protein, 9 aa, Mus musculus
QQSKEDPYT

SEQ ID NO: 836 - protein, 10 aa, Mus musculus
ESVDSFGNSF

SEQ ID NO: 837 - protein, 9 aa, Mus musculus
QQSNEDPRT

SEQ ID NO: 838 - protein, 10 aa, Mus musculus
ESVDNFGNSF

SEQ ID NO: 839 - protein, 10 aa, Mus musculus
ESVDNYGISF

SEQ ID NO: 840 - protein, 8 aa, Mus musculus
QQSKEVPT

SEQ ID NO: 841 - protein, 10 aa, Mus musculus
ESVDSFGHSF

SEQ ID NO: 842 - protein, 3 aa, Mus musculus
IAS

SEQ ID NO: 843 - protein, 10 aa, Mus musculus
ESVDSSGNSF

SEQ ID NO: 844 - protein, 3 aa, Mus musculus
RAS

SEQ ID NO: 845 - protein, 9 aa, Mus musculus
QQSNEDPFT

SEQ ID NO: 846 - protein, 10 aa, Mus musculus
ESVDSYGNSF

SEQ ID NO: 847 - protein, 9 aa, Mus musculus
QQSNEDPYT

SEQ ID NO: 848 - protein, 10 aa, Mus musculus
ESVESNGNSF

SEQ ID NO: 849 - protein, 9 aa, Mus musculus
QQTDEDPFT

SEQ ID NO: 850 - protein, 10 aa, Mus musculus
KSVSSSGYNY

SEQ ID NO: 851 - protein, 9 aa, Mus musculus
QHSGELPLT

SEQ ID NO: 852 - protein, 10 aa, Mus musculus
KSVSTSAYSY

SEQ ID NO: 853 - protein, 8 aa, Mus musculus
QHSRELLT

SEQ ID NO: 854 - protein, 8 aa, Mus musculus
QHSRELYT

SEQ ID NO: 855 - protein, 10 aa, Mus musculus
KSVSTSNYSY

SEQ ID NO: 856 - protein, 8 aa, Mus musculus
QHSRELPT

SEQ ID NO: 857 - protein, 10 aa, Mus musculus
QSVDYDGDSY

SEQ ID NO: 858 - protein, 10 aa, Mus musculus
ESVDSYGYSF

SEQ ID NO: 859 - protein, 9 aa, Mus musculus
QQNNEDPFT

SEQ ID NO: 860 - protein, 9 aa, Mus musculus
QHSRELPST

SEQ ID NO: 861 - protein, 6 aa, Mus musculus
QRINSY

SEQ ID NO: 862 - protein, 9 aa, Mus musculus
QQSSTWPLT

SEQ ID NO: 863 - protein, 6 aa, Mus musculus
QSISNY

SEQ ID NO: 864 - protein, 10 aa, Mus musculus
ETVDSYGSSF

SEQ ID NO: 865 - protein, 12 aa, Mus musculus
LSLLYSSNQKNY

SEQ ID NO: 866 - protein, 3 aa, Mus musculus
WAS

SEQ ID NO: 867 - protein, 9 aa, Mus musculus
QQYYSYPLT

SEQ ID NO: 868 - protein, 11 aa, Mus musculus
KSLLHSNGNTF

SEQ ID NO: 869 - protein, 3 aa, Mus musculus
RMS

SEQ ID NO: 870 - protein, 9 aa, Mus musculus
MQHLEFPYT

SEQ ID NO: 871 - protein, 9 aa, Mus musculus
MQHLEYPYT

SEQ ID NO: 872 - protein, 11 aa, Mus musculus
KSLLHSNGNTY

SEQ ID NO: 873 - protein, 6 aa, Mus musculus
QNVDTN

SEQ ID NO: 874 - protein, 3 aa, Mus musculus
SGS

SEQ ID NO: 875 - protein, 9 aa, Mus musculus
QQYNTYPYT

SEQ ID NO: 876 - protein, 6 aa, Mus musculus
QDVNTA

SEQ ID NO: 877 - protein, 3 aa, Mus musculus
STS

SEQ ID NO: 878 - protein, 9 aa, Mus musculus
QQHYSSPWT

SEQ ID NO: 879 - protein, 6 aa, Mus musculus
QDVGTA

SEQ ID NO: 880 - protein, 8 aa, Mus musculus
QQYSTYYT

SEQ ID NO: 881 - protein, 9 aa, Mus musculus
QQYSSYPYT

SEQ ID NO: 882 - protein, 3 aa, Mus musculus
SAS

SEQ ID NO: 883 - protein, 9 aa, Mus musculus
QQHYSTPFT

SEQ ID NO: 884 - protein, 6 aa, Mus musculus
QDVSTA

SEQ ID NO: 885 - protein, 9 aa, Mus musculus
QQHYSTPWT

SEQ ID NO: 886 - protein, 6 aa, Mus musculus
QDVTTA

SEQ ID NO: 887 - protein, 9 aa, Mus musculus
QQHYSAPWT

SEQ ID NO: 888 - protein, 6 aa, Mus musculus
QSISDY

SEQ ID NO: 889 - protein, 9 aa, Mus musculus
QNGHSFPFT

SEQ ID NO: 890 - protein, 6 aa, Mus musculus
QNISDY

SEQ ID NO: 891 - protein, 9 aa, Mus musculus
QNGHSFPLT

SEQ ID NO: 892 - protein, 9 aa, Mus musculus
QNAHSFPLT

SEQ ID NO: 893 - protein, 6 aa, Mus musculus
QRISDY

SEQ ID NO: 894 - protein, 9 aa, Mus musculus
QNSHSFPFT

SEQ ID NO: 895 - protein, 10 aa, Mus musculus
QNGHSFPPFT

SEQ ID NO: 896 - protein, 9 aa, Mus musculus
QSGHSFPLT

SEQ ID NO: 897 - protein, 3 aa, Mus musculus
YVS

SEQ ID NO: 898 - protein, 6 aa, Mus musculus
QTISDY

SEQ ID NO: 899 - protein, 6 aa, Mus musculus
QNVGTN

SEQ ID NO: 900 - protein, 9 aa, Mus musculus
QQYNNYPYT

SEQ ID NO: 901 - protein, 6 aa, Mus musculus
QNVDSN

SEQ ID NO: 902 - protein, 9 aa, Mus musculus
QQYNSYPYT

SEQ ID NO: 903 - protein, 9 aa, Mus musculus
QQYNIYPYT

SEQ ID NO: 904 - protein, 6 aa, Mus musculus
QNVGTD

SEQ ID NO: 905 - protein, 9 aa, Mus musculus
QQYNSYPFT

SEQ ID NO: 906 - protein, 11 aa, Mus musculus
QSIVHTNGNTY

SEQ ID NO: 907 - protein, 3 aa, Mus musculus
KVS

SEQ ID NO: 908 - protein, 9 aa, Mus musculus
FQGSHVPYT

SEQ ID NO: 909 - protein, 6 aa, Mus musculus
KSISKY

SEQ ID NO: 910 - protein, 9 aa, Mus musculus
QQHNEYPLT

SEQ ID NO: 911 - protein, 11 aa, Mus musculus
QSLLDSDGETY

SEQ ID NO: 912 - protein, 3 aa, Mus musculus
LVS

SEQ ID NO: 913 - protein, 8 aa, Mus musculus
WEGTHFWP

SEQ ID NO: 914 - protein, 11 aa, Mus musculus
QSLVHSNGITY

SEQ ID NO: 915 - protein, 3 aa, Mus musculus
KVF

SEQ ID NO: 916 - protein, 9 aa, Mus musculus
SQSTHVPWT

SEQ ID NO: 917 - protein, 9 aa, Mus musculus
SQSTHFPWT

SEQ ID NO: 918 - protein, 11 aa, Mus musculus
QSLVHSDGNTY

SEQ ID NO: 919 - protein, 11 aa, Mus musculus
QSLVHSNGNTY

SEQ ID NO: 920 - protein, 9 aa, Mus musculus
SQSTHAPWT

SEQ ID NO: 921 - protein, 9 aa, Mus musculus
SQSTHGPWT

SEQ ID NO: 922 - protein, 8 aa, Mus musculus
SQSTHVWT

SEQ ID NO: 923 - protein, 11 aa, Mus musculus
QSLVHSYGNTY

SEQ ID NO: 924 - protein, 11 aa, Mus musculus
QSLVYSNGNTY

SEQ ID NO: 925 - protein, 9 aa, Mus musculus
SQSTHVPYT

SEQ ID NO: 926 - protein, 11 aa, Mus musculus
QSLLDSDGKTY

SEQ ID NO: 927 - protein, 9 aa, Mus musculus
WQGTHFPFT

SEQ ID NO: 928 - protein, 7 aa, Mus musculus
SSISSNY

SEQ ID NO: 929 - protein, 3 aa, Mus musculus
RTS

SEQ ID NO: 930 - protein, 9 aa, Mus musculus
QQGSSLPRT

SEQ ID NO: 931 - protein, 7 aa, Mus musculus
SNISSNY

SEQ ID NO: 932 - protein, 9 aa, Mus musculus
QQGSSIPRT

SEQ ID NO: 933 - protein, 7 aa, Mus musculus
SSINSNY

SEQ ID NO: 934 - protein, 7 aa, Mus musculus
SIISSNY

SEQ ID NO: 935 - protein, 7 aa, Mus musculus
SSIISNY

SEQ ID NO: 936 - protein, 7 aa, Mus musculus
SSISSNH

SEQ ID NO: 937 - protein, 7 aa, Mus musculus
SSVSSSY

SEQ ID NO: 938 - protein, 9 aa, Mus musculus
QQYSGYPLT

SEQ ID NO: 939 - protein, 6 aa, Mus musculus
TDIDDD

SEQ ID NO: 940 - protein, 3 aa, Mus musculus
EGN

SEQ ID NO: 941 - protein, 9 aa, Mus musculus
LQSDNLPFT

SEQ ID NO: 942 - protein, 6 aa, Mus musculus
QTVTND

SEQ ID NO: 943 - protein, 9 aa, Mus musculus
QQDYISPYT

SEQ ID NO: 944 - protein, 5 aa, Mus musculus
SSVSY

SEQ ID NO: 945 - protein, 3 aa, Mus musculus
ATS

SEQ ID NO: 946 - protein, 9 aa, Mus musculus
QQWSSTPPT

SEQ ID NO: 947 - protein, 9 aa, Mus musculus
QQWSSNPLT

SEQ ID NO: 948 - protein, 9 aa, Mus musculus
QQYHSFPRT

SEQ ID NO: 949 - protein, 5 aa, Mus musculus
SSVNY

SEQ ID NO: 950 - protein, 9 aa, Mus musculus
QQRSTYPRT

SEQ ID NO: 951 - protein, 7 aa, Mus musculus
SSVSSSH

SEQ ID NO: 952 - protein, 9 aa, Mus musculus
HQWSSYPYT

SEQ ID NO: 953 - protein, 3 aa, Mus musculus
DTS

SEQ ID NO: 954 - protein, 5 aa, Mus musculus
SSISY

SEQ ID NO: 955 - protein, 3 aa, Mus musculus
GTS

SEQ ID NO: 956 - protein, 9 aa, Mus musculus
QQWSSYPLT

SEQ ID NO: 957 - protein, 3 aa, Mus musculus
LTS

SEQ ID NO: 958 - protein, 9 aa, Mus musculus
HQWSSNPLT

SEQ ID NO: 959 - protein, 6 aa, Mus musculus
QNVDNY

SEQ ID NO: 960 - protein, 9 aa, Mus musculus
QQDYSSPYT

SEQ ID NO: 961 - protein, 6 aa, Mus musculus
QSVIND

SEQ ID NO: 962 - protein, 9 aa, Mus musculus
QQDYNSPYT

SEQ ID NO: 963 - protein, 6 aa, Mus musculus
QFVSNF

SEQ ID NO: 964 - protein, 6 aa, Mus musculus
QSVNND

SEQ ID NO: 965 - protein, 9 aa, Mus musculus
QQDYNSPYA

SEQ ID NO: 966 - protein, 6 aa, Mus musculus
QSVSND

SEQ ID NO: 967 - protein, 9 aa, Mus musculus
QQDYSSPPT

SEQ ID NO: 968 - protein, 9 aa, Mus musculus
QQDYSSPWT

SEQ ID NO: 969 - protein, 6 aa, Mus musculus
QSVSNY

SEQ ID NO: 970 - protein, 6 aa, Mus musculus
QSVTND

SEQ ID NO: 971 - protein, 9 aa, Mus musculus
QQDYTSPPT

SEQ ID NO: 972 - protein, 9 aa, Mus musculus
QQDYNSPFT

SEQ ID NO: 973 - protein, 120 aa, Artificial Sequence
  Heavy chain variable domain
EVQLVESGGDLVQPGGSLRLSCKTSGFTFSSFYISWVRQAPGKGLEWIAWIYAGPGYTSYADSVKGRFTISRDNSRNTLYLQMNSLRAEDTAVYYCARHVGRQRGYFDVWGQGTLVTVSS

SEQ ID NO: 974 - protein, 107 aa, Artificial Sequence
  Light chain variable domain
DIQMTQSPSSLSASVGDRVTITCRASQDVSTAVAWYQQKPGKAPKLLIYSASYRYSGVPSRFSGSGSGTDFTLTISSLQPEDFATYYCQQHYSTPWTFGQGTKVEIK

SEQ ID NO: 975 - protein, 107 aa, Artificial Sequence
  Light chain variable domain
DIVMTQSQRFMSTTVGDRVSITCKASQDVSTAVAWYQQKPGQSPKLLIYSASYRYTGVPDRFTGSGSGTDFTLTISNMQSEDLADFFCQQHYSTPWTFGGGTKLEIK

SEQ ID NO: 976 - protein, 122 aa, Artificial Sequence
  Heavy chain variable domain
EVQLQQSGPELEKPGASVKLSCKASGYTFTEYTMHWVKQSHGKSLEWIGVINPNNGGTTYNQKFRGKATLTVDKSSSTAYMQLKSLTSEDSAVYYCARPHSLLRLRREFDYWGAGTTVTVSS

SEQ ID NO: 977 - protein, 106 aa, Artificial Sequence
  Light chain variable domain
DIQLTQSPSSLSASVGDRVTMTCRASSSVSYIHWFQQKPGKAPKPWIYLTSNLASGVPVRFSGSGSGTDYTFTISSLQPEDIATYYCQQWSSNPLTFGGGTKLEIK

SEQ ID NO: 978 - protein, 106 aa, Artificial Sequence
  Light chain variable domain
QIVLIQSPAIMSASPGEKVTMTCSASSSVSYMYWYQQKPGSSPRLLIYLTSNLASGVPVRFSGSGSGTSYSLTISRMEAEDAATYYCQQWSSNPLTFGGGTKLEIK

SEQ ID NO: 979 - protein, 106 aa, Artificial Sequence
  Light chain variable domain
QIVSTQSPAIMSASPGEKVTMTCSASSSVSYMQWYQQKPGTSPKRWIYLTSKLASGVPARFSGSGSGTSYSLTISSMEAEDAATYYCQQWSSNPLTFGGGTKLEIK

SEQ ID NO: 980 - protein, 122 aa, Artificial Sequence
  Heavy chain variable domain
EVQLVQSGAEVKKPGASVKVSCKTSGYTFTEYTMHWVKQAPGQGLEWIGVINPNNGGTTYNQKFQGRVTMTRDTSISTAYMELGSLRSDDTAVYYCARPHSLLRLRREFDYWGQGTMVTVSS

SEQ ID NO: 981 - protein, 117 aa, Artificial Sequence
  Heavy chain variable domain
QVQLQQPGAELVKPGASVKMSCKASGVNIKDTYIHWVKQTPGRGLEWIGRIDPANGNRKYNQKFKGKATLTADKSSSTAYMQLSSLTSEDSAVYYCARDGYYAPGYWGAGTTVTVSA

SEQ ID NO: 982 - protein, 107 aa, Artificial Sequence
  Light chain variable domain
DIQMTQSPSSLSASVGDRVTITCKASQNVGTDVAWYQQKPGKAPKALIYSASYRFSGVPYRFSGSGSGTDFTLTISSLQPEDFATYFCQQYNSYPYTFGQGTKVEIK

SEQ ID NO: 983 - protein, 107 aa, Artificial Sequence
  Light chain variable domain
DIVMTQSQRFMSTTVGDRVSITCKASQNVGTDVAWYQQKPGQSPKLLIYSASYRYTGVPDRFTGSGSGTDFTLTISNMQSEDLADFFCQQYNSYPYTFGGGTKLEIK

SEQ ID NO: 984 - protein, 117 aa, Artificial Sequence
  Heavy chain variable domain
EVQLVQSGAEVKKPGASVKVSCKASGVNIKDTYIHWVRQAPGQRLEWIGRIDPANGNRKYDPKFQGRVTITADTSASTAYMELSSLRSEDTAVYYCARDGYYAPGYWGQGTLVTVSS

(19) United States
(12) Patent Application Publication
(10) Pub. No.: US 2022/0235134 A1
(43) Pub. Date: Jul. 28, 2022

(54) ANTI-B7-H3 ANTIBODIES

(71) Applicants: Single Cell Technology, Inc., San Jose, CA (US); ACROBiosystems, Inc., Beijing (CN)

(72) Inventors: Leyan TANG, San Jose, CA (US); Allison SCHULKINS, San Jose, CA (US); Kimberly THAN, San Jose, CA (US); Chun-Nan CHEN, San Jose, CA (US); Jingyun MIAO, Beijing (CN); Xiaohui ZHANG, Beijing (CN); Xiaojuan SHI, Beijing (CN); Lin ZHANG, Beijing (CN); Pingju GE, Beijing (CN)

(73) Assignees: Single Cell Technology, Inc., San Jose, CA (US); ACROBiosystems, Inc., Beijing (CN)

(21) Appl. No.: 17/613,724

(22) PCT Filed: May 27, 2020
(86) PCT No.: PCT/CN2020/092472

§ 371 Date: Nov. 23, 2021

Related U.S. Application Data
(60) Provisional application No. 62/853,454, filed on May 28, 2019.

(51) Int. Cl. C07K 16/28; A61P 35/02

(52) U.S. Cl. CPC C07K 16/2827; C07K 2317/565; C07K 2317/24; A61P 35/02

(57) ABSTRACT

Provided are antibodies that specifically recognize B7 homology 3 protein (B7-H3).

### CROSS-REFERENCE TO RELATED APPLICATIONS

This application claims priority to U.S. Provisional Application No. 62/853,454 filed 28 May 2019, which is incorporated herein by reference in its entirety for all purposes.

### INCORPORATION OF SEQUENCE LISTING

The instant application contains a Sequence Listing which has been submitted via EFS-Web and is hereby incorporated by reference in its entirety. The ASCII copy, created on May 26, 2020, is named SCT0007-401-PC_Sequence_Listing_ST5.txt, and is 581 kilobytes in size.

### BACKGROUND

The B7 homology 3 protein (B7-H3) (also known as CD276 and B7RP-2, referred to herein as “B7-H3”) is a type I transmembrane glycoprotein of the immunoglobulin superfamily. Human B7-H3 contains a putative signal peptide, V-like and C-like Ig domains, a transmembrane region and a cytoplasmic domain. Exon duplication in humans results in the expression of two B7-H3 isoforms having either a single IgV-IgC-like domain (2IgB7-H3 isoform) or an IgV-IgC-IgV-IgC-like domain (4IgB7-H3 isoform) containing several conserved cysteine residues. The predominant B7-H3 isoform in human tissues and cell lines is the 4IgB7-H3 isoform (Steinberger et al., 2004).

B7-H3 has been reported as having both co-stimulatory and co-inhibitory signaling functions (Chapoval et al., 2001; Suh et al., 2003; Prasad et al., 2004; Wang et al., 2005). For example, in vitro studies have shown B7-H3's co-stimulatory function since B7-H3 was able to increase proliferation of cytotoxic T-lymphocytes (CTLs) and upregulate interferon gamma (IFN-7) production in the presence of anti-CD3 antibody to mimic the T cell receptor signal (Chapoval et al., 2001). Moreover, in vivo studies using cardiac allografts in B7-H3 −/− mice showed decreased production of key cytokine, chemokine and chemokine receptor mRNA transcripts {e.g., IL-2, IFN-γ, monocyte chemoattractant protein (MCP-1) and IFN-inducible protein (IP)-10) as compared to wild-type control (Wang et al., 2005). In contrast, B7-H3 co-inhibitory function has been observed, for example, in mice where B7-H3 protein inhibited T-cell activation and effector cytokine production (Suh et al., 2003). Although no ligands have been identified for human B7-H3, murine B7-H3 has been found to bind to the triggering receptor expressed on myeloid cells (TREM-) like transcript 2 (TLT-2), a modulator of adaptive and innate immunity cellular responses. Binding of murine B7-H3 to TLT-2 on CD8+ T-cells enhances T-cell effector functions such as proliferation, cytotoxicity and cytokine production (Hashiguchi et al., 2008).

B7-H3 is not constitutively expressed in many immune cells (e.g., natural killer (NK) cells, T-cells, and antigen-presenting cells (APCs)), however, its expression can be induced. Further, the expression of B7-H3 is not restricted to immune cells. B7-H3 transcripts are expressed in a variety of human tissues including colon, heart, liver, placenta, prostate, small intestine, testis, and uterus, as well as osteoblasts, fibroblasts, epithelial cells, and other cells of non-lymphoid lineage, potentially indicating immunological and non-immunological functions (Nygren et al., 2011). However, protein expression in normal tissue is typically maintained at a low level and thus, may be subject to post-transcriptional regulation.

B7-H3 is also expressed in a variety of human cancers, including prostate cancer, clear cell renal cell carcinoma, glioma, melanoma, lung cancer, non-small cell lung cancer (NSCLC), small cell lung cancer, pancreatic cancer, gastric cancer, acute myeloid leukemia (AML), non-Hodgkin's lymphoma (NHL), ovarian cancer, colorectal cancer, colon cancer, renal cancer, hepatocellular carcinoma, kidney cancer, head and neck cancer, hypopharyngeal squamous cell carcinoma, glioblastoma, neuroblastoma, breast cancer, endometrial cancer, and urothelial cell carcinoma (Chapoval et al., 2001; Saatian et al., 2004; Castriconi et al., 2004; Sun et al., 2002).

Although the role of B7-H3 in cancer cells is unclear, its expression may orchestrate signaling events that may protect cancer cells from innate and adaptive immune responses. For example, B7-H3 is overexpressed in high-grade prostatic intraepithelial neoplasia and adenocarcinomas of the prostate, and high expression levels of B7-H3 in these cancerous cells is associated with an increased risk of cancer progression after surgery (Roth et al., 2007). Further, tumor B7-H3 expression in NSCLC inversely correlated with the number of tumor-infiltrating lymphocytes and significantly correlated with lymph node metastasis (Sun et al., 2006). The level of circulating soluble B7-H3 in NSCLC patients has also been associated with higher tumor stage, tumor size, lymph node metastasis, and distant metastasis (Yamato et al., 2009). These lines of evidence suggest that B7-H3 is exploited by tumors as an immune evasion pathway (Hofmeyer et al., 2008).

B7-H3 may also play an important role in T-cell-mediated antitumor responses in a context dependent manner. For example, gastric cancer tumor cell expression of B7-H3 positively correlated with survival time, infiltration depth, and tissue type (Wu et al., 2006). Further, high expression of B7-H3 in pancreatic tumor cells was associated with patient survival after surgical resection and significantly correlated with the number of tumor-infiltrating CD8+ T-cells (Loos et al., 2009).

Thus, despite all prior advances, a need remains for improved compositions capable of binding to cancer cells and of facilitating or mediating an immune response against cancer cells. Such compositions may be used to diagnose and treat such cancers either by reducing or enhancing the capabilities of B7-H3 to mediate T-cell activation or by recognizing and killing cancer cells that express B7-H3. The present invention is directed to such compositions and to their uses in diagnostics and in the treatment of diseases such as cancer.

### SUMMARY

Provided are antibodies that recognize and specifically bind to human B7-B3. In one embodiment, the disclosure provides an antibody that binds to SEQ ID NO:1.

### BRIEF DESCRIPTION OF THE SEQUENCES

TABLE 1

| SEQ ID NO. | Description of sequence |
|---|---|
| 1 | Human B7-H3 protein sequence |
| 2 | Human B7-H3 (4Ig) His tagged protein sequence |
| 3 | Human B7-H3 (4Ig) Human Fc tagged protein sequence |
| 4 | SCT-Da001 mature heavy chain variable domain protein sequence |
| 5 | SCT-Da002 mature heavy chain variable domain protein sequence |
| 6 | SCT-Da003 mature heavy chain variable domain protein sequence |
| 7 | SCT-Da004 mature heavy chain variable domain protein sequence |
| 8 | SCT-Da005 mature heavy chain variable domain protein sequence |
| 9 | SCT-Da006 mature heavy chain variable domain protein sequence |
| 9 | SCT-Da007 mature heavy chain variable domain protein sequence |
| 9 | SCT-Da008 mature heavy chain variable domain protein sequence |
| 9 | SCT-Da009 mature heavy chain variable domain protein sequence |
| 10 | SCT-Da010 mature heavy chain variable domain protein sequence |
| 11 | SCT-Da011 mature heavy chain variable domain protein sequence |
| 11 | SCT-Da012 mature heavy chain variable domain protein sequence |
| 12 | SCT-Da013 mature heavy chain variable domain protein sequence |
| 13 | SCT-Da014 mature heavy chain variable domain protein sequence |
| 14 | SCT-Da015 mature heavy chain variable domain protein sequence |
| 15 | SCT-Da016 mature heavy chain variable domain protein sequence |
| 16 | SCT-Da017 mature heavy chain variable domain protein sequence |
| 16 | SCT-Da018 mature heavy chain variable domain protein sequence |
| 17 | SCT-Da019 mature heavy chain variable domain protein sequence |
| 18 | SCT-Da020 mature heavy chain variable domain protein sequence |
| 19 | SCT-Da021 mature heavy chain variable domain protein sequence |
| 19 | SCT-Da022 mature heavy chain variable domain protein sequence |
| 21 | SCT-Da023 mature heavy chain variable domain protein sequence |
| 22 | SCT-Da024 mature heavy chain variable domain protein sequence |
| 22 | SCT-Da025 mature heavy chain variable domain protein sequence |
| 22 | SCT-Da026 mature heavy chain variable domain protein sequence |
| 22 | SCT-Da027 mature heavy chain variable domain protein sequence |
| 23 | SCT-Da028 mature heavy chain variable domain protein sequence |
| 24 | SCT-Da029 mature heavy chain variable domain protein sequence |
| 25 | SCT-Da030 mature heavy chain variable domain protein sequence |
| 26 | SCT-Da031 mature heavy chain variable domain protein sequence |
| 27 | SCT-Da032 mature heavy chain variable domain protein sequence |
| 28 | SCT-Da033 mature heavy chain variable domain protein sequence |
| 29 | SCT-Da034 mature heavy chain variable domain protein sequence |
| 30 | SCT-Da035 mature heavy chain variable domain protein sequence |
| 31 | SCT-Da036 mature heavy chain variable domain protein sequence |
| 32 | SCT-Da037 mature heavy chain variable domain protein sequence |
| 33 | SCT-Da038 mature heavy chain variable domain protein sequence |
| 34 | SCT-Da039 mature heavy chain variable domain protein sequence |
| 35 | SCT-Da040 mature heavy chain variable domain protein sequence |
| 36 | SCT-Da041 mature heavy chain variable domain protein sequence |
| 37 | SCT-Da042 mature heavy chain variable domain protein sequence |
| 38 | SCT-Da043 mature heavy chain variable domain protein sequence |
| 39 | SCT-Da044 mature heavy chain variable domain protein sequence |
| 39 | SCT-Da045 mature heavy chain variable domain protein sequence |
| 40 | SCT-Da046 mature heavy chain variable domain protein sequence |
| 41 | SCT-Da047 mature heavy chain variable domain protein sequence |
| 41 | SCT-Da048 mature heavy chain variable domain protein sequence |
| 42 | SCT-Da049 mature heavy chain variable domain protein sequence |
| 43 | SCT-Da050 mature heavy chain variable domain protein sequence |
| 44 | SCT-Da051 mature heavy chain variable domain protein sequence |
| 45 | SCT-Da052 mature heavy chain variable domain protein sequence |
| 46 | SCT-Da053 mature heavy chain variable domain protein sequence |
| 47 | SCT-Da054 mature heavy chain variable domain protein sequence |
| 48 | SCT-Da055 mature heavy chain variable domain protein sequence |
| 49 | SCT-Da056 mature heavy chain variable domain protein sequence |
| 50 | SCT-Da057 mature heavy chain variable domain protein sequence |
| 51 | SCT-Da058 mature heavy chain variable domain protein sequence |
| 52 | SCT-Da059 mature heavy chain variable domain protein sequence |
| 53 | SCT-Da060 mature heavy chain variable domain protein sequence |
| 54 | SCT-Da061 mature heavy chain variable domain protein sequence |
| 55 | SCT-Da062 mature heavy chain variable domain protein sequence |
| 56 | SCT-Da063 mature heavy chain variable domain protein sequence |
| 57 | SCT-Da064 mature heavy chain variable domain protein sequence |
| 57 | SCT-Da065 mature heavy chain variable domain protein sequence |
| 58 | SCT-Da066 mature heavy chain variable domain protein sequence |
| 59 | SCT-Da067 mature heavy chain variable domain protein sequence |
| 59 | SCT-Da068 mature heavy chain variable domain protein sequence |
| 60 | SCT-Da069 mature heavy chain variable domain protein sequence |
| 60 | SCT-Da070 mature heavy chain variable domain protein sequence |
| 60 | SCT-Da071 mature heavy chain variable domain protein sequence |
| 60 | SCT-Da072 mature heavy chain variable domain protein sequence |
| 61 | SCT-Da073 mature heavy chain variable domain protein sequence |
| 62 | SCT-Da074 mature heavy chain variable domain protein sequence |
| 62 | SCT-Da075 mature heavy chain variable domain protein sequence |
| 62 | SCT-Da076 mature heavy chain variable domain protein sequence |
| 63 | SCT-Da077 mature heavy chain variable domain protein sequence |
| 63 | SCT-Da078 mature heavy chain variable domain protein sequence |
| 64 | SCT-Da079 mature heavy chain variable domain protein sequence |
| 65 | SCT-Da080 mature heavy chain variable domain protein sequence |
| 66 | SCT-Da081 mature heavy chain variable domain protein sequence |
| 67 | SCT-Da082 mature heavy chain variable domain protein sequence |
| 68 | SCT-Da083 mature heavy chain variable domain protein sequence |
| 68 | SCT-Da084 mature heavy chain variable domain protein sequence |
| 69 | SCT-Da085 mature heavy chain variable domain protein sequence |
| 70 | SCT-Da086 mature heavy chain variable domain protein sequence |
| 71 | SCT-Da087 mature heavy chain variable domain protein sequence |
| 72 | SCT-Da088 mature heavy chain variable domain protein sequence |
| 72 | SCT-Da089 mature heavy chain variable domain protein sequence |
| 72 | SCT-Da090 mature heavy chain variable domain protein sequence |
| 73 | SCT-Da091 mature heavy chain variable domain protein sequence |
| 74 | SCT-Da092 mature heavy chain variable domain protein sequence |
| 75 | SCT-Da093 mature heavy chain variable domain protein sequence |
| 75 | SCT-Da094 mature heavy chain variable domain protein sequence |
| 75 | SCT-Da095 mature heavy chain variable domain protein sequence |
| 76 | SCT-Da096 mature heavy chain variable domain protein sequence |
| 77 | SCT-Da097 mature heavy chain variable domain protein sequence |
| 77 | SCT-Da098 mature heavy chain variable domain protein sequence |
| 77 | SCT-Da099 mature heavy chain variable domain protein sequence |
| 77 | SCT-Da100 mature heavy chain variable domain protein sequence |
| 78 | SCT-Da101 mature heavy chain variable domain protein sequence |
| 79 | SCT-Da102 mature heavy chain variable domain protein sequence |
| 80 | SCT-Da103 mature heavy chain variable domain protein sequence |
| 80 | SCT-Da104 mature heavy chain variable domain protein sequence |
| 80 | SCT-Da105 mature heavy chain variable domain protein sequence |
| 81 | SCT-Da106 mature heavy chain variable domain protein sequence |
| 82 | SCT-Da107 mature heavy chain variable domain protein sequence |
| 82 | SCT-Da108 mature heavy chain variable domain protein sequence |
| 82 | SCT-Da109 mature heavy chain variable domain protein sequence |
| 82 | SCT-Da110 mature heavy chain variable domain protein sequence |
| 83 | SCT-Da111 mature heavy chain variable domain protein sequence |
| 83 | SCT-Da112 mature heavy chain variable domain protein sequence |
| 83 | SCT-Da113 mature heavy chain variable domain protein sequence |
| 83 | SCT-Da114 mature heavy chain variable domain protein sequence |
| 84 | SCT-Da115 mature heavy chain variable domain protein sequence |
| 85 | SCT-Da116 mature heavy chain variable domain protein sequence |
| 85 | SCT-Da117 mature heavy chain variable domain protein sequence |
| 85 | SCT-Da118 mature heavy chain variable domain protein sequence |
| 85 | SCT-Da119 mature heavy chain variable domain protein sequence |
| 86 | SCT-Da120 mature heavy chain variable domain protein sequence |
| 87 | SCT-Da121 mature heavy chain variable domain protein sequence |
| 87 | SCT-Da122 mature heavy chain variable domain protein sequence |
| 87 | SCT-Da123 mature heavy chain variable domain protein sequence |
| 87 | SCT-Da124 mature heavy chain variable domain protein sequence |
| 87 | SCT-Da125 mature heavy chain variable domain protein sequence |
| 87 | SCT-Da126 mature heavy chain variable domain protein sequence |
| 87 | SCT-Da127 mature heavy chain variable domain protein sequence |
| 87 | SCT-Da128 mature heavy chain variable domain protein sequence |
| 88 | SCT-Da129 mature heavy chain variable domain protein sequence |
| 89 | SCT-Da130 mature heavy chain variable domain protein sequence |
| 89 | SCT-Da131 mature heavy chain variable domain protein sequence |
| 90 | SCT-Da132 mature heavy chain variable domain protein sequence |
| 91 | SCT-Da133 mature heavy chain variable domain protein sequence |
| 91 | SCT-Da134 mature heavy chain variable domain protein sequence |
| 91 | SCT-Da135 mature heavy chain variable domain protein sequence |
| 91 | SCT-Da136 mature heavy chain variable domain protein sequence |
| 92 | SCT-Da137 mature heavy chain variable domain protein sequence |
| 92 | SCT-Da138 mature heavy chain variable domain protein sequence |
| 92 | SCT-Da139 mature heavy chain variable domain protein sequence |
| 93 | SCT-Da140 mature heavy chain variable domain protein sequence |
| 94 | SCT-Da141 mature heavy chain variable domain protein sequence |
| 95 | SCT-Da142 mature heavy chain variable domain protein sequence |
| 96 | SCT-Da143 mature heavy chain variable domain protein sequence |
| 97 | SCT-Da144 mature heavy chain variable domain protein sequence |
| 98 | SCT-Da145 mature heavy chain variable domain protein sequence |
| 99 | SCT-Da146 mature heavy chain variable domain protein sequence |
| 99 | SCT-Da147 mature heavy chain variable domain protein sequence |
| 99 | SCT-Da148 mature heavy chain variable domain protein sequence |
| 99 | SCT-Da149 mature heavy chain variable domain protein sequence |
| 99 | SCT-Da150 mature heavy chain variable domain protein sequence |
| 99 | SCT-Da151 mature heavy chain variable domain protein sequence |
| 99 | SCT-Da152 mature heavy chain variable domain protein sequence |
| 99 | SCT-Da153 mature heavy chain variable domain protein sequence |
| 99 | SCT-Da154 mature heavy chain variable domain protein sequence |
| 100 | SCT-Da155 mature heavy chain variable domain protein sequence |
| 101 | SCT-Da156 mature heavy chain variable domain protein sequence |
| 101 | SCT-Da157 mature heavy chain variable domain protein sequence |
| 101 | SCT-Da158 mature heavy chain variable domain protein sequence |
| 101 | SCT-Da159 mature heavy chain variable domain protein sequence |
| 102 | SCT-Da160 mature heavy chain variable domain protein sequence |
| 103 | SCT-Da161 mature heavy chain variable domain protein sequence |
| 104 | SCT-Da162 mature heavy chain variable domain protein sequence |
| 105 | SCT-Da163 mature heavy chain variable domain protein sequence |
| 105 | SCT-Da164 mature heavy chain variable domain protein sequence |
| 105 | SCT-Da165 mature heavy chain variable domain protein sequence |
| 105 | SCT-Da166 mature heavy chain variable domain protein sequence |
| 106 | SCT-Da167 mature heavy chain variable domain protein sequence |
| 106 | SCT-Da168 mature heavy chain variable domain protein sequence |
| 106 | SCT-Da169 mature heavy chain variable domain protein sequence |
| 106 | SCT-Da170 mature heavy chain variable domain protein sequence |
| 106 | SCT-Da171 mature heavy chain variable domain protein sequence |
| 106 | SCT-Da172 mature heavy chain variable domain protein sequence |
| 106 | SCT-Da173 mature heavy chain variable domain protein sequence |
| 106 | SCT-Da174 mature heavy chain variable domain protein sequence |
| 107 | SCT-Da175 mature heavy chain variable domain protein sequence |
| 108 | SCT-Da176 mature heavy chain variable domain protein sequence |
| 108 | SCT-Da177 mature heavy chain variable domain protein sequence |
| 108 | SCT-Da178 mature heavy chain variable domain protein sequence |
| 109 | SCT-Da179 mature heavy chain variable domain protein sequence |
| 110 | SCT-Da180 mature heavy chain variable domain protein sequence |
| 111 | SCT-Da181 mature heavy chain variable domain protein sequence |
| 112 | SCT-Da182 mature heavy chain variable domain protein sequence |
| 113 | SCT-Da183 mature heavy chain variable domain protein sequence |
| 114 | SCT-Da184 mature heavy chain variable domain protein sequence |
| 115 | SCT-Da185 mature heavy chain variable domain protein sequence |
| 116 | SCT-Da186 mature heavy chain variable domain protein sequence |
| 117 | SCT-Da187 mature heavy chain variable domain protein sequence |
| 118 | SCT-Da188 mature heavy chain variable domain protein sequence |
| 119 | SCT-Da189 mature heavy chain variable domain protein sequence |
| 120 | SCT-Da190 mature heavy chain variable domain protein sequence |
| 121 | SCT-Da191 mature heavy chain variable domain protein sequence |
| 121 | SCT-Da192 mature heavy chain variable domain protein sequence |
| 122 | SCT-Da193 mature heavy chain variable domain protein sequence |
| 123 | SCT-Da194 mature heavy chain variable domain protein sequence |
| 124 | SCT-Da195 mature heavy chain variable domain protein sequence |
| 124 | SCT-Da196 mature heavy chain variable domain protein sequence |
| 125 | SCT-Da197 mature heavy chain variable domain protein sequence |
| 126 | SCT-Da198 mature heavy chain variable domain protein sequence |
| 126 | SCT-Da199 mature heavy chain variable domain protein sequence |
| 126 | SCT-Da200 mature heavy chain variable domain protein sequence |
| 127 | SCT-Da201 mature heavy chain variable domain protein sequence |
| 128 | SCT-Da202 mature heavy chain variable domain protein sequence |
| 128 | SCT-Da203 mature heavy chain variable domain protein sequence |
| 128 | SCT-Da204 mature heavy chain variable domain protein sequence |
| 128 | SCT-Da205 mature heavy chain variable domain protein sequence |
| 129 | SCT-Da206 mature heavy chain variable domain protein sequence |
| 130 | SCT-Da207 mature heavy chain variable domain protein sequence |
| 131 | SCT-Da208 mature heavy chain variable domain protein sequence |
| 132 | SCT-Da209 mature heavy chain variable domain protein sequence |
| 133 | SCT-Da210 mature heavy chain variable domain protein sequence |
| 134 | SCT-Da211 mature heavy chain variable domain protein sequence |
| 135 | SCT-Da212 mature heavy chain variable domain protein sequence |
| 136 | SCT-Da213 mature heavy chain variable domain protein sequence |
| 137 | SCT-Da214 mature heavy chain variable domain protein sequence |
| 138 | SCT-Da215 mature heavy chain variable domain protein sequence |
| 139 | SCT-Da216 mature heavy chain variable domain protein sequence |
| 139 | SCT-Da217 mature heavy chain variable domain protein sequence |
| 140 | SCT-Da218 mature heavy chain variable domain protein sequence |
| 141 | SCT-Da219 mature heavy chain variable domain protein sequence |
| 142 | SCT-Da220 mature heavy chain variable domain protein sequence |
| 143 | SCT-Da221 mature heavy chain variable domain protein sequence |
| 144 | SCT-Da222 mature heavy chain variable domain protein sequence |
| 144 | SCT-Da223 mature heavy chain variable domain protein sequence |
| 145 | SCT-Da224 mature heavy chain variable domain protein sequence |
| 146 | SCT-Da225 mature heavy chain variable domain protein sequence |
| 147 | SCT-Da226 mature heavy chain variable domain protein sequence |
| 148 | SCT-Da227 mature heavy chain variable domain protein sequence |
| 149 | SCT-Da228 mature heavy chain variable domain protein sequence |
| 150 | SCT-Da229 mature heavy chain variable domain protein sequence |
| 151 | SCT-Da230 mature heavy chain variable domain protein sequence |
| 152 | SCT-Da231 mature heavy chain variable domain protein sequence |
| 153 | SCT-Da232 mature heavy chain variable domain protein sequence |
| 154 | SCT-Da233 mature heavy chain variable domain protein sequence |
| 155 | SCT-Da234 mature heavy chain variable domain protein sequence |
| 156 | SCT-Da235 mature heavy chain variable domain protein sequence |
| 157 | SCT-Da236 mature heavy chain variable domain protein sequence |
| 157 | SCT-Da237 mature heavy chain variable domain protein sequence |
| 158 | SCT-Da238 mature heavy chain variable domain protein sequence |
| 159 | SCT-Da239 mature heavy chain variable domain protein sequence |
| 160 | SCT-Da240 mature heavy chain variable domain protein sequence |
| 161 | SCT-Da241 mature heavy chain variable domain protein sequence |
| 162 | SCT-Da242 mature heavy chain variable domain protein sequence |
| 163 | SCT-Da243 mature heavy chain variable domain protein sequence |
| 164 | SCT-Da244 mature heavy chain variable domain protein sequence |
| 165 | SCT-Da245 mature heavy chain variable domain protein sequence |
| 165 | SCT-Da246 mature heavy chain variable domain protein sequence |
| 166 | SCT-Da247 mature heavy chain variable domain protein sequence |
| 167 | SCT-Da248 mature heavy chain variable domain protein sequence |
| 168 | SCT-Da249 mature heavy chain variable domain protein sequence |
| 169 | SCT-Da250 mature heavy chain variable domain protein sequence |
| 170 | SCT-Da251 mature heavy chain variable domain protein sequence |
| 171 | SCT-Da252 mature heavy chain variable domain protein sequence |
| 172 | SCT-Da253 mature heavy chain variable domain protein sequence |
| 173 | SCT-Da254 mature heavy chain variable domain protein sequence |
| 173 | SCT-Da255 mature heavy chain variable domain protein sequence |
| 174 | SCT-Da256 mature heavy chain variable domain protein sequence |
| 175 | SCT-Da257 mature heavy chain variable domain protein sequence |
| 176 | SCT-Da258 mature heavy chain variable domain protein sequence |
| 177 | SCT-Da259 mature heavy chain variable domain protein sequence |
| 178 | SCT-Da260 mature heavy chain variable domain protein sequence |
| 179 | SCT-Da261 mature heavy chain variable domain protein sequence |
| 180 | SCT-Da262 mature heavy chain variable domain protein sequence |
| 181 | SCT-Da263 mature heavy chain variable domain protein sequence |
| 182 | SCT-Da264 mature heavy chain variable domain protein sequence |
| 183 | SCT-Da265 mature heavy chain variable domain protein sequence |
| 184 | SCT-Da266 mature heavy chain variable domain protein sequence |
| 185 | SCT-Da267 mature heavy chain variable domain protein sequence |
| 186 | SCT-Da268 mature heavy chain variable domain protein sequence |
| 187 | SCT-Da269 mature heavy chain variable domain protein sequence |
| 188 | SCT-Da270 mature heavy chain variable domain protein sequence |
| 188 | SCT-Da271 mature heavy chain variable domain protein sequence |
| 189 | SCT-Da272 mature heavy chain variable domain protein sequence |
| 190 | SCT-Da273 mature heavy chain variable domain protein sequence |
| 190 | SCT-Da274 mature heavy chain variable domain protein sequence |
| 191 | SCT-Da275 mature heavy chain variable domain protein sequence |
| 192 | SCT-Da276 mature heavy chain variable domain protein sequence |
| 193 | SCT-Da277 mature heavy chain variable domain protein sequence |
| 194 | SCT-Da278 mature heavy chain variable domain protein sequence |
| 194 | SCT-Da279 mature heavy chain variable domain protein sequence |
| 196 | SCT-Da280 mature heavy chain variable domain protein sequence |
| 197 | SCT-Da281 mature heavy chain variable domain protein sequence |
| 198 | SCT-Da282 mature heavy chain variable domain protein sequence |
| 199 | SCT-Da283 mature heavy chain variable domain protein sequence |
| 199 | SCT-Da284 mature heavy chain variable domain protein sequence |
| 199 | SCT-Da285 mature heavy chain variable domain protein sequence |
| 199 | SCT-Da286 mature heavy chain variable domain protein sequence |
| 199 | SCT-Da287 mature heavy chain variable domain protein sequence |
| 199 | SCT-Da288 mature heavy chain variable domain protein sequence |
| 199 | SCT-Da289 mature heavy chain variable domain protein sequence |
| 199 | SCT-Da290 mature heavy chain variable domain protein sequence |
| 199 | SCT-Da291 mature heavy chain variable domain protein sequence |
| 199 | SCT-Da292 mature heavy chain variable domain protein sequence |
| 199 | SCT-Da293 mature heavy chain variable domain protein sequence |
| 200 | SCT-Da294 mature heavy chain variable domain protein sequence |
| 200 | SCT-Da295 mature heavy chain variable domain protein sequence |
| 200 | SCT-Da296 mature heavy chain variable domain protein sequence |
| 200 | SCT-Da297 mature heavy chain variable domain protein sequence |
| 200 | SCT-Da298 mature heavy chain variable domain protein sequence |
| 200 | SCT-Da299 mature heavy chain variable domain protein sequence |
| 200 | SCT-Da300 mature heavy chain variable domain protein sequence |
| 200 | SCT-Da301 mature heavy chain variable domain protein sequence |
| 200 | SCT-Da302 mature heavy chain variable domain protein sequence |
| 200 | SCT-Da303 mature heavy chain variable domain protein sequence |
| 201 | SCT-Da304 mature heavy chain variable domain protein sequence |
| 202 | SCT-Da305 mature heavy chain variable domain protein sequence |
| 203 | SCT-Da306 mature heavy chain variable domain protein sequence |
| 204 | SCT-Da307 mature heavy chain variable domain protein sequence |
| 204 | SCT-Da308 mature heavy chain variable domain protein sequence |
| 205 | SCT-Da309 mature heavy chain variable domain protein sequence |
| 205 | SCT-Da310 mature heavy chain variable domain protein sequence |
| 206 | SCT-Da311 mature heavy chain variable domain protein sequence |
| 207 | SCT-Da312 mature heavy chain variable domain protein sequence |
| 208 | SCT-Da313 mature heavy chain variable domain protein sequence |
| 209 | SCT-Da314 mature heavy chain variable domain protein sequence |
| 210 | SCT-Da315 mature heavy chain variable domain protein sequence |
| 211 | SCT-Da316 mature heavy chain variable domain protein sequence |
| 212 | SCT-Da317 mature heavy chain variable domain protein sequence |
| 213 | SCT-Da318 mature heavy chain variable domain protein sequence |
| 214 | SCT-Da319 mature heavy chain variable domain protein sequence |
| 215 | SCT-Da320 mature heavy chain variable domain protein sequence |
| 215 | SCT-Da321 mature heavy chain variable domain protein sequence |
| 216 | SCT-Da322 mature heavy chain variable domain protein sequence |
| 217 | SCT-Da323 mature heavy chain variable domain protein sequence |
| 218 | SCT-Da324 mature heavy chain variable domain protein sequence |
| 219 | SCT-Da325 mature heavy chain variable domain protein sequence |
| 220 | SCT-Da326 mature heavy chain variable domain protein sequence |
| 221 | SCT-Da327 mature heavy chain variable domain protein sequence |
| 222 | SCT-Da328 mature heavy chain variable domain protein sequence |
| 222 | SCT-Da329 mature heavy chain variable domain protein sequence |
| 222 | SCT-Da330 mature heavy chain variable domain protein sequence |
| 223 | SCT-Da331 mature heavy chain variable domain protein sequence |
| 224 | SCT-Da332 mature heavy chain variable domain protein sequence |
| 225 | SCT-Da333 mature heavy chain variable domain protein sequence |
| 225 | SCT-Da334 mature heavy chain variable domain protein sequence |
| 225 | SCT-Da335 mature heavy chain variable domain protein sequence |
| 226 | SCT-Da336 mature heavy chain variable domain protein sequence |
| 227 | SCT-Da337 mature heavy chain variable domain protein sequence |
| 228 | SCT-Da338 mature heavy chain variable domain protein sequence |
| 229 | SCT-Da339 mature heavy chain variable domain protein sequence |
| 230 | SCT-Da340 mature heavy chain variable domain protein sequence |
| 231 | SCT-Da341 mature heavy chain variable domain protein sequence |
| 232 | SCT-Da342 mature heavy chain variable domain protein sequence |
| 233 | SCT-Da343 mature heavy chain variable domain protein sequence |
| 234 | SCT-Da344 mature heavy chain variable domain protein sequence |
| 235 | SCT-Da345 mature heavy chain variable domain protein sequence |
| 236 | SCT-Da346 mature heavy chain variable domain protein sequence |
| 237 | SCT-Da347 mature heavy chain variable domain protein sequence |
| 238 | SCT-Da348 mature heavy chain variable domain protein sequence |
| 239 | SCT-Da349 mature heavy chain variable domain protein sequence |
| 239 | SCT-Da350 mature heavy chain variable domain protein sequence |
| 239 | SCT-Da351 mature heavy chain variable domain protein sequence |
| 240 | SCT-Da352 mature heavy chain variable domain protein sequence |
| 241 | SCT-Da353 mature heavy chain variable domain protein sequence |
| 242 | SCT-Da354 mature heavy chain variable domain protein sequence |
| 242 | SCT-Da355 mature heavy chain variable domain protein sequence |
| 242 | SCT-Da356 mature heavy chain variable domain protein sequence |
| 242 | SCT-Da357 mature heavy chain variable domain protein sequence |
| 243 | SCT-Da358 mature heavy chain variable domain protein sequence |
| 244 | SCT-Da359 mature heavy chain variable domain protein sequence |
| 245 | SCT-Da360 mature heavy chain variable domain protein sequence |
| 246 | SCT-Da361 mature heavy chain variable domain protein sequence |
| 246 | SCT-Da362 mature heavy chain variable domain protein sequence |
| 247 | SCT-Da001 mature light chain variable domain protein sequence |
| 248 | SCT-Da002 mature light chain variable domain protein sequence |
| 249 | SCT-Da003 mature light chain variable domain protein sequence |
| 249 | SCT-Da071 mature light chain variable domain protein sequence |
| 249 | SCT-Da073 mature light chain variable domain protein sequence |
| 249 | SCT-Da076 mature light chain variable domain protein sequence |
| 250 | SCT-Da004 mature light chain variable domain protein sequence |
| 250 | SCT-Da021 mature light chain variable domain protein sequence |
| 250 | SCT-Da022 mature light chain variable domain protein sequence |
| 250 | SCT-Da025 mature light chain variable domain protein sequence |
| 251 | SCT-Da005 mature light chain variable domain protein sequence |
| 252 | SCT-Da006 mature light chain variable domain protein sequence |
| 252 | SCT-Da283 mature light chain variable domain protein sequence |
| 253 | SCT-Da007 mature light chain variable domain protein sequence |
| 254 | SCT-Da008 mature light chain variable domain protein sequence |
| 254 | SCT-Da011 mature light chain variable domain protein sequence |
| 254 | SCT-Da013 mature light chain variable domain protein sequence |
| 255 | SCT-Da009 mature light chain variable domain protein sequence |
| 256 | SCT-Da010 mature light chain variable domain protein sequence |
| 256 | SCT-Da012 mature light chain variable domain protein sequence |
| 257 | SCT-Da014 mature light chain variable domain protein sequence |
| 257 | SCT-Da019 mature light chain variable domain protein sequence |
| 257 | SCT-Da080 mature light chain variable domain protein sequence |
| 258 | SCT-Da015 mature light chain variable domain protein sequence |
| 259 | SCT-Da016 mature light chain variable domain protein sequence |
| 260 | SCT-Da017 mature light chain variable domain protein sequence |
| 261 | SCT-Da018 mature light chain variable domain protein sequence |
| 261 | SCT-Da164 mature light chain variable domain protein sequence |
| 262 | SCT-Da020 mature light chain variable domain protein sequence |
| 262 | SCT-Da175 mature light chain variable domain protein sequence |
| 263 | SCT-Da023 mature light chain variable domain protein sequence |
| 263 | SCT-Da024 mature light chain variable domain protein sequence |
| 264 | SCT-Da026 mature light chain variable domain protein sequence |
| 264 | SCT-Da063 mature light chain variable domain protein sequence |
| 264 | SCT-Da068 mature light chain variable domain protein sequence |
| 264 | SCT-Da077 mature light chain variable domain protein sequence |
| 264 | SCT-Da285 mature light chain variable domain protein sequence |
| 264 | SCT-Da296 mature light chain variable domain protein sequence |
| 265 | SCT-Da027 mature light chain variable domain protein sequence |
| 265 | SCT-Da303 mature light chain variable domain protein sequence |
| 265 | SCT-Da357 mature light chain variable domain protein sequence |
| 266 | SCT-Da028 mature light chain variable domain protein sequence |
| 266 | SCT-Da034 mature light chain variable domain protein sequence |
| 267 | SCT-Da029 mature light chain variable domain protein sequence |
| 267 | SCT-Da163 mature light chain variable domain protein sequence |
| 268 | SCT-Da030 mature light chain variable domain protein sequence |
| 269 | SCT-Da031 mature light chain variable domain protein sequence |
| 270 | SCT-Da032 mature light chain variable domain protein sequence |
| 271 | SCT-Da033 mature light chain variable domain protein sequence |
| 272 | SCT-Da035 mature light chain variable domain protein sequence |
| 273 | SCT-Da036 mature light chain variable domain protein sequence |
| 274 | SCT-Da037 mature light chain variable domain protein sequence |
| 275 | SCT-Da038 mature light chain variable domain protein sequence |
| 276 | SCT-Da039 mature light chain variable domain protein sequence |
| 277 | SCT-Da040 mature light chain variable domain protein sequence |
| 278 | SCT-Da041 mature light chain variable domain protein sequence |
| 279 | SCT-Da042 mature light chain variable domain protein sequence |
| 280 | SCT-Da043 mature light chain variable domain protein sequence |
| 281 | SCT-Da044 mature light chain variable domain protein sequence |
| 282 | SCT-Da045 mature light chain variable domain protein sequence |
| 283 | SCT-Da046 mature light chain variable domain protein sequence |
| 283 | SCT-Da083 mature light chain variable domain protein sequence |
| 283 | SCT-Da176 mature light chain variable domain protein sequence |
| 284 | SCT-Da047 mature light chain variable domain protein sequence |
| 284 | SCT-Da146 mature light chain variable domain protein sequence |
| 285 | SCT-Da048 mature light chain variable domain protein sequence |
| 286 | SCT-Da049 mature light chain variable domain protein sequence |
| 287 | SCT-Da050 mature light chain variable domain protein sequence |
| 287 | SCT-Da053 mature light chain variable domain protein sequence |
| 288 | SCT-Da051 mature light chain variable domain protein sequence |
| 289 | SCT-Da052 mature light chain variable domain protein sequence |
| 290 | SCT-Da054 mature light chain variable domain protein sequence |
| 291 | SCT-Da055 mature light chain variable domain protein sequence |
| 292 | SCT-Da056 mature light chain variable domain protein sequence |
| 293 | SCT-Da057 mature light chain variable domain protein sequence |
| 293 | SCT-Da086 mature light chain variable domain protein sequence |
| 293 | SCT-Da090 mature light chain variable domain protein sequence |
| 293 | SCT-Da286 mature light chain variable domain protein sequence |
| 294 | SCT-Da058 mature light chain variable domain protein sequence |
| 294 | SCT-Da059 mature light chain variable domain protein sequence |
| 294 | SCT-Da134 mature light chain variable domain protein sequence |
| 294 | SCT-Da198 mature light chain variable domain protein sequence |
| 294 | SCT-Da202 mature light chain variable domain protein sequence |
| 294 | SCT-Da288 mature light chain variable domain protein sequence |
| 295 | SCT-Da060 mature light chain variable domain protein sequence |
| 296 | SCT-Da061 mature light chain variable domain protein sequence |
| 296 | SCT-Da147 mature light chain variable domain protein sequence |
| 297 | SCT-Da062 mature light chain variable domain protein sequence |
| 298 | SCT-Da064 mature light chain variable domain protein sequence |
| 299 | SCT-Da065 mature light chain variable domain protein sequence |
| 300 | SCT-Da066 mature light chain variable domain protein sequence |
| 301 | SCT-Da067 mature light chain variable domain protein sequence |
| 301 | SCT-Da284 mature light chain variable domain protein sequence |
| 302 | SCT-Da069 mature light chain variable domain protein sequence |
| 303 | SCT-Da070 mature light chain variable domain protein sequence |
| 304 | SCT-Da072 mature light chain variable domain protein sequence |
| 304 | SCT-Da116 mature light chain variable domain protein sequence |
| 304 | SCT-Da145 mature light chain variable domain protein sequence |
| 304 | SCT-Da148 mature light chain variable domain protein sequence |
| 304 | SCT-Da157 mature light chain variable domain protein sequence |
| 304 | SCT-Da299 mature light chain variable domain protein sequence |
| 304 | SCT-Da349 mature light chain variable domain protein sequence |
| 305 | SCT-Da074 mature light chain variable domain protein sequence |
| 305 | SCT-Da089 mature light chain variable domain protein sequence |
| 305 | SCT-Da247 mature light chain variable domain protein sequence |
| 306 | SCT-Da075 mature light chain variable domain protein sequence |
| 307 | SCT-Da078 mature light chain variable domain protein sequence |
| 308 | SCT-Da079 mature light chain variable domain protein sequence |
| 309 | SCT-Da081 mature light chain variable domain protein sequence |
| 310 | SCT-Da082 mature light chain variable domain protein sequence |
| 311 | SCT-Da084 mature light chain variable domain protein sequence |
| 312 | SCT-Da085 mature light chain variable domain protein sequence |
| 312 | SCT-Da172 mature light chain variable domain protein sequence |
| 312 | SCT-Da291 mature light chain variable domain protein sequence |
| 313 | SCT-Da087 mature light chain variable domain protein sequence |
| 313 | SCT-Da131 mature light chain variable domain protein sequence |
| 314 | SCT-Da088 mature light chain variable domain protein sequence |
| 314 | SCT-Da156 mature light chain variable domain protein sequence |
| 315 | SCT-Da091 mature light chain variable domain protein sequence |
| 316 | SCT-Da092 mature light chain variable domain protein sequence |
| 316 | SCT-Da140 mature light chain variable domain protein sequence |
| 317 | SCT-Da093 mature light chain variable domain protein sequence |
| 317 | SCT-Da209 mature light chain variable domain protein sequence |
| 317 | SCT-Da211 mature light chain variable domain protein sequence |
| 318 | SCT-Da094 mature light chain variable domain protein sequence |
| 318 | SCT-Da101 mature light chain variable domain protein sequence |
| 318 | SCT-Da105 mature light chain variable domain protein sequence |
| 318 | SCT-Da106 mature light chain variable domain protein sequence |
| 318 | SCT-Da112 mature light chain variable domain protein sequence |
| 318 | SCT-Da118 mature light chain variable domain protein sequence |
| 318 | SCT-Da129 mature light chain variable domain protein sequence |
| 318 | SCT-Da151 mature light chain variable domain protein sequence |
| 318 | SCT-Da289 mature light chain variable domain protein sequence |
| 318 | SCT-Da300 mature light chain variable domain protein sequence |
| 319 | SCT-Da095 mature light chain variable domain protein sequence |
| 320 | SCT-Da096 mature light chain variable domain protein sequence |
| 321 | SCT-Da097 mature light chain variable domain protein sequence |
| 321 | SCT-Da102 mature light chain variable domain protein sequence |
| 322 | SCT-Da098 mature light chain variable domain protein sequence |
| 322 | SCT-Da159 mature light chain variable domain protein sequence |
| 323 | SCT-Da099 mature light chain variable domain protein sequence |
| 324 | SCT-Da100 mature light chain variable domain protein sequence |
| 324 | SCT-Da301 mature light chain variable domain protein sequence |
| 325 | SCT-Da103 mature light chain variable domain protein sequence |
| 326 | SCT-Da104 mature light chain variable domain protein sequence |
| 326 | SCT-Da117 mature light chain variable domain protein sequence |
| 326 | SCT-Da123 mature light chain variable domain protein sequence |
| 327 | SCT-Da107 mature light chain variable domain protein sequence |
| 328 | SCT-Da108 mature light chain variable domain protein sequence |
| 328 | SCT-Da295 mature light chain variable domain protein sequence |
| 329 | SCT-Da109 mature light chain variable domain protein sequence |
| 329 | SCT-Da133 mature light chain variable domain protein sequence |
| 329 | SCT-Da191 mature light chain variable domain protein sequence |
| 329 | SCT-Da222 mature light chain variable domain protein sequence |
| 330 | SCT-Da110 mature light chain variable domain protein sequence |
| 331 | SCT-Da111 mature light chain variable domain protein sequence |
| 331 | SCT-Da327 mature light chain variable domain protein sequence |
| 332 | SCT-Da113 mature light chain variable domain protein sequence |
| 332 | SCT-Da119 mature light chain variable domain protein sequence |
| 332 | SCT-Da158 mature light chain variable domain protein sequence |
| 333 | SCT-Da114 mature light chain variable domain protein sequence |
| 333 | SCT-Da128 mature light chain variable domain protein sequence |
| 333 | SCT-Da135 mature light chain variable domain protein sequence |
| 333 | SCT-Da181 mature light chain variable domain protein sequence |
| 333 | SCT-Da183 mature light chain variable domain protein sequence |
| 333 | SCT-Da188 mature light chain variable domain protein sequence |
| 333 | SCT-Da189 mature light chain variable domain protein sequence |
| 333 | SCT-Da192 mature light chain variable domain protein sequence |
| 333 | SCT-Da200 mature light chain variable domain protein sequence |
| 333 | SCT-Da201 mature light chain variable domain protein sequence |
| 333 | SCT-Da205 mature light chain variable domain protein sequence |
| 333 | SCT-Da206 mature light chain variable domain protein sequence |
| 333 | SCT-Da207 mature light chain variable domain protein sequence |
| 333 | SCT-Da216 mature light chain variable domain protein sequence |
| 333 | SCT-Da220 mature light chain variable domain protein sequence |
| 333 | SCT-Da223 mature light chain variable domain protein sequence |
| 333 | SCT-Da255 mature light chain variable domain protein sequence |
| 333 | SCT-Da302 mature light chain variable domain protein sequence |
| 333 | SCT-Da321 mature light chain variable domain protein sequence |
| 333 | SCT-Da362 mature light chain variable domain protein sequence |
| 334 | SCT-Da115 mature light chain variable domain protein sequence |
| 335 | SCT-Da120 mature light chain variable domain protein sequence |
| 336 | SCT-Da121 mature light chain variable domain protein sequence |
| 337 | SCT-Da122 mature light chain variable domain protein sequence |
| 337 | SCT-Da287 mature light chain variable domain protein sequence |
| 338 | SCT-Da124 mature light chain variable domain protein sequence |
| 339 | SCT-Da125 mature light chain variable domain protein sequence |
| 340 | SCT-Da126 mature light chain variable domain protein sequence |
| 341 | SCT-Da127 mature light chain variable domain protein sequence |
| 342 | SCT-Da130 mature light chain variable domain protein sequence |
| 342 | SCT-Da132 mature light chain variable domain protein sequence |
| 342 | SCT-Da144 mature light chain variable domain protein sequence |
| 343 | SCT-Da136 mature light chain variable domain protein sequence |
| 344 | SCT-Da137 mature light chain variable domain protein sequence |
| 345 | SCT-Da138 mature light chain variable domain protein sequence |
| 346 | SCT-Da139 mature light chain variable domain protein sequence |
| 347 | SCT-Da141 mature light chain variable domain protein sequence |
| 348 | SCT-Da142 mature light chain variable domain protein sequence |
| 349 | SCT-Da143 mature light chain variable domain protein sequence |
| 349 | SCT-Da226 mature light chain variable domain protein sequence |
| 350 | SCT-Da149 mature light chain variable domain protein sequence |
| 351 | SCT-Da150 mature light chain variable domain protein sequence |
| 352 | SCT-Da152 mature light chain variable domain protein sequence |
| 353 | SCT-Da153 mature light chain variable domain protein sequence |
| 354 | SCT-Da154 mature light chain variable domain protein sequence |
| 354 | SCT-Da350 mature light chain variable domain protein sequence |
| 355 | SCT-Da155 mature light chain variable domain protein sequence |
| 356 | SCT-Da160 mature light chain variable domain protein sequence |
| 356 | SCT-Da161 mature light chain variable domain protein sequence |
| 357 | SCT-Da162 mature light chain variable domain protein sequence |
| 358 | SCT-Da165 mature light chain variable domain protein sequence |
| 358 | SCT-Da290 mature light chain variable domain protein sequence |
| 359 | SCT-Da166 mature light chain variable domain protein sequence |
| 359 | SCT-Da177 mature light chain variable domain protein sequence |
| 359 | SCT-Da203 mature light chain variable domain protein sequence |
| 360 | SCT-Da167 mature light chain variable domain protein sequence |
| 361 | SCT-Da168 mature light chain variable domain protein sequence |
| 362 | SCT-Da169 mature light chain variable domain protein sequence |
| 363 | SCT-Da170 mature light chain variable domain protein sequence |
| 364 | SCT-Da171 mature light chain variable domain protein sequence |
| 365 | SCT-Da173 mature light chain variable domain protein sequence |
| 366 | SCT-Da174 mature light chain variable domain protein sequence |
| 366 | SCT-Da293 mature light chain variable domain protein sequence |
| 366 | SCT-Da330 mature light chain variable domain protein sequence |
| 367 | SCT-Da178 mature light chain variable domain protein sequence |
| 368 | SCT-Da179 mature light chain variable domain protein sequence |
| 368 | SCT-Da180 mature light chain variable domain protein sequence |
| 369 | SCT-Da182 mature light chain variable domain protein sequence |
| 370 | SCT-Da184 mature light chain variable domain protein sequence |
| 371 | SCT-Da185 mature light chain variable domain protein sequence |
| 371 | SCT-Da292 mature light chain variable domain protein sequence |
| 372 | SCT-Da186 mature light chain variable domain protein sequence |
| 373 | SCT-Da187 mature light chain variable domain protein sequence |
| 374 | SCT-Da190 mature light chain variable domain protein sequence |
| 375 | SCT-Da193 mature light chain variable domain protein sequence |
| 376 | SCT-Da194 mature light chain variable domain protein sequence |
| 377 | SCT-Da195 mature light chain variable domain protein sequence |
| 377 | SCT-Da199 mature light chain variable domain protein sequence |
| 378 | SCT-Da196 mature light chain variable domain protein sequence |
| 379 | SCT-Da197 mature light chain variable domain protein sequence |
| 380 | SCT-Da204 mature light chain variable domain protein sequence |
| 381 | SCT-Da208 mature light chain variable domain protein sequence |
| 382 | SCT-Da210 mature light chain variable domain protein sequence |
| 383 | SCT-Da212 mature light chain variable domain protein sequence |
| 384 | SCT-Da213 mature light chain variable domain protein sequence |
| 385 | SCT-Da214 mature light chain variable domain protein sequence |
| 386 | SCT-Da215 mature light chain variable domain protein sequence |
| 386 | SCT-Da217 mature light chain variable domain protein sequence |
| 387 | SCT-Da218 mature light chain variable domain protein sequence |
| 387 | SCT-Da274 mature light chain variable domain protein sequence |
| 388 | SCT-Da219 mature light chain variable domain protein sequence |
| 389 | SCT-Da221 mature light chain variable domain protein sequence |
| 390 | SCT-Da224 mature light chain variable domain protein sequence |
| 391 | SCT-Da225 mature light chain variable domain protein sequence |
| 392 | SCT-Da227 mature light chain variable domain protein sequence |
| 393 | SCT-Da228 mature light chain variable domain protein sequence |
| 394 | SCT-Da229 mature light chain variable domain protein sequence |
| 395 | SCT-Da230 mature light chain variable domain protein sequence |
| 396 | SCT-Da231 mature light chain variable domain protein sequence |
| 397 | SCT-Da232 mature light chain variable domain protein sequence |
| 397 | SCT-Da244 mature light chain variable domain protein sequence |
| 397 | SCT-Da246 mature light chain variable domain protein sequence |
| 397 | SCT-Da248 mature light chain variable domain protein sequence |
| 398 | SCT-Da233 mature light chain variable domain protein sequence |
| 399 | SCT-Da234 mature light chain variable domain protein sequence |
| 400 | SCT-Da235 mature light chain variable domain protein sequence |
| 400 | SCT-Da333 mature light chain variable domain protein sequence |
| 401 | SCT-Da236 mature light chain variable domain protein sequence |
| 402 | SCT-Da237 mature light chain variable domain protein sequence |
| 403 | SCT-Da238 mature light chain variable domain protein sequence |
| 404 | SCT-Da239 mature light chain variable domain protein sequence |
| 405 | SCT-Da240 mature light chain variable domain protein sequence |
| 406 | SCT-Da241 mature light chain variable domain protein sequence |
| 407 | SCT-Da242 mature light chain variable domain protein sequence |
| 408 | SCT-Da243 mature light chain variable domain protein sequence |
| 409 | SCT-Da245 mature light chain variable domain protein sequence |
| 410 | SCT-Da249 mature light chain variable domain protein sequence |
| 410 | SCT-Da319 mature light chain variable domain protein sequence |
| 411 | SCT-Da250 mature light chain variable domain protein sequence |
| 411 | SCT-Da297 mature light chain variable domain protein sequence |
| 412 | SCT-Da251 mature light chain variable domain protein sequence |
| 413 | SCT-Da252 mature light chain variable domain protein sequence |
| 413 | SCT-Da298 mature light chain variable domain protein sequence |
| 414 | SCT-Da253 mature light chain variable domain protein sequence |
| 415 | SCT-Da254 mature light chain variable domain protein sequence |
| 416 | SCT-Da256 mature light chain variable domain protein sequence |
| 417 | SCT-Da257 mature light chain variable domain protein sequence |
| 418 | SCT-Da258 mature light chain variable domain protein sequence |
| 419 | SCT-Da259 mature light chain variable domain protein sequence |
| 419 | SCT-Da260 mature light chain variable domain protein sequence |
| 420 | SCT-Da261 mature light chain variable domain protein sequence |
| 421 | SCT-Da262 mature light chain variable domain protein sequence |
| 422 | SCT-Da263 mature light chain variable domain protein sequence |
| 423 | SCT-Da264 mature light chain variable domain protein sequence |
| 424 | SCT-Da265 mature light chain variable domain protein sequence |
| 425 | SCT-Da266 mature light chain variable domain protein sequence |
| 426 | SCT-Da267 mature light chain variable domain protein sequence |
| 427 | SCT-Da268 mature light chain variable domain protein sequence |
| 428 | SCT-Da269 mature light chain variable domain protein sequence |
| 429 | SCT-Da270 mature light chain variable domain protein sequence |
| 429 | SCT-Da273 mature light chain variable domain protein sequence |
| 429 | SCT-Da280 mature light chain variable domain protein sequence |
| 429 | SCT-Da304 mature light chain variable domain protein sequence |
| 430 | SCT-Da271 mature light chain variable domain protein sequence |
| 430 | SCT-Da275 mature light chain variable domain protein sequence |
| 430 | SCT-Da294 mature light chain variable domain protein sequence |
| 431 | SCT-Da272 mature light chain variable domain protein sequence |
| 432 | SCT-Da276 mature light chain variable domain protein sequence |
| 433 | SCT-Da277 mature light chain variable domain protein sequence |
| 434 | SCT-Da278 mature light chain variable domain protein sequence |
| 434 | SCT-Da279 mature light chain variable domain protein sequence |
| 434 | SCT-Da281 mature light chain variable domain protein sequence |
| 435 | SCT-Da282 mature light chain variable domain protein sequence |
| 436 | SCT-Da305 mature light chain variable domain protein sequence |
| 437 | SCT-Da306 mature light chain variable domain protein sequence |
| 438 | SCT-Da307 mature light chain variable domain protein sequence |
| 438 | SCT-Da310 mature light chain variable domain protein sequence |
| 438 | SCT-Da331 mature light chain variable domain protein sequence |
| 439 | SCT-Da308 mature light chain variable domain protein sequence |
| 440 | SCT-Da309 mature light chain variable domain protein sequence |
| 441 | SCT-Da311 mature light chain variable domain protein sequence |
| 442 | SCT-Da312 mature light chain variable domain protein sequence |
| 443 | SCT-Da313 mature light chain variable domain protein sequence |
| 443 | SCT-Da314 mature light chain variable domain protein sequence |
| 444 | SCT-Da315 mature light chain variable domain protein sequence |
| 445 | SCT-Da316 mature light chain variable domain protein sequence |
| 446 | SCT-Da317 mature light chain variable domain protein sequence |
| 447 | SCT-Da318 mature light chain variable domain protein sequence |
| 448 | SCT-Da320 mature light chain variable domain protein sequence |
| 449 | SCT-Da322 mature light chain variable domain protein sequence |
| 450 | SCT-Da323 mature light chain variable domain protein sequence |
| 451 | SCT-Da324 mature light chain variable domain protein sequence |
| 452 | SCT-Da325 mature light chain variable domain protein sequence |
| 453 | SCT-Da326 mature light chain variable domain protein sequence |
| 454 | SCT-Da328 mature light chain variable domain protein sequence |
| 455 | SCT-Da329 mature light chain variable domain protein sequence |
| 456 | SCT-Da332 mature light chain variable domain protein sequence |
| 456 | SCT-Da336 mature light chain variable domain protein sequence |
| 457 | SCT-Da334 mature light chain variable domain protein sequence |
| 457 | SCT-Da338 mature light chain variable domain protein sequence |
| 458 | SCT-Da335 mature light chain variable domain protein sequence |
| 459 | SCT-Da337 mature light chain variable domain protein sequence |
| 460 | SCT-Da339 mature light chain variable domain protein sequence |
| 461 | SCT-Da340 mature light chain variable domain protein sequence |
| 462 | SCT-Da341 mature light chain variable domain protein sequence |
| 463 | SCT-Da342 mature light chain variable domain protein sequence |
| 464 | SCT-Da343 mature light chain variable domain protein sequence |
| 465 | SCT-Da344 mature light chain variable domain protein sequence |
| 466 | SCT-Da345 mature light chain variable domain protein sequence |
| 467 | SCT-Da346 mature light chain variable domain protein sequence |
| 468 | SCT-Da347 mature light chain variable domain protein sequence |
| 468 | SCT-Da359 mature light chain variable domain protein sequence |
| 469 | SCT-Da348 mature light chain variable domain protein sequence |
| 470 | SCT-Da351 mature light chain variable domain protein sequence |
| 471 | SCT-Da352 mature light chain variable domain protein sequence |
| 472 | SCT-Da353 mature light chain variable domain protein sequence |
| 473 | SCT-Da354 mature light chain variable domain protein sequence |
| 474 | SCT-Da355 mature light chain variable domain protein sequence |
| 475 | SCT-Da356 mature light chain variable domain protein sequence |
| 476 | SCT-Da358 mature light chain variable domain protein sequence |
| 477 | SCT-Da360 mature light chain variable domain protein sequence |
| 478 | SCT-Da361 mature light chain variable domain protein sequence |
| 973 | SCT-Da363 mature heavy chain variable domain protein sequence |
| 973 | SCT-Da364 mature heavy chain variable domain protein sequence |
| 974 | SCT-Da363 mature light chain variable domain protein sequence |
| 975 | SCT-Da364 mature light chain variable domain protein sequence |
| 976 | SCT-Da365 mature heavy chain variable domain protein sequence |
| 976 | SCT-Da366 mature heavy chain variable domain protein sequence |
| 976 | SCT-Da367 mature heavy chain variable domain protein sequence |
| 977 | SCT-Da365 mature light chain variable domain protein sequence |
| 977 | SCT-Da368 mature light chain variable domain protein sequence |
| 978 | SCT-Da366 mature light chain variable domain protein sequence |
| 978 | SCT-Da369 mature light chain variable domain protein sequence |
| 979 | SCT-Da367 mature light chain variable domain protein sequence |
| 979 | SCT-Da370 mature light chain variable domain protein sequence |
| 980 | SCT-Da368 mature heavy chain variable domain protein sequence |
| 980 | SCT-Da369 mature heavy chain variable domain protein sequence |
| 980 | SCT-Da370 mature heavy chain variable domain protein sequence |
| 981 | SCT-Da371 mature heavy chain variable domain protein sequence |
| 981 | SCT-Da372 mature heavy chain variable domain protein sequence |
| 982 | SCT-Da371 mature light chain variable domain protein sequence |
| 982 | SCT-Da373 mature light chain variable domain protein sequence |
| 983 | SCT-Da372 mature light chain variable domain protein sequence |
| 983 | SCT-Da374 mature light chain variable domain protein sequence |
| 984 | SCT-Da373 mature heavy chain variable domain protein sequence |
| 984 | SCT-Da374 mature heavy chain variable domain protein sequence |

### DETAILED DESCRIPTION

The disclosure provides antibodies that bind to B7-H3. In one embodiment, the disclosure provides an isolated antibody that binds to SEQ ID NO:1.

### Antibodies

The disclosure provides antibodies that bind specifically to SEQ ID NO:1. The term “antibody” as used herein, includes both full-length immunoglobulins and antibody fragments that bind to the same antigens. The antibodies can be, e.g., a monoclonal, polyclonal, chimeric, humanized, or single chain antibody. As used herein, the terms “antigen binding fragment,” “fragment,” and “antibody fragment” are used interchangeably to refer to any fragment that comprises a portion of a full-length antibody, generally at least the antigen binding portion or the variable region thereof. Examples of antibody fragments include, but are not limited to, diabodies, single-chain antibody molecules, multi-specific antibodies, Fab, Fab′, F(ab′)₂, Fv or scFv.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

The terms “treating,” “treatment,” and the like, as used herein, mean ameliorating a disease, so as to reduce, ameliorate, or eliminate its cause, its progression, its severity, or one or more of its symptoms, or otherwise beneficially alter the disease in a subject. Reference to “treating,” or “treatment” of a patient is intended to include prophylaxis. Treatment may also be preemptive in nature, i.e., it may include prevention of disease in a subject exposed to or at risk for the disease. Prevention of a disease may involve complete protection from disease, for example as in the case of prevention of infection with a pathogen or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The terms “subject” and “patient” are used interchangeably herein to mean all mammals including humans. Examples of subjects include, but are not limited to, humans, monkeys, dogs, cats, horses, cows, goats, sheep, pigs, and rabbits. In one embodiment, the subject or patient is a human.

In certain embodiments, the cancer treated with the anti-B7-H3 antibody molecule, alone or in combination, includes but is not limited to, a solid tumor, a hematological cancer (e.g., leukemia, lymphoma, myeloma), and a metastatic lesion thereof. In one embodiment, the cancer is a solid tumor. Examples of solid tumors include malignancies, e.g., sarcomas and carcinomas (e.g., adenocarcinomas), of the various organ systems, such as those affecting lung, breast, lymphoid, gastrointestinal or colorectal, genitals and genitourinary tract (e.g., renal, urothelial, bladder cells), pharynx, CNS (e.g., brain, neural or glial cells), skin (e.g., melanoma), head and neck (e.g., head and neck squamous cell carcinoma (HNCC)), and pancreas. For example, melanoma, colon cancers, gastric cancer, rectal cancer, renal-cell carcinoma, breast cancer (e.g., a breast cancer that does not express one, two or all of estrogen receptor, progesterone receptor, or Her2/neu, e.g., a triple negative breast cancer), liver cancer, a lung cancer (e.g., a non-small cell lung cancer (NSCLC) (e.g., a NSCLC with squamous and/or non-squamous histology) or small cell lung cancer), prostate cancer, cancer of head or neck (e.g., HPV+ squamous cell carcinoma), cancer of the small intestine and cancer of the esophagus. Examples of hematological cancer include, but is not limited to, leukemia (e.g., a myeloid leukemia, lymphoid leukemia, or chronic lymphocytic leukemia (CLL)), lymphoma (e.g., Hogdkin lymphoma (HL), non-Hogdkin lymphoma (NHL), Diffuse large B-cell lymphoma (DLBCL), T-cell lymphoma, or mantle cell lymphoma (MCL)), and myeloma, e.g., multiple myeloma. The cancer may be at an early, intermediate, late stage or metastatic cancer. For discovering and developing antibody therapeutics to B7-H3 generally known in the art, please see U.S. Pat. No. 9,207,238, which is incorporated in its entirety by reference.

The provided antibodies disclosed herein can be used to treat, prevent and/or diagnose cancerous or malignant disorders (e.g., cancers such melanoma, e.g., advanced stage melanoma; pancreatic cancer, e.g., advanced pancreatic cancer; solid tumors; breast cancer, e.g., metastatic breast carcinoma; renal cell carcinoma, e.g., advanced or metastatic renal cell carcinoma (MRCC) or clear cell renal cell carcinoma), as well as infectious diseases (e.g., hepatitis, e.g., hepatitis B; influenza).

The disclosure provides the antibodies SCT-Da001, SCT-Da002, SCT-Da003, SCT-Da004, SCT-Da005, SCT-Da006, SCT-Da007, SCT-Da008, SCT-Da009, SCT-Da010, SCT-Da011, SCT-Da012, SCT-Da013, SCT-Da014, SCT-Da015, SCT-Da016, SCT-Da017, SCT-Da018, SCT-Da019, SCT-Da020, SCT-Da021, SCT-Da022, SCT-Da023, SCT-Da024, SCT-Da025, SCT-Da026, SCT-Da027, SCT-Da028, SCT-Da029, SCT-Da030, SCT-Da031, SCT-Da032, SCT-Da033, SCT-Da034, SCT-Da035, SCT-Da036, SCT-Da037, SCT-Da038, SCT-Da039, SCT-Da040, SCT-Da041, SCT-Da042, SCT-Da043, SCT-Da044, SCT-Da045, SCT-Da046, SCT-Da047, SCT-Da048, SCT-Da049, SCT-Da050, SCT-Da051, SCT-Da052, SCT-Da053, SCT-Da054, SCT-Da055, SCT-Da056, SCT-Da057, SCT-Da058, SCT-Da059, SCT-Da060, SCT-Da061, SCT-Da062, SCT-Da063, SCT-Da064, SCT-Da065, SCT-Da066, SCT-Da067, SCT-Da068, SCT-Da069, SCT-Da070, SCT-Da071, SCT-Da072, SCT-Da073, SCT-Da074, SCT-Da075, SCT-Da076, SCT-Da077, SCT-Da078, SCT-Da079, SCT-Da080, SCT-Da081, SCT-Da082, SCT-Da083, SCT-Da084, SCT-Da085, SCT-Da086, SCT-Da087, SCT-Da088, SCT-Da089, SCT-Da090, SCT-Da091, SCT-Da092, SCT-Da093, SCT-Da094, SCT-Da095, SCT-Da096, SCT-Da097, SCT-Da098, SCT-Da099, SCT-Da100, SCT-Da101, SCT-Da102, SCT-Da103, SCT-Da104, SCT-Da105, SCT-Da106, SCT-Da107, SCT-Da108, SCT-Da109, SCT-Da110, SCT-Da111, SCT-Da112, SCT-Da113, SCT-Da114, SCT-Da115, SCT-Da116, SCT-Da117, SCT-Da118, SCT-Da119, SCT-Da120, SCT-Da121, SCT-Da122, SCT-Da123, SCT-Da124, SCT-Da125, SCT-Da126, SCT-Da127, SCT-Da128, SCT-Da129, SCT-Da130, SCT-Da131, SCT-Da132, SCT-Da133, SCT-Da134, SCT-Da135, SCT-Da136, SCT-Da137, SCT-Da138, SCT-Da139, SCT-Da140, SCT-Da141, SCT-Da142, SCT-Da143, SCT-Da144, SCT-Da145, SCT-Da146, SCT-Da147, SCT-Da148, SCT-Da149, SCT-Da150, SCT-Da151, SCT-Da152, SCT-Da153, SCT-Da154, SCT-Da155, SCT-Da156, SCT-Da157, SCT-Da158, SCT-Da159, SCT-Da160, SCT-Da161, SCT-Da162, SCT-Da163, SCT-Da164, SCT-Da165, SCT-Da166, SCT-Da167, SCT-Da168, SCT-Da169, SCT-Da170, SCT-Da171, SCT-Da172, SCT-Da173, SCT-Da174, SCT-Da175, SCT-Da176, SCT-Da177, SCT-Da178, SCT-Da179, SCT-Da180, SCT-Da181, SCT-Da182, SCT-Da183, SCT-Da184, SCT-Da185, SCT-Da186, SCT-Da187, SCT-Da188, SCT-Da189, SCT-Da190, SCT-Da191, SCT-Da192, SCT-Da193, SCT-Da194, SCT-Da195, SCT-Da196, SCT-Da197, SCT-Da198, SCT-Da199, SCT-Da200, SCT-Da201, SCT-Da202, SCT-Da203, SCT-Da204, SCT-Da205, SCT-Da206, SCT-Da207, SCT-Da208, SCT-Da209, SCT-Da210, SCT-Da211, SCT-Da212, SCT-Da213, SCT-Da214, SCT-Da215, SCT-Da216, SCT-Da217, SCT-Da218, SCT-Da219, SCT-Da220, SCT-Da221, SCT-Da222, SCT-Da223, SCT-Da224, SCT-Da225, SCT-Da226, SCT-Da227, SCT-Da228, SCT-Da229, SCT-Da230, SCT-Da231, SCT-Da232, SCT-Da233, SCT-Da234, SCT-Da235, SCT-Da236, SCT-Da237, SCT-Da238, SCT-Da239, SCT-Da240, SCT-Da241, SCT-Da242, SCT-Da243, SCT-Da244, SCT-Da245, SCT-Da246, SCT-Da247, SCT-Da248, SCT-Da249, SCT-Da250, SCT-Da251, SCT-Da252, SCT-Da253, SCT-Da254, SCT-Da255, SCT-Da256, SCT-Da257, SCT-Da258, SCT-Da259, SCT-Da260, SCT-Da261, SCT-Da262, SCT-Da263, SCT-Da264, SCT-Da265, SCT-Da266, SCT-Da267, SCT-Da268, SCT-Da269, SCT-Da270, SCT-Da271, SCT-Da272, SCT-Da273, SCT-Da274, SCT-Da275, SCT-Da276, SCT-Da277, SCT-Da278, SCT-Da279, SCT-Da280, SCT-Da281, SCT-Da282, SCT-Da283, SCT-Da284, SCT-Da285, SCT-Da286, SCT-Da287, SCT-Da288, SCT-Da289, SCT-Da290, SCT-Da291, SCT-Da292, SCT-Da293, SCT-Da294, SCT-Da295, SCT-Da296, SCT-Da297, SCT-Da298, SCT-Da299, SCT-Da300, SCT-Da301, SCT-Da302, SCT-Da303, SCT-Da304, SCT-Da305, SCT-Da306, SCT-Da307, SCT-Da308, SCT-Da309, SCT-Da310, SCT-Da311, SCT-Da312, SCT-Da313, SCT-Da314, SCT-Da315, SCT-Da316, SCT-Da317, SCT-Da318, SCT-Da319, SCT-Da320, SCT-Da321, SCT-Da322, SCT-Da323, SCT-Da324, SCT-Da325, SCT-Da326, SCT-Da327, SCT-Da328, SCT-Da329, SCT-Da330, SCT-Da331, SCT-Da332, SCT-Da333, SCT-Da334, SCT-Da335, SCT-Da336, SCT-Da337, SCT-Da338, SCT-Da339, SCT-Da340, SCT-Da341, SCT-Da342, SCT-Da343, SCT-Da344, SCT-Da345, SCT-Da346, SCT-Da347, SCT-Da348, SCT-Da349, SCT-Da350, SCT-Da351, SCT-Da352, SCT-Da353, SCT-Da354, SCT-Da355, SCT-Da356, SCT-Da357, SCT-Da358, SCT-Da359, SCT-Da360, SCT-Da361, and SCT-Da362. Each of these is a murine monoclonal antibody. In addition, the disclosure provides the antibodies SCT-Da363, SCT-Da364, SCT-Da365, SCT-Da366, SCT-Da367, SCT-Da368, SCT-Da369, SCT-Da370, SCT-Da371, SCT-Da372, SCT-Da373, and SCT-Da374. Each of these is a humanized monoclonal antibody.

Additionally, recombinant anti-B7-H3 antibodies, such as chimeric and humanized monoclonal antibodies, comprising both human and non-human portions, which can be made using standard recombinant DNA techniques, are within the scope of the disclosure. Such chimeric and humanized monoclonal antibodies can be produced by recombinant DNA techniques such as, for example, the methods described in U.S. Pat. No. 7,112,421; Better et al. (1988) Science 240:1041-1043; or Liu et al. (1987) Proc. Natl. Acad. Sci. USA 84:3439-3443.

### Antibody Variable Domain Sequence

The antibodies of the disclosure may comprise the heavy chain variable domain sequences of SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:973, SEQ ID NO:976, SEQ ID NO:980, SEQ ID NO:981, or SEQ ID NO:984. The heavy chain variable domain sequences may consist essentially of SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:973, SEQ ID NO:976, SEQ ID NO:980, SEQ ID NO:981, or SEQ ID NO:984.

The antibodies of the disclosure may comprise the light chain variable domain sequences of SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, SEQ ID NO:282, SEQ ID NO:283, SEQ ID NO:284, SEQ ID NO:285, SEQ ID NO:286, SEQ ID NO:287, SEQ ID NO:288, SEQ ID NO:289, SEQ ID NO:290, SEQ ID NO:291, SEQ ID NO:292, SEQ ID NO:293, SEQ ID NO:294, SEQ ID NO:295, SEQ ID NO:296, SEQ ID NO:297, SEQ ID NO:298, SEQ ID NO:299, SEQ ID NO:300, SEQ ID NO:301, SEQ ID NO:302, SEQ ID NO:303, SEQ ID NO:304, SEQ ID NO:305, SEQ ID NO:306, SEQ ID NO:307, SEQ ID NO:308, SEQ ID NO:309, SEQ ID NO:310, SEQ ID NO:311, SEQ ID NO:312, SEQ ID NO:313, SEQ ID NO:314, SEQ ID NO:315, SEQ ID NO:316, SEQ ID NO:317, SEQ ID NO:318, SEQ ID NO:319, SEQ ID NO:320, SEQ ID NO:321, SEQ ID NO:322, SEQ ID NO:323, SEQ ID NO:324, SEQ ID NO:325, SEQ ID NO:326, SEQ ID NO:327, SEQ ID NO:328, SEQ ID NO:329, SEQ ID NO:330, SEQ ID NO:331, SEQ ID NO:332, SEQ ID NO:333, SEQ ID NO:334, SEQ ID NO:335, SEQ ID NO:336, SEQ ID NO:337, SEQ ID NO:338, SEQ ID NO:339, SEQ ID NO:340, SEQ ID NO:341, SEQ ID NO:342, SEQ ID NO:343, SEQ ID NO:344, SEQ ID NO:345, SEQ ID NO:346, SEQ ID NO:347, SEQ ID NO:348, SEQ ID NO:349, SEQ ID NO:350, SEQ ID NO:351, SEQ ID NO:352, SEQ ID NO:353, SEQ ID NO:354, SEQ ID NO:355, SEQ ID NO:356, SEQ ID NO:357, SEQ ID NO:358, SEQ ID NO:359, SEQ ID NO:360, SEQ ID NO:361, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:370, SEQ ID NO:371, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:391, SEQ ID NO:392, SEQ ID NO:393, SEQ ID NO:394, SEQ ID NO:395, SEQ ID NO:396, SEQ ID NO:397, SEQ ID NO:398, SEQ ID NO:399, SEQ ID NO:400, SEQ ID NO:401, SEQ ID NO:402, SEQ ID NO:403, SEQ ID NO:404, SEQ ID NO:405, SEQ ID NO:406, SEQ ID NO:407, SEQ ID NO:408, SEQ ID NO:409, SEQ ID NO:410, SEQ ID NO:411, SEQ ID NO:412, SEQ ID NO:413, SEQ ID NO:414, SEQ ID NO:415, SEQ ID NO:416, SEQ ID NO:417, SEQ ID NO:418, SEQ ID NO:419, SEQ ID NO:420, SEQ ID NO:421, SEQ ID NO:422, SEQ ID NO:423, SEQ ID NO:424, SEQ ID NO:425, SEQ ID NO:426, SEQ ID NO:427, SEQ ID NO:428, SEQ ID NO:429, SEQ ID NO:430, SEQ ID NO:431, SEQ ID NO:432, SEQ ID NO:433, SEQ ID NO:434, SEQ ID NO:435, SEQ ID NO:436, SEQ ID NO:437, SEQ ID NO:438, SEQ ID NO:439, SEQ ID NO:440, SEQ ID NO:441, SEQ ID NO:442, SEQ ID NO:443, SEQ ID NO:444, SEQ ID NO:445, SEQ ID NO:446, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:974, SEQ ID NO:975, SEQ ID NO:977, SEQ ID NO:978, SEQ ID NO:979, SEQ ID NO:982, or SEQ ID NO:983. The light chain variable domain sequences may consist essentially of SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, SEQ ID NO:282, SEQ ID NO:283, SEQ ID NO:284, SEQ ID NO:285, SEQ ID NO:286, SEQ ID NO:287, SEQ ID NO:288, SEQ ID NO:289, SEQ ID NO:290, SEQ ID NO:291, SEQ ID NO:292, SEQ ID NO:293, SEQ ID NO:294, SEQ ID NO:295, SEQ ID NO:296, SEQ ID NO:297, SEQ ID NO:298, SEQ ID NO:299, SEQ ID NO:300, SEQ ID NO:301, SEQ ID NO:302, SEQ ID NO:303, SEQ ID NO:304, SEQ ID NO:305, SEQ ID NO:306, SEQ ID NO:307, SEQ ID NO:308, SEQ ID NO:309, SEQ ID NO:310, SEQ ID NO:311, SEQ ID NO:312, SEQ ID NO:313, SEQ ID NO:314, SEQ ID NO:315, SEQ ID NO:316, SEQ ID NO:317, SEQ ID NO:318, SEQ ID NO:319, SEQ ID NO:320, SEQ ID NO:321, SEQ ID NO:322, SEQ ID NO:323, SEQ ID NO:324, SEQ ID NO:325, SEQ ID NO:326, SEQ ID NO:327, SEQ ID NO:328, SEQ ID NO:329, SEQ ID NO:330, SEQ ID NO:331, SEQ ID NO:332, SEQ ID NO:333, SEQ ID NO:334, SEQ ID NO:335, SEQ ID NO:336, SEQ ID NO:337, SEQ ID NO:338, SEQ ID NO:339, SEQ ID NO:340, SEQ ID NO:341, SEQ ID NO:342, SEQ ID NO:343, SEQ ID NO:344, SEQ ID NO:345, SEQ ID NO:346, SEQ ID NO:347, SEQ ID NO:348, SEQ ID NO:349, SEQ ID NO:350, SEQ ID NO:351, SEQ ID NO:352, SEQ ID NO:353, SEQ ID NO:354, SEQ ID NO:355, SEQ ID NO:356, SEQ ID NO:357, SEQ ID NO:358, SEQ ID NO:359, SEQ ID NO:360, SEQ ID NO:361, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:370, SEQ ID NO:371, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:391, SEQ ID NO:392, SEQ ID NO:393, SEQ ID NO:394, SEQ ID NO:395, SEQ ID NO:396, SEQ ID NO:397, SEQ ID NO:398, SEQ ID NO:399, SEQ ID NO:400, SEQ ID NO:401, SEQ ID NO:402, SEQ ID NO:403, SEQ ID NO:404, SEQ ID NO:405, SEQ ID NO:406, SEQ ID NO:407, SEQ ID NO:408, SEQ ID NO:409, SEQ ID NO:410, SEQ ID NO:411, SEQ ID NO:412, SEQ ID NO:413, SEQ ID NO:414, SEQ ID NO:415, SEQ ID NO:416, SEQ ID NO:417, SEQ ID NO:418, SEQ ID NO:419, SEQ ID NO:420, SEQ ID NO:421, SEQ ID NO:422, SEQ ID NO:423, SEQ ID NO:424, SEQ ID NO:425, SEQ ID NO:426, SEQ ID NO:427, SEQ ID NO:428, SEQ ID NO:429, SEQ ID NO:430, SEQ ID NO:431, SEQ ID NO:432, SEQ ID NO:433, SEQ ID NO:434, SEQ ID NO:435, SEQ ID NO:436, SEQ ID NO:437, SEQ ID NO:438, SEQ ID NO:439, SEQ ID NO:440, SEQ ID NO:441, SEQ ID NO:442, SEQ ID NO:443, SEQ ID NO:444, SEQ ID NO:445, SEQ ID NO:446, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:974, SEQ ID NO:975, SEQ ID NO:977, SEQ ID NO:978, SEQ ID NO:979, SEQ ID NO:982, or SEQ ID NO:983.

The disclosure also provides a variable domain sequence comprising a sequence that is at least 80%, at least 85%, at least 90%, or at least 95% identical to a sequence selected from SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, SEQ ID NO:242, SEQ ID NO:243, SEQ ID NO:244, SEQ ID NO:245, SEQ ID NO:246, SEQ ID NO:973, SEQ ID NO:976, SEQ ID NO:980, SEQ ID NO:981, or SEQ ID NO:984. The disclosure also provides a variable domain sequence comprising a sequence that is at least 80%, at least 85%, at least 90%, or at least 95% identical to a sequence selected from SEQ ID NO:247, SEQ ID NO:248, SEQ ID NO:249, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:272, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, SEQ ID NO:282, SEQ ID NO:283, SEQ ID NO:284, SEQ ID NO:285, SEQ ID NO:286, SEQ ID NO:287, SEQ ID NO:288, SEQ ID NO:289, SEQ ID NO:290, SEQ ID NO:291, SEQ ID NO:292, SEQ ID NO:293, SEQ ID NO:294, SEQ ID NO:295, SEQ ID NO:296, SEQ ID NO:297, SEQ ID NO:298, SEQ ID NO:299, SEQ ID NO:300, SEQ ID NO:301, SEQ ID NO:302, SEQ ID NO:303, SEQ ID NO:304, SEQ ID NO:305, SEQ ID NO:306, SEQ ID NO:307, SEQ ID NO:308, SEQ ID NO:309, SEQ ID NO:310, SEQ ID NO:311, SEQ ID NO:312, SEQ ID NO:313, SEQ ID NO:314, SEQ ID NO:315, SEQ ID NO:316, SEQ ID NO:317, SEQ ID NO:318, SEQ ID NO:319, SEQ ID NO:320, SEQ ID NO:321, SEQ ID NO:322, SEQ ID NO:323, SEQ ID NO:324, SEQ ID NO:325, SEQ ID NO:326, SEQ ID NO:327, SEQ ID NO:328, SEQ ID NO:329, SEQ ID NO:330, SEQ ID NO:331, SEQ ID NO:332, SEQ ID NO:333, SEQ ID NO:334, SEQ ID NO:335, SEQ ID NO:336, SEQ ID NO:337, SEQ ID NO:338, SEQ ID NO:339, SEQ ID NO:340, SEQ ID NO:341, SEQ ID NO:342, SEQ ID NO:343, SEQ ID NO:344, SEQ ID NO:345, SEQ ID NO:346, SEQ ID NO:347, SEQ ID NO:348, SEQ ID NO:349, SEQ ID NO:350, SEQ ID NO:351, SEQ ID NO:352, SEQ ID NO:353, SEQ ID NO:354, SEQ ID NO:355, SEQ ID NO:356, SEQ ID NO:357, SEQ ID NO:358, SEQ ID NO:359, SEQ ID NO:360, SEQ ID NO:361, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:370, SEQ ID NO:371, SEQ ID NO:372, SEQ ID NO:373, SEQ ID NO:374, SEQ ID NO:375, SEQ ID NO:376, SEQ ID NO:377, SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:391, SEQ ID NO:392, SEQ ID NO:393, SEQ ID NO:394, SEQ ID NO:395, SEQ ID NO:396, SEQ ID NO:397, SEQ ID NO:398, SEQ ID NO:399, SEQ ID NO:400, SEQ ID NO:401, SEQ ID NO:402, SEQ ID NO:403, SEQ ID NO:404, SEQ ID NO:405, SEQ ID NO:406, SEQ ID NO:407, SEQ ID NO:408, SEQ ID NO:409, SEQ ID NO:410, SEQ ID NO:411, SEQ ID NO:412, SEQ ID NO:413, SEQ ID NO:414, SEQ ID NO:415, SEQ ID NO:416, SEQ ID NO:417, SEQ ID NO:418, SEQ ID NO:419, SEQ ID NO:420, SEQ ID NO:421, SEQ ID NO:422, SEQ ID NO:423, SEQ ID NO:424, SEQ ID NO:425, SEQ ID NO:426, SEQ ID NO:427, SEQ ID NO:428, SEQ ID NO:429, SEQ ID NO:430, SEQ ID NO:431, SEQ ID NO:432, SEQ ID NO:433, SEQ ID NO:434, SEQ ID NO:435, SEQ ID NO:436, SEQ ID NO:437, SEQ ID NO:438, SEQ ID NO:439, SEQ ID NO:440, SEQ ID NO:441, SEQ ID NO:442, SEQ ID NO:443, SEQ ID NO:444, SEQ ID NO:445, SEQ ID NO:446, SEQ ID NO:447, SEQ ID NO:448, SEQ ID NO:449, SEQ ID NO:450, SEQ ID NO:451, SEQ ID NO:452, SEQ ID NO:453, SEQ ID NO:454, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:974, SEQ ID NO:975, SEQ ID NO:977, SEQ ID NO:978, SEQ ID NO:979, SEQ ID NO:982, or SEQ ID NO:983. The disclosure also provides antibodies comprising a heavy chain variable domain sequence that is at least 80%, at least 85%, at least 90%, or at least 95% identical to SEQ ID NO:4 and a light chain variable domain sequence that is at least 80%, at least 85%, at least 90%, or at least 95% identical to SEQ ID NO:247. The disclosure also provides antibodies comprising a heavy chain variable domain sequence that is at least 80%, at least 85%, at least 90%, or at least 95% identical to SEQ ID NO:5 and a light chain variable domain sequence that is at least 80%, at least 85%, at least 90%, or at least 95% identical to SEQ ID NO:248. The disclosure also provides antibodies comprising a heavy chain variable domain sequence that is at least 80%, at least 85%, at least 90%, or at least 95% identical to SEQ ID NO:6 and a light chain variable domain sequence that is at least 80%, at least 85%, at least 90%, or at least 95% identical to SEQ ID NO:249. The disclosure also provides antibodies comprising a heavy chain variable domain sequence that is at least 80%, at least 85%, at least 90%, or at least 95% identical to SEQ ID NO:7 and a light chain variable domain sequence that is at least 80%, at least 85%, at least 90%, or at least 95% identical to SEQ ID NO:250. The disclosure also provides antibodies comprising a heavy chain variable domain sequence that is at least 80%, at least 85%, at least 90%, or at least 95% identical to SEQ ID NO:8 and a light chain variable domain sequence that is at least 80%, at least 85%, at least 90%, or at least 95% identical to SEQ ID NO:251. The disclosure also provides antibodies comprising a heavy chain variable domain sequence that is at least 80%, at least 85%, at least 90%, or at least 95% identical to SEQ ID NO:9 and a light chain variable domain sequence that is at least 80%, at least 85%, at least 90%, or at least 95% identical to SEQ ID NO:252. The disclosure also provides antibodies comprising a heavy chain variable domain sequence that is at least 80%, at least 85%, at least 90%, or at least 95% identical to SEQ ID NO:9 and a light chain variable domain sequence that is at least 80%, at least 85%, at least 90%, or at least 95% identical to SEQ ID NO:253. The disclosure also provides antibodies comprising a heavy chain variable domain sequence that is at least 80%, at least 85%, at least 90%, or at least 95% identical to SEQ ID NO:9 and a light chain variable domain sequence that is at least 80%, at least 85%, at least 90%, or at least 95% identical to SEQ ID NO:254. The disclosure also provides antibodies comprising a heavy chain variable domain sequence that is at least 80%, at least 85%, at least 90%, or at least 95% identical to SEQ ID NO:9 and a light chain variable domain sequence that is at least 80%, at least 85%, at least 90%, or at least 95% identical to SEQ ID NO:255. The disclosure also provides antibodies comprising a heavy chain variable domain sequence that is at least 80%, at least 85%, at least 90%, or at least 95% identical to SEQ ID NO:10 and a light chain variable domain sequence that is at least 80%, at least 85%, at least 90%, or at least 95% identical to SEQ ID NO:256. The disclosure also provides antibodies comprising a heavy chain variable domain sequence that is at least 80%, at least 85%, at least 90%, or at least 95% identical to SEQ ID NO:11 and a light chain variable domain sequence that is at least 80%, at least 85%, at least 90%, or at least 95% identical to SEQ ID NO:254. The disclosure also provides antibodies comprising a heavy chain variable domain sequence that is at least 80%, at least 85%, at least 90%, or at least 95% identical to SEQ ID NO:11 and a light chain variable domain sequence that is at least 80%, at least 85%, at least 90%, or at least 95% identical to SEQ ID NO:256. The disclosure also provides antibodies comprising a heavy chain variable domain sequence that is at least 80%, at least 85%, at least 90%, or at least 95% identical to SEQ ID NO:12 and a light chain variable domain sequence that is at least 80%, at least 85%, at least 90%, or at least 95% identical to SEQ ID NO:254. The disclosure also provides antibodies comprising a heavy chain variable domain sequence that is at least 80%, at least 85%, at least 90%, or at least 95% identical to SEQ ID NO:13 and a light chain variable domain sequence that is at least 80%, at least 85%, at least 90%, or at least 95% identical to SEQ ID NO:257. The disclosure also provides antibodies comprising a heavy chain variable domain sequence that is at least 80%, at least 85%, at least 90%, or at least 95% identical to SEQ ID NO:14 and a light chain variable domain sequence that is at least 80%, at least 85%, at least 90%, or at least 95% identical to SEQ ID NO:258. The disclosure also provides antibodies comprising a heavy chain variable domain sequence that is at least 80%, at least 85%, at least 90%, or at least 95% identical to SEQ ID NO:15 and a light chain variable domain sequence that is at least 80%, at least 85%, at least 90%, or at least 95% identical to SEQ ID NO:259. The disclosure also provides antibodies comprising a heavy chain variable domain sequence that is at least 80%, at least 85%, at least 90%, or at least 95% identical to SEQ ID NO:16 and a light chain variable domain sequence that is at least 80%, at least 85%, at least 90%, or at least 95% identical to SEQ ID NO:260. The disclosure also provides antibodies comprising a heavy chain variable domain sequence that is at least 80%, at least 85%, at least 90%, or at least 95% identical to SEQ ID NO:16 and a light chain variable domain sequence that is at least 80%, at least 85%, at least 90%, or at least 95% identical to SEQ ID NO:261. The disclosure also provides antibodies comprising a heavy chain variable domain sequence that is at least 80%, at least 85%, at least 90%, or at least 95% identical to SEQ ID NO:17 and a light chain variable domain sequence that is at least 80%, at least 85%, at least 90%, or at least 95% identical to SEQ ID NO:257. The disclosure also provides antibodies comprising a heavy chain variable domain sequence that is at least 80%, at least 85%, at least 90%, or at least 95% identical to SEQ ID NO:18 and a light chain variable domain sequence that is at least 80%, at least 85%, at least 90%, or at least 95% identical to SEQ ID NO:262. The disclosure also provides antibodies comprising a heavy chain variable domain sequence that is at least 80%, at least 85%, at least 90%, or at least 95% identical to SEQ ID NO:19 and a light chain variable domain sequence that is at least 80%, at least 85%, at least 90%, or at least 95% identical to SEQ ID NO:250. The disclosure also provides antibodies comprising a heavy chain variable domain sequence that is at least 80%, at least 85%, at least 90%, or at least 95% identical to SEQ ID NO:19 and a light chain variable domain sequence that is at least 80%, at least 85%, at least 90%, or at least 95% identical to SEQ ID NO:250. The disclosure also provides antibodies comprising a heavy chain variable domain sequence that is at least 80%, at least 85%, at least 90%, or at least 95% identical to SEQ ID NO:21 and a light chain variable domain sequence that is at least 80%, at least 85%, at least 90%, or at least 95% identical to SEQ ID NO:263. The disclosure also provides antibodies comprising a heavy chain variable domain sequence that is at least 80%, at least 85%, at least 90%, or at least 95% identical to SEQ ID NO:22 and a light chain variable domain sequence that is at least 80%, at least 85%, at least 90%, or at least 95% identical to SEQ ID NO:263. The disclosure also provides antibodies comprising a heavy chain variable domain sequence that is at least 80%, at least 85%, at least 90%, or at least 95% identical to SEQ ID NO:22 and a light chain variable domain sequence that is at least 80%, at least 85%, at least 90%, or at least 95% identical to SEQ ID NO:250. The disclosure also provides antibodies comprising a heavy chain variable domain sequence that is at least 80%, at least 85%, at least 90%, or at least 95% identical to SEQ ID NO:22 and a light chain variable domain sequence that is at least 80%, at least 85%, at least 90%, or at least 95% identical to SEQ ID NO:264. The disclosure also provides antibodies comprising a heavy chain variable domain sequence that is at least 80%, at least 85%, at least 90%, or at least 95% identical to SEQ ID NO:22 and a light chain variable domain sequence that is at least 80%, at least 85%, at least 90%, or at least 95% identical to SEQ ID NO:265. The disclosure also provides antibodies comprising a heavy chain variable domain sequence that is at least 80%, at least 85%, at least 90%, or at least 95% identical to SEQ ID NO:23 and a light chain variable domain sequence that is at least 80%, at least 85%, at least 90%, or at least 95% identical to SEQ ID NO:266. The disclosure also provides antibodies comprising a heavy chain variable domain sequence that is at least 80%, at least 85%, at least 90%, or at least 95% identical to SEQ ID NO:24 and a light chain variable domain sequence that is at least 80%, at least 85%, at least 90%, or at least 95% identical to SEQ ID NO:267. The disclosure also provides antibodies comprising a heavy chain variable domain sequence that is at least 80%, at least 85%, at least 90%, or at least 95% identical to SEQ ID NO:25 and a light chain variable domain sequence that is at least 80%, at least 85%, at least 90%, or at least 95% identical to SEQ ID NO:268. The disclosure also provides antibodies comprising a heavy chain variable domain sequence that is at least 80%, at least 85%, at least 90%, or at least 95% identical to SEQ ID NO:26 and a light chain variable domain sequence that is at least 80%, at least 85%, at least 90%, or at least 95% identical to SEQ ID NO:269. The disclosure also provides antibodies comprising a heavy chain variable domain sequence that is at least 80%, at least 85%, at least 90%, or at least 95% identical to SEQ ID NO:27 and a light chain variable domain sequence that is at least 80%, at least 85%, at least 90%, or at least 95% identical to SEQ ID NO:270. The disclosure also provides antibodies comprising a heavy chain variable domain sequence that is at least 80%, at least 85%, at least 90%, or at least 95% identical to SEQ ID NO:28 and a light chain variable domain sequence that is at least 80%, at least 85%, at least 90%, or at least 95% identical to SEQ ID NO:271. The disclosure also provides antibodies comprising a heavy chain variable domain sequence that is at least 80%, at least 85%, at least 90%, or at least 95% identical to SEQ ID NO:29 and a light chain variable domain sequence that is at least 80%, at least 85%, at least 90%, or at least 95% identical to SEQ ID NO:266. The disclosure also provides antibodies comprising a heavy chain variable domain sequence that is at least 80%, at least 85%, at least 90%, or at least 95% identical to SEQ ID NO:30 and a light chain variable domain sequence that is at least 80%, at least 85%, at least 90%, or at least 95% identical to SEQ ID NO:272. The disclosure also provides antibodies comprising a heavy chain variable domain sequence that is at least 80%, at least 85%, at least 90%, or at least 95% identical to SEQ ID NO:31 and a light chain variable domain sequence that is at least 80%, at least 85%, at least 90%, or at least 95% identical to SEQ ID NO:273. The disclosure also provides antibodies comprising a heavy chain variable domain sequence that is at least 80%, at least 85%, at least 90%, or at least 95% identical to SEQ ID NO:32 and a light chain variable domain sequence that is at least 80%, at least 85%, at least 90%, or at least 95% identical to SEQ ID NO:274. The disclosure also provides antibodies comprising a heavy chain variable domain sequence that is at least 80%, at least 85%, at least 90%, or at least 95% identical to SEQ ID NO:33 and a light chain variable domain sequence that is at least 80%, at least 85%, at least 90%, or at least 95% identical to SEQ ID NO:275. The disclosure also provides antibodies comprising a heavy chain variable domain sequence that is at least 80%, at least 85%, at least 90%, or at least 95% identical to SEQ ID NO:34 and a light chain variable domain sequence that is at least 80%, at least 85%, at least 90%, or at least 95% identical to SEQ ID NO:276. The disclosure also provides antibodies comprising a heavy chain variable domain sequence that is at least 80%, at least 85%, at least 90%, or at least 95% identical to SEQ ID NO:35 and a light chain variable domain sequence that is at least 80%, at least 85%, at least 90%, or at least 95% identical to SEQ ID NO:277. The disclosure also provides antibodies comprising a heavy chain variable domain sequence that is at least 80%, at least 85%, at least 90%, or at least 95% identical to SEQ ID NO:36 and a light chain variable domain sequence that is at least 80%, at least 85%, at least 90%, or at least 95% identical to SEQ ID NO:278. The disclosure also provides antibodies comprising a heavy chain variable domain sequence that is at least 80%, at least 85%, at least 90%, or at least 95% identical to SEQ ID NO:37 and a light chain variable domain sequence that is at least 80%, at least 85%, at least 90%, or at least 95% identical to SEQ ID NO:279. The disclosure also provides antibodies comprising a heavy chain variable domain sequence that is at least 80%, at least 85%, at least 90%, or at least 95% identical to SEQ ID NO:38 and a light chain variable domain sequence that is at least 80%, at least 85%, at least 90%, or at least 95% identical to SEQ ID NO:280. The disclosure also provides antibodies comprising a heavy chain variable domain sequence that is at least 80%, at least 85%, at least 90%, or at least 95% identical to SEQ ID NO:39 and a light chain variable domain sequence that is at least 80%, at least 85%, at least 90%, or at least 95% identical to SEQ ID NO:281. The disclosure also provides antibodies comprising a heavy chain variable domain sequence that is at least 80%, at least 85%, at least 90%, or at least 95% identical to SEQ ID NO:39 and a light chain variable domain sequence that is at least 80%, at least 85%, at least 90%, or at least 95% identical to SEQ ID NO:282. The disclosure also provides antibodies comprising a heavy chain variable domain sequence that is at least 80%, at least 85%, at least 90%, or at least 95% identical to SEQ ID NO:40 and a light chain variable domain sequence that is at least 80%, at least 85%, at least 90%, or at least 95% identical to SEQ ID NO:283. The disclosure also provides antibodies comprising a heavy chain variable domain sequence that is at least 80%, at least 85%, at least 90%, or at least 95% identical to SEQ ID NO:41 and a light chain variable domain sequence that is at least 80%, at least 85%, at least 90%, or at least 95% identical to SEQ ID NO:284. The disclosure also provides antibodies comprising a heavy chain variable domain sequence that is at least 80%, at least 85%, at least 90%, or at least 95% identical to SEQ ID NO:41 and a light chain variable domain sequence that is at least 80%, at least 85%, at least 90%, or at least 95% identical to SEQ ID NO:285. The disclosure also provides antibodies comprising a heavy chain variable domain sequence that is at least 80%, at least 85%, at least 90%, or at least 95% identical to SEQ ID NO:42 and a light chain variable domain sequence that is at least 80%, at least 85%, at least 90%, or at least 95% identical to SEQ ID NO:286. The disclosure also provides antibodies comprising a heavy chain variable domain sequence that is at least 80%, at least 85%, at least 90%, or at least 95% identical to SEQ ID NO:43 and a light chain variable domain sequence that is at least 80%, at least 85%, at least 90%, or at least 95% identical to SEQ ID NO:287. The disclosure also provides antibodies comprising a heavy chain variable domain sequence that is at least 80%, at least 85%, at least 90%, or at least 95% identical to SEQ ID NO:44 and a light chain variable domain sequence that is at least 80%, at least 85%, at least 90%, or at least 95% identical to SEQ ID NO:288. The disclosure also provides antibodies comprising a heavy chain variable domain sequence that is at least 80%, at least 85%, at least 90%, or at least 95% identical to SEQ ID NO:45 and a light chain variable domain sequence that is at least 80%, at least 85%, at least 90%, or at least 95% identical to SEQ ID NO:289. The disclosure also provides antibodies comprising a heavy chain variable domain sequence that is at least 80%, at least 85%, at least 90%, or at least 95% identical to SEQ ID NO:46 and a light chain variable domain sequence that is at least 80%, at least 85%, at least 90%, or at least 95% identical to SEQ ID NO:287. The disclosure also provides antibodies comprising a heavy chain variable domain sequence that is at least 80%, at least 85%, at least 90%, or at least 95% identical to SEQ ID NO:47 and a light chain variable domain sequence that is at least 80%, at least 85%, at least 90%, or at least 95% identical to SEQ ID NO:290. The disclosure also provides antibodies comprising a heavy chain variable domain sequence that is at least 80%, at least 85%, at least 90%, or at least 95% identical to SEQ ID NO:48 and a light chain variable domain sequence that is at least 80%, at least 85%, at least 90%, or at least 95% identical to SEQ ID NO:291. The disclosure also provides antibodies comprising a heavy chain variable domain sequence that is at least 80%, at least 85%, at least 90%, or at least 95% identical to SEQ ID NO:49 and a light chain variable domain sequence that is at least 80%, at least 85%, at least 90%, or at least 95% identical to SEQ ID NO:292. The disclosure also provides antibodies comprising a heavy chain variable domain sequence that is at least 80%, at least 85%, at least 90%, or at least 95% identical to SEQ ID NO:50 and a light chain variable domain sequence that is at least 80%, at least 85%, at least 90%, or at least 95% identical to SEQ ID NO:293. The disclosure also provides antibodies comprising a heavy chain variable domain sequence that is at least 80%, at least 85%, at least 90%, or at least 95% identical to SEQ ID NO:51 and a light chain variable domain sequence that is at least 80%, at least 85%, at least 90%, or at least 95% identical to SEQ ID NO:294. The disclosure also provides antibodies comprising a heavy chain variable domain sequence that is at least 80%, at least 85%, at least 90%, or at least 95% identical to SEQ ID NO:52 and a light chain variable domain sequence that is at least 80%, at least 85%, at least 90%, or at least 95% identical to SEQ ID NO:294. The disclosure also provides antibodies comprising a heavy chain variable domain sequence that is at least 80%, at least 85%, at least 90%, or at least 95% identical to SEQ ID NO:53 and a light chain variable domain sequence that is at least 80%, at least 85%, at least 90%, or at least 95% identical to SEQ ID NO:295. The disclosure also provides antibodies comprising a heavy chain variable domain sequence that is at least 80%, at least 85%, at least 90%, or at least 95% identical to SEQ ID NO:54 and a light chain variable domain sequence that is at least 80%, at least 85%, at least 90%, or at least 95% identical to SEQ ID NO:296. The disclosure also provides antibodies comprising a heavy chain variable domain sequence that is at least 80%, at least 85%, at least 90%, or at least 95% identical to SEQ ID NO:55 and a light chain variable domain sequence that is at least 80%, at least 85%, at least 90%, or at least 95% identical to SEQ ID NO:297. The disclosure also provides antibodies comprising a heavy chain variable domain sequence that is at least 80%, at least 85%, at least 90%, or at least 95% identical to SEQ ID NO:56 and a light chain variable domain sequence that is at least 80%, at least 85%, at least 90%, or at least 95% identical to SEQ ID NO:264. The disclosure also provides antibodies comprising a heavy chain variable domain sequence that is at least 80%, at least 85%, at least 90%, or at least 95% identical to SEQ ID NO:57 and a light chain variable domain sequence that is at least 80%, at least 85%, at least 90%, or at least 95% identical to SEQ ID NO:298. The disclosure also provides antibodies comprising a heavy chain variable domain sequence that is at least 80%, at least 85%, at least 90%, or at least 95% identical to SEQ ID NO:57 and a light chain variable domain sequence that is at least 80%, at least 85%, at least 90%, or at least 95% identical to SEQ ID NO:299. The disclosure also provides antibodies comprising a heavy chain variable domain sequence that is at least 80%, at least 85%, at least 90%, or at least 95% identical to SEQ ID NO:58 and a light chain variable domain sequence that is at least 80%, at least 85%, at least 90%, or at least 95% identical to SEQ ID NO:300. The disclosure also provides antibodies comprising a heavy chain variable domain sequence that is at least 80%, at least 85%, at least 90%, or at least 95% identical to SEQ ID NO:59 and a light chain variable domain sequence that is at least 80%, at least 85%, at least 90%, or at least 95% identical to SEQ ID NO:301. The disclosure also provides antibodies comprising a heavy chain variable domain sequence that is at least 80%, at least 85%, at least 90%, or at least 95% identical to SEQ ID NO:59 and a light chain variable domain sequence that is at least 80%, at least 85%, at least 90%, or at least 95% identical to SEQ ID NO:264. The disclosure also provides antibodies comprising a heavy chain variable domain sequence that is at least 80%, at least 85%, at least 90%, or at least 95% identical to SEQ ID NO:60 and a light chain variable domain sequence that is at least 80%, at least 85%, at least 90%, or at least 95% identical to SEQ ID NO:302. The disclosure also provides antibodies comprising a heavy chain variable domain sequence that is at least 80%, at least 85%, at least 90%, or at least 95% identical to SEQ ID NO:60 and a light chain variable domain sequence that is at least 80%, at least 85%, at least 90%, or at least 95% identical to SEQ ID NO:303. The disclosure also provides antibodies comprising a heavy chain variable domain sequence that is at least 80%, at least 85%, at least 90%, or at least 95% identical to SEQ ID NO:60 and a light chain variable domain sequence that is at least 80%, at least 85%, at least 90%, or at least 95% identical to SEQ ID NO:249. The disclosure also provides antibodies comprising a heavy chain variable domain sequence that is at least 80%, at least 85%, at least 90%, or at least 95% identical to SEQ ID NO:60 and a light chain variable domain sequence that is at least 80%, at least 85%, at least 90%, or at least 95% identical to SEQ ID NO:304. The disclosure also provides antibodies comprising a heavy chain variable domain sequence that is at least 80%, at least 85%, at least 90%, or at least 95% identical to SEQ ID NO:61 and a light chain variable domain sequence that is at least 80%, at least 85%, at least 90%, or at least 95% identical to SEQ ID NO:249. The disclosure also provides antibodies comprising a heavy chain variable domain sequence that is at least 80%, at least 85%, at least 90%, or at least 95% identical to SEQ ID NO:62 and a light chain variable domain sequence that is at least 80%, at least 85%, at least 90%, or at least 95% identical to SEQ ID NO:305. The disclosure also provides antibodies comprising a heavy chain variable domain sequence that is at least 80%, at least 85%, at least 90%, or at least 95% identical to SEQ ID NO:62 and a light chain variable domain sequence that is at least 80%, at least 85%, at least 90%, or at least 95% identical to SEQ ID NO:306. The disclosure also provides antibodies comprising a heavy chain variable domain sequence that is at least 80%, at least 85%, at least 90%, or at least 95% identical to SEQ ID NO:62 and a light chain variable domain sequence that is at least 80%, at least 85%, at least 90%, or at least 95% identical to SEQ ID NO:249. The disclosure also provides antibodies comprising a heavy chain variable domain sequence that is at least 80%, at least 85%, at least 90%, or at least 95% identical to SEQ ID NO:63 and a light chain variable domain sequence that is at least 80%, at least 85%, at least 90%, or at least 95% identical to SEQ ID NO:264. The disclosure also provides antibodies comprising a heavy chain variable domain sequence that is at least 80%, at least 85%, at least 90%, or at least 95% identical to SEQ ID NO:63 and a light chain variable domain sequence that is at least 80%, at least 85%, at least 90%, or at least 95% identical to SEQ ID NO:307. The disclosure also provides antibodies comprising a heavy chain variable domain sequence that is at least 80%, at least 85%, at least 90%, or at least 95% identical to SEQ ID NO:64 and a light chain variable domain sequence that is at least 80%, at least 85%, at least 90%, or at least 95% identical to SEQ ID NO:308. The disclosure also provides antibodies comprising a heavy chain variable domain sequence that is at least 80%, at least 85%, at least 90%, or at least 95% identical to SEQ ID NO:65 and a light chain variable domain sequence that is at least 80%, at least 85%, at least 90%, or at least 95% identical to SEQ ID NO:257. The disclosure also provides antibodies comprising a heavy chain variable domain sequence that is at least 80%, at least 85%, at least 90%, or at least 95% identical to SEQ ID NO:66 and a light chain variable domain sequence that is at least 80%, at least 85%, at least 90%, or at least 95% identical to SEQ ID NO:309. The disclosure also provides antibodies comprising a heavy chain variable domain sequence that is at least 80%, at least 85%, at least 90%, or at least 95% identical to SEQ ID NO:67 and a light chain variable domain sequence that is at least 80%, at least 85%, at least 90%, or at least 95% identical to SEQ ID NO:310. The disclosure also provides antibodies comprising a heavy chain variable domain sequence that is at least 80%, at least 85%, at least 90%, or at least 95% identical to SEQ ID NO:68 and a light chain variable domain sequence that is at least 80%, at least 85%, at least 90%, or at least 95% identical to SEQ ID NO:283. The disclosure also provides antibodies comprising a heavy chain variable domain sequence that is at least 80%, at least 85%, at least 90%, or at least 95% identical to SEQ ID NO:68 and a light chain variable domain sequence that is at least 80%, at least 85%, at least 90%, or at least 95% identical to SEQ ID NO:311. The disclosure also provides antibodies comprising a heavy chain variable domain sequence that is at least 80%, at least 85%, at least 90%, or at least 95% identical to SEQ ID NO:69 and a light chain variable domain sequence that is at least 80%, at least 85%, at least 90%, or at least 95% identical to SEQ ID NO:312. The disclosure also provides antibodies comprising a heavy chain variable domain sequence that is at least 80%, at least 85%, at least 90%, or at least 95% identical to SEQ ID NO:70 and a light chain variable domain sequence that is at least 80%, at least 85%, at least 90%, or at least 95% identical to SEQ ID NO:293. The disclosure also provides antibodies comprising a heavy chain variable domain sequence that is at least 80%, at least 85%, at least 90%, or at least 95% identical to SEQ ID NO:71 and a light chain variable domain sequence that is at least 80%, at least 85%, at least 90%, or at least 95% identical to SEQ ID NO:313. The disclosure also provides antibodies comprising a heavy chain variable domain sequence that is at least 80%, at least 85%, at least 90%, or at least 95% identical to SEQ ID NO:72 and a light chain variable domain sequence that is at least 80%, at least 85%, at least 90%, or at least 95% identical to SEQ ID NO:314. The disclosure also provides antibodies comprising a heavy chain variable domain sequence that is at least 80%, at least 85%, at least 90%, or at least 95% identical to SEQ ID NO:72 and a light chain variable domain sequence that is at least 80%, at least 85%, at least 90%, or at least 95% identical to SEQ ID NO:305. The disclosure also provides antibodies comprising a heavy chain variable domain sequence that is at least 80%, at least 85%, at least 90%, or at least 95% identical to SEQ ID NO:72 and a light chain variable domain sequence that is at least 80%, at least 85%, at least 90%, or at least 95% identical to SEQ ID NO:293. The disclosure also provides antibodies comprising a heavy chain variable domain sequence that is at least 80%, at least 85%, at least 90%, or at least 95% identical to SEQ ID NO:73 and a light chain variable domain sequence that is at least 80%, at least 85%, at least 90%, or at least 95% identical to SEQ ID NO:315. The disclosure also provides antibodies comprising a heavy chain variable domain sequence that is at least 80%, at least 85%, at least 90%, or at least 95% identical to SEQ ID NO:74 and a light chain variable domain sequence that is at least 80%, at least 85%, at least 90%, or at least 95% identical to SEQ ID NO:316. The disclosure also provides antibodies comprising a heavy chain variable domain sequence that is at least 80%, at least 85%, at least 90%, or at least 95% identical to SEQ ID NO:75 and a light chain variable domain sequence that is at least 80%, at least 85%, at least 90%, or at least 95% identical to SEQ ID NO:317. The disclosure also provides antibodies comprising a heavy chain variable domain sequence that is at least 80%, at least 85%, at least 90%, or at least 95% identical to SEQ ID NO:75 and a light chain variable domain sequence that is at least 80%, at least 85%, at least 90%, or at least 95% identical to SEQ ID NO:318. The disclosure also provides antibodies comprising a heavy chain variable domain sequence that is at least 80%, at least 85%, at least 90%, or at least 95% identical to SEQ ID NO:75 and a light chain variable domain sequence that is at least 80%, at least 85%, at least 90%, or at least 95% identical to SEQ ID NO:319. The disclosure also provides antibodies comprising a heavy chain variable domain sequence that is at least 80%, at least 85%, at least 90%, or at least 95% identical to SEQ ID NO:76 and a light chain variable domain sequence that is at least 80%, at least 85%, at least 90%, or at least 95% identical to SEQ ID NO:320. The disclosure also provides antibodies comprising a heavy chain variable domain sequence that is at least 80%, at least 85%, at least 90%, or at least 95% identical to SEQ ID NO:77 and a light chain variable domain sequence that is at least 80%, at least 85%, at least 90%, or at least 95% identical to SEQ ID NO:321. The disclosure also provides antibodies comprising a heavy chain variable domain sequence that is at least 80%, at least 85%, at least 90%, or at least 95% identical to SEQ ID NO:77 and a light chain variable domain sequence that is at least 80%, at least 85%, at least 90%, or at least 95% identical to SEQ ID NO:322. The disclosure also provides antibodies comprising a heavy chain variable domain sequence that is at least 80%, at least 85%, at least 90%, or at least 95% identical to SEQ ID NO:77 and a light chain variable domain sequence that is at least 80%, at least 85%, at least 90%, or at least 95% identical to SEQ ID NO:323. The disclosure also provides antibodies comprising a heavy chain variable domain sequence that is at least 80%, at least 85%, at least 90%, or at least 95% identical to SEQ ID NO:77 and a light chain variable domain sequence that is at least 80%, at least 85%, at least 90%, or at least 95% identical to SEQ ID NO:324. The disclosure also provides antibodies comprising a heavy chain variable domain sequence that is at least 80%, at least 85%, at least 90%, or at least 95% identical to SEQ ID NO:78 and a light chain variable domain sequence that is at least 80%, at least 85%, at least 90%, or at least 95% identical to SEQ ID NO:318. The disclosure also provides antibodies comprising a heavy chain variable domain sequence that is at least 80%, at least 85%, at least 90%, or at least 95% identical to SEQ ID NO:79 and a light chain variable domain sequence that is at least 80%, at least 85%, at least 90%, or at least 95% identical to SEQ ID NO:321. The disclosure also provides antibodies comprising a heavy chain variable domain sequence that is at least 80%, at least 85%, at least 90%, or at least 95% identical to SEQ ID NO:80 and a light chain variable domain sequence that is at least 80%, at least 85%, at least 90%, or at least 95% identical to SEQ ID NO:325. The disclosure also provides antibodies comprising a heavy chain variable domain sequence that is at least 80%, at least 85%, at least 90%, or at least 95% identical to SEQ ID NO:80 and a light chain variable domain sequence that is at least 80%, at least 85%, at least 90%, or at least 95% identical to SEQ ID NO:326. The disclosure also provides antibodies comprising a heavy chain variable domain sequence that is at least 80%, at least 85%, at least 90%, or at least 95% identical to SEQ ID NO:80 and a light chain variable domain sequence that is at least 80%, at least 85%, at least 90%, or at least 95% identical to SEQ ID NO:318. The disclosure also provides antibodies comprising a heavy chain variable domain sequence that is at least 80%, at least 85%, at least 90%, or at least 95% identical to SEQ ID NO:81 and a light chain variable domain sequence that is at least 80%, at least 85%, at least 90%, or at least 95% identical to SEQ ID NO:318. The disclosure also provides antibodies comprising a heavy chain variable domain sequence that is at least 80%, at least 85%, at least 90%, or at least 95% identical to SEQ ID NO:82 and a light chain variable domain sequence that is at least 80%, at least 85%, at least 90%, or at least 95% identical to SEQ ID NO:327. The disclosure also provides antibodies comprising a heavy chain variable domain sequence that is at least 80%, at least 85%, at least 90%, or at least 95% identical to SEQ ID NO:82 and a light chain variable domain sequence that is at least 80%, at least 85%, at least 90%, or at least 95% identical to SEQ ID NO:328. The disclosure also provides antibodies comprising a heavy chain variable domain sequence that is at least 80%, at least 85%, at least 90%, or at least 95% identical to SEQ ID NO:82 and a light chain variable domain sequence that is at least 80%, at least 85%, at least 90%, or at least 95% identical to SEQ ID NO:329. The disclosure also provides antibodies comprising a heavy chain variable domain sequence that is at least 80%, at least 85%, at least 90%, or at least 95% identical to SEQ ID NO:82 and a light chain variable domain sequence that is at least 80%, at least 85%, at least 90%, or at least 95% identical to SEQ ID NO:330. The disclosure also provides antibodies comprising a heavy chain variable domain sequence that is at least 80%, at least 85%, at least 90%, or at least 95% identical to SEQ ID NO:83 and a light chain variable domain sequence that is at least 80%, at least 85%, at least 90%, or at least 95% identical to SEQ ID NO:331. The disclosure also provides antibodies comprising a heavy chain variable domain sequence that is at least 80%, at least 85%, at least 90%, or at least 95% identical to SEQ ID NO:83 and a light chain variable domain sequence that is at least 80%, at least 85%, at least 90%, or at least 95% identical to SEQ ID NO:318. The disclosure also provides antibodies comprising a heavy chain variable domain sequence that is at least 80%, at least 85%, at least 90%, or at least 95% identical to SEQ ID NO:83 and a light chain variable domain sequence that is at least 80%, at least 85%, at least 90%, or at least 95% identical to SEQ ID NO:332. The disclosure also provides antibodies comprising a heavy chain variable domain sequence that is at least 80%, at least 85%, at least 90%, or at least 95% identical to SEQ ID NO:83 and a light chain variable domain sequence that is at least 80%, at least 85%, at least 90%, or at least 95% identical to SEQ ID NO:333. The disclosure also provides antibodies comprising a heavy chain variable domain sequence that is at least 80%, at least 85%, at least 90%, or at least 95% identical to SEQ ID NO:84 and a light chain variable domain sequence that is at least 80%, at least 85%, at least 90%, or at least 95% identical to SEQ ID NO:334. The disclosure also provides antibodies comprising a heavy chain variable domain sequence that is at least 80%, at least 85%, at least 90%, or at least 95% identical to SEQ ID NO:85 and a light chain variable domain sequence that is at least 80%, at least 85%, at least 90%, or at least 95% identical to SEQ ID NO:304. The disclosure also provides antibodies comprising a heavy chain variable domain sequence that is at least 80%, at least 85%, at least 90%, or at least 95% identical to SEQ ID NO:85 and a light chain variable domain sequence that is at least 80%, at least 85%, at least 90%, or at least 95% identical to SEQ ID NO:326. The disclosure also provides antibodies comprising a heavy chain variable domain sequence that is at least 80%, at least 85%, at least 90%, or at least 95% identical to SEQ ID NO:85 and a light chain variable domain sequence that is at least 80%, at least 85%, at least 90%, or at least 95% identical to SEQ ID NO:318. The disclosure also provides antibodies comprising a heavy chain variable domain sequence that is at least 80%, at least 85%, at least 90%, or at least 95% identical to SEQ ID NO:85 and a light chain variable domain sequence that is at least 80%, at least 85%, at least 90%, or at least 95% identical to SEQ ID NO:332. The disclosure also provides antibodies comprising a heavy chain variable domain sequence that is at least 80%, at least 85%, at least 90%, or at least 95% identical to SEQ ID NO:86 and a light chain variable domain sequence that is at least 80%, at least 85%, at least 90%, or at least 95% identical to SEQ ID NO:335. The disclosure also provides antibodies comprising a heavy chain variable domain sequence that is at least 80%, at least 85%, at least 90%, or at least 95% identical to SEQ ID NO:87 and a light chain variable domain sequence that is at least 80%, at least 85%, at least 90%, or at least 95% identical to SEQ ID NO:336. The disclosure also provides antibodies comprising a heavy chain variable domain sequence that is at least 80%, at least 85%, at least 90%, or at least 95% identical to SEQ ID NO:87 and a light chain variable domain sequence that is at least 80%, at least 85%, at least 90%, or at least 95% identical to SEQ ID NO:337. The disclosure also provides antibodies comprising a heavy chain variable domain sequence that is at least 80%, at least 85%, at least 90%, or at least 95% identical to SEQ ID NO:87 and a light chain variable domain sequence that is at least 80%, at least 85%, at least 90%, or at least 95% identical to SEQ ID NO:326. The disclosure also provides antibodies comprising a heavy chain variable domain sequence that is at least 80%, at least 85%, at least 90%, or at least 95% identical to SEQ ID NO:87 and a light chain variable domain sequence that is at least 80%, at least 85%, at least 90%, or at least 95% identical to SEQ ID NO:338. The disclosure also provides antibodies comprising a heavy chain variable domain sequence that is at least 80%, at least 85%, at least 90%, or at least 95% identical to SEQ ID NO:87 and a light chain variable domain sequence that is at least 80%, at least 85%, at least 90%, or at least 95% identical to SEQ ID NO:339. The disclosure also provides antibodies comprising a heavy chain variable domain sequence that is at least 80%, at least 85%, at least 90%, or at least 95% identical to SEQ ID NO:87 and a light chain variable domain sequence that is at least 80%, at least 85%, at least 90%, or at least 95% identical to SEQ ID NO:340. The disclosure also provides antibodies comprising a heavy chain variable domain sequence that is at least 80%, at least 85%, at least 90%, or at least 95% identical to SEQ ID NO:87 and a light chain variable domain sequence that is at least 80%, at least 85%, at least 90%, or at least 95% identical to SEQ ID NO:341. The disclosure also provides antibodies comprising a heavy chain variable domain sequence that is at least 80%, at least 85%, at least 90%, or at least 95% identical to SEQ ID NO:87 and a light chain variable domain sequence that is at least 80%, at least 85%, at least 90%, or at least 95% identical to SEQ ID NO:333. The disclosure also provides antibodies comprising a heavy chain variable domain sequence that is at least 80%, at least 85%, at least 90%, or at least 95% identical to SEQ ID NO:88 and a light chain variable domain sequence that is at least 80%, at least 85%, at least 90%, or at least 95% identical to SEQ ID NO:318. The disclosure also provides antibodies comprising a heavy chain variable domain sequence that is at least 80%, at least 85%, at least 90%, or at least 95% identical to SEQ ID NO:89 and a light chain variable domain sequence that is at least 80%, at least 85%, at least 90%, or at least 95% identical to SEQ ID NO:342. The disclosure also provides antibodies comprising a heavy chain variable domain sequence that is at least 80%, at least 85%, at least 90%, or at least 95% identical to SEQ ID NO:89 and a light chain variable domain sequence that is at least 80%, at least 85%, at least 90%, or at least 95% identical to SEQ ID NO:313. The disclosure also provides antibodies comprising a heavy chain variable domain sequence that is at least 80%, at least 85%, at least 90%, or at least 95% identical to SEQ ID NO:90 and a light chain variable domain sequence that is at least 80%, at least 85%, at least 90%, or at least 95% identical to SEQ ID NO:342. The disclosure also provides antibodies comprising a heavy chain variable domain sequence that is at least 80%, at least 85%, at least 90%, or at least 95% identical to SEQ ID NO:91 and a light chain variable domain sequence that is at least 80%, at least 85%, at least 90%, or at least 95% identical to SEQ ID NO:329. The disclosure also provides antibodies comprising a heavy chain variable domain sequence that is at least 80%, at least 85%, at least 90%, or at least 95% identical to SEQ ID NO:91 and a light chain variable domain sequence that is at least 80%, at least 85%, at least 90%, or at least 95% identical to SEQ ID NO:294. The disclosure also provides antibodies comprising a heavy chain variable domain sequence that is at least 80%, at least 85%, at least 90%, or at least 95% identical to SEQ ID NO:91 and a light chain variable domain sequence that is at least 80%, at least 85%, at least 90%, or at least 95% identical to SEQ ID NO:333. The disclosure also provides antibodies comprising a heavy chain variable domain sequence that is at least 80%, at least 85%, at least 90%, or at least 95% identical to SEQ ID NO:91 and a light chain variable domain sequence that is at least 80%, at least 85%, at least 90%, or at least 95% identical to SEQ ID NO:343. The disclosure also provides antibodies comprising a heavy chain variable domain sequence that is at least 80%, at least 85%, at least 90%, or at least 95% identical to SEQ ID NO:92 and a light chain variable domain sequence that is at least 80%, at least 85%, at least 90%, or at least 95% identical to SEQ ID NO:344. The disclosure also provides antibodies comprising a heavy chain variable domain sequence that is at least 80%, at least 85%, at least 90%, or at least 95% identical to SEQ ID NO:92 and a light chain variable domain sequence that is at least 80%, at least 85%, at least 90%, or at least 95% identical to SEQ ID NO:345. The disclosure also provides antibodies comprising a heavy chain variable domain sequence that is at least 80%, at least 85%, at least 90%, or at least 95% identical to SEQ ID NO:92 and a light chain variable domain sequence that is at least 80%, at least 85%, at least 90%, or at least 95% identical to SEQ ID NO:346. The disclosure also provides antibodies comprising a heavy chain variable domain sequence that is at least 80%, at least 85%, at least 90%, or at least 95% identical to SEQ ID NO:93 and a light chain variable domain sequence that is at least 80%, at least 85%, at least 90%, or at least 95% identical to SEQ ID NO:316. The disclosure also provides antibodies comprising a heavy chain variable domain sequence that is at least 80%, at least 85%, at least 90%, or at least 95% identical to SEQ ID NO:94 and a light chain variable domain sequence that is at least 80%, at least 85%, at least 90%, or at least 95% identical to SEQ ID NO:347. The disclosure also provides antibodies comprising a heavy chain variable domain sequence that is at least 80%, at least 85%, at least 90%, or at least 95% identical to SEQ ID NO:95 and a light chain variable domain sequence that is at least 80%, at least 85%, at least 90%, or at least 95% identical to SEQ ID NO:348. The disclosure also provides antibodies comprising a heavy chain variable domain sequence that is at least 80%, at least 85%, at least 90%, or at least 95% identical to SEQ ID NO:96 and a light chain variable domain sequence that is at least 80%, at least 85%, at least 90%, or at least 95% identical to SEQ ID NO:349. The disclosure also provides antibodies comprising a heavy chain variable domain sequence that is at least 80%, at least 85%, at least 90%, or at least 95% identical to SEQ ID NO:97 and a light chain variable domain sequence that is at least 80%, at least 85%, at least 90%, or at least 95% identical to SEQ ID NO:342. The disclosure also provides antibodies comprising a heavy chain variable domain sequence that is at least 80%, at least 85%, at least 90%, or at least 95% identical to SEQ ID NO:98 and a light chain variable domain sequence that is at least 80%, at least 85%, at least 90%, or at least 95% identical to SEQ ID NO:304. The disclosure also provides antibodies comprising a heavy chain variable domain sequence that is at least 80%, at least 85%, at least 90%, or at least 95% identical to SEQ ID NO:99 and a light chain variable domain sequence that is at least 80%, at least 85%, at least 90%, or at least 95% identical to SEQ ID NO:284. The disclosure also provides antibodies comprising a heavy chain variable domain sequence that is at least 80%, at least 85%, at least 90%, or at least 95% identical to SEQ ID NO:99 and a light chain variable domain sequence that is at least 80%, at least 85%, at least 90%, or at least 95% identical to SEQ ID NO:296. The disclosure also provides antibodies comprising a heavy chain variable domain sequence that is at least 80%, at least 85%, at least 90%, or at least 95% identical to SEQ ID NO:99 and a light chain variable domain sequence that is at least 80%, at least 85%, at least 90%, or at least 95% identical to SEQ ID NO:304. The disclosure also provides antibodies comprising a heavy chain variable domain sequence that is at least 80%, at least 85%, at least 90%, or at least 95% identical to SEQ ID NO:99 and a light chain variable domain sequence that is at least 80%, at least 85%, at least 90%, or at least 95% identical to SEQ ID NO:350. The disclosure also provides antibodies comprising a heavy chain variable domain sequence that is at least 80%, at least 85%, at least 90%, or at least 95% identical to SEQ ID NO:99 and a light chain variable domain sequence that is at least 80%, at least 85%, at least 90%, or at least 95% identical to SEQ ID NO:351. The disclosure also provides antibodies comprising a heavy chain variable domain sequence that is at least 80%, at least 85%, at least 90%, or at least 95% identical to SEQ ID NO:99 and a light chain variable domain sequence that is at least 80%, at least 85%, at least 90%, or at least 95% identical to SEQ ID NO:318. The disclosure also provides antibodies comprising a heavy chain variable domain sequence that is at least 80%, at least 85%, at least 90%, or at least 95% identical to SEQ ID NO:99 and a light chain variable domain sequence that is at least 80%, at least 85%, at least 90%, or at least 95% identical to SEQ ID NO:352. The disclosure also provides antibodies comprising a heavy chain variable domain sequence that is at least 80%, at least 85%, at least 90%, or at least 95% identical to SEQ ID NO:99 and a light chain variable domain sequence that is at least 80%, at least 85%, at least 90%, or at least 95% identical to SEQ ID NO:353. The disclosure also provides antibodies comprising a heavy chain variable domain sequence that is at least 80%, at least 85%, at least 90%, or at least 95% identical to SEQ ID NO:99 and a light chain variable domain sequence that is at least 80%, at least 85%, at least 90%, or at least 95% identical to SEQ ID NO:354. The disclosure also provides antibodies comprising a heavy chain variable domain sequence that is at least 80%, at least 85%, at least 90%, or at least 95% identical to SEQ ID NO:100 and a light chain variable domain sequence that is at least 80%, at least 85%, at least 90%, or at least 95% identical to SEQ ID NO:355. The disclosure also provides antibodies comprising a heavy chain variable domain sequence that is at least 80%, at least 85%, at least 90%, or at least 95% identical to SEQ ID NO:101 and a light chain variable domain sequence that is at least 80%, at least 85%, at least 90%, or at least 95% identical to SEQ ID NO:314. The disclosure also provides antibodies comprising a heavy chain variable domain sequence that is at least 80%, at least 85%, at least 90%, or at least 95% identical to SEQ ID NO:101 and a light chain variable domain sequence that is at least 80%, at least 85%, at least 90%, or at least 95% identical to SEQ ID NO:304. The disclosure also provides antibodies comprising a heavy chain variable domain sequence that is at least 80%, at least 85%, at least 90%, or at least 95% identical to SEQ ID NO:101 and a light chain variable domain sequence that is at least 80%, at least 85%, at least 90%, or at least 95% identical to SEQ ID NO:332. The disclosure also provides antibodies comprising a heavy chain variable domain sequence that is at least 80%, at least 85%, at least 90%, or at least 95% identical to SEQ ID NO:101 and a light chain variable domain sequence that is at least 80%, at least 85%, at least 90%, or at least 95% identical to SEQ ID NO:322. The disclosure also provides antibodies comprising a heavy chain variable domain sequence that is at least 80%, at least 85%, at least 90%, or at least 95% identical to SEQ ID NO:102 and a light chain variable domain sequence that is at least 80%, at least 85%, at least 90%, or at least 95% identical to SEQ ID NO:356. The disclosure also provides antibodies comprising a heavy chain variable domain sequence that is at least 80%, at least 85%, at least 90%, or at least 95% identical to SEQ ID NO:103 and a light chain variable domain sequence that is at least 80%, at least 85%, at least 90%, or at least 95% identical to SEQ ID NO:356. The disclosure also provides antibodies comprising a heavy chain variable domain sequence that is at least 80%, at least 85%, at least 90%, or at least 95% identical to SEQ ID NO:104 and a light chain variable domain sequence that is at least 80%, at least 85%, at least 90%, or at least 95% identical to SEQ ID NO:357. The disclosure also provides antibodies comprising a heavy chain variable domain sequence that is at least 80%, at least 85%, at least 90%, or at least 95% identical to SEQ ID NO:105 and a light chain variable domain sequence that is at least 80%, at least 85%, at least 90%, or at least 95% identical to SEQ ID NO:267. The disclosure also provides antibodies comprising a heavy chain variable domain sequence that is at least 80%, at least 85%, at least 90%, or at least 95% identical to SEQ ID NO:105 and a light chain variable domain sequence that is at least 80%, at least 85%, at least 90%, or at least 95% identical to SEQ ID NO:261. The disclosure also provides antibodies comprising a heavy chain variable domain sequence that is at least 80%, at least 85%, at least 90%, or at least 95% identical to SEQ ID NO:105 and a light chain variable domain sequence that is at least 80%, at least 85%, at least 90%, or at least 95% identical to SEQ ID NO:358. The disclosure also provides antibodies comprising a heavy chain variable domain sequence that is at least 80%, at least 85%, at least 90%, or at least 95% identical to SEQ ID NO:105 and a light chain variable domain sequence that is at least 80%, at least 85%, at least 90%, or at least 95% identical to SEQ ID NO:359. The disclosure also provides antibodies comprising a heavy chain variable domain sequence that is at least 80%, at least 85%, at least 90%, or at least 95% identical to SEQ ID NO:106 and a light chain variable domain sequence that is at least 80%, at least 85%, at least 90%, or at least 95% identical to SEQ ID NO:360. The disclosure also provides antibodies comprising a heavy chain variable domain sequence that is at least 80%, at least 85%, at least 90%, or at least 95% identical to SEQ ID NO:106 and a light chain variable domain sequence that is at least 80%, at least 85%, at least 90%, or at least 95% identical to SEQ ID NO:361. The disclosure also provides antibodies comprising a heavy chain variable domain sequence that is at least 80%, at least 85%, at least 90%, or at least 95% identical to SEQ ID NO:106 and a light chain variable domain sequence that is at least 80%, at least 85%, at least 90%, or at least 95% identical to SEQ ID NO:362. The disclosure also provides antibodies comprising a heavy chain variable domain sequence that is at least 80%, at least 85%, at least 90%, or at least 95% identical to SEQ ID NO:106 and a light chain variable domain sequence that is at least 80%, at least 85%, at least 90%, or at least 95% identical to SEQ ID NO:363. The disclosure also provides antibodies comprising a heavy chain variable domain sequence that is at least 80%, at least 85%, at least 90%, or at least 95% identical to SEQ ID NO:106 and a light chain variable domain sequence that is at least 80%, at least 85%, at least 90%, or at least 95% identical to SEQ ID NO:364. The disclosure also provides antibodies comprising a heavy chain variable domain sequence that is at least 80%, at least 85%, at least 90%, or at least 95% identical to SEQ ID NO:106 and a light chain variable domain sequence that is at least 80%, at least 85%, at least 90%, or at least 95% identical to SEQ ID NO:312. The disclosure also provides antibodies comprising a heavy chain variable domain sequence that is at least 80%, at least 85%, at least 90%, or at least 95% identical to SEQ ID NO:106 and a light chain variable domain sequence that is at least 80%, at least 85%, at least 90%, or at least 95% identical to SEQ ID NO:365. The disclosure also provides antibodies comprising a heavy chain variable domain sequence that is at least 80%, at least 85%, at least 90%, or at least 95% identical to SEQ ID NO:106 and a light chain variable domain sequence that is at least 80%, at least 85%, at least 90%, or at least 95% identical to SEQ ID NO:366. The disclosure also provides antibodies comprising a heavy chain variable domain sequence that is at least 80%, at least 85%, at least 90%, or at least 95% identical to SEQ ID NO:107 and a light chain variable domain sequence that is at least 80%, at least 85%, at least 90%, or at least 95% identical to SEQ ID NO:262. The disclosure also provides antibodies comprising a heavy chain variable domain sequence that is at least 80%, at least 85%, at least 90%, or at least 95% identical to SEQ ID NO:108 and a light chain variable domain sequence that is at least 80%, at least 85%, at least 90%, or at least 95% identical to SEQ ID NO:283. The disclosure also provides antibodies comprising a heavy chain variable domain sequence that is at least 80%, at least 85%, at least 90%, or at least 95% identical to SEQ ID NO:108 and a light chain variable domain sequence that is at least 80%, at least 85%, at least 90%, or at least 95% identical to SEQ ID NO:359. The disclosure also provides antibodies comprising a heavy chain variable domain sequence that is at least 80%, at least 85%, at least 90%, or at least 95% identical to SEQ ID NO:108 and a light chain variable domain sequence that is at least 80%, at least 85%, at least 90%, or at least 95% identical to SEQ ID NO:367. The disclosure also provides antibodies comprising a heavy chain variable domain sequence that is at least 80%, at least 85%, at least 90%, or at least 95% identical to SEQ ID NO:109 and a light chain variable domain sequence that is at least 80%, at least 85%, at least 90%, or at least 95% identical to SEQ ID NO:368. The disclosure also provides antibodies comprising a heavy chain variable domain sequence that is at least 80%, at least 85%, at least 90%, or at least 95% identical to SEQ ID NO:110 and a light chain variable domain sequence that is at least 80%, at least 85%, at least 90%, or at least 95% identical to SEQ ID NO:368. The disclosure also provides antibodies comprising a heavy chain variable domain sequence that is at least 80%, at least 85%, at least 90%, or at least 95% identical to SEQ ID NO:111 and a light chain variable domain sequence that is at least 80%, at least 85%, at least 90%, or at least 95% identical to SEQ ID NO:333. The disclosure also provides antibodies comprising a heavy chain variable domain sequence that is at least 80%, at least 85%, at least 90%, or at least 95% identical to SEQ ID NO:112 and a light chain variable domain sequence that is at least 80%, at least 85%, at least 90%, or at least 95% identical to SEQ ID NO:369. The disclosure also provides antibodies comprising a heavy chain variable domain sequence that is at least 80%, at least 85%, at least 90%, or at least 95% identical to SEQ ID NO:113 and a light chain variable domain sequence that is at least 80%, at least 85%, at least 90%, or at least 95% identical to SEQ ID NO:333. The disclosure also provides antibodies comprising a heavy chain variable domain sequence that is at least 80%, at least 85%, at least 90%, or at least 95% identical to SEQ ID NO:114 and a light chain variable domain sequence that is at least 80%, at least 85%, at least 90%, or at least 95% identical to SEQ ID NO:370. The disclosure also provides antibodies comprising a heavy chain variable domain sequence that is at least 80%, at least 85%, at least 90%, or at least 95% identical to SEQ ID NO:115 and a light chain variable domain sequence that is at least 80%, at least 85%, at least 90%, or at least 95% identical to SEQ ID NO:371. The disclosure also provides antibodies comprising a heavy chain variable domain sequence that is at least 80%, at least 85%, at least 90%, or at least 95% identical to SEQ ID NO:116 and a light chain variable domain sequence that is at least 80%, at least 85%, at least 90%, or at least 95% identical to SEQ ID NO:372. The disclosure also provides antibodies comprising a heavy chain variable domain sequence that is at least 80%, at least 85%, at least 90%, or at least 95% identical to SEQ ID NO:117 and a light chain variable domain sequence that is at least 80%, at least 85%, at least 90%, or at least 95% identical to SEQ ID NO:373. The disclosure also provides antibodies comprising a heavy chain variable domain sequence that is at least 80%, at least 85%, at least 90%, or at least 95% identical to SEQ ID NO:118 and a light chain variable domain sequence that is at least 80%, at least 85%, at least 90%, or at least 95% identical to SEQ ID NO:333. The disclosure also provides antibodies comprising a heavy chain variable domain sequence that is at least 80%, at least 85%, at least 90%, or at least 95% identical to SEQ ID NO:119 and a light chain variable domain sequence that is at least 80%, at least 85%, at least 90%, or at least 95% identical to SEQ ID NO:333. The disclosure also provides antibodies comprising a heavy chain variable domain sequence that is at least 80%, at least 85%, at least 90%, or at least 95% identical to SEQ ID NO:120 and a light chain variable domain sequence that is at least 80%, at least 85%, at least 90%, or at least 95% identical to SEQ ID NO:374. The disclosure also provides antibodies comprising a heavy chain variable domain sequence that is at least 80%, at least 85%, at least 90%, or at least 95% identical to SEQ ID NO:121 and a light chain variable domain sequence that is at least 80%, at least 85%, at least 90%, or at least 95% identical to SEQ ID NO:329. The disclosure also provides antibodies comprising a heavy chain variable domain sequence that is at least 80%, at least 85%, at least 90%, or at least 95% identical to SEQ ID NO:121 and a light chain variable domain sequence that is at least 80%, at least 85%, at least 90%, or at least 95% identical to SEQ ID NO:333. The disclosure also provides antibodies comprising a heavy chain variable domain sequence that is at least 80%, at least 85%, at least 90%, or at least 95% identical to SEQ ID NO:122 and a light chain variable domain sequence that is at least 80%, at least 85%, at least 90%, or at least 95% identical to SEQ ID NO:375. The disclosure also provides antibodies comprising a heavy chain variable domain sequence that is at least 80%, at least 85%, at least 90%, or at least 95% identical to SEQ ID NO:123 and a light chain variable domain sequence that is at least 80%, at least 85%, at least 90%, or at least 95% identical to SEQ ID NO:376. The disclosure also provides antibodies comprising a heavy chain variable domain sequence that is at least 80%, at least 85%, at least 90%, or at least 95% identical to SEQ ID NO:124 and a light chain variable domain sequence that is at least 80%, at least 85%, at least 90%, or at least 95% identical to SEQ ID NO:377. The disclosure also provides antibodies comprising a heavy chain variable domain sequence that is at least 80%, at least 85%, at least 90%, or at least 95% identical to SEQ ID NO:124 and a light chain variable domain sequence that is at least 80%, at least 85%, at least 90%, or at least 95% identical to SEQ ID NO:378. The disclosure also provides antibodies comprising a heavy chain variable domain sequence that is at least 80%, at least 85%, at least 90%, or at least 95% identical to SEQ ID NO:125 and a light chain variable domain sequence that is at least 80%, at least 85%, at least 90%, or at least 95% identical to SEQ ID NO:379. The disclosure also provides antibodies comprising a heavy chain variable domain sequence that is at least 80%, at least 85%, at least 90%, or at least 95% identical to SEQ ID NO:126 and a light chain variable domain sequence that is at least 80%, at least 85%, at least 90%, or at least 95% identical to SEQ ID NO:294. The disclosure also provides antibodies comprising a heavy chain variable domain sequence that is at least 80%, at least 85%, at least 90%, or at least 95% identical to SEQ ID NO:126 and a light chain variable domain sequence that is at least 80%, at least 85%, at least 90%, or at least 95% identical to SEQ ID NO:377. The disclosure also provides antibodies comprising a heavy chain variable domain sequence that is at least 80%, at least 85%, at least 90%, or at least 95% identical to SEQ ID NO:126 and a light chain variable domain sequence that is at least 80%, at least 85%, at least 90%, or at least 95% identical to SEQ ID NO:333. The disclosure also provides antibodies comprising a heavy chain variable domain sequence that is at least 80%, at least 85%, at least 90%, or at least 95% identical to SEQ ID NO:127 and a light chain variable domain sequence that is at least 80%, at least 85%, at least 90%, or at least 95% identical to SEQ ID NO:333. The disclosure also provides antibodies comprising a heavy chain variable domain sequence that is at least 80%, at least 85%, at least 90%, or at least 95% identical to SEQ ID NO:128 and a light chain variable domain sequence that is at least 80%, at least 85%, at least 90%, or at least 95% identical to SEQ ID NO:294. The disclosure also provides antibodies comprising a heavy chain variable domain sequence that is at least 80%, at least 85%, at least 90%, or at least 95% identical to SEQ ID NO:128 and a light chain variable domain sequence that is at least 80%, at least 85%, at least 90%, or at least 95% identical to SEQ ID NO:359. The disclosure also provides antibodies comprising a heavy chain variable domain sequence that is at least 80%, at least 85%, at least 90%, or at least 95% identical to SEQ ID NO:128 and a light chain variable domain sequence that is at least 80%, at least 85%, at least 90%, or at least 95% identical to SEQ ID NO:380. The disclosure also provides antibodies comprising a heavy chain variable domain sequence that is at least 80%, at least 85%, at least 90%, or at least 95% identical to SEQ ID NO:128 and a light chain variable domain sequence that is at least 80%, at least 85%, at least 90%, or at least 95% identical to SEQ ID NO:333. The disclosure also provides antibodies comprising a heavy chain variable domain sequence that is at least 80%, at least 85%, at least 90%, or at least 95% identical to SEQ ID NO:129 and a light chain variable domain sequence that is at least 80%, at least 85%, at least 90%, or at least 95% identical to SEQ ID NO:333. The disclosure also provides antibodies comprising a heavy chain variable domain sequence that is at least 80%, at least 85%, at least 90%, or at least 95% identical to SEQ ID NO:130 and a light chain variable domain sequence that is at least 80%, at least 85%, at least 90%, or at least 95% identical to SEQ ID NO:333. The disclosure also provides antibodies comprising a heavy chain variable domain sequence that is at least 80%, at least 85%, at least 90%, or at least 95% identical to SEQ ID NO:131 and a light chain variable domain sequence that is at least 80%, at least 85%, at least 90%, or at least 95% identical to SEQ ID NO:381. The disclosure also provides antibodies comprising a heavy chain variable domain sequence that is at least 80%, at least 85%, at least 90%, or at least 95% identical to SEQ ID NO:132 and a light chain variable domain sequence that is at least 80%, at least 85%, at least 90%, or at least 95% identical to SEQ ID NO:317. The disclosure also provides antibodies comprising a heavy chain variable domain sequence that is at least 80%, at least 85%, at least 90%, or at least 95% identical to SEQ ID NO:133 and a light chain variable domain sequence that is at least 80%, at least 85%, at least 90%, or at least 95% identical to SEQ ID NO:382. The disclosure also provides antibodies comprising a heavy chain variable domain sequence that is at least 80%, at least 85%, at least 90%, or at least 95% identical to SEQ ID NO:134 and a light chain variable domain sequence that is at least 80%, at least 85%, at least 90%, or at least 95% identical to SEQ ID NO:317. The disclosure also provides antibodies comprising a heavy chain variable domain sequence that is at least 80%, at least 85%, at least 90%, or at least 95% identical to SEQ ID NO:135 and a light chain variable domain sequence that is at least 80%, at least 85%, at least 90%, or at least 95% identical to SEQ ID NO:383. The disclosure also provides antibodies comprising a heavy chain variable domain sequence that is at least 80%, at least 85%, at least 90%, or at least 95% identical to SEQ ID NO:136 and a light chain variable domain sequence that is at least 80%, at least 85%, at least 90%, or at least 95% identical to SEQ ID NO:384. The disclosure also provides antibodies comprising a heavy chain variable domain sequence that is at least 80%, at least 85%, at least 90%, or at least 95% identical to SEQ ID NO:137 and a light chain variable domain sequence that is at least 80%, at least 85%, at least 90%, or at least 95% identical to SEQ ID NO:385. The disclosure also provides antibodies comprising a heavy chain variable domain sequence that is at least 80%, at least 85%, at least 90%, or at least 95% identical to SEQ ID NO:138 and a light chain variable domain sequence that is at least 80%, at least 85%, at least 90%, or at least 95% identical to SEQ ID NO:386. The disclosure also provides antibodies comprising a heavy chain variable domain sequence that is at least 80%, at least 85%, at least 90%, or at least 95% identical to SEQ ID NO:139 and a light chain variable domain sequence that is at least 80%, at least 85%, at least 90%, or at least 95% identical to SEQ ID NO:333. The disclosure also provides antibodies comprising a heavy chain variable domain sequence that is at least 80%, at least 85%, at least 90%, or at least 95% identical to SEQ ID NO:139 and a light chain variable domain sequence that is at least 80%, at least 85%, at least 90%, or at least 95% identical to SEQ ID NO:386. The disclosure also provides antibodies comprising a heavy chain variable domain sequence that is at least 80%, at least 85%, at least 90%, or at least 95% identical to SEQ ID NO:140 and a light chain variable domain sequence that is at least 80%, at least 85%, at least 90%, or at least 95% identical to SEQ ID NO:387. The disclosure also provides antibodies comprising a heavy chain variable domain sequence that is at least 80%, at least 85%, at least 90%, or at least 95% identical to SEQ ID NO:141 and a light chain variable domain sequence that is at least 80%, at least 85%, at least 90%, or at least 95% identical to SEQ ID NO:388. The disclosure also provides antibodies comprising a heavy chain variable domain sequence that is at least 80%, at least 85%, at least 90%, or at least 95% identical to SEQ ID NO:142 and a light chain variable domain sequence that is at least 80%, at least 85%, at least 90%, or at least 95% identical to SEQ ID NO:333. The disclosure also provides antibodies comprising a heavy chain variable domain sequence that is at least 80%, at least 85%, at least 90%, or at least 95% identical to SEQ ID NO:143 and a light chain variable domain sequence that is at least 80%, at least 85%, at least 90%, or at least 95% identical to SEQ ID NO:389. The disclosure also provides antibodies comprising a heavy chain variable domain sequence that is at least 80%, at least 85%, at least 90%, or at least 95% identical to SEQ ID NO:144 and a light chain variable domain sequence that is at least 80%, at least 85%, at least 90%, or at least 95% identical to SEQ ID NO:329. The disclosure also provides antibodies comprising a heavy chain variable domain sequence that is at least 80%, at least 85%, at least 90%, or at least 95% identical to SEQ ID NO:144 and a light chain variable domain sequence that is at least 80%, at least 85%, at least 90%, or at least 95% identical to SEQ ID NO:333. The disclosure also provides antibodies comprising a heavy chain variable domain sequence that is at least 80%, at least 85%, at least 90%, or at least 95% identical to SEQ ID NO:145 and a light chain variable domain sequence that is at least 80%, at least 85%, at least 90%, or at least 95% identical to SEQ ID NO:390. The disclosure also provides antibodies comprising a heavy chain variable domain sequence that is at least 80%, at least 85%, at least 90%, or at least 95% identical to SEQ ID NO:146 and a light chain variable domain sequence that is at least 80%, at least 85%, at least 90%, or at least 95% identical to SEQ ID NO:391. The disclosure also provides antibodies comprising a heavy chain variable domain sequence that is at least 80%, at least 85%, at least 90%, or at least 95% identical to SEQ ID NO:147 and a light chain variable domain sequence that is at least 80%, at least 85%, at least 90%, or at least 95% identical to SEQ ID NO:349. The disclosure also provides antibodies comprising a heavy chain variable domain sequence that is at least 80%, at least 85%, at least 90%, or at least 95% identical to SEQ ID NO:148 and a light chain variable domain sequence that is at least 80%, at least 85%, at least 90%, or at least 95% identical to SEQ ID NO:392. The disclosure also provides antibodies comprising a heavy chain variable domain sequence that is at least 80%, at least 85%, at least 90%, or at least 95% identical to SEQ ID NO:149 and a light chain variable domain sequence that is at least 80%, at least 85%, at least 90%, or at least 95% identical to SEQ ID NO:393. The disclosure also provides antibodies comprising a heavy chain variable domain sequence that is at least 80%, at least 85%, at least 90%, or at least 95% identical to SEQ ID NO:150 and a light chain variable domain sequence that is at least 80%, at least 85%, at least 90%, or at least 95% identical to SEQ ID NO:394. The disclosure also provides antibodies comprising a heavy chain variable domain sequence that is at least 80%, at least 85%, at least 90%, or at least 95% identical to SEQ ID NO:151 and a light chain variable domain sequence that is at least 80%, at least 85%, at least 90%, or at least 95% identical to SEQ ID NO:395. The disclosure also provides antibodies comprising a heavy chain variable domain sequence that is at least 80%, at least 85%, at least 90%, or at least 95% identical to SEQ ID NO:152 and a light chain variable domain sequence that is at least 80%, at least 85%, at least 90%, or at least 95% identical to SEQ ID NO:396. The disclosure also provides antibodies comprising a heavy chain variable domain sequence that is at least 80%, at least 85%, at least 90%, or at least 95% identical to SEQ ID NO:153 and a light chain variable domain sequence that is at least 80%, at least 85%, at least 90%, or at least 95% identical to SEQ ID NO:397. The disclosure also provides antibodies comprising a heavy chain variable domain sequence that is at least 80%, at least 85%, at least 90%, or at least 95% identical to SEQ ID NO:154 and a light chain variable domain sequence that is at least 80%, at least 85%, at least 90%, or at least 95% identical to SEQ ID NO:398. The disclosure also provides antibodies comprising a heavy chain variable domain sequence that is at least 80%, at least 85%, at least 90%, or at least 95% identical to SEQ ID NO:155 and a light chain variable domain sequence that is at least 80%, at least 85%, at least 90%, or at least 95% identical to SEQ ID NO:399. The disclosure also provides antibodies comprising a heavy chain variable domain sequence that is at least 80%, at least 85%, at least 90%, or at least 95% identical to SEQ ID NO:156 and a light chain variable domain sequence that is at least 80%, at least 85%, at least 90%, or at least 95% identical to SEQ ID NO:400. The disclosure also provides antibodies comprising a heavy chain variable domain sequence that is at least 80%, at least 85%, at least 90%, or at least 95% identical to SEQ ID NO:157 and a light chain variable domain sequence that is at least 80%, at least 85%, at least 90%, or at least 95% identical to SEQ ID NO:401. The disclosure also provides antibodies comprising a heavy chain variable domain sequence that is at least 80%, at least 85%, at least 90%, or at least 95% identical to SEQ ID NO:157 and a light chain variable domain sequence that is at least 80%, at least 85%, at least 90%, or at least 95% identical to SEQ ID NO:402. The disclosure also provides antibodies comprising a heavy chain variable domain sequence that is at least 80%, at least 85%, at least 90%, or at least 95% identical to SEQ ID NO:158 and a light chain variable domain sequence that is at least 80%, at least 85%, at least 90%, or at least 95% identical to SEQ ID NO:403. The disclosure also provides antibodies comprising a heavy chain variable domain sequence that is at least 80%, at least 85%, at least 90%, or at least 95% identical to SEQ ID NO:159 and a light chain variable domain sequence that is at least 80%, at least 85%, at least 90%, or at least 95% identical to SEQ ID NO:404. The disclosure also provides antibodies comprising a heavy chain variable domain sequence that is at least 80%, at least 85%, at least 90%, or at least 95% identical to SEQ ID NO:160 and a light chain variable domain sequence that is at least 80%, at least 85%, at least 90%, or at least 95% identical to SEQ ID NO:405. The disclosure also provides antibodies comprising a heavy chain variable domain sequence that is at least 80%, at least 85%, at least 90%, or at least 95% identical to SEQ ID NO:161 and a light chain variable domain sequence that is at least 80%, at least 85%, at least 90%, or at least 95% identical to SEQ ID NO:406. The disclosure also provides antibodies comprising a heavy chain variable domain sequence that is at least 80%, at least 85%, at least 90%, or at least 95% identical to SEQ ID NO:162 and a light chain variable domain sequence that is at least 80%, at least 85%, at least 90%, or at least 95% identical to SEQ ID NO:407. The disclosure also provides antibodies comprising a heavy chain variable domain sequence that is at least 80%, at least 85%, at least 90%, or at least 95% identical to SEQ ID NO:163 and a light chain variable domain sequence that is at least 80%, at least 85%, at least 90%, or at least 95% identical to SEQ ID NO:408. The disclosure also provides antibodies comprising a heavy chain variable domain sequence that is at least 80%, at least 85%, at least 90%, or at least 95% identical to SEQ ID NO:164 and a light chain variable domain sequence that is at least 80%, at least 85%, at least 90%, or at least 95% identical to SEQ ID NO:397. The disclosure also provides antibodies comprising a heavy chain variable domain sequence that is at least 80%, at least 85%, at least 90%, or at least 95% identical to SEQ ID NO:165 and a light chain variable domain sequence that is at least 80%, at least 85%, at least 90%, or at least 95% identical to SEQ ID NO:409. The disclosure also provides antibodies comprising a heavy chain variable domain sequence that is at least 80%, at least 85%, at least 90%, or at least 95% identical to SEQ ID NO:165 and a light chain variable domain sequence that is at least 80%, at least 85%, at least 90%, or at least 95% identical to SEQ ID NO:397. The disclosure also provides antibodies comprising a heavy chain variable domain sequence that is at least 80%, at least 85%, at least 90%, or at least 95% identical to SEQ ID NO:166 and a light chain variable domain sequence that is at least 80%, at least 85%, at least 90%, or at least 95% identical to SEQ ID NO:305. The disclosure also provides antibodies comprising a heavy chain variable domain sequence that is at least 80%, at least 85%, at least 90%, or at least 95% identical to SEQ ID NO:167 and a light chain variable domain sequence that is at least 80%, at least 85%, at least 90%, or at least 95% identical to SEQ ID NO:397. The disclosure also provides antibodies comprising a heavy chain variable domain sequence that is at least 80%, at least 85%, at least 90%, or at least 95% identical to SEQ ID NO:168 and a light chain variable domain sequence that is at least 80%, at least 85%, at least 90%, or at least 95% identical to SEQ ID NO:410. The disclosure also provides antibodies comprising a heavy chain variable domain sequence that is at least 80%, at least 85%, at least 90%, or at least 95% identical to SEQ ID NO:169 and a light chain variable domain sequence that is at least 80%, at least 85%, at least 90%, or at least 95% identical to SEQ ID NO:411. The disclosure also provides antibodies comprising a heavy chain variable domain sequence that is at least 80%, at least 85%, at least 90%, or at least 95% identical to SEQ ID NO:170 and a light chain variable domain sequence that is at least 80%, at least 85%, at least 90%, or at least 95% identical to SEQ ID NO:412. The disclosure also provides antibodies comprising a heavy chain variable domain sequence that is at least 80%, at least 85%, at least 90%, or at least 95% identical to SEQ ID NO:171 and a light chain variable domain sequence that is at least 80%, at least 85%, at least 90%, or at least 95% identical to SEQ ID NO:413. The disclosure also provides antibodies comprising a heavy chain variable domain sequence that is at least 80%, at least 85%, at least 90%, or at least 95% identical to SEQ ID NO:172 and a light chain variable domain sequence that is at least 80%, at least 85%, at least 90%, or at least 95% identical to SEQ ID NO:414. The disclosure also provides antibodies comprising a heavy chain variable domain sequence that is at least 80%, at least 85%, at least 90%, or at least 95% identical to SEQ ID NO:173 and a light chain variable domain sequence that is at least 80%, at least 85%, at least 90%, or at least 95% identical to SEQ ID NO:415. The disclosure also provides antibodies comprising a heavy chain variable domain sequence that is at least 80%, at least 85%, at least 90%, or at least 95% identical to SEQ ID NO:173 and a light chain variable domain sequence that is at least 80%, at least 85%, at least 90%, or at least 95% identical to SEQ ID NO:333. The disclosure also provides antibodies comprising a heavy chain variable domain sequence that is at least 80%, at least 85%, at least 90%, or at least 95% identical to SEQ ID NO:174 and a light chain variable domain sequence that is at least 80%, at least 85%, at least 90%, or at least 95% identical to SEQ ID NO:416. The disclosure also provides antibodies comprising a heavy chain variable domain sequence that is at least 80%, at least 85%, at least 90%, or at least 95% identical to SEQ ID NO:175 and a light chain variable domain sequence that is at least 80%, at least 85%, at least 90%, or at least 95% identical to SEQ ID NO:417. The disclosure also provides antibodies comprising a heavy chain variable domain sequence that is at least 80%, at least 85%, at least 90%, or at least 95% identical to SEQ ID NO:176 and a light chain variable domain sequence that is at least 80%, at least 85%, at least 90%, or at least 95% identical to SEQ ID NO:418. The disclosure also provides antibodies comprising a heavy chain variable domain sequence that is at least 80%, at least 85%, at least 90%, or at least 95% identical to SEQ ID NO:177 and a light chain variable domain sequence that is at least 80%, at least 85%, at least 90%, or at least 95% identical to SEQ ID NO:419. The disclosure also provides antibodies comprising a heavy chain variable domain sequence that is at least 80%, at least 85%, at least 90%, or at least 95% identical to SEQ ID NO:178 and a light chain variable domain sequence that is at least 80%, at least 85%, at least 90%, or at least 95% identical to SEQ ID NO:419. The disclosure also provides antibodies comprising a heavy chain variable domain sequence that is at least 80%, at least 85%, at least 90%, or at least 95% identical to SEQ ID NO:179 and a light chain variable domain sequence that is at least 80%, at least 85%, at least 90%, or at least 95% identical to SEQ ID NO:420. The disclosure also provides antibodies comprising a heavy chain variable domain sequence that is at least 80%, at least 85%, at least 90%, or at least 95% identical to SEQ ID NO:180 and a light chain variable domain sequence that is at least 80%, at least 85%, at least 90%, or at least 95% identical to SEQ ID NO:421. The disclosure also provides antibodies comprising a heavy chain variable domain sequence that is at least 80%, at least 85%, at least 90%, or at least 95% identical to SEQ ID NO:181 and a light chain variable domain sequence that is at least 80%, at least 85%, at least 90%, or at least 95% identical to SEQ ID NO:422. The disclosure also provides antibodies comprising a heavy chain variable domain sequence that is at least 80%, at least 85%, at least 90%, or at least 95% identical to SEQ ID NO:182 and a light chain variable domain sequence that is at least 80%, at least 85%, at least 90%, or at least 95% identical to SEQ ID NO:423. The disclosure also provides antibodies comprising a heavy chain variable domain sequence that is at least 80%, at least 85%, at least 90%, or at least 95% identical to SEQ ID NO:183 and a light chain variable domain sequence that is at least 80%, at least 85%, at least 90%, or at least 95% identical to SEQ ID NO:424. The disclosure also provides antibodies comprising a heavy chain variable domain sequence that is at least 80%, at least 85%, at least 90%, or at least 95% identical to SEQ ID NO:184 and a light chain variable domain sequence that is at least 80%, at least 85%, at least 90%, or at least 95% identical to SEQ ID NO:425. The disclosure also provides antibodies comprising a heavy chain variable domain sequence that is at least 80%, at least 85%, at least 90%, or at least 95% identical to SEQ ID NO:185 and a light chain variable domain sequence that is at least 80%, at least 85%, at least 90%, or at least 95% identical to SEQ ID NO:426. The disclosure also provides antibodies comprising a heavy chain variable domain sequence that is at least 80%, at least 85%, at least 90%, or at least 95% identical to SEQ ID NO:186 and a light chain variable domain sequence that is at least 80%, at least 85%, at least 90%, or at least 95% identical to SEQ ID NO:427. The disclosure also provides antibodies comprising a heavy chain variable domain sequence that is at least 80%, at least 85%, at least 90%, or at least 95% identical to SEQ ID NO:187 and a light chain variable domain sequence that is at least 80%, at least 85%, at least 90%, or at least 95% identical to SEQ ID NO:428. The disclosure also provides antibodies comprising a heavy chain variable domain sequence that is at least 80%, at least 85%, at least 90%, or at least 95% identical to SEQ ID NO:188 and a light chain variable domain sequence that is at least 80%, at least 85%, at least 90%, or at least 95% identical to SEQ ID NO:429. The disclosure also provides antibodies comprising a heavy chain variable domain sequence that is at least 80%, at least 85%, at least 90%, or at least 95% identical to SEQ ID NO:188 and a light chain variable domain sequence that is at least 80%, at least 85%, at least 90%, or at least 95% identical to SEQ ID NO:430. The disclosure also provides antibodies comprising a heavy chain variable domain sequence that is at least 80%, at least 85%, at least 90%, or at least 95% identical to SEQ ID NO:189 and a light chain variable domain sequence that is at least 80%, at least 85%, at least 90%, or at least 95% identical to SEQ ID NO:431. The disclosure also provides antibodies comprising a heavy chain variable domain sequence that is at least 80%, at least 85%, at least 90%, or at least 95% identical to SEQ ID NO:190 and a light chain variable domain sequence that is at least 80%, at least 85%, at least 90%, or at least 95% identical to SEQ ID NO:429. The disclosure also provides antibodies comprising a heavy chain variable domain sequence that is at least 80%, at least 85%, at least 90%, or at least 95% identical to SEQ ID NO:190 and a light chain variable domain sequence that is at least 80%, at least 85%, at least 90%, or at least 95% identical to SEQ ID NO:387. The disclosure also provides antibodies comprising a heavy chain variable domain sequence that is at least 80%, at least 85%, at least 90%, or at least 95% identical to SEQ ID NO:191 and a light chain variable domain sequence that is at least 80%, at least 85%, at least 90%, or at least 95% identical to SEQ ID NO:430. The disclosure also provides antibodies comprising a heavy chain variable domain sequence that is at least 80%, at least 85%, at least 90%, or at least 95% identical to SEQ ID NO:192 and a light chain variable domain sequence that is at least 80%, at least 85%, at least 90%, or at least 95% identical to SEQ ID NO:432. The disclosure also provides antibodies comprising a heavy chain variable domain sequence that is at least 80%, at least 85%, at least 90%, or at least 95% identical to SEQ ID NO:193 and a light chain variable domain sequence that is at least 80%, at least 85%, at least 90%, or at least 95% identical to SEQ ID NO:433. The disclosure also provides antibodies comprising a heavy chain variable domain sequence that is at least 80%, at least 85%, at least 90%, or at least 95% identical to SEQ ID NO:194 and a light chain variable domain sequence that is at least 80%, at least 85%, at least 90%, or at least 95% identical to SEQ ID NO:434. The disclosure also provides antibodies comprising a heavy chain variable domain sequence that is at least 80%, at least 85%, at least 90%, or at least 95% identical to SEQ ID NO:194 and a light chain variable domain sequence that is at least 80%, at least 85%, at least 90%, or at least 95% identical to SEQ ID NO:434. The disclosure also provides antibodies comprising a heavy chain variable domain sequence that is at least 80%, at least 85%, at least 90%, or at least 95% identical to SEQ ID NO:196 and a light chain variable domain sequence that is at least 80%, at least 85%, at least 90%, or at least 95% identical to SEQ ID NO:429. The disclosure also provides antibodies comprising a heavy chain variable domain sequence that is at least 80%, at least 85%, at least 90%, or at least 95% identical to SEQ ID NO:197 and a light chain variable domain sequence that is at least 80%, at least 85%, at least 90%, or at least 95% identical to SEQ ID NO:434. The disclosure also provides antibodies comprising a heavy chain variable domain sequence that is at least 80%, at least 85%, at least 90%, or at least 95% identical to SEQ ID NO:198 and a light chain variable domain sequence that is at least 80%, at least 85%, at least 90%, or at least 95% identical to SEQ ID NO:435. The disclosure also provides antibodies comprising a heavy chain variable domain sequence that is at least 80%, at least 85%, at least 90%, or at least 95% identical to SEQ ID NO:199 and a light chain variable domain sequence that is at least 80%, at least 85%, at least 90%, or at least 95% identical to SEQ ID NO:252. The disclosure also provides antibodies comprising a heavy chain variable domain sequence that is at least 80%, at least 85%, at least 90%, or at least 95% identical to SEQ ID NO:199 and a light chain variable domain sequence that is at least 80%, at least 85%, at least 90%, or at least 95% identical to SEQ ID NO:301. The disclosure also provides antibodies comprising a heavy chain variable domain sequence that is at least 80%, at least 85%, at least 90%, or at least 95% identical to SEQ ID NO:199 and a light chain variable domain sequence that is at least 80%, at least 85%, at least 90%, or at least 95% identical to SEQ ID NO:264. The disclosure also provides antibodies comprising a heavy chain variable domain sequence that is at least 80%, at least 85%, at least 90%, or at least 95% identical to SEQ ID NO:199 and a light chain variable domain sequence that is at least 80%, at least 85%, at least 90%, or at least 95% identical to SEQ ID NO:293. The disclosure also provides antibodies comprising a heavy chain variable domain sequence that is at least 80%, at least 85%, at least 90%, or at least 95% identical to SEQ ID NO:199 and a light chain variable domain sequence that is at least 80%, at least 85%, at least 90%, or at least 95% identical to SEQ ID NO:337. The disclosure also provides antibodies comprising a heavy chain variable domain sequence that is at least 80%, at least 85%, at least 90%, or at least 95% identical to SEQ ID NO:199 and a light chain variable domain sequence that is at least 80%, at least 85%, at least 90%, or at least 95% identical to SEQ ID NO:294. The disclosure also provides antibodies comprising a heavy chain variable domain sequence that is at least 80%, at least 85%, at least 90%, or at least 95% identical to SEQ ID NO:199 and a light chain variable domain sequence that is at least 80%, at least 85%, at least 90%, or at least 95% identical to SEQ ID NO:318. The disclosure also provides antibodies comprising a heavy chain variable domain sequence that is at least 80%, at least 85%, at least 90%, or at least 95% identical to SEQ ID NO:199 and a light chain variable domain sequence that is at least 80%, at least 85%, at least 90%, or at least 95% identical to SEQ ID NO:358. The disclosure also provides antibodies comprising a heavy chain variable domain sequence that is at least 80%, at least 85%, at least 90%, or at least 95% identical to SEQ ID NO:199 and a light chain variable domain sequence that is at least 80%, at least 85%, at least 90%, or at least 95% identical to SEQ ID NO:312. The disclosure also provides antibodies comprising a heavy chain variable domain sequence that is at least 80%, at least 85%, at least 90%, or at least 95% identical to SEQ ID NO:199 and a light chain variable domain sequence that is at least 80%, at least 85%, at least 90%, or at least 95% identical to SEQ ID NO:371. The disclosure also provides antibodies comprising a heavy chain variable domain sequence that is at least 80%, at least 85%, at least 90%, or at least 95% identical to SEQ ID NO:199 and a light chain variable domain sequence that is at least 80%, at least 85%, at least 90%, or at least 95% identical to SEQ ID NO:366. The disclosure also provides antibodies comprising a heavy chain variable domain sequence that is at least 80%, at least 85%, at least 90%, or at least 95% identical to SEQ ID NO:200 and a light chain variable domain sequence that is at least 80%, at least 85%, at least 90%, or at least 95% identical to SEQ ID NO:430. The disclosure also provides antibodies comprising a heavy chain variable domain sequence that is at least 80%, at least 85%, at least 90%, or at least 95% identical to SEQ ID NO:200 and a light chain variable domain sequence that is at least 80%, at least 85%, at least 90%, or at least 95% identical to SEQ ID NO:328. The disclosure also provides antibodies comprising a heavy chain variable domain sequence that is at least 80%, at least 85%, at least 90%, or at least 95% identical to SEQ ID NO:200 and a light chain variable domain sequence that is at least 80%, at least 85%, at least 90%, or at least 95% identical to SEQ ID NO:264. The disclosure also provides antibodies comprising a heavy chain variable domain sequence that is at least 80%, at least 85%, at least 90%, or at least 95% identical to SEQ ID NO:200 and a light chain variable domain sequence that is at least 80%, at least 85%, at least 90%, or at least 95% identical to SEQ ID NO:411. The disclosure also provides antibodies comprising a heavy chain variable domain sequence that is at least 80%, at least 85%, at least 90%, or at least 95% identical to SEQ ID NO:200 and a light chain variable domain sequence that is at least 80%, at least 85%, at least 90%, or at least 95% identical to SEQ ID NO:413. The disclosure also provides antibodies comprising a heavy chain variable domain sequence that is at least 80%, at least 85%, at least 90%, or at least 95% identical to SEQ ID NO:200 and a light chain variable domain sequence that is at least 80%, at least 85%, at least 90%, or at least 95% identical to SEQ ID NO:304. The disclosure also provides antibodies comprising a heavy chain variable domain sequence that is at least 80%, at least 85%, at least 90%, or at least 95% identical to SEQ ID NO:200 and a light chain variable domain sequence that is at least 80%, at least 85%, at least 90%, or at least 95% identical to SEQ ID NO:318. The disclosure also provides antibodies comprising a heavy chain variable domain sequence that is at least 80%, at least 85%, at least 90%, or at least 95% identical to SEQ ID NO:200 and a light chain variable domain sequence that is at least 80%, at least 85%, at least 90%, or at least 95% identical to SEQ ID NO:324. The disclosure also provides antibodies comprising a heavy chain variable domain sequence that is at least 80%, at least 85%, at least 90%, or at least 95% identical to SEQ ID NO:200 and a light chain variable domain sequence that is at least 80%, at least 85%, at least 90%, or at least 95% identical to SEQ ID NO:333. The disclosure also provides antibodies comprising a heavy chain variable domain sequence that is at least 80%, at least 85%, at least 90%, or at least 95% identical to SEQ ID NO:200 and a light chain variable domain sequence that is at least 80%, at least 85%, at least 90%, or at least 95% identical to SEQ ID NO:265. The disclosure also provides antibodies comprising a heavy chain variable domain sequence that is at least 80%, at least 85%, at least 90%, or at least 95% identical to SEQ ID NO:201 and a light chain variable domain sequence that is at least 80%, at least 85%, at least 90%, or at least 95% identical to SEQ ID NO:429. The disclosure also provides antibodies comprising a heavy chain variable domain sequence that is at least 80%, at least 85%, at least 90%, or at least 95% identical to SEQ ID NO:202 and a light chain variable domain sequence that is at least 80%, at least 85%, at least 90%, or at least 95% identical to SEQ ID NO:436. The disclosure also provides antibodies comprising a heavy chain variable domain sequence that is at least 80%, at least 85%, at least 90%, or at least 95% identical to SEQ ID NO:203 and a light chain variable domain sequence that is at least 80%, at least 85%, at least 90%, or at least 95% identical to SEQ ID NO:437. The disclosure also provides antibodies comprising a heavy chain variable domain sequence that is at least 80%, at least 85%, at least 90%, or at least 95% identical to SEQ ID NO:204 and a light chain variable domain sequence that is at least 80%, at least 85%, at least 90%, or at least 95% identical to SEQ ID NO:438. The disclosure also provides antibodies comprising a heavy chain variable domain sequence that is at least 80%, at least 85%, at least 90%, or at least 95% identical to SEQ ID NO:204 and a light chain variable domain sequence that is at least 80%, at least 85%, at least 90%, or at least 95% identical to SEQ ID NO:439. The disclosure also provides antibodies comprising a heavy chain variable domain sequence that is at least 80%, at least 85%, at least 90%, or at least 95% identical to SEQ ID NO:205 and a light chain variable domain sequence that is at least 80%, at least 85%, at least 90%, or at least 95% identical to SEQ ID NO:440. The disclosure also provides antibodies comprising a heavy chain variable domain sequence that is at least 80%, at least 85%, at least 90%, or at least 95% identical to SEQ ID NO:205 and a light chain variable domain sequence that is at least 80%, at least 85%, at least 90%, or at least 95% identical to SEQ ID NO:438. The disclosure also provides antibodies comprising a heavy chain variable domain sequence that is at least 80%, at least 85%, at least 90%, or at least 95% identical to SEQ ID NO:206 and a light chain variable domain sequence that is at least 80%, at least 85%, at least 90%, or at least 95% identical to SEQ ID NO:441. The disclosure also provides antibodies comprising a heavy chain variable domain sequence that is at least 80%, at least 85%, at least 90%, or at least 95% identical to SEQ ID NO:207 and a light chain variable domain sequence that is at least 80%, at least 85%, at least 90%, or at least 95% identical to SEQ ID NO:442. The disclosure also provides antibodies comprising a heavy chain variable domain sequence that is at least 80%, at least 85%, at least 90%, or at least 95% identical to SEQ ID NO:208 and a light chain variable domain sequence that is at least 80%, at least 85%, at least 90%, or at least 95% identical to SEQ ID NO:443. The disclosure also provides antibodies comprising a heavy chain variable domain sequence that is at least 80%, at least 85%, at least 90%, or at least 95% identical to SEQ ID NO:209 and a light chain variable domain sequence that is at least 80%, at least 85%, at least 90%, or at least 95% identical to SEQ ID NO:443. The disclosure also provides antibodies comprising a heavy chain variable domain sequence that is at least 80%, at least 85%, at least 90%, or at least 95% identical to SEQ ID NO:210 and a light chain variable domain sequence that is at least 80%, at least 85%, at least 90%, or at least 95% identical to SEQ ID NO:444. The disclosure also provides antibodies comprising a heavy chain variable domain sequence that is at least 80%, at least 85%, at least 90%, or at least 95% identical to SEQ ID NO:211 and a light chain variable domain sequence that is at least 80%, at least 85%, at least 90%, or at least 95% identical to SEQ ID NO:445. The disclosure also provides antibodies comprising a heavy chain variable domain sequence that is at least 80%, at least 85%, at least 90%, or at least 95% identical to SEQ ID NO:212 and a light chain variable domain sequence that is at least 80%, at least 85%, at least 90%, or at least 95% identical to SEQ ID NO:446. The disclosure also provides antibodies comprising a heavy chain variable domain sequence that is at least 80%, at least 85%, at least 90%, or at least 95% identical to SEQ ID NO:213 and a light chain variable domain sequence that is at least 80%, at least 85%, at least 90%, or at least 95% identical to SEQ ID NO:447. The disclosure also provides antibodies comprising a heavy chain variable domain sequence that is at least 80%, at least 85%, at least 90%, or at least 95% identical to SEQ ID NO:214 and a light chain variable domain sequence that is at least 80%, at least 85%, at least 90%, or at least 95% identical to SEQ ID NO:410. The disclosure also provides antibodies comprising a heavy chain variable domain sequence that is at least 80%, at least 85%, at least 90%, or at least 95% identical to SEQ ID NO:215 and a light chain variable domain sequence that is at least 80%, at least 85%, at least 90%, or at least 95% identical to SEQ ID NO:448. The disclosure also provides antibodies comprising a heavy chain variable domain sequence that is at least 80%, at least 85%, at least 90%, or at least 95% identical to SEQ ID NO:215 and a light chain variable domain sequence that is at least 80%, at least 85%, at least 90%, or at least 95% identical to SEQ ID NO:333. The disclosure also provides antibodies comprising a heavy chain variable domain sequence that is at least 80%, at least 85%, at least 90%, or at least 95% identical to SEQ ID NO:216 and a light chain variable domain sequence that is at least 80%, at least 85%, at least 90%, or at least 95% identical to SEQ ID NO:449. The disclosure also provides antibodies comprising a heavy chain variable domain sequence that is at least 80%, at least 85%, at least 90%, or at least 95% identical to SEQ ID NO:217 and a light chain variable domain sequence that is at least 80%, at least 85%, at least 90%, or at least 95% identical to SEQ ID NO:450. The disclosure also provides antibodies comprising a heavy chain variable domain sequence that is at least 80%, at least 85%, at least 90%, or at least 95% identical to SEQ ID NO:218 and a light chain variable domain sequence that is at least 80%, at least 85%, at least 90%, or at least 95% identical to SEQ ID NO:451. The disclosure also provides antibodies comprising a heavy chain variable domain sequence that is at least 80%, at least 85%, at least 90%, or at least 95% identical to SEQ ID NO:219 and a light chain variable domain sequence that is at least 80%, at least 85%, at least 90%, or at least 95% identical to SEQ ID NO:452. The disclosure also provides antibodies comprising a heavy chain variable domain sequence that is at least 80%, at least 85%, at least 90%, or at least 95% identical to SEQ ID NO:220 and a light chain variable domain sequence that is at least 80%, at least 85%, at least 90%, or at least 95% identical to SEQ ID NO:453. The disclosure also provides antibodies comprising a heavy chain variable domain sequence that is at least 80%, at least 85%, at least 90%, or at least 95% identical to SEQ ID NO:221 and a light chain variable domain sequence that is at least 80%, at least 85%, at least 90%, or at least 95% identical to SEQ ID NO:331. The disclosure also provides antibodies comprising a heavy chain variable domain sequence that is at least 80%, at least 85%, at least 90%, or at least 95% identical to SEQ ID NO:222 and a light chain variable domain sequence that is at least 80%, at least 85%, at least 90%, or at least 95% identical to SEQ ID NO:454. The disclosure also provides antibodies comprising a heavy chain variable domain sequence that is at least 80%, at least 85%, at least 90%, or at least 95% identical to SEQ ID NO:222 and a light chain variable domain sequence that is at least 80%, at least 85%, at least 90%, or at least 95% identical to SEQ ID NO:455. The disclosure also provides antibodies comprising a heavy chain variable domain sequence that is at least 80%, at least 85%, at least 90%, or at least 95% identical to SEQ ID NO:222 and a light chain variable domain sequence that is at least 80%, at least 85%, at least 90%, or at least 95% identical to SEQ ID NO:366. The disclosure also provides antibodies comprising a heavy chain variable domain sequence that is at least 80%, at least 85%, at least 90%, or at least 95% identical to SEQ ID NO:223 and a light chain variable domain sequence that is at least 80%, at least 85%, at least 90%, or at least 95% identical to SEQ ID NO:438. The disclosure also provides antibodies comprising a heavy chain variable domain sequence that is at least 80%, at least 85%, at least 90%, or at least 95% identical to SEQ ID NO:224 and a light chain variable domain sequence that is at least 80%, at least 85%, at least 90%, or at least 95% identical to SEQ ID NO:456. The disclosure also provides antibodies comprising a heavy chain variable domain sequence that is at least 80%, at least 85%, at least 90%, or at least 95% identical to SEQ ID NO:225 and a light chain variable domain sequence that is at least 80%, at least 85%, at least 90%, or at least 95% identical to SEQ ID NO:400. The disclosure also provides antibodies comprising a heavy chain variable domain sequence that is at least 80%, at least 85%, at least 90%, or at least 95% identical to SEQ ID NO:225 and a light chain variable domain sequence that is at least 80%, at least 85%, at least 90%, or at least 95% identical to SEQ ID NO:457. The disclosure also provides antibodies comprising a heavy chain variable domain sequence that is at least 80%, at least 85%, at least 90%, or at least 95% identical to SEQ ID NO:225 and a light chain variable domain sequence that is at least 80%, at least 85%, at least 90%, or at least 95% identical to SEQ ID NO:458. The disclosure also provides antibodies comprising a heavy chain variable domain sequence that is at least 80%, at least 85%, at least 90%, or at least 95% identical to SEQ ID NO:226 and a light chain variable domain sequence that is at least 80%, at least 85%, at least 90%, or at least 95% identical to SEQ ID NO:456. The disclosure also provides antibodies comprising a heavy chain variable domain sequence that is at least 80%, at least 85%, at least 90%, or at least 95% identical to SEQ ID NO:227 and a light chain variable domain sequence that is at least 80%, at least 85%, at least 90%, or at least 95% identical to SEQ ID NO:459. The disclosure also provides antibodies comprising a heavy chain variable domain sequence that is at least 80%, at least 85%, at least 90%, or at least 95% identical to SEQ ID NO:228 and a light chain variable domain sequence that is at least 80%, at least 85%, at least 90%, or at least 95% identical to SEQ ID NO:457. The disclosure also provides antibodies comprising a heavy chain variable domain sequence that is at least 80%, at least 85%, at least 90%, or at least 95% identical to SEQ ID NO:229 and a light chain variable domain sequence that is at least 80%, at least 85%, at least 90%, or at least 95% identical to SEQ ID NO:460. The disclosure also provides antibodies comprising a heavy chain variable domain sequence that is at least 80%, at least 85%, at least 90%, or at least 95% identical to SEQ ID NO:230 and a light chain variable domain sequence that is at least 80%, at least 85%, at least 90%, or at least 95% identical to SEQ ID NO:461. The disclosure also provides antibodies comprising a heavy chain variable domain sequence that is at least 80%, at least 85%, at least 90%, or at least 95% identical to SEQ ID NO:231 and a light chain variable domain sequence that is at least 80%, at least 85%, at least 90%, or at least 95% identical to SEQ ID NO:462. The disclosure also provides antibodies comprising a heavy chain variable domain sequence that is at least 80%, at least 85%, at least 90%, or at least 95% identical to SEQ ID NO:232 and a light chain variable domain sequence that is at least 80%, at least 85%, at least 90%, or at least 95% identical to SEQ ID NO:463. The disclosure also provides antibodies comprising a heavy chain variable domain sequence that is at least 80%, at least 85%, at least 90%, or at least 95% identical to SEQ ID NO:233 and a light chain variable domain sequence that is at least 80%, at least 85%, at least 90%, or at least 95% identical to SEQ ID NO:464. The disclosure also provides antibodies comprising a heavy chain variable domain sequence that is at least 80%, at least 85%, at least 90%, or at least 95% identical to SEQ ID NO:234 and a light chain variable domain sequence that is at least 80%, at least 85%, at least 90%, or at least 95% identical to SEQ ID NO:465. The disclosure also provides antibodies comprising a heavy chain variable domain sequence that is at least 80%, at least 85%, at least 90%, or at least 95% identical to SEQ ID NO:235 and a light chain variable domain sequence that is at least 80%, at least 85%, at least 90%, or at least 95% identical to SEQ ID NO:466. The disclosure also provides antibodies comprising a heavy chain variable domain sequence that is at least 80%, at least 85%, at least 90%, or at least 95% identical to SEQ ID NO:236 and a light chain variable domain sequence that is at least 80%, at least 85%, at least 90%, or at least 95% identical to SEQ ID NO:467. The disclosure also provides antibodies comprising a heavy chain variable domain sequence that is at least 80%, at least 85%, at least 90%, or at least 95% identical to SEQ ID NO:237 and a light chain variable domain sequence that is at least 80%, at least 85%, at least 90%, or at least 95% identical to SEQ ID NO:468. The disclosure also provides antibodies comprising a heavy chain variable domain sequence that is at least 80%, at least 85%, at least 90%, or at least 95% identical to SEQ ID NO:238 and a light chain variable domain sequence that is at least 80%, at least 85%, at least 90%, or at least 95% identical to SEQ ID NO:469. The disclosure also provides antibodies comprising a heavy chain variable domain sequence that is at least 80%, at least 85%, at least 90%, or at least 95% identical to SEQ ID NO:239 and a light chain variable domain sequence that is at least 80%, at least 85%, at least 90%, or at least 95% identical to SEQ ID NO:304. The disclosure also provides antibodies comprising a heavy chain variable domain sequence that is at least 80%, at least 85%, at least 90%, or at least 95% identical to SEQ ID NO:239 and a light chain variable domain sequence that is at least 80%, at least 85%, at least 90%, or at least 95% identical to SEQ ID NO:354. The disclosure also provides antibodies comprising a heavy chain variable domain sequence that is at least 80%, at least 85%, at least 90%, or at least 95% identical to SEQ ID NO:239 and a light chain variable domain sequence that is at least 80%, at least 85%, at least 90%, or at least 95% identical to SEQ ID NO:470. The disclosure also provides antibodies comprising a heavy chain variable domain sequence that is at least 80%, at least 85%, at least 90%, or at least 95% identical to SEQ ID NO:240 and a light chain variable domain sequence that is at least 80%, at least 85%, at least 90%, or at least 95% identical to SEQ ID NO:471. The disclosure also provides antibodies comprising a heavy chain variable domain sequence that is at least 80%, at least 85%, at least 90%, or at least 95% identical to SEQ ID NO:241 and a light chain variable domain sequence that is at least 80%, at least 85%, at least 90%, or at least 95% identical to SEQ ID NO:472. The disclosure also provides antibodies comprising a heavy chain variable domain sequence that is at least 80%, at least 85%, at least 90%, or at least 95% identical to SEQ ID NO:242 and a light chain variable domain sequence that is at least 80%, at least 85%, at least 90%, or at least 95% identical to SEQ ID NO:473. The disclosure also provides antibodies comprising a heavy chain variable domain sequence that is at least 80%, at least 85%, at least 90%, or at least 95% identical to SEQ ID NO:242 and a light chain variable domain sequence that is at least 80%, at least 85%, at least 90%, or at least 95% identical to SEQ ID NO:474. The disclosure also provides antibodies comprising a heavy chain variable domain sequence that is at least 80%, at least 85%, at least 90%, or at least 95% identical to SEQ ID NO:242 and a light chain variable domain sequence that is at least 80%, at least 85%, at least 90%, or at least 95% identical to SEQ ID NO:475. The disclosure also provides antibodies comprising a heavy chain variable domain sequence that is at least 80%, at least 85%, at least 90%, or at least 95% identical to SEQ ID NO:242 and a light chain variable domain sequence that is at least 80%, at least 85%, at least 90%, or at least 95% identical to SEQ ID NO:265. The disclosure also provides antibodies comprising a heavy chain variable domain sequence that is at least 80%, at least 85%, at least 90%, or at least 95% identical to SEQ ID NO:243 and a light chain variable domain sequence that is at least 80%, at least 85%, at least 90%, or at least 95% identical to SEQ ID NO:476. The disclosure also provides antibodies comprising a heavy chain variable domain sequence that is at least 80%, at least 85%, at least 90%, or at least 95% identical to SEQ ID NO:244 and a light chain variable domain sequence that is at least 80%, at least 85%, at least 90%, or at least 95% identical to SEQ ID NO:468. The disclosure also provides antibodies comprising a heavy chain variable domain sequence that is at least 80%, at least 85%, at least 90%, or at least 95% identical to SEQ ID NO:245 and a light chain variable domain sequence that is at least 80%, at least 85%, at least 90%, or at least 95% identical to SEQ ID NO:477. The disclosure also provides antibodies comprising a heavy chain variable domain sequence that is at least 80%, at least 85%, at least 90%, or at least 95% identical to SEQ ID NO:246 and a light chain variable domain sequence that is at least 80%, at least 85%, at least 90%, or at least 95% identical to SEQ ID NO:478. The disclosure also provides antibodies comprising a heavy chain variable domain sequence that is at least 80%, at least 85%, at least 90%, or at least 95% identical to SEQ ID NO:246 and a light chain variable domain sequence that is at least 80%, at least 85%, at least 90%, or at least 95% identical to SEQ ID NO:333. The disclosure also provides antibodies comprising a heavy chain variable domain sequence that is at least 80%, at least 85%, at least 90%, or at least 95% identical to SEQ ID NO:973 and a light chain variable domain sequence that is at least 80%, at least 85%, at least 90%, or at least 95% identical to SEQ ID NO:974. The disclosure also provides antibodies comprising a heavy chain variable domain sequence that is at least 80%, at least 85%, at least 90%, or at least 95% identical to SEQ ID NO:973 and a light chain variable domain sequence that is at least 80%, at least 85%, at least 90%, or at least 95% identical to SEQ ID NO:975. The disclosure also provides antibodies comprising a heavy chain variable domain sequence that is at least 80%, at least 85%, at least 90%, or at least 95% identical to SEQ ID NO:976 and a light chain variable domain sequence that is at least 80%, at least 85%, at least 90%, or at least 95% identical to SEQ ID NO:977. The disclosure also provides antibodies comprising a heavy chain variable domain sequence that is at least 80%, at least 85%, at least 90%, or at least 95% identical to SEQ ID NO:976 and a light chain variable domain sequence that is at least 80%, at least 85%, at least 90%, or at least 95% identical to SEQ ID NO:978. The disclosure also provides antibodies comprising a heavy chain variable domain sequence that is at least 80%, at least 85%, at least 90%, or at least 95% identical to SEQ ID NO:976 and a light chain variable domain sequence that is at least 80%, at least 85%, at least 90%, or at least 95% identical to SEQ ID NO:979. The disclosure also provides antibodies comprising a heavy chain variable domain sequence that is at least 80%, at least 85%, at least 90%, or at least 95% identical to SEQ ID NO:980 and a light chain variable domain sequence that is at least 80%, at least 85%, at least 90%, or at least 95% identical to SEQ ID NO:977. The disclosure also provides antibodies comprising a heavy chain variable domain sequence that is at least 80%, at least 85%, at least 90%, or at least 95% identical to SEQ ID NO:980 and a light chain variable domain sequence that is at least 80%, at least 85%, at least 90%, or at least 95% identical to SEQ ID NO:978. The disclosure also provides antibodies comprising a heavy chain variable domain sequence that is at least 80%, at least 85%, at least 90%, or at least 95% identical to SEQ ID NO:980 and a light chain variable domain sequence that is at least 80%, at least 85%, at least 90%, or at least 95% identical to SEQ ID NO:979. The disclosure also provides antibodies comprising a heavy chain variable domain sequence that is at least 80%, at least 85%, at least 90%, or at least 95% identical to SEQ ID NO:981 and a light chain variable domain sequence that is at least 80%, at least 85%, at least 90%, or at least 95% identical to SEQ ID NO:982. The disclosure also provides antibodies comprising a heavy chain variable domain sequence that is at least 80%, at least 85%, at least 90%, or at least 95% identical to SEQ ID NO:981 and a light chain variable domain sequence that is at least 80%, at least 85%, at least 90%, or at least 95% identical to SEQ ID NO:983. The disclosure also provides antibodies comprising a heavy chain variable domain sequence that is at least 80%, at least 85%, at least 90%, or at least 95% identical to SEQ ID NO:984 and a light chain variable domain sequence that is at least 80%, at least 85%, at least 90%, or at least 95% identical to SEQ ID NO:982. The disclosure also provides antibodies comprising a heavy chain variable domain sequence that is at least 80%, at least 85%, at least 90%, or at least 95% identical to SEQ ID NO:984 and a light chain variable domain sequence that is at least 80%, at least 85%, at least 90%, or at least 95% identical to SEQ ID NO:983.

Table 1 provides a summary of the B7-H3-specific antibodies described herein:

### Pharmaceutical Compositions

Pharmaceutical compositions comprising the antibodies or antibody fragments of the present disclosure are also contemplated and can be used in the methods disclosed herein. Pharmaceutical compositions can comprise one or more of the antibodies or antibody fragments described herein and a pharmaceutically acceptable carrier or excipient. Although the carrier or excipient may facilitate administration, it should not itself induce the production of antibodies harmful to the subject or individual receiving the composition; nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles, and are known to one of skill in the art.

The antibodies or an antigen binding fragments described herein, or the pharmaceutical compositions disclosed herein, may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Typically, the therapeutic compositions may be prepared as injectables, either as liquid solutions or suspensions. Solid forms suitable for solution in, or suspension in, liquid vehicles prior to injection may also be prepared.

In one embodiment, the antibody, or an antigen binding fragment thereof, or pharmaceutical composition is administered intravenously. In another embodiment, the antibody, or an antigen binding fragment thereof, or pharmaceutical composition is administered by intravenous infusion.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule. Known antibody-based pharmaceuticals provide guidance relating to frequency of administration e.g., whether a pharmaceutical should be delivered daily, weekly, monthly, etc. Frequency and dosage may also depend on the severity of symptoms.

It will be appreciated that the active ingredient in the composition will be an antibody molecule, an antibody fragment or variants and derivatives thereof. As such, it will be susceptible to degradation in the gastrointestinal tract. Thus, if the composition is to be administered by a route using the gastrointestinal tract, the composition will need to contain agents which protect the antibody from degradation, but which release the antibody once it has been absorbed from the gastrointestinal tract.

The methods of the present invention can use an antibody, or an antigen binding fragment thereof, as described above, alone or in combination with other pharmaceutically active compounds, to treat conditions such as those disclosed hereinabove. The additional pharmaceutically active compound(s) can be administered simultaneously (either in the same dosage form or in separate dosage forms) or sequentially. Accordingly, in one embodiment, the present invention comprises methods for treating a condition by administering to the subject a therapeutically effective amount of an antibody, or an antigen binding fragment thereof, of the present invention and one or more additional pharmaceutically active compounds.

In some embodiments, the antibody, or an antigen binding fragment thereof, of the present invention is used in combination with existing LAG-3-related disease therapies.

### EXAMPLES

The following example is put forth so as to provide those of ordinary skill in the art with a complete description of how to make and use the present disclosure, and is not intended to limit the scope of what the inventors regard as their disclosure nor is it intended to represent that the experiment below is all or the only experiment that could be performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1: Isolation of Murine Anti-Human B7-113 Antibodies

### Immunization, & Single Cell Suspension Generation

Recombinant human B7-H3 (4Ig) His tagged, catalog no: B7B-H52E7, ACROBiosystems, Beijing, China, SEQ ID NO:2) was used to immunize young C57/BL6 mice each with 80 μg of the protein in Sigma Adjuvant System® (Sigma-Aldrich, St. Louis, Mo.) over a period of 35 days using a rapid immunization protocol of Antibody Solutions (Sunnyvale, Calif.). The lymph nodes were harvested on day 35. Single cell suspension of the lymph node was generated, and the suspension was filtered through a 70 μm mesh (BD Bioscience) to remove clumps.

### Plasma Cell Isolation, Antibody Capture, and Antigen Interrogation

The filtered lymphocyte suspension was enriched for plasma cells actively secreting IgGs using a kit based on cell surface expression of CD138 (Miltenyi, Auburn, Calif.). Using a method described in U.S. Pat. No. 9,328,172, freshly enriched plasma cells were deposited on a PDMS device to allow a single cell settled in the microwells on the device. Antibody secreted from each plasma cell was captured on a derivatized microscope slide. Antigen-specific antibody secreting cells were identified by interrogating the antibody capture slide with varying concentrations of fluorescently labeled B7-H3 (4Ig) protein tagged with human Fc (ACROBiosystems, Beijing, China, catalog no: B7B-H5258, SEQ ID NO:3). Labeling was done using a kit (AnaSpec, Fremont, Calif., AS-72046, AnaTag™ HiLyte™ Fluor 555 Microscale Protein Labeling Kit *Ultra Convenient*).

mRNA Capture

After antibody capture, the medium was removed, and replaced with lysis buffer followed by prompt closure of the top of the microwells with a custom oligonucleotide microarray (Agilent, Santa Clara, Calif.). This procedure was previously described in U.S. Pat. No. 9,328,172. The custom oligonucleotide microarray is prepared such that each feature contains not only a unique tag specifying its coordinate but also capture probes for all subclasses (1, 2a, 2b, and 3) of murine IgG heavy chain, murine Ig kappa light chain.

cDNA Synthesis, PCR Amplification, and Next Generation Sequencing

Captured mRNA on the custom microarray was further processed to synthesize cDNA of each mRNA incorporating the unique tag originally on each feature. The cDNA is then amplify using a Taq polymerase (Promega, Madison, Wis.) and appropriate set of primers to allow amplification of the following genes: variable domain of IgG heavy chain subclasses and variable domain of Ig kappa light chain. Though now released from cells, these fragments of each gene are now labeled with the unique tag from the custom oligonucleotide microarray manifesting their originating locations. The amplicons were further manipulated to have appropriate sequence attached at both ends to enable sequencing on an Illumina MiSeq instrument using 2×250 bp chemistry at SeqMatic LLC (Fremont, Calif.).

### Bioinformatic Analysis of Images and DNA Sequences

Sequencing reads from MiSeq were processed and the embedded tag in each read was identified and converted into coordinates. The coordinates were plotted to yield a synthetic map of the mRNA recovered. Most of the coordinates form clusters that designate the location of the originating cell for the recovered mRNA sequences. Next, CDR3 motif present in each read with the coordinates was identified and collated according to the clusters that matched the location of an antibody spot visualized by an appropriate fluorescently labeled secondary antibody. Identical or nearly identical CDR3s for a given antibody spot were organized and form consistent pair of V_{H} and V_{L} sequences. The remaining part of V_{H} or V_{L} sequence containing the identified CDR3s was identified and the associated sequencing reads were assembled into full-length cDNA sequences for V_{H} and V_{L}. The pair of full-length cDNA was correlated with the affinity measurements associated with each of the antigen-specific antibody spot.

### Example 2: Molecular Reconstruction and Recombinant Expression of Anti-B7-H3 Antibodies

The paired V_{H} and V_{L} anti-B7-H3 antibody sequences were used to synthesize corresponding gene fragments by a service provider according to the known art. The resulting gene fragments were cloned into an appropriate plasmid vector and transfected into an appropriate mammalian host, such as HEK293, for recombinant expression to produce an antibody preparation in full-IgG format. The antibody preparations were characterized by measurements at OD280 to assess the amount produced and by gel electrophoresis on PAGE to assess the size of the antibody chains produced.

### Example 3: Characterization of Recombinant Anti-B7-H3 Antibodies

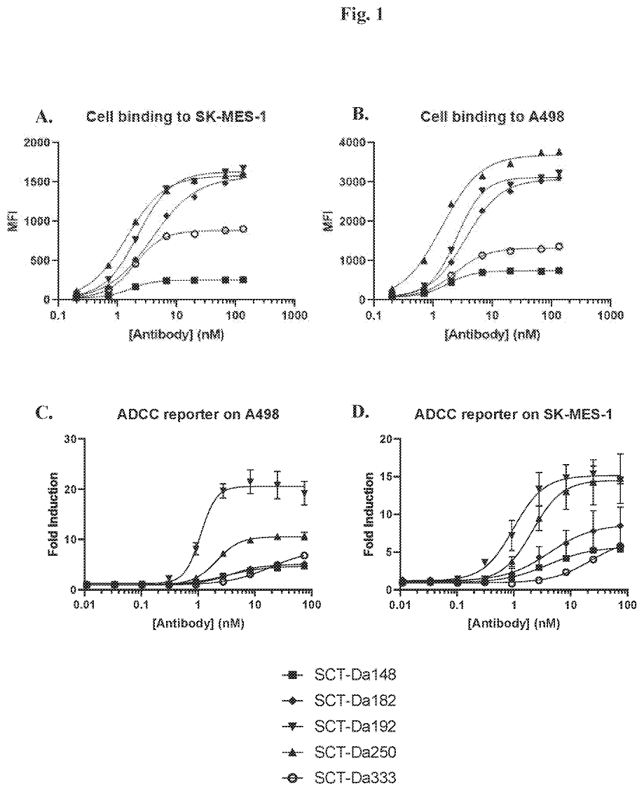

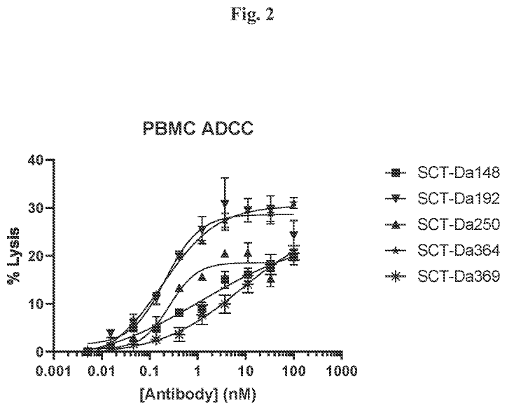

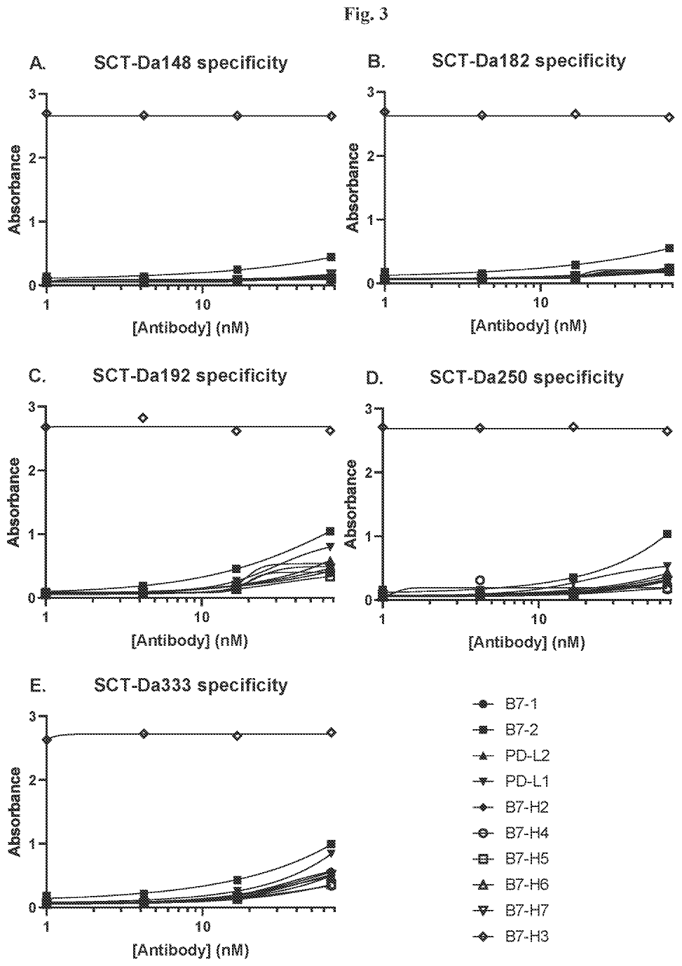

The recombinantly expressed antibodies were used to assess binding activity to B7-H3 by conventional ELISA according to the known art. Recombinant human B7-H3 and cyno B7-H3 were coated onto ELISA plates to detect binding at serially diluted concentrations of the anti-B7-H3 antibody preparations. The EC50s based on ELISA is shown in table 2. The binding affinity of select anti-B7-H3 antibodies were measured on a surface plasmon resonance (SPR) instrument, such as a Biacore T200™, against human B7-H3 (table 3) and cyno B7-H3 (table 4). Cell lines expressing B7-H3, A498 and SK-MES-1, were used to assess binding by anti-B7-H3 antibodies on the native protein on the cell surface by flow cytometry according to the known art. The EC50s for these binding to these 2 cell lines are displayed in table 2 under the heading “FACS” and FIGS. 1A and 1B. In addition, the antibody-dependent cellular cytotoxicity (ADCC) activity of select anti-B7-H3 antibodies were assessed using an engineered surrogate effector cell line, kit reagents, and a luminometer (Promega, Madison, Wis.) on A498 and SK-MES-1 as target cells in table 2 under the heading “ADCC (reporter) and FIGS. 1C and 1D. Finally, specificity of each of the antibodies examined thus far were assessed by interacting with proteins from the B7 family (human B7-1, B7-2, PD-L2, PD-L1, B7-H2, B7-H4, B7-H5, B7-H6, B7-H7) against B7-H3 using ELISA. The data were plotted for each antibody on FIGS. 3A through 3E.

Antibodies recovered from the antibody campaign described above are listed herein. The CDR sequences and the V_{H} and V_{L} sequences for the anti-B7-H3 antibodies described herein are depicted in Tables 5 and 6, respectively.

TABLE 2

| | | | | | | ADCC | | |
|---|---|---|---|---|---|---|---|---|
| | ELISA | | FACS | | | (reporter) | | ADCC |
| | Human | Cyno | | SK- | | | SK- | (PBMC) |
| | B7-H3 | B7-H3 | A498 | MES-1 | Raji | A498 | MES-1 | A549 |
| | EC50 | EC50 | EC50 | EC50 | EC50 | EC50 | EC50 | EC50 |
| Anti-B7-H3 mAb | (pM) | (pM) | (nM) | (nM) | (nM) | (nM) | (nM) | (nM) |
| SCT-Da148 | 7.60 | 11.43 | 1.60 | 1.49 | >100 | 3.6 | 4.2 | 0.75 |
| SCT-Da182 | 10.46 | 8.82 | 3.70 | 4.09 | >100 | 4.5 | 4.6 | ND |
| SCT-Da192 | 10.86 | 17.64 | 2.62 | 2.32 | >100 | 1.1 | 0.9 | 0.21 |
| SCT-Da250 | 22.55 | 23.74 | 1.43 | 1.46 | >100 | 2.4 | 2.4 | 0.20 |
| SCT-Da333 | 8.33 | 3048.00 | 3.29 | 1.94 | >100 | 23.3 | 32.8 | ND |
| SCT-Da364 | ND | ND | ND | ND | ND | ND | ND | 0.18 |
| SCT-Da369 | ND | ND | ND | ND | ND | ND | ND | 8.05 |

ND: Not determined.

TABLE 3

| Anti-B7-H3 mAb | Kₐ (1/Ms) | K_{d} (1/s) | K_{D} (M) | Rₘₐₓ (RU) |
|---|---|---|---|---|
| SCT-Da148 | 3.66E+05 | 2.34E−03 | 6.41E−09 | 50.4 |
| SCT-Da182 | 9.27E+04 | 1.01E−03 | 1.09E−08 | 39.2 |
| SCT-Da192 | 8.51E+04 | 2.03E−03 | 2.38E−08 | 118.2 |
| SCT-Da250 | 4.02E+05 | 1.10E−03 | 2.75E−09 | 71.3 |
| SCT-Da333 | 1.35E+05 | 5.12E−02 | 3.80E−07 | 51.0 |

TABLE 4

| Anti-B7-H3 mAb | Kₐ (1/Ms) | K_{d} (1/s) | K_{D} (M) | Rₘₐₓ (RU) |
|---|---|---|---|---|
| SCT-Da148 | 7.31E+05 | 3.68E−04 | 5.03E−10 | 138.1 |
| SCT-Da182 | 2.32E+05 | 2.83E−04 | 1.22E−09 | 206.1 |
| SCT-Da192 | 3.07E+05 | 5.05E−04 | 1.64E−09 | 132.2 |
| SCT-Da250 | 9.05E+05 | 3.61E−04 | 3.98E−10 | 171.0 |

| | |
|---|---|
| SCT-Da333 | No binding |

TABLE 5
CDR sequences of mAbs generated against human B7-H3
(SEQ ID Nos for each listed sequence are provided in parenthesis)

| mAb AA ID | HCDR1 | HCDR2 | HCDR3 | LCDR1 | LCDR2 | LCDR3 |
|---|---|---|---|---|---|---|
| SCT-Da001 | GYSITSNYN | IHYSDDT | ARWSYYYGGPYYYSMDY | QDINSY | RAN | LQYDEFPPT |
| | (479) | (480) | (481) | (763) | (764) | (765) |
| SCT-Da002 | GYSITSGYN | IHYSGGT | GRWSYYYGSPYYYSMDY | QDINTY | RAN | LQYDEFPPT |
| | (482) | (483) | (484) | (766) | (764) | (765) |
| SCT-Da003 | GFSITSGYY | ISYDGSN | ARDYYGYPAWFTY | QSVDYDGDSY | AAS | QQSNEDPRT |
| | (485) | (486) | (487) | (857) | (794) | (837) |
| SCT-Da004 | GYSITDGYF | LNYDGSN | ARRKGWLYAMDY | QDISNY | YTS | QQGNALPWT |
| | (488) | (489) | (490) | (773) | (771) | (807) |
| SCT-Da005 | GYSITSAYF | INYDGSN | ARRKGWLYAMDY | QDISNY | YTS | QQGKSLPWT |
| | (491) | (492) | (490) | (773) | (771) | (814) |
| SCT-Da006 | GYSITSAYF | INFDGSS | ARRKGWLYAMDY | QDISNY | YTS | QQGNTLPWT |
| | (491) | (493) | (490) | (773) | (771) | (772) |
| SCT-Da007 | GYSITSAYF | INFDGSS | ARRKGWLYAMDY | QDISNY | YTS | QQGNTLPWT |
| | (491) | (493) | (490) | (773) | (771) | (772) |
| SCT-Da008 | GYSITSAYF | INFDGSS | ARRKGWLYAMDY | QDINNY | YTS | QQGNTLPWT |
| | (491) | (493) | (490) | (770) | (771) | (772) |
| SCT-Da009 | GYSITSAYF | INFDGSS | ARRKGWLYAMDY | QDISNY | YTS | QQGNTLPWT |
| | (491) | (493) | (490) | (773) | (771) | (772) |
| SCT-Da010 | GYSITSAYF | INYDGTN | ARRKGWLYAMDY | QDISNY | YTS | QQGKSLPWT |
| | (491) | (494) | (490) | (773) | (771) | (814) |
| SCT-Da011 | GYSITSAYF | INYDGTN | ARRKGWLYAMDY | QDINNY | YTS | QQGNTLPWT |
| | (491) | (494) | (490) | (770) | (771) | (772) |
| SCT-Da012 | GYSITSAYF | INYDGTN | ARRKGWLYAMDY | QDISNY | YTS | QQGKSLPWT |
| | (491) | (494) | (490) | (773) | (771) | (814) |
| SCT-Da013 | GYSITSDSF | ISYDGSN | ARRKGWLYAMDY | QDINNY | YTS | QQGNTLPWT |
| | (495) | (486) | (490) | (770) | (771) | (772) |
| SCT-Da014 | GYSITSDYF | ISYDGSN | ARRKGWLYAMDY | QDISNY | YTS | QQGNTLPWT |
| | (496) | (486) | (490) | (773) | (771) | (772) |
| SCT-Da015 | GYSITSDYF | ISYDGSN | ARRKGWLYAMDY | QDISNF | YTS | QQGKTLPWT |
| | (496) | (486) | (490) | (812) | (771) | (813) |
| SCT-Da016 | GYSITSGYF | INYDGSN | ARRKGWLYAMDY | QDISNY | YTS | QQGNTLPWT |
| | (497) | (492) | (490) | (773) | (771) | (772) |
| SCT-Da017 | GYSITSGYF | INYDGSD | ARRKGWLYAMDS | QDINNY | YTS | QQGNTLPWT |
| | (497) | (498) | (499) | (770) | (771) | (772) |
| SCT-Da018 | GYSITSGYF | INYDGSD | ARRKGWLYAMDS | HDIHNY | YTS | QQGNTLPWT |
| | (497) | (498) | (499) | (811) | (771) | (772) |
| SCT-Da019 | GYSITSGYF | INYDGSN | ARRKGWLYAMDY | QDISNY | YTS | QQGNTLPWT |
| | (497) | (492) | (490) | (773) | (771) | (772) |
| SCT-Da020 | GYSITSGYF | INYDGSS | ARRKGWLYAMDY | SSISSNY | RTS | QQGSSIPRT |
| | (497) | (500) | (490) | (928) | (929) | (932) |
| SCT-Da021 | GYSITSGYF | LNYDGSN | ARRKGWLYAMDY | QDISNY | YTS | QQGNALPWT |
| | (497) | (489) | (490) | (773) | (771) | (807) |
| SCT-Da022 | GYSITSGYF | LNYDGSN | ARRKGWLYAMDY | QDISNY | YTS | QQGNALPWT |
| | (497) | (489) | (490) | (773) | (771) | (807) |
| SCT-Da023 | GYSITSGYF | LNYDGSN | ARRKGWLYAMDY | QDISNY | YTS | QQGIALPWT |
| | (497) | (489) | (490) | (773) | (771) | (808) |
| SCT-Da024 | GYSITSGYF | LNYDGSN | ARRKGWLYAMDY | QDISNY | YTS | QQGIALPWT |
| | (497) | (489) | (490) | (773) | (771) | (808) |
| SCT-Da025 | GYSITSGYF | LNYDGSN | ARRKGWLYAMDY | QDISNY | YTS | QQGNALPWT |
| | (497) | (489) | (490) | (773) | (771) | (807) |
| SCT-Da026 | GYSITSGYF | LNYDGSN | ARRKGWLYAMDY | ESVDSYGNSF | IAS | QQSNEDPRT |
| | (497) | (489) | (490) | (846) | (842) | (837) |
| SCT-Da027 | GYSITSGYF | LNYDGSN | ARRKGWLYAMDY | QSVTND | YAS | QQDYNSPYT |
| | (497) | (489) | (490) | (970) | (818) | (962) |
| SCT-Da028 | GYSITSGYF | LNYDGSN | ARRKGWLYAMDY | QDISNY | YTS | QQGNTLPWT |
| | (497) | (489) | (490) | (773) | (771) | (772) |
| SCT-Da029 | GYSITSGYF | LNYDGSS | ARRKGWLYAMDY | QDISNY | YTS | QQGNALPWT |
| | (497) | (501) | (490) | (773) | (771) | (807) |
| SCT-Da030 | GYSITSGYF | INYDGSN | ARRKGWLYAMDY | QDISNY | YTS | QQGNTLPWT |
| | (497) | (492) | (490) | (773) | (771) | (772) |
| SCT-Da031 | GYSITSGYY | ISYDGSN | AREGDYGSY | QDVGTA | WAS | QQYSSYPYT |
| | (502) | (486) | (503) | (879) | (866) | (881) |
| SCT-Da032 | GYSITSGYY | ISYDGSN | TRRKGWLYAMDY | QDISNY | YTS | QQGNALPWT |
| | (502) | (486) | (504) | (773) | (771) | (807) |
| SCT-Da033 | GYSITSDYA | ISYSGST | ASGYYGIHYYAMDY | QDVNTA | SAS | QQHYSTPFT |
| | (505) | (506) | (507) | (876) | (882) | (883) |
| SCT-Da034 | GYSITSGYF | INYDGSS | ARRKGWLYAMDY | QDISNY | YTS | QQGNTLPWT |
| | (497) | (500) | (490) | (773) | (771) | (772) |
| SCT-Da035 | GYSFTAYI | INPYYGST | ARGPPYYYGSSRDYALDY | QSIVHTNGNTY | KVS | FQGSHVPYT |
| | (508) | (509) | (510) | (906) | (907) | (908) |
| SCT-Da036 | GYSFTDYI | INPYYDTT | ATYDGFAY | QSLVHSNGNTY | KVS | SQSTHGPWT |
| | (511) | (512) | (513) | (919) | (907) | (921) |
| SCT-Da037 | GYSFTDYI | INPYYGTT | ATYDGFAY | QSLVHSYGNTY | KVS | SQSTHGPWT |
| | (511) | (514) | (513) | (923) | (907) | (921) |
| SCT-Da038 | GYSFTDYI | INPYYYTT | ATYDGFAY | QSLVHSNGNTY | KVS | SQSTHGPWT |
| | (511) | (515) | (513) | (919) | (907) | (921) |
| SCT-Da039 | GFNIKDYY | IDPENGNT | TIGVRRDYFDY | SSISSNY | RTS | QQGSSLPRT |
| | (516) | (517) | (518) | (928) | (929) | (930) |
| SCT-Da040 | GFTFSNYW | IRLKFNNY | IKFGGMDY | SSVNY | STS | QQRSTYPRT |
| | (519) | (520) | (521) | (949) | (877) | (950) |
| SCT-Da041 | GFAFNSYD | ISDGGSFT | ARHQITTLRMDY | QGISSN | HGT | VQYAQFPYT |
| | (522) | (523) | (524) | (767) | (768) | (769) |
| SCT-Da042 | GFAFSSYD | ISDGGSYT | ARHRVTTVRMDY | QGISSN | HGT | VQYAQFPYT |
| | (525) | (526) | (527) | (767) | (768) | (769) |
| SCT-Da043 | GFAFSSYD | ISDGGSYT | ARHRVTTVRMDY | QGISSN | HGT | VQYAQFPYT |
| | (525) | (526) | (527) | (767) | (768) | (769) |
| SCT-Da044 | GFAFSDYD | ISDGGSYT | ARHLITTLRMDY | QGISSN | HGT | VQYAQFPYT |
| | (528) | (526) | (529) | (767) | (768) | (769) |
| SCT-Da045 | GFAFSDYD | ISDGGSYT | ARHLITTLRMDY | QGISSN | HGT | VQYAQFPYT |
| | (528) | (526) | (529) | (767) | (768) | (769) |
| SCT-Da046 | GFAFSDYD | ISDGGSYT | ARHLITTLRMDY | QGISSN | HGT | VQYAQFPYT |
| | (528) | (526) | (529) | (767) | (768) | (769) |
| SCT-Da047 | GFAFSDYD | ISNDGSYT | ARHLITTLRMDY | QGISSN | HGT | VQYAQFPYT |
| | (528) | (530) | (529) | (767) | (768) | (769) |
| SCT-Da048 | GFAFSDYD | ISNDGSYT | ARHLITTLRMDY | QGISSN | HGT | VQYAQFPYT |
| | (528) | (530) | (529) | (767) | (768) | (769) |
| SCT-Da049 | GFTFSSYT | IISGGGDI | ARWYSAGYFDV | KSVSTSAYSY | LAS | QHSRELLT |
| | (531) | (532) | (533) | (852) | (829) | (853) |
| SCT-Da050 | GFTFSSYT | ISGGGVTY | ARWYSVGYFDV | KSVSTSAYSY | LAS | QHSRELLT |
| | (531) | (534) | (535) | (852) | (829) | (853) |
| SCT-Da051 | GFTFSSYT | ITGGGDTY | ARWYSVGYFDV | KSVSTSAYSY | LAS | QHSRELLT |
| | (531) | (536) | (535) | (852) | (829) | (853) |
| SCT-Da052 | GFTFSSYT | ITSGGGDT | ARWYSAGYFDV | KSVSTSAYSY | LAS | QHSRELLT |
| | (531) | (537) | (533) | (852) | (829) | (853) |
| SCT-Da053 | GFTFSSYT | ISGGGVTY | ARWYSVGYFDV | KSVSTSAYSY | LAS | QHSRELLT |
| | (531) | (534) | (535) | (852) | (829) | (853) |
| SCT-Da054 | GFTFSSYA | IFFGSSVT | ARQGTSPWFAY | KSVSTSNYSY | LAS | QHSRELPT |
| | (538) | (539) | (540) | (855) | (829) | (856) |
| SCT-Da055 | GFTFSSYT | IISGGGNT | ARWYSAGYFDV | KSVSTSGYSY | LAS | QHSRELLT |
| | (531) | (541) | (533) | (828) | (829) | (853) |
| SCT-Da056 | GFTFSSYT | ISGGVGDT | ARQGGFPWFAY | KSVSTSGYSY | LAS | QHSRELPYT |
| | (531) | (542) | (543) | (828) | (829) | (830) |
| SCT-Da057 | GFNIEDTY | IDPANGNT | ARDGYYAPGY | QNVDTN | SGS | QQYNTYPYT |
| | (544) | (545) | (546) | (873) | (874) | (875) |
| SCT-Da058 | GFNIEDTY | IDPANGNT | ARDGYYAPGY | QNVDTN | SAS | QQYNSYPYT |
| | (544) | (545) | (546) | (873) | (882) | (902) |
| SCT-Da059 | GFNIEDTY | IDPANGYT | ARDGYYAPGY | QNVDTN | SAS | QQYNSYPYT |
| | (544) | (547) | (546) | (873) | (882) | (902) |
| SCT-Da060 | GYTFTDYN | INPNYDST | ARSGFDGYYYAMDY | ETVDSYGSSF | RAS | QQSNEDPFT |
| | (548) | (549) | (550) | (864) | (844) | (845) |
| SCT-Da061 | DYTFTDYN | INPNNGGT | ARRHYGNYFYAMDY | QGISNY | YTS | QQYSKLPLT |
| | (551) | (552) | (553) | (815) | (771) | (816) |
| SCT-Da062 | GYTFTDYN | INPNGDFT | ARRHYGNYFYAMDY | ESVDSYGNSF | IAS | QQSNEDPRT |
| | (548) | (554) | (553) | (846) | (842) | (837) |
| SCT-Da063 | GYTFTDYN | INPNNGGT | ARRHYGNYFYAMDY | ESVDSYGNSF | IAS | QQSNEDPRT |
| | (548) | (552) | (553) | (846) | (842) | (837) |
| SCT-Da064 | GYTFTDYN | INPNDDST | ARRHYGNYFYAMDY | DHINNW | GAT | QQYWSTPT |
| | (548) | (555) | (553) | (799) | (800) | (804) |
| SCT-Da065 | GYTFTDYN | INPNDDST | ARRHYGNYFYAMDY | ESVDSFGHSF | LAS | QQSNEDPRT |
| | (548) | (555) | (553) | (841) | (829) | (837) |
| SCT-Da066 | GYTFTDYN | INPNDDST | ARRHYGNYFYAMDY | ESVDSFGNSF | IAS | QQSNEDPRT |
| | (548) | (555) | (553) | (836) | (842) | (837) |
| SCT-Da067 | GYTFTDYN | INPNNGGT | ARRHYGNYFYAMDY | ESVDNFGNSF | LAS | QQSNEDPRT |
| | (548) | (552) | (553) | (838) | (829) | (837) |
| SCT-Da068 | GYTFTDYN | INPNNGGT | ARRHYGNYFYAMDY | ESVDSYGNSF | IAS | QQSNEDPRT |
| | (548) | (552) | (553) | (846) | (842) | (837) |
| SCT-Da069 | GYTFTDYN | INPNNGGT | ARRHYGNYFYAMDY | ESVDSFGNSF | LAS | QQSNEDPRT |
| | (548) | (552) | (553) | (836) | (829) | (837) |
| SCT-Da070 | GYTFTDYN | INPNNGGT | ARRHYGNYFYAMDY | ESVDSYGNSF | LAS | QQSNEDPRT |
| | (548) | (552) | (553) | (846) | (829) | (837) |
| SCT-Da071 | GYTFTDYN | INPNNGGT | ARRHYGNYFYAMDY | QSVDYDGDSY | AAS | QQSNEDPRT |
| | (548) | (552) | (553) | (857) | (794) | (837) |
| SCT-Da072 | GYTFTDYN | INPNNGGT | ARRHYGNYFYAMDY | QNVGTD | SAS | QQYNSYPYT |
| | (548) | (552) | (553) | (904) | (882) | (902) |
| SCT-Da073 | GYTFTDYN | INPNNGGT | ARRHYGNYFYAMDY | QSVDYDGDSY | AAS | QQSNEDPRT |
| | (548) | (552) | (553) | (857) | (794) | (837) |
| SCT-Da074 | GYTFTDYN | INPNYDST | ARRHYGNYFYAMDY | DHINNW | GAT | QQYWSIPYT |
| | (548) | (549) | (553) | (799) | (800) | (802) |
| SCT-Da075 | GYTFTDYN | INPNYDST | ARRHYGNYFYAMDY | ESVDSFGNSF | IAS | QQSNEDPRT |
| | (548) | (549) | (553) | (836) | (842) | (837) |
| SCT-Da076 | GYTFTDYN | INPNYDST | ARRHYGNYFYAMDY | QSVDYDGDSY | AAS | QQSNEDPRT |
| | (548) | (549) | (553) | (857) | (794) | (837) |
| SCT-Da077 | GYTFTDYN | ISPNYEST | ARRHYGNYFYAMDY | ESVDSYGNSF | IAS | QQSNEDPRT |
| | (548) | (556) | (553) | (846) | (842) | (837) |
| SCT-Da078 | GYTFTDYN | ISPNYEST | ARRHYGNYFYAMDY | ESVDSFGHSF | LAS | QQSNEDPRT |
| | (548) | (556) | (553) | (841) | (829) | (837) |
| SCT-Da079 | GYTFTDYN | INPNDDST | ARRHYGNYFYAMDY | ESVDSFGNSF | LAS | QQSNEDPRT |
| | (548) | (555) | (553) | (836) | (829) | (837) |
| SCT-Da080 | GYTFTDYN | INPNYDST | ARGGFYGNFFDY | QDISNY | YTS | QQGNTLPWT |
| | (548) | (549) | (557) | (773) | (771) | (772) |
| SCT-Da081 | GYTFTDYN | INPNYDST | ARGGFYGNFFDY | ESVDSSGNSF | RAS | QQSNEDPFT |
| | (548) | (549) | (557) | (843) | (844) | (845) |
| SCT-Da082 | GFNIKDYY | IDPENGNT | SIGVRRDYFAY | SSISSNY | RTS | QQGSSLPRT |
| | (516) | (517) | (558) | (928) | (929) | (930) |
| SCT-Da083 | GFNIKDYY | IDPENGNT | TIGVRRDYFDY | QGISSN | HGT | VQYAQFPYT |
| | (516) | (517) | (518) | (767) | (768) | (769) |
| SCT-Da084 | GFNIKDYY | IDPENGNT | TIGVRRDYFDY | SSISSNY | RTS | QQGSSIPRT |
| | (516) | (517) | (518) | (928) | (929) | (932) |
| SCT-Da085 | GFNIEDTY | IDPANGNP | ARDGYYAPGY | SSISSNY | RTS | QQGSSIPRT |
| | (544) | (559) | (546) | (928) | (929) | (932) |
| SCT-Da086 | GFNIEDTY | IDPANGNP | ARDGYYAPGY | QNVDTN | SGS | QQYNTYPYT |
| | (544) | (559) | (546) | (873) | (874) | (875) |
| SCT-Da087 | GFNIEDTY | IDPANGNR | ARDGYYAPGY | QNVGTN | SAS | QQYNSYPYT |
| | (544) | (560) | (546) | (899) | (882) | (902) |
| SCT-Da088 | GFNIEDTY | IDPANGNT | ARDGYYAPGY | QDINKY | YTS | LQYDNLRT |
| | (544) | (545) | (546) | (796) | (771) | (797) |
| SCT-Da089 | GFNIEDTY | IDPANGNT | ARDGYYAPGY | DHINNW | GAT | QQYWSIPYT |
| | (544) | (545) | (546) | (799) | (800) | (802) |
| SCT-Da090 | GFNIEDTY | IDPANGNT | ARDGYYAPGY | QNVDTN | SGS | QQYNTYPYT |
| | (544) | (545) | (546) | (873) | (874) | (875) |
| SCT-Da091 | GFNIEDTY | NDPANGNT | ASLWVDY | KSLLHSNGNTF | RMS | MQHLEFPYT |
| | (544) | (561) | (562) | (868) | (869) | (870) |
| SCT-Da092 | GFNIKDTY | IDPANANT | AHDGYYAPGY | QNVGTN | SAS | QQYNNYPYT |
| | (563) | (564) | (565) | (899) | (882) | (900) |
| SCT-Da093 | GFNIKDTY | IDPANGNT | AHDGYYAPGY | QNVGTD | SAS | QQYNIYPYT |
| | (563) | (545) | (565) | (904) | (882) | (903) |
| SCT-Da094 | GFNIKDTY | IDPANGNT | AHDGYYAPGY | QNVGTD | SAS | QQYNSYPYT |
| | (563) | (545) | (565) | (904) | (882) | (902) |
| SCT-Da095 | GFNIKDTY | IDPANGNT | AHDGYYAPGY | TDIDDD | EGN | LQSDNLPFT |
| | (563) | (545) | (565) | (939) | (940) | (941) |
| SCT-Da096 | GFNIKDTY | IDPANGNT | AHDGYYAPGY | QNVGTN | SAS | QQYNSYPYT |
| | (563) | (545) | (565) | (899) | (882) | (902) |
| SCT-Da097 | GFNIKDTY | IDPANGNS | AHDGYYAPGY | QNVGTD | SAS | QQYNSYPYT |
| | (563) | (566) | (565) | (904) | (882) | (902) |
| SCT-Da098 | GFNIKDTY | IDPANGNS | AHDGYYAPGY | QNVGTN | SAS | QQYNSYPYT |
| | (563) | (566) | (565) | (899) | (882) | (902) |
| SCT-Da099 | GFNIKDTY | IDPANGNS | AHDGYYAPGY | QNVGTN | SAS | QQYNSYPYT |
| | (563) | (566) | (565) | (899) | (882) | (902) |
| SCT-Da100 | GFNIKDTY | IDPANGNS | AHDGYYAPGY | QNVGTN | SAS | QQYNSYPYT |
| | (563) | (566) | (565) | (899) | (882) | (902) |
| SCT-Da101 | GFNIKDTY | IDPANGNT | AHDGYYAPGY | QNVGTD | SAS | QQYNSYPYT |
| | (563) | (545) | (565) | (904) | (882) | (902) |
| SCT-Da102 | GFNIKDTY | IDPANGNT | AHDGYYAPGN | QNVGTD | SAS | QQYNSYPYT |
| | (563) | (545) | (567) | (904) | (882) | (902) |
| SCT-Da103 | GFNIKDTY | IDPANGNT | AHDGYYAPGY | QGISNY | YTS | QQYSKLPYT |
| | (563) | (545) | (565) | (815) | (771) | (810) |
| SCT-Da104 | GFNIKDTY | IDPANGNT | AHDGYYAPGY | QNVGTD | SAS | QQYNSYPYT |
| | (563) | (545) | (565) | (904) | (882) | (902) |
| SCT-Da105 | GFNIKDTY | IDPANGNT | AHDGYYAPGY | QNVGTD | SAS | QQYNSYPYT |
| | (563) | (545) | (565) | (904) | (882) | (902) |
| SCT-Da106 | GFNIKDTY | IDPANGNT | AHDGYYAPGY | QNVGTD | SAS | QQYNSYPYT |
| | (563) | (545) | (565) | (904) | (882) | (902) |
| SCT-Da107 | GFNIKDTY | IDPANGNR | ARDGYYAPGY | QGISSN | HGT | VQYAQFPYT |
| | (563) | (560) | (546) | (767) | (768) | (769) |
| SCT-Da108 | GFNIKDTY | IDPANGNR | ARDGYYAPGY | QSVDYDGDNY | AAS | QQSYEDPWT |
| | (563) | (560) | (546) | (831) | (794) | (832) |
| SCT-Da109 | GFNIKDTY | IDPANGNR | ARDGYYAPGY | QNVDSN | SAS | QQYNSYPYT |
| | (563) | (560) | (546) | (901) | (882) | (902) |
| SCT-Da110 | GFNIKDTY | IDPANGNR | ARDGYYAPGY | QNVGTN | SAS | QQYNTYPYT |
| | (563) | (560) | (546) | (899) | (882) | (875) |
| SCT-Da111 | GFNIKDTY | IDPANGNR | ARDGYYAPGY | QSISDY | YVS | QNGHSFPFT |
| | (563) | (560) | (546) | (888) | (897) | (889) |
| SCT-Da112 | GFNIKDTY | IDPANGNR | ARDGYYAPGY | QNVGTD | SAS | QQYNSYPYT |
| | (563) | (560) | (546) | (904) | (882) | (902) |
| SCT-Da113 | GFNIKDTY | IDPANGNR | ARDGYYAPGY | QNVGTD | SAS | QQYNSYPYT |
| | (563) | (560) | (546) | (904) | (882) | (902) |
| SCT-Da114 | GFNIKDTY | IDPANGNR | ARDGYYAPGY | SSVSY | LTS | QQWSSNPLT |
| | (563) | (560) | (546) | (944) | (957) | (947) |
| SCT-Da115 | GFNIKDTY | IDPANGNR | ARDGYYAPGY | QNVGTN | SAS | QQYNSYPYT |
| | (563) | (560) | (546) | (899) | (882) | (902) |
| SCT-Da116 | GFNIKDTY | IDPANGNR | ARDGYYAPGH | QNVGTD | SAS | QQYNSYPYT |
| | (563) | (560) | (568) | (904) | (882) | (902) |
| SCT-Da117 | GFNIKDTY | IDPANGNR | ARDGYYAPGH | QNVGTD | SAS | QQYNSYPYT |
| | (563) | (560) | (568) | (904) | (882) | (902) |
| SCT-Da118 | GFNIKDTY | IDPANGNR | ARDGYYAPGH | QNVGTD | SAS | QQYNSYPYT |
| | (563) | (560) | (568) | (904) | (882) | (902) |
| SCT-Da119 | GFNIKDTY | IDPANGNR | ARDGYYAPGH | QNVGTD | SAS | QQYNSYPYT |
| | (563) | (560) | (568) | (904) | (882) | (902) |
| SCT-Da120 | GFNIKDTY | IDPANGNR | ARDGYSAPGY | QNVGTD | SAS | QQYNSYPYT |
| | (563) | (560) | (569) | (904) | (882) | (902) |
| SCT-Da121 | GFNIKDTY | IDPANGNR | ARDGYYAPGY | ESVESNGNSF | RAS | QQTDEDPFT |
| | (563) | (560) | (546) | (848) | (844) | (849) |
| SCT-Da122 | GFNIKDTY | IDPANGNR | ARDGYYAPGY | QRISDY | YAS | QNGHSFPFT |
| | (563) | (560) | (546) | (893) | (818) | (889) |
| SCT-Da123 | GFNIKDTY | IDPANGNR | ARDGYYAPGY | QNVGTD | SAS | QQYNSYPYT |
| | (563) | (560) | (546) | (904) | (882) | (902) |
| SCT-Da124 | GFNIKDTY | IDPANGNR | ARDGYYAPGY | QNVGTD | SAS | QQYNSYPYT |
| | (563) | (560) | (546) | (904) | (882) | (902) |
| SCT-Da125 | GFNIKDTY | IDPANGNR | ARDGYYAPGY | QNVGTD | SAS | QQYNSYPYT |
| | (563) | (560) | (546) | (904) | (882) | (902) |
| SCT-Da126 | GFNIKDTY | IDPANGNR | ARDGYYAPGY | QNVGTD | SAS | QQYNTYPYT |
| | (563) | (560) | (546) | (904) | (882) | (875) |
| SCT-Da127 | GFNIKDTY | IDPANGNR | ARDGYYAPGY | KSISKY | SGS | QQHNEYPLT |
| | (563) | (560) | (546) | (909) | (874) | (910) |
| SCT-Da128 | GFNIKDTY | IDPANGNR | ARDGYYAPGY | SSVSY | LTS | QQWSSNPLT |
| | (563) | (560) | (546) | (944) | (957) | (947) |
| SCT-Da129 | GFNIKDTY | IDPANGNR | ARDGYYAPGY | QNVGTD | SAS | QQYNSYPYT |
| | (563) | (560) | (546) | (904) | (882) | (902) |
| SCT-Da130 | GFNIKDTY | IDPANGNR | ARDGYYAPGY | QNVGTN | SAS | QQYNSYPYT |
| | (563) | (560) | (546) | (899) | (882) | (902) |
| SCT-Da131 | GFNIKDTY | IDPANGNR | ARDGYYAPGY | QNVGTN | SAS | QQYNSYPYT |
| | (563) | (560) | (546) | (899) | (882) | (902) |
| SCT-Da132 | GFNIKDTY | IDPANGNR | ARDGYYAPGY | QNVGTN | SAS | QQYNSYPYT |
| | (563) | (560) | (546) | (899) | (882) | (902) |
| SCT-Da133 | GFNIKDTY | IDPANGNT | AHDGYYAPGY | QNVDSN | SAS | QQYNSYPYT |
| | (563) | (545) | (565) | (901) | (882) | (902) |
| SCT-Da134 | GFNIKDTY | IDPANGNT | AHDGYYAPGY | QNVDTN | SAS | QQYNSYPYT |
| | (563) | (545) | (565) | (873) | (882) | (902) |
| SCT-Da135 | GFNIKDTY | IDPANGNT | AHDGYYAPGY | SSVSY | LTS | QQWSSNPLT |
| | (563) | (545) | (565) | (944) | (957) | (947) |
| SCT-Da136 | GFNIKDTY | IDPANGNT | AHDGYYAPGY | QSVSND | YAS | QQDYSSPWT |
| | (563) | (545) | (565) | (966) | (818) | (968) |
| SCT-Da137 | GFNIKDTY | IDPANGNT | THDGYYAPGY | QNVDTN | SAS | QQYNIYPYT |
| | (563) | (545) | (570) | (873) | (882) | (903) |
| SCT-Da138 | GFNIKDTY | IDPANGNT | THDGYYAPGY | QNVDTN | SAS | QQYNSYPYT |
| | (563) | (545) | (570) | (873) | (882) | (902) |
| SCT-Da139 | GFNIKDTY | IDPANGNT | THDGYYAPGY | QSLVHSNGNTY | KVS | SQSTHAPWT |
| | (563) | (545) | (570) | (919) | (907) | (920) |
| SCT-Da140 | GFNIKDTY | IDPANGNT | AHDGYYAPGY | QNVGTN | SAS | QQYNNYPYT |
| | (563) | (545) | (565) | (899) | (882) | (900) |
| SCT-Da141 | GFNIKDTY | IDPANGNT | AHDGYYAPGY | QNVGTN | SAS | QQYNNYPYT |
| | (563) | (545) | (565) | (899) | (882) | (900) |
| SCT-Da142 | GFNIKDTY | IDPANGNT | AHDGYYAPGF | QNVGTN | SAS | QQYNSYPFT |
| | (563) | (545) | (571) | (899) | (882) | (905) |
| SCT-Da143 | GFNIKDTY | IDPANGNT | APFSYGTLAY | SSIISNY | RTS | QQGSSLPRT |
| | (563) | (545) | (572) | (935) | (929) | (930) |
| SCT-Da144 | GLNIKDTY | IDPANGNR | ARDGYYAPGY | QNVGTN | SAS | QQYNSYPYT |
| | (573) | (560) | (546) | (899) | (882) | (902) |
| SCT-Da145 | GVNIKDTY | IDPANGNR | ARDGYYAPGY | QNVGTD | SAS | QQYNSYPYT |
| | (574) | (560) | (546) | (904) | (882) | (902) |
| SCT-Da146 | GVNIKDTY | IDPANGNR | ARDGYYAPGY | QGISSN | HGT | VQYAQFPYT |
| | (574) | (560) | (546) | (767) | (768) | (769) |
| SCT-Da147 | GVNIKDTY | IDPANGNR | ARDGYYAPGY | QGISNY | YTS | QQYSKLPLT |
| | (574) | (560) | (546) | (815) | (771) | (816) |
| SCT-Da148 | GVNIKDTY | IDPANGNR | ARDGYYAPGY | QNVGTD | SAS | QQYNSYPYT |
| | (574) | (560) | (546) | (904) | (882) | (902) |
| SCT-Da149 | GVNIKDTY | IDPANGNR | ARDGYYAPGY | QNVGTD | SAS | QQYNSYPYT |
| | (574) | (560) | (546) | (904) | (882) | (902) |
| SCT-Da150 | GVNIKDTY | IDPANGNR | ARDGYYAPGY | QNVGTD | SAS | QQYNSYPYT |
| | (574) | (560) | (546) | (904) | (882) | (902) |
| SCT-Da151 | GVNIKDTY | IDPANGNR | ARDGYYAPGY | QNVGTD | SAS | QQYNSYPYT |
| | (574) | (560) | (546) | (904) | (882) | (902) |
| SCT-Da152 | GVNIKDTY | IDPANGNR | ARDGYYAPGY | QNVGTN | SAS | QQYNSYPYT |
| | (574) | (560) | (546) | (899) | (882) | (902) |
| SCT-Da153 | GVNIKDTY | IDPANGNR | ARDGYYAPGY | QNVGTN | SAS | QQYNSYPYT |
| | (574) | (560) | (546) | (899) | (882) | (902) |
| SCT-Da154 | GVNIKDTY | IDPANGNR | ARDGYYAPGY | QNVDNY | YAS | QQDYSSPYT |
| | (574) | (560) | (546) | (959) | (818) | (960) |
| SCT-Da155 | GFNIKDTY | IDPANFNT | ASLWVDY | KSLLHSNGNTF | RMS | MQHLEYPYT |
| | (563) | (575) | (562) | (868) | (869) | (871) |
| SCT-Da156 | GFNIKDTY | IDPANGNR | ARDGYYAPGH | QDINKY | YTS | LQYDNLRT |
| | (563) | (560) | (568) | (796) | (771) | (797) |
| SCT-Da157 | GFNIKDTY | IDPANGNR | ARDGYYAPGH | QNVGTD | SAS | QQYNSYPYT |
| | (563) | (560) | (568) | (904) | (882) | (902) |
| SCT-Da158 | GFNIKDTY | IDPANGNR | ARDGYYAPGH | QNVGTD | SAS | QQYNSYPYT |
| | (563) | (560) | (568) | (904) | (882) | (902) |
| SCT-Da159 | GFNIKDTY | IDPANGNR | ARDGYYAPGH | QNVGTN | SAS | QQYNSYPYT |
| | (563) | (560) | (568) | (899) | (882) | (902) |
| SCT-Da160 | GCNIKDYY | IDPENGNT | TIGVRRDYFAY | SSISSNY | RTS | QQGSSIPRT |
| | (576) | (517) | (577) | (928) | (929) | (932) |
| SCT-Da161 | GCNIKDYY | IDPENGNT | AIGVRRDYFAY | SSISSNY | RTS | QQGSSIPRT |
| | (576) | (517) | (578) | (928) | (929) | (932) |
| SCT-Da162 | GFNIKDFY | IDPENGNT | AIGVRRDYFGY | SIISSNY | RTS | QQGSSIPRT |
| | (579) | (517) | (580) | (934) | (929) | (932) |
| SCT-Da163 | GFNIKDYY | IDPENGNT | AIGVRRDYFAY | QDISNY | YTS | QQGNALPWT |
| | (516) | (517) | (578) | (773) | (771) | (807) |
| SCT-Da164 | GFNIKDYY | IDPENGNT | AIGVRRDYFAY | HDIHNY | YTS | QQGNTLPWT |
| | (516) | (517) | (578) | (811) | (771) | (772) |
| SCT-Da165 | GFNIKDYY | IDPENGNT | AIGVRRDYFAY | SSISSNY | RTS | QQGSSLPRT |
| | (516) | (517) | (578) | (928) | (929) | (930) |
| SCT-Da166 | GFNIKDYY | IDPENGNT | AIGVRRDYFAY | SNISSNY | RTS | QQGSSIPRT |
| | (516) | (517) | (578) | (931) | (929) | (932) |
| SCT-Da167 | GFNIKDYY | IDPENGNT | AIGVRRDYFAY | QDIKSY | YAT | LQHGESPFT |
| | (516) | (517) | (578) | (760) | (761) | (762) |
| SCT-Da168 | GFNIKDYY | IDPENGNT | AIGVRRDYFAY | KSVSSSGYNY | LAS | QHSGELPLT |
| | (516) | (517) | (578) | (850) | (829) | (851) |
| SCT-Da169 | GFNIKDYY | IDPENGNT | AIGVRRDYFAY | QSISDY | YAS | QNGHSFPPFT |
| | (516) | (517) | (578) | (888) | (818) | (895) |
| SCT-Da170 | GFNIKDYY | IDPENGNT | AIGVRRDYFAY | QSLVHSDGNTY | KVS | SQSTHVPWT |
| | (516) | (517) | (578) | (918) | (907) | (916) |
| SCT-Da171 | GFNIKDYY | IDPENGNT | AIGVRRDYFAY | SSINSNY | RTS | QQGSSIPRT |
| | (516) | (517) | (578) | (933) | (929) | (932) |
| SCT-Da172 | GFNIKDYY | IDPENGNT | AIGVRRDYFAY | SSISSNY | RTS | QQGSSIPRT |
| | (516) | (517) | (578) | (928) | (929) | (932) |
| SCT-Da173 | GFNIKDYY | IDPENGNT | AIGVRRDYFAY | SSISSNY | RTS | QQGSSIPRT |
| | (516) | (517) | (578) | (928) | (929) | (932) |
| SCT-Da174 | GFNIKDYY | IDPENGNT | AIGVRRDYFAY | SSISY | GTS | QQWSSYPLT |
| | (516) | (517) | (578) | (954) | (955) | (956) |
| SCT-Da175 | GFNIKDYY | IDPENGNT | AIGVRRDYFDY | SSISSNY | RTS | QQGSSIPRT |
| | (516) | (517) | (581) | (928) | (929) | (932) |
| SCT-Da176 | GFNIKDYY | IDPENGNT | AIGVRRDYFAY | QGISSN | HGT | VQYAQFPYT |
| | (516) | (517) | (578) | (767) | (768) | (769) |
| SCT-Da177 | GFNIKDYY | IDPENGNT | AIGVRRDYFAY | SNISSNY | RTS | QQGSSIPRT |
| | (516) | (517) | (578) | (931) | (929) | (932) |
| SCT-Da178 | GFNIKDYY | IDPENGNT | AIGVRRDYFAY | SSISSNY | RTS | QQGSSIPRT |
| | (516) | (517) | (578) | (928) | (929) | (932) |
| SCT-Da179 | GFNIKDYY | IDPENGNT | AIGVRRDYFDY | SSISSNH | RTS | QQGSSIPRT |
| | (516) | (517) | (581) | (936) | (929) | (932) |
| SCT-Da180 | GFNIKDYY | IDPENGNT | AIGVRRDYFDY | SSISSNH | RTS | QQGSSIPRT |
| | (516) | (517) | (581) | (936) | (929) | (932) |
| SCT-Da181 | GYTFTEYT | INPNSGGT | TRPHSLLRLRREFDY | SSVSY | LTS | QQWSSNPLT |
| | (582) | (583) | (584) | (944) | (957) | (947) |
| SCT-Da182 | GFSFTGYT | INTYNGGT | ARSPGRYSMDY | QSVDYNGYSY | PAS | QQSKEDPYT |
| | (585) | (586) | (587) | (833) | (834) | (835) |
| SCT-Da183 | GYTFTEYT | INPSTGGT | ARPHSLLRLRREFDY | SSVSY | LTS | QQWSSNPLT |
| | (582) | (588) | (589) | (944) | (957) | (947) |
| SCT-Da184 | GYTFAEYT | INPNNGGT | TRPHSLLRLRREFDY | SSVSY | LTS | QQWSSNPLT |
| | (590) | (552) | (584) | (944) | (957) | (947) |
| SCT-Da185 | GYTFTEYP | INPNNGGT | ALYGNYWYFDV | SSVSY | DTS | QQWSSNPLT |
| | (591) | (552) | (592) | (944) | (953) | (947) |
| SCT-Da186 | GYTFTEYT | INPNSGGT | ARPHSLLRLRREFDY | SSVSY | LTS | QQWSSNPLT |
| | (582) | (583) | (589) | (944) | (957) | (947) |
| SCT-Da187 | GYTFTEYT | INPNSGGT | ARPHSLLRLRREFDY | SSVSY | LTS | QQWSSNPLT |
| | (582) | (583) | (589) | (944) | (957) | (947) |
| SCT-Da188 | GYTFTEYTM | INPNNGGT | ARPHSLLRLRREFDY | SSVSY | LTS | QQWSSNPLT |
| | (593) | (552) | (589) | (944) | (957) | (947) |
| SCT-Da189 | GYTFTEYT | VNPNTGGT | ARPHSLLRLRREFDY | SSVSY | LTS | QQWSSNPLT |
| | (582) | (594) | (589) | (944) | (957) | (947) |
| SCT-Da190 | GYTFTEYT | INPNNGGT | ARPHSLLRLRREFDY | SSVSY | LTS | HQWSSNPLT |
| | (582) | (552) | (589) | (944) | (957) | (958) |
| SCT-Da191 | GYTFTEYT | INPNNGGT | ARPHSLLRLRREFDY | QNVDSN | SAS | QQYNSYPYT |
| | (582) | (552) | (589) | (901) | (882) | (902) |
| SCT-Da192 | GYTFTEYT | INPNNGGT | ARPHSLLRLRREFDY | SSVSY | LTS | QQWSSNPLT |
| | (582) | (552) | (589) | (944) | (957) | (947) |
| SCT-Da193 | GYTFTEYT | INPNNGGT | TRPHSLLRLRREFDY | SSVSY | LTS | QQWSSNPLT |
| | (582) | (552) | (584) | (944) | (957) | (947) |
| SCT-Da194 | GYTFTEYT | INPNSGGT | TRPHSLLRLRREFDY | SSVSY | LTS | QQWSSNPLT |
| | (582) | (583) | (584) | (944) | (957) | (947) |
| SCT-Da195 | GYTFTEYT | INPNSGGT | ARPHSLLRLRREFDY | SSVSY | LTS | QQWSSNPLT |
| | (582) | (583) | (589) | (944) | (957) | (947) |
| SCT-Da196 | GYTFTEYT | INPNSGGT | ARPHSLLRLRREFDY | SSVSY | LTS | HQWSSNPLT |
| | (582) | (583) | (589) | (944) | (957) | (958) |
| SCT-Da197 | GYTFTEYT | INPNSGGT | ARPHSLLRLRREFDY | SSVSY | LTS | QQWSSNPLT |
| | (582) | (583) | (589) | (944) | (957) | (947) |
| SCT-Da198 | GYTFTEYT | INPNSGGT | ARPHSLLRLRREFDY | QNVDTN | SAS | QQYNSYPYT |
| | (582) | (583) | (589) | (873) | (882) | (902) |
| SCT-Da199 | GYTFTEYT | INPNSGGT | ARPHSLLRLRREFDY | SSVSY | LTS | QQWSSNPLT |
| | (582) | (583) | (589) | (944) | (957) | (947) |
| SCT-Da200 | GYTFTEYT | INPNSGGT | ARPHSLLRLRREFDY | SSVSY | LTS | QQWSSNPLT |
| | (582) | (583) | (589) | (944) | (957) | (947) |
| SCT-Da201 | GYTFTEYT | INPNSGGT | ARPHSLLRLRREFDY | SSVSY | LTS | QQWSSNPLT |
| | (582) | (583) | (589) | (944) | (957) | (947) |
| SCT-Da202 | GYTFTEYT | INPNSGGT | ARPHSLLRLRREFDY | QNVDTN | SAS | QQYNSYPYT |
| | (582) | (583) | (589) | (873) | (882) | (902) |
| SCT-Da203 | GYTFTEYT | INPNSGGT | ARPHSLLRLRREFDY | SNISSNY | RTS | QQGSSIPRT |
| | (582) | (583) | (589) | (931) | (929) | (932) |
| SCT-Da204 | GYTFTEYT | INPNSGGT | ARPHSLLRLRREFDY | SSVSY | ATS | QQWSSNPLT |
| | (582) | (583) | (589) | (944) | (945) | (947) |
| SCT-Da205 | GYTFTEYT | INPNSGGT | ARPHSLLRLRREFDY | SSVSY | LTS | QQWSSNPLT |
| | (582) | (583) | (589) | (944) | (957) | (947) |
| SCT-Da206 | GYTFTEYT | INPNTGGT | ARPHSLLRLRREFDY | SSVSY | LTS | QQWSSNPLT |
| | (582) | (595) | (589) | (944) | (957) | (947) |
| SCT-Da207 | GYTFTEYT | INPNTGGT | ARPHSLLRLRREFDY | SSVSY | LTS | QQWSSNPLT |
| | (582) | (595) | (589) | (944) | (957) | (947) |
| SCT-Da208 | GYTFTEYT | VNLNTGGT | ARPHSLLRLRREFDY | SSVSY | LTS | QQWSSNPLT |
| | (582) | (596) | (589) | (944) | (957) | (947) |
| SCT-Da209 | GYTFTSYV | INPYHDGT | ARSRTVIGFDY | QNVGTD | SAS | QQYNIYPYT |
| | (597) | (598) | (599) | (904) | (882) | (903) |
| SCT-Da210 | GYTFTSYV | INPYHDGT | ARSRTVIGFDY | QSISDY | YAS | QSGHSFPLT |
| | (597) | (598) | (599) | (888) | (818) | (896) |
| SCT-Da211 | GYTFTSYV | INPYHDGT | ARSRTVIGFDY | QNVGTD | SAS | QQYNIYPYT |
| | (597) | (598) | (599) | (904) | (882) | (903) |
| SCT-Da212 | GYTFTSYV | INPYNDAT | ARGVRLLDY | ENIYSY | NAK | QHHYVIPPT |
| | (597) | (600) | (601) | (774) | (775) | (785) |
| SCT-Da213 | GYTFTSYV | INPYTDGT | AKSRTVIGFDY | QNISDY | YAS | QNGHSFPLT |
| | (597) | (602) | (603) | (890) | (818) | (891) |
| SCT-Da214 | GYTFTSYV | INPYNDGT | ARSRTVVGFDY | QNISDY | YAS | QNAHSFPLT |
| | (597) | (604) | (605) | (890) | (818) | (892) |
| SCT-Da215 | GYTFTTYV | INPYNDGS | ARWVYYDFGGSMDY | SSVSY | LTS | QQWSSNPLT |
| | (606) | (607) | (608) | (944) | (957) | (947) |
| SCT-Da216 | GYTFTTYV | INPYNDGS | ARWVYYDFGGSMDY | SSVSY | LTS | QQWSSNPLT |
| | (606) | (607) | (608) | (944) | (957) | (947) |
| SCT-Da217 | GYTFTTYV | INPYNDGS | ARWVYYDFGGSMDY | SSVSY | LTS | QQWSSNPLT |
| | (606) | (607) | (608) | (944) | (957) | (947) |
| SCT-Da218 | GYTFTEYT | VNPNTGGT | ARPHSLLRLRREFDY | SSVSY | LTS | QQWSSNPLT |
| | (582) | (594) | (589) | (944) | (957) | (947) |
| SCT-Da219 | GYTFTSYV | INPYHDGT | ARSRTVIGFDY | QSISDY | YAS | QNGHSFPLT |
| | (597) | (598) | (599) | (888) | (818) | (891) |
| SCT-Da220 | GYTFTEYT | INPNSGGT | TRPHSLLRLRREFDY | SSVSY | LTS | QQWSSNPLT |
| | (582) | (583) | (584) | (944) | (957) | (947) |
| SCT-Da221 | GYTFTEYT | INPNSGGT | ARPHSLLRLRREFDY | SSVSY | LTS | QQWSSNPLT |
| | (582) | (583) | (589) | (944) | (957) | (947) |
| SCT-Da222 | GYTFTEYT | INPNNGGT | ARPHSLLRLRREFDY | QNVDSN | SAS | QQYNSYPYT |
| | (582) | (552) | (589) | (901) | (882) | (902) |
| SCT-Da223 | GYTFTEYT | INPNNGGT | ARPHSLLRLRREFDY | SSVSY | LTS | QQWSSNPLT |
| | (582) | (552) | (589) | (944) | (957) | (947) |
| SCT-Da224 | GFTLSRYG | SSGGGIYT | ARHDGVPMDY | ENIYSY | NAK | QHHYGIPPWT |
| | (609) | (610) | (611) | (774) | (775) | (776) |
| SCT-Da225 | GFTFSSYG | IFSGDSNT | SRQGHRSWFAY | KSVSTSGYSY | LAS | QHSRELPYT |
| | (612) | (613) | (614) | (828) | (829) | (830) |
| SCT-Da226 | GFTFSTYG | ISGGGIYT | VRMITTYFDH | SSIISNY | RTS | QQGSSLPRT |
| | (615) | (616) | (617) | (935) | (929) | (930) |
| SCT-Da227 | GFTFSSYG | IFSGDSYT | ARQGHRSWFAY | KSVSTSGYSY | LAS | QHSRELPST |
| | (612) | (618) | (619) | (828) | (829) | (860) |
| SCT-Da228 | GFTFSSYA | ISSGGSYT | ARHEDTTYYYAMDY | ENIYSY | YAK | QHHYGTPLT |
| | (538) | (620) | (621) | (774) | (786) | (787) |
| SCT-Da229 | GFTFNTYA | IRIKSNNY | VRQAYGNSYFFAMDY | SSVSSSH | STS | HQWSSYPYT |
| | (622) | (623) | (624) | (951) | (877) | (952) |
| SCT-Da230 | GFTFNTNA | IRSKSNNY | TAMDY | QSLVYSNGNTY | KVS | SQSTHVPYT |
| | (625) | (626) | (627) | (924) | (907) | (925) |
| SCT-Da231 | GYIFITYW | IFPASGST | ASYDGYYSWFAY | SSVSY | RTS | QQYHSFPRT |
| | (628) | (629) | (630) | (944) | (929) | (948) |
| SCT-Da232 | GFTFSHSY | IYAGTGGT | ARHETYGSSPDY | DHINNW | GAT | QQYWSIPYT |
| | (631) | (632) | (633) | (799) | (800) | (802) |
| SCT-Da233 | GFTFSSFY | IDAGTGGT | ARHVGRLRGYFDV | QDVNTA | STS | QQHYSSPWT |
| | (634) | (635) | (636) | (876) | (877) | (878) |
| SCT-Da234 | GFTFSSNY | IYAGTGST | ASHDGHDAMDY | QSISST | YAS | HQSYSWPHT |
| | (637) | (638) | (639) | (826) | (818) | (827) |
| SCT-Da235 | GFTFSSNY | IDAGTGGS | ARHVGRLRGYFDV | QDVSTA | SAS | QQHYSTPWT |
| | (637) | (640) | (636) | (884) | (882) | (885) |
| SCT-Da236 | GFTFSSRY | IYAGTGGT | ASHDGHDAMDY | QSISNT | YAS | QQSNDWPHT |
| | (641) | (632) | (639) | (821) | (818) | (822) |
| SCT-Da237 | GFTFSSRY | IYAGTGGT | ASHDGHDAMDY | QSISNT | YAS | QQSYDWPHT |
| | (641) | (632) | (639) | (821) | (818) | (824) |
| SCT-Da238 | GFTFSSRY | IYAGTGGS | ASHDGHDAMDY | QSISNT | YAS | QQSYNWPHT |
| | (641) | (642) | (639) | (821) | (818) | (825) |
| SCT-Da239 | GFTFSSSY | IYAGTGDT | ASPTGINWYFDV | ENIYSF | NAR | QHHYGPPFT |
| | (643) | (644) | (645) | (782) | (783) | (784) |
| SCT-Da240 | GFTFSSSY | IYAGTGDT | ASPTGVNWYFDV | ENIYSF | NAR | QHHYGPPFT |
| | (643) | (644) | (646) | (782) | (783) | (784) |
| SCT-Da241 | GFTFSSSY | IYAGTGGT | ARPNGYGYGMDY | DHISNW | GAT | QQYWSLPYT |
| | (643) | (632) | (647) | (805) | (800) | (806) |
| SCT-Da242 | GFTFSSSY | IYAGTGGT | ARQDSYGRGPDC | DHINNW | GAT | QQYWTIPYT |
| | (643) | (632) | (648) | (799) | (800) | (803) |
| SCT-Da243 | GFTFSSSY | IYAGTGGT | ARQDSYGRSPDY | DHINNW | GAT | QQYWSIPYT |
| | (643) | (632) | (649) | (799) | (800) | (802) |
| SCT-Da244 | GFTFSSSY | IYAGTGGT | ARHETYGSGPDY | DHINNW | GAT | QQYWSIPYT |
| | (643) | (632) | (650) | (799) | (800) | (802) |
| SCT-Da245 | GFTFSSSY | IYAGTGGT | ARHETYGSSPDY | GNIHNY | NAK | QHFWSTPWT |
| | (643) | (632) | (633) | (788) | (775) | (789) |
| SCT-Da246 | GFTFSSSY | IYAGTGGT | ARHETYGSSPDY | DHINNW | GAT | QQYWSIPYT |
| | (643) | (632) | (633) | (799) | (800) | (802) |
| SCT-Da247 | GFTFSSSY | IYAGTGGT | ARQDSYGRSPDY | DHINNW | GAT | QQYWSIPYT |
| | (643) | (632) | (649) | (799) | (800) | (802) |
| SCT-Da248 | GFTFSSSY | IYAGTGGT | ARQDSYGRSPDY | DHINNW | GAT | QQYWSIPYT |
| | (643) | (632) | (649) | (799) | (800) | (802) |
| SCT-Da249 | GFTFSSRY | IYAGTGGT | ASHDGLDAMDY | QSINNY | YAS | QQSNSWPLT |
| | (641) | (632) | (651) | (820) | (818) | (819) |
| SCT-Da250 | GFTFSSFY | IYAGPGYT | ARHVGRQRGYFDV | QDVSTA | SAS | QQHYSTPWT |
| | (634) | (652) | (653) | (884) | (882) | (885) |
| SCT-Da251 | GFTFSSSY | IYAGTGGT | GRHNGYGIGMDY | DHINNW | GAT | QQYWSLPYM |
| | (643) | (632) | (654) | (799) | (800) | (801) |
| SCT-Da252 | GLTFSSSY | IDAGTGGT | ARHVGRLRGYFDV | QDVTTA | SAS | QQHYSAPWT |
| | (655) | (635) | (636) | (886) | (882) | (887) |
| SCT-Da253 | GFTFSSNY | IYAGTGGT | ASHDGHDAMDY | QSISNT | YAS | QQSNSWPHT |
| | (637) | (632) | (639) | (821) | (818) | (823) |
| SCT-Da254 | GYTFTDYP | IITYYGDA | ASHRGKGYFDV | KSVSTSGYSY | LAS | QHSRELYT |
| | (656) | (657) | (658) | (828) | (829) | (854) |
| SCT-Da255 | GYTFTDYP | IITYYGDA | ASHRGKGYFDV | SSVSY | LTS | QQWSSNPLT |
| | (656) | (657) | (658) | (944) | (957) | (947) |
| SCT-Da256 | GYTFTDYF | IYPGNINT | AGGNYALDY | QSISNY | YAS | QQSNSWPLT |
| | (659) | (660) | (661) | (863) | (818) | (819) |
| SCT-Da257 | GYTFTDYY | IYPGNDNS | AGGNYAMDY | QSISNY | YAS | QQSNSWPLT |
| | (662) | (663) | (664) | (863) | (818) | (819) |
| SCT-Da258 | GYTFTAYY | IYPGNDNT | AGGNYAMDY | QSISNY | YAS | QQSNSWPLT |
| | (665) | (666) | (664) | (863) | (818) | (819) |
| SCT-Da259 | GYTFTNSG | INTYTGEP | APTIQVRRLFDY | SSVSY | ATS | QQWSSTPPT |
| | (667) | (668) | (669) | (944) | (945) | (946) |
| SCT-Da260 | GYTFTNSG | INTYTGEP | APTIQVRRLVDY | SSVSY | ATS | QQWSSTPPT |
| | (667) | (668) | (670) | (944) | (945) | (946) |
| SCT-Da261 | GYTFTNYG | INTNTGEP | ARRGYYGRGDY | ESVDSYGNSF | RAS | QQSNEDPYT |
| | (671) | (672) | (673) | (846) | (844) | (847) |
| SCT-Da262 | GFSLTSYD | IWTGGGT | VRWGDDYDWFAY | QSVSND | YAS | QQDYSSPPT |
| | (674) | (675) | (676) | (966) | (818) | (967) |
| SCT-Da263 | GFSLTTYG | IWRGGGTD | AIYSDHDTYALDY | LSLLYSSNQKNY | WAS | QQYYSYPLT |
| | (677) | (678) | (679) | (865) | (866) | (867) |
| SCT-Da264 | GYTFTDYA | ISNYNGDV | ARRHGNRAMDY | QDVGTA | RAS | QQYSTYYT |
| | (680) | (681) | (682) | (879) | (844) | (880) |
| SCT-Da265 | GYSFTSYY | INPSNGGT | TREGNYGYSWYAY | QDVSTA | SAS | QQHYSTPWT |
| | (683) | (684) | (685) | (884) | (882) | (885) |
| SCT-Da266 | GYTFTSYW | IDPSDSYT | AGGIHFDY | QSLLDSDGKTY | LVS | WQGTHFPFT |
| | (686) | (687) | (688) | (926) | (912) | (927) |
| SCT-Da267 | GYTFTSYN | IYPGKDDT | ARVYYGNYGGFDY | ESVDSYGYSF | LAS | QQNNEDPFT |
| | (689) | (690) | (691) | (858) | (829) | (859) |
| SCT-Da268 | GYTFTSYW | IYPGSGSV | TRGGIGVRSPFDV | QSLVHSNGNTY | KVS | SQSTHVWT |
| | (686) | (692) | (693) | (919) | (907) | (922) |
| SCT-Da269 | GYTFTSSW | IHPNSGNT | VRLGLFDF | DHINNW | GAT | QQYWSIPYT |
| | (694) | (695) | (696) | (799) | (800) | (802) |
| SCT-Da270 | GYTFTNYW | ISPRTGNT | TRGEAY | ENIYYS | NAN | KQSYDVPYT |
| | (697) | (698) | (699) | (780) | (778) | (781) |
| SCT-Da271 | GYTFTNYW | ISPRTGNT | TRGEAY | ENIYYS | NAN | KQSYDVPYT |
| | (697) | (698) | (699) | (780) | (778) | (781) |
| SCT-Da272 | GYTFTNYW | ISPRTGNT | TRGEAY | ENIYYS | NAN | KQSYDVPYT |
| | (697) | (698) | (699) | (780) | (778) | (781) |
| SCT-Da273 | GYTFTNYW | ISPRTGNT | TRGEAY | ENIYYS | NAN | KQSYDVPYT |
| | (697) | (698) | (699) | (780) | (778) | (781) |
| SCT-Da274 | GYTFTNYW | ISPRTGNT | TRGEAY | SSVSY | LTS | QQWSSNPLT |
| | (697) | (698) | (699) | (944) | (957) | (947) |
| SCT-Da275 | GYTFTNYW | ISPRTGNT | ARGEAY | ENIYYS | NAN | KQSYDVPYT |
| | (697) | (698) | (700) | (780) | (778) | (781) |
| SCT-Da276 | GYTFTNYW | ISPRTGNT | TRGEAY | ENIYYS | NAN | KQSYDVPYT |
| | (697) | (698) | (699) | (780) | (778) | (781) |
| SCT-Da277 | GYTFTNYW | ISPRTGNT | TRGEAY | ENIYYS | NAK | KQSYDVPYT |
| | (697) | (698) | (699) | (780) | (775) | (781) |
| SCT-Da278 | GYTFTNYW | ISPRTGNT | ARGEAY | ENIYYS | NAN | KQAYDVPYT |
| | (697) | (698) | (700) | (780) | (778) | (779) |
| SCT-Da279 | GYTFTNYW | ISPRTGNT | ARGEAY | ENIYYS | NAN | KQAYDVPYT |
| | (697) | (698) | (700) | (780) | (778) | (779) |
| SCT-Da280 | GYTFTNYW | ISPRTGNT | TRGEAY | ENIYYS | NAN | KQSYDVPYT |
| | (697) | (698) | (699) | (780) | (778) | (781) |
| SCT-Da281 | GYTFTNYW | ISPRTGNT | ARGEAY | ENIYYS | NAN | KQAYDVPYT |
| | (697) | (698) | (700) | (780) | (778) | (779) |
| SCT-Da282 | GYTFTNYW | ISPRTTYT | TRGEAY | ENIYYT | NAN | KQAYDVPYT |
| | (697) | (701) | (699) | (777) | (778) | (779) |
| SCT-Da283 | GYTFTTYW | INPSTGYT | ASYYGTSQAWFAY | QDISNY | YTS | QQGNTLPWT |
| | (702) | (703) | (704) | (773) | (771) | (772) |
| SCT-Da284 | GYTFTTYW | INPSTGYT | ASYYGTSQAWFAY | ESVDNFGNSF | LAS | QQSNEDPRT |
| | (702) | (703) | (704) | (838) | (829) | (837) |
| SCT-Da285 | GYTFTTYW | INPSTGYT | ASYYGTSQAWFAY | ESVDSYGNSF | IAS | QQSNEDPRT |
| | (702) | (703) | (704) | (846) | (842) | (837) |
| SCT-Da286 | GYTFTTYW | INPSTGYT | ASYYGTSQAWFAY | QNVDTN | SGS | QQYNTYPYT |
| | (702) | (703) | (704) | (873) | (874) | (875) |
| SCT-Da287 | GYTFTTYW | INPSTGYT | ASYYGTSQAWFAY | QRISDY | YAS | QNGHSFPFT |
| | (702) | (703) | (704) | (893) | (818) | (889) |
| SCT-Da288 | GYTFTTYW | INPSTGYT | ASYYGTSQAWFAY | QNVDTN | SAS | QQYNSYPYT |
| | (702) | (703) | (704) | (873) | (882) | (902) |
| SCT-Da289 | GYTFTTYW | INPSTGYT | ASYYGTSQAWFAY | QNVGTD | SAS | QQYNSYPYT |
| | (702) | (703) | (704) | (904) | (882) | (902) |
| SCT-Da290 | GYTFTTYW | INPSTGYT | ASYYGTSQAWFAY | SSISSNY | RTS | QQGSSLPRT |
| | (702) | (703) | (704) | (928) | (929) | (930) |
| SCT-Da291 | GYTFTTYW | INPSTGYT | ASYYGTSQAWFAY | SSISSNY | RTS | QQGSSIPRT |
| | (702) | (703) | (704) | (928) | (929) | (932) |
| SCT-Da292 | GYTFTTYW | INPSTGYT | ASYYGTSQAWFAY | SSVSY | DTS | QQWSSNPLT |
| | (702) | (703) | (704) | (944) | (953) | (947) |
| SCT-Da293 | GYTFTTYW | INPSTGYT | ASYYGTSQAWFAY | SSISY | GTS | QQWSSYPLT |
| | (702) | (703) | (704) | (954) | (955) | (956) |
| SCT-Da294 | GYTFTSYW | IYPGDGDT | ARVEADDLWYFDV | ENIYYS | NAN | KQSYDVPYT |
| | (686) | (705) | (706) | (780) | (778) | (781) |
| SCT-Da295 | GYTFTSYW | IYPGDGDT | ARVEADDLWYFDV | QSVDYDGDNY | AAS | QQSYEDPWT |
| | (686) | (705) | (706) | (831) | (794) | (832) |
| SCT-Da296 | GYTFTSYW | IYPGDGDT | ARVEADDLWYFDV | ESVDSYGNSF | IAS | QQSNEDPRT |
| | (686) | (705) | (706) | (846) | (842) | (837) |
| SCT-Da297 | GYTFTSYW | IYPGDGDT | ARVEADDLWYFDV | QDVSTA | SAS | QQHYSTPWT |
| | (686) | (705) | (706) | (884) | (882) | (885) |
| SCT-Da298 | GYTFTSYW | IYPGDGDT | ARVEADDLWYFDV | QDVTTA | SAS | QQHYSAPWT |
| | (686) | (705) | (706) | (886) | (882) | (887) |
| SCT-Da299 | GYTFTSYW | IYPGDGDT | ARVEADDLWYFDV | QNVGTD | SAS | QQYNSYPYT |
| | (686) | (705) | (706) | (904) | (882) | (902) |
| SCT-Da300 | GYTFTSYW | IYPGDGDT | ARVEADDLWYFDV | QNVGTD | SAS | QQYNSYPYT |
| | (686) | (705) | (706) | (904) | (882) | (902) |
| SCT-Da301 | GYTFTSYW | IYPGDGDT | ARVEADDLWYFDV | QNVGTN | SAS | QQYNSYPYT |
| | (686) | (705) | (706) | (899) | (882) | (902) |
| SCT-Da302 | GYTFTSYW | IYPGDGDT | ARVEADDLWYFDV | SSVSY | LTS | QQWSSNPLT |
| | (686) | (705) | (706) | (944) | (957) | (947) |
| SCT-Da303 | GYTFTSYW | IYPGDGDT | ARVEADDLWYFDV | QSVTND | YAS | QQDYNSPYT |
| | (686) | (705) | (706) | (970) | (818) | (962) |
| SCT-Da304 | GYTFTTYW | INPSTGYT | ASYYGTSQAWFAY | ENIYYS | NAN | KQSYDVPYT |
| | (702) | (703) | (704) | (780) | (778) | (781) |
| SCT-Da305 | GYAFTNYL | INPGSGGP | ASDNYGNYDVLDY | QEISGY | AAS | LQSASYPPT |
| | (707) | (708) | (709) | (793) | (794) | (795) |
| SCT-Da306 | GYAFTNYL | INPGSGGT | DREDAY | KSLLHSNGNTY | RMS | MQHLEYPYT |
| | (707) | (710) | (711) | (872) | (869) | (871) |
| SCT-Da307 | GYTFTDYA | INTYSSNT | ARRRGSHRDAYFDY | QSISDY | YAS | QNGHSFPFT |
| | (680) | (712) | (713) | (888) | (818) | (889) |
| SCT-Da308 | GYTFTDYA | INTYSSNT | ARRRGSHRDAYFDY | QSISDY | YAS | QNGHSFPFT |
| | (680) | (712) | (713) | (888) | (818) | (889) |
| SCT-Da309 | GYTFTDYA | INTYSSNT | ARRRGSHRDAYFDY | QSISDY | YAS | QNGHSFPFT |
| | (680) | (712) | (713) | (888) | (818) | (889) |
| SCT-Da310 | GYTFTDYA | INTYSSNT | ARRRGSHRDAYFDY | QSISDY | YAS | QNGHSFPFT |
| | (680) | (712) | (713) | (888) | (818) | (889) |
| SCT-Da311 | GYTFTDYV | IYPGGNRT | ARFNDWAMDH | QSLVHSNGITY | KVF | SQSTHVPWT |
| | (714) | (715) | (716) | (914) | (915) | (916) |
| SCT-Da312 | GYTFTDYV | IYPGGNST | ARFNDWSMDY | QSLVHSNGITY | KVS | SQSTHVPWT |
| | (714) | (717) | (718) | (914) | (907) | (916) |
| SCT-Da313 | GYTFTDYV | IYPGGSSI | ARFNDWAMDY | QSLVHSNGITY | KVS | SQSTHFPWT |
| | (714) | (719) | (720) | (914) | (907) | (917) |
| SCT-Da314 | GYTFTDYV | IYPGGSSI | ARFNDWAMDY | QSLVHSNGITY | KVS | SQSTHFPWT |
| | (714) | (719) | (720) | (914) | (907) | (917) |
| SCT-Da315 | GYTFTSYF | IYPGNVNT | ARDYAMDY | QSINNY | YAS | QQSSTWPLT |
| | (721) | (722) | (723) | (820) | (818) | (862) |
| SCT-Da316 | GYTFTTYF | IYPGNVNV | SRDYAMDY | QGINNY | YAS | QQSNSWPLT |
| | (724) | (725) | (726) | (817) | (818) | (819) |
| SCT-Da317 | GYTFTTYY | IYPGNVNT | TRDYAMDY | QSINNY | YAS | QQSNSWPLT |
| | (727) | (722) | (728) | (820) | (818) | (819) |
| SCT-Da318 | GYTFTTYY | IYPGNVNV | TRDYAMDY | QSINNY | YAS | QQSNSWPLT |
| | (727) | (725) | (728) | (820) | (818) | (819) |
| SCT-Da319 | GYTFTTYY | IYPGNVNV | TRDYAMDY | QSINNY | YAS | QQSNSWPLT |
| | (727) | (725) | (728) | (820) | (818) | (819) |
| SCT-Da320 | GYTFTTYY | IYPGNVNT | ARDYAMDY | QSINNY | YAS | QQSNSWPLT |
| | (727) | (722) | (723) | (820) | (818) | (819) |
| SCT-Da321 | GYTFTTYY | IYPGNVNT | ARDYAMDY | SSVSY | LTS | QQWSSNPLT |
| | (727) | (722) | (723) | (944) | (957) | (947) |
| SCT-Da322 | GYTFTTYY | IYPGNVNV | SRDYAMDY | QRINSY | YAS | QQSNSWPLT |
| | (727) | (725) | (726) | (861) | (818) | (819) |
| SCT-Da323 | GYTFTDYA | INTYSGNT | ARRRGSHRDAYFDY | QSISDY | SAS | QNGHSFPFT |
| | (680) | (729) | (713) | (888) | (882) | (889) |
| SCT-Da324 | GYTFTDYA | INTHSDNT | ARPNYDGHWYFDV | QSLLDSDGETY | LVS | WEGTHFWP |
| | (680) | (730) | (731) | (911) | (912) | (913) |
| SCT-Da325 | GYTFTDYA | INTYSGNT | ARRRGIHRDAYFDY | QSISDY | YAS | QNGHSFPFT |
| | (680) | (729) | (732) | (888) | (818) | (889) |
| SCT-Da326 | GYTFTDYA | INSYSGNT | ARWAITTGVYYAMDY | SSVSSSY | STS | QQYSGYPLT |
| | (680) | (733) | (734) | (937) | (877) | (938) |
| SCT-Da327 | GYTFTDYA | INTYSANT | ARRRGSHRDAYFDY | QSISDY | YVS | QNGHSFPFT |
| | (680) | (735) | (713) | (888) | (897) | (889) |
| SCT-Da328 | GYTFTDYA | INTYSGNT | ARRRGSHRDAYFDY | ESVDNYGISF | AAS | QQSKEVPT |
| | (680) | (729) | (713) | (839) | (794) | (840) |
| SCT-Da329 | GYTFTDYA | INTYSGNT | ARRRGSHRDAYFDY | QSISDY | YAS | QNGHSFPFT |
| | (680) | (729) | (713) | (888) | (818) | (889) |
| SCT-Da330 | GYTFTDYA | INTYSGNT | ARRRGSHRDAYFDY | SSISY | GTS | QQWSSYPLT |
| | (680) | (729) | (713) | (954) | (955) | (956) |
| SCT-Da331 | GYTFTDYA | INTYSGNT | ARRRGSHQDAYFDY | QSISDY | YAS | QNGHSFPFT |
| | (680) | (729) | (736) | (888) | (818) | (889) |
| SCT-Da332 | GYTFTDYA | INTYSGNT | ARRRGSHRDAYFDY | QSISDY | YAS | QNSHSFPFT |
| | (680) | (729) | (713) | (888) | (818) | (894) |
| SCT-Da333 | GYTFTDYA | INTYSGNT | ARRRGSHRDAYFDY | QDVSTA | SAS | QQHYSTPWT |
| | (680) | (729) | (713) | (884) | (882) | (885) |
| SCT-Da334 | GYTFTDYA | INTYSGNT | ARRRGSHRDAYFDY | QSISDY | YAS | QNSHSFPFT |
| | (680) | (729) | (713) | (888) | (818) | (894) |
| SCT-Da335 | GYTFTDYA | INTYSGNT | ARRRGSHRDAYFDY | QTISDY | YAS | QNSHSFPFT |
| | (680) | (729) | (713) | (898) | (818) | (894) |
| SCT-Da336 | GYTFTDYA | INTYSGNT | ARRRGSHRDAYFDY | QSISDY | YAS | QNSHSFPFT |
| | (680) | (729) | (713) | (888) | (818) | (894) |
| SCT-Da337 | GYTFTDYA | INTYSGNT | ARRRGSHRDAYFDY | QSISDY | YAS | QNGHSFPFT |
| | (680) | (729) | (713) | (888) | (818) | (889) |
| SCT-Da338 | GYTFTDYA | INTYSGNT | ARRRGSHRDAYFDY | QSISDY | YAS | QNSHSFPFT |
| | (680) | (729) | (713) | (888) | (818) | (894) |
| SCT-Da339 | GYTFTDYV | IYPGGNR | ARFNDWAMDY | QSLVHSNGITY | KVS | SQSTHVPWT |
| | (714) | (737) | (720) | (914) | (907) | (916) |
| SCT-Da340 | GYTFTDYE | IDPETGAT | TARQLGLPFAY | QSVSND | YAS | QQDYTSPPT |
| | (738) | (739) | (740) | (966) | (818) | (971) |
| SCT-Da341 | GFSLNTSNL | AHIWWDDD | ARKNHYYGHWYFDV | QFVSNF | YAS | QQDYSSPYT |
| | (741) | (742) | (743) | (963) | (818) | (960) |
| SCT-Da342 | GFSLNTSNM | AHIWWDDD | ARKNHYYGHWYFDV | QFVSNF | YAS | QQDYSSPYT |
| | (744) | (742) | (743) | (963) | (818) | (960) |
| SCT-Da343 | GFSLSTPGL | AHIWWDDD | ARKNHYYGHWYFDV | QSVNND | YAS | QQDYNSPYA |
| | (745) | (742) | (743) | (964) | (818) | (965) |
| SCT-Da344 | GFSLSTPNM | AHIWWDDD | ARKIHYYGHWYFDV | QSVIND | YAS | QQDYSSPYT |
| | (746) | (742) | (747) | (961) | (818) | (960) |
| SCT-Da345 | GFSLSTPSM | AHIWWDDD | ARKNHYSGHWFFDV | QSVIND | YAS | QQDYSSPYT |
| | (748) | (742) | (749) | (961) | (818) | (960) |
| SCT-Da346 | GFSLSTSGM | AHIWWDDD | ARKNHYYGHWYFDV | QSVSNY | YAS | QQDYSSPYT |
| | (750) | (742) | (743) | (969) | (818) | (960) |
| SCT-Da347 | GFSLSTSNL | AHIWWDDD | ARKSHYYGHWYFDV | QSVIND | YAS | QQDYSSPYT |
| | (751) | (742) | (752) | (961) | (818) | (960) |
| SCT-Da348 | GFSLSTSNM | AHIWWDDD | ARKSHYYGHWYFDF | QSVIND | YAS | QQDYNSPFT |
| | (753) | (742) | (754) | (961) | (818) | (972) |
| SCT-Da349 | GFSLSTSNM | AHIWWDDD | GRKSHYYGHWYFDV | QNVGTD | SAS | QQYNSYPYT |
| | (753) | (742) | (755) | (904) | (882) | (902) |
| SCT-Da350 | GFSLSTSNM | AHIWWDDD | GRKSHYYGHWYFDV | QNVDNY | YAS | QQDYSSPYT |
| | (753) | (742) | (755) | (959) | (818) | (960) |
| SCT-Da351 | GFSLSTSNM | AHIWWDDD | GRKSHYYGHWYFDV | QNVDNY | YAS | QQDYSSPYT |
| | (753) | (742) | (755) | (959) | (818) | (960) |
| SCT-Da352 | GFSLSTSNM | AHIWWDDD | ARKNHYYGHWYFDF | QSVIND | YAS | QQDYNSPYT |
| | (753) | (742) | (756) | (961) | (818) | (962) |
| SCT-Da353 | GFSLSTSNM | AHIWWDDD | ARKNHYYGHWYFDF | QSVIND | YAS | QQDYNSPYT |
| | (753) | (742) | (756) | (961) | (818) | (962) |
| SCT-Da354 | GFSLSTSNM | AHIWWDDD | ARKNHYYGHWYFDV | ENIYSN | AAT | QHFWGTPFT |
| | (753) | (742) | (743) | (790) | (791) | (792) |
| SCT-Da355 | GFSLSTSNM | AHIWWDDD | ARKNHYYGHWYFDV | QDINKY | YTS | LQYDNLWT |
| | (753) | (742) | (743) | (796) | (771) | (798) |
| SCT-Da356 | GFSLSTSNM | AHIWWDDD | ARKNHYYGHWYFDV | QSVIND | YAS | QQDYSSPYT |
| | (753) | (742) | (743) | (961) | (818) | (960) |
| SCT-Da357 | GFSLSTSNM | AHIWWDDD | ARKNHYYGHWYFDV | QSVTND | YAS | QQDYNSPYT |
| | (753) | (742) | (743) | (970) | (818) | (962) |
| SCT-Da358 | GFSLSTSNM | AHIWWDDD | ARKSHYYGHWYFDV | QSVSND | YAS | QQDYNSPYT |
| | (753) | (742) | (752) | (966) | (818) | (962) |
| SCT-Da359 | GFSLSTSNM | AHIWWDDD | GRKSHYYGHWYFDV | QSVIND | YAS | QQDYSSPYT |
| | (753) | (742) | (755) | (961) | (818) | (960) |
| SCT-Da360 | GFSLSTSNM | AHIWWDDD | ARKNHYYGHWYFDV | QTVTND | YAS | QQDYISPYT |
| | (753) | (742) | (743) | (942) | (818) | (943) |
| SCT-Da361 | GFSLSTYGI | AHIWWNDN | ARIDYWFAY | QGIINY | YTS | QQYSKLPYT |
| | (757) | (758) | (759) | (809) | (771) | (810) |
| SCT-Da362 | GFSLSTYGI | AHIWWNDN | ARIDYWFAY | SSVSY | LTS | QQWSSNPLT |
| | (757) | (758) | (759) | (944) | (957) | (947) |
| SCT-Da363 | GFTFSSFY | IYAGPGYT | ARHVGRQRGYFDV | QDVSTA | SAS | QQHYSTPWT |
| | (634) | (652) | (653) | (884) | (882) | (885) |
| SCT-Da364 | GFTFSSFY | IYAGPGYT | ARHVGRQRGYFDV | QDVSTA | SAS | QQHYSTPWT |
| | (634) | (652) | (653) | (884) | (882) | (885) |
| SCT-Da365 | GYTFTEYT | INPNNGGT | ARPHSLLRLRREFDY | SSVSY | LTS | QQWSSNPLT |
| | (582) | (552) | (589) | (944) | (957) | (947) |
| SCT-Da366 | GYTFTEYT | INPNNGGT | ARPHSLLRLRREFDY | SSVSY | LTS | QQWSSNPLT |
| | (582) | (552) | (589) | (944) | (957) | (947) |
| SCT-Da367 | GYTFTEYT | INPNNGGT | ARPHSLLRLRREFDY | SSVSY | LTS | QQWSSNPLT |
| | (582) | (552) | (589) | (944) | (957) | (947) |
| SCT-Da368 | GYTFTEYT | INPNNGGT | ARPHSLLRLRREFDY | SSVSY | LTS | QQWSSNPLT |
| | (582) | (552) | (589) | (944) | (957) | (947) |
| SCT-Da369 | GYTFTEYT | INPNNGGT | ARPHSLLRLRREFDY | SSVSY | LTS | QQWSSNPLT |
| | (582) | (552) | (589) | (944) | (957) | (947) |
| SCT-Da370 | GYTFTEYT | INPNNGGT | ARPHSLLRLRREFDY | SSVSY | LTS | QQWSSNPLT |
| | (582) | (552) | (589) | (944) | (957) | (947) |
| SCT-Da371 | GVNIKDTY | IDPANGNR | ARDGYYAPGY | QNVGTD | SAS | QQYNSYPYT |
| | (574) | (560) | (546) | (904) | (882) | (902) |
| SCT-Da372 | GVNIKDTY | IDPANGNR | ARDGYYAPGY | QNVGTD | SAS | QQYNSYPYT |
| | (574) | (560) | (546) | (904) | (882) | (902) |
| SCT-Da373 | GVNIKDTY | IDPANGNR | ARDGYYAPGY | QNVGTD | SAS | QQYNSYPYT |
| | (574) | (560) | (546) | (904) | (882) | (902) |
| SCT-Da374 | GVNIKDTY | IDPANGNR | ARDGYYAPGY | QNVGTD | SAS | QQYNSYPYT |
| | (574) | (560) | (546) | (904) | (882) | (902) |

TABLE 6

| | | SEQ | | SEQ |
|---|---|---|---|---|
| mAb | | ID | | ID |
| AA ID | VH Amino Acid Sequence | NO: | VL Amino Acid Sequence | NO: |
| SCT-Da001 | AVQLQESGPDLVKPSQSLSLTCTVTGYSITSNY | 4 | DIKMTQSPSSMYASLGERVTITCKASQDINSYL | 247 |
| | NWHWIRQFPGNKLEWMGYIHYSDDTNYNPSLKS | | SWFQQKTGKSPKTLIYRANRLVDGVPSRFSGSG | |
| | RISITRDPSKNQFFLQLNSVTTEDTATYYCARW | | SGQDYSLTISSLEYEDMGIYYCLQYDEFPPTFG | |
| | SYYYGGPYYYSMDYWGQGTSVTVSS | | GGTKLEIK | |
| SCT-Da002 | DVQLQESGPDLVKPSQSLSLTCTVTGYSITSGY | 5 | DIKMTQSPSSMYASLGERVTITCKASQDINTYL | 248 |
| | NWHWIRQFPGNKLEWMGYIHYSGGTNYNPSLKS | | SWFQQKPGKSPKTLIYRANILVDGVPSRFSGSG | |
| | RISITRDASKNQFFLQLNSVTTEDTATYYCGRW | | SGQDYSLTISSLEYEDMGIYYCLQYDEFPPTFG | |
| | SYYYGSPYYYSMDYWGQGTSVTVSS | | GGTKLEIK | |
| SCT-Da003 | DVQLQESGPGLVKPSQSLSLTCSVTGFSITSGY | 6 | DIVLTQSPASLAVSLGQRVTISCKASQSVDYDG | 249 |
| | YWNWIRQFPGNKLEWMGYISYDGSNNYNPSLKN | | DSYMNWYQQKPGQPPKVLIYAASNLESGIPARF | |
| | RISITRDTSKNQFFLNLNSVTTEDTATYYCARD | | SGSGSGTDFTLNIHPVEEEDAATYYCQQSNEDP | |
| | YYGYPAWFTYWGQGTLVTVSA | | RTFGGGTKLEIK | |
| SCT-Da004 | DVQLQESGPGLVKPSQSLSLTCSVTGYSITDGY | 7 | DIQMTQSTSSLSASLGDRVTISCRASQDISNYL | 250 |
| | FWNWFRQFPGNKLEWMGFLNYDGSNNYNPSLKN | | NWYQQKPDGTVKLLIYYTSRLHSGVPSRFSGSG | |
| | RLSITRDTSENQFFLRLSSVTTEDTATYYCARR | | SGTDYSLTISSLEQEDIATYFCQQGNALPWTFG | |
| | KGWLYAMDYWGQGTSVTVSS | | GGTKLEIK | |
| SCT-Da005 | DVQLQESGPGLVKPSQSLSLTCSVTGYSITSAY | 8 | DIQMTQTTSSLSASLGDRVTISCRASQDISNYL | 251 |
| | FWNWFRQFPGNKLEWLGFINYDGSNNYNPSLKN | | NWYQQKPDGTVKLLIYYTSRLHSGVPSRFSGSG | |
| | RISITRDTSKNQFFLRLSSVTTEDTATYYCARR | | SGTAYSLTISNLEEEDIATYFCQQGKSLPWTFG | |
| | KGWLYAMDYWGQGTSVTVSS | | GGTKLEIK | |
| SCT-Da006 | DVQLQESGPGLVKPSQSLSLTCSVTGYSITSAY | 9 | DIQMTQPTSSLSASLGDRVTISCRASQDISNYL | 252 |
| | FWNWFRQFPGNKLEWMGYINFDGSSNYNPSLKN | | NWYQQKPDGTVKLLIYYTSRLHSGVPSRFSGSG | |
| | RISITRDTSKNQFFLRLSSVTSEDTATYFCARR | | SGTDYSLTISNLDQEDIATYFCQQGNTLPWTFG | |
| | KGWLYAMDYWGQGTSITVSS | | GGTKLEIK | |
| SCT-Da007 | DVQLQESGPGLVKPSQSLSLTCSVTGYSITSAY | 9 | DIQMTQPTSSLSASLGDRVTISCRASQDISNYL | 253 |
| | FWNWFRQFPGNKLEWMGYINFDGSSNYNPSLKN | | NWYQQKPDGTVKLLIYYTSRLHSGVPSRFSGSG | |
| | RISITRDTSKNQFFLRLSSVTSEDTATYFCARR | | SGTDYSLTISNLEQEDIATYFCQQGNTLPWTFG | |
| | KGWLYAMDYWGQGTSITVSS | | GGTKLEIK | |
| SCT-Da008 | DVQLQESGPGLVKPSQSLSLTCSVTGYSITSAY | 9 | DIQMTQTTSSLSASLGDRVTISCRASQDINNYL | 254 |
| | FWNWFRQFPGNKLEWMGYINFDGSSNYNPSLKN | | NWYQQKPDGIVKLLIYYTSRLNSGVPSRFSGSG | |
| | RISITRDTSKNQFFLRLSSVTSEDTATYFCARR | | SGTDYSLTISNLEQEDIATYFCQQGNTLPWTFG | |
| | KGWLYAMDYWGQGTSITVSS | | GGTKLEIK | |
| SCT-Da009 | DVQLQESGPGLVKPSQSLSLTCSVTGYSITSAY | 9 | DIQMTQTTSSLSASLGDRVTISCRASQDISNYL | 255 |
| | FWNWFRQFPGNKLEWMGYINFDGSSNYNPSLKN | | NWYQQKPDGTVKLLIYYTSRLHSGVPSRFSGSG | |
| | RISITRDTSKNQFFLRLSSVTSEDTATYFCARR | | SGTDYSLTISNLEQEDIATYFCQQGNTLPWTFG | |
| | KGWLYAMDYWGQGTSITVSS | | GGTKLEIK | |
| SCT-Da010 | DVQLQESGPGLVKPSQSLSLTCSVTGYSITSAY | 10 | DIQMTQTTSSLSASLGDRVTISCRASQDISNYL | 256 |
| | FWNWFRQFPGNRLEWLGFINYDGTNTYNPSLKN | | NWYQQKPDGTVKLLIYYTSRLHSGVPSRFSGSG | |
| | RISITRDTSKNQFFLRLSSVTTEDTATYYCARR | | SGTAYSLTISNLEEADIATYFCQQGKSLPWTFG | |
| | KGWLYAMDYWGQGTSVTVSS | | GGTKLEIK | |
| SCT-Da011 | DVQLQESGPGLVKPSQSLSLTCSVTGYSITSAY | 11 | DIQMTQTTSSLSASLGDRVTISCRASQDINNYL | 254 |
| | FWNWFRQFPGNRLEWMGFINYDGTNTYNPSLKN | | NWYQQKPDGIVKLLIYYTSRLNSGVPSRFSGSG | |
| | RISITRDTSKNQFFLRLSSVTTEDTATYYCARR | | SGTDYSLTISNLEQEDIATYFCQQGNTLPWTFG | |
| | KGWLYAMDYWGQGTSVTVSS | | GGTKLEIK | |
| SCT-Da012 | DVQLQESGPGLVKPSQSLSLTCSVTGYSITSAY | 11 | DIQMTQTTSSLSASLGDRVTISCRASQDISNYL | 256 |
| | FWNWFRQFPGNRLEWMGFINYDGTNTYNPSLKN | | NWYQQKPDGTVKLLIYYTSRLHSGVPSRFSGSG | |
| | RISITRDTSKNQFFLRLSSVTTEDTATYYCARR | | SGTAYSLTISNLEEADIATYFCQQGKSLPWTFG | |
| | KGWLYAMDYWGQGTSVTVSS | | GGTKLEIK | |
| SCT-Da013 | DVQLQESGPGLVKPSQSLSLTCSVTGYSITSDS | 12 | DIQMTQTTSSLSASLGDRVTISCRASQDINNYL | 254 |
| | FWNWIRQVPGNKLEWMGFISYDGSNNYNPSLKN | | NWYQQKPDGIVKLLIYYTSRLNSGVPSRFSGSG | |
| | RISITRDTSKNQFFLKLNSVTTEDTATYYCARR | | SGTDYSLTISNLEQEDIATYFCQQGNTLPWTFG | |
| | KGWLYAMDYWGQGTSVTVSS | | GGTKLEIK | |
| SCT-Da014 | DVQLQESGPGLVKPSQSLSLTCSVTGYSITSDY | 13 | DIQMTQTTSSLSASLGDRVTISCRASQDISNYL | 257 |
| | FWNWIRQFPGNKLEWMGFISYDGSNNYNPSLKN | | NWYQQKPDGIVKLLIYYTSRLNSGVPSRFSGSG | |
| | RISITRDTSKNQFFLKLNSVTSEDTATYFCARR | | SGTDYSLTISNLEQEDIATYFCQQGNTLPWTFG | |
| | KGWLYAMDYWGQGTSVTVSS | | GGTKLEIK | |
| SCT-Da015 | DVQLQESGPGLVKPSQSLSLTCSVTGYSITSDY | 14 | DIQMTQTTSSLSASLGDRVTISCRASQDISNFL | 258 |
| | FWNWIRQFPGNKLEWMGFISYDGSNYCNPSLKN | | NWFQQKPDGTVKLLIYYTSRLHSGVPSRFSGSG | |
| | RISITRDTSKNQFFLKLNSVTTEDTATYYCARR | | SGTDYSLTISNLEQEDIASYFCQQGKTLPWTFG | |
| | KGWLYAMDYWGQGTSVTVSS | | GGTKLEIK | |
| SCT-Da016 | DVQLQESGPGLVKPSQSLSLTCSVTGYSITSGY | 15 | DIQMTQTTSSLSASLGDRVTISCRASQDISNYL | 259 |
| | FWNWFRQFPGDKLEWMGYINYDGSNNYNPSLKN | | NWYQQKPDGTVKLLIYYTSRLHSGVPSRFSGSG | |
| | RVSISRDTSKNQFFLRLSSVTAEDTATYYCARR | | SGTDYSLTISNLEQEDLATYFCQQGNTLPWTFG | |
| | KGWLYAMDYWGQGTSVTVSS | | GGTKLEIK | |
| SCT-Da017 | DVQLQESGPGLVKPSQSLSLTCSVTGYSITSGY | 16 | DIQMTQPTSSLSASLGDRVTISCRASQDINNYL | 260 |
| | FWNWFRQFPGNKLEWMGFINYDGSDNYNPSLKN | | NWYQQKPDGTVKLLIYYTSRLHSGVPSRFSGSG | |
| | RISITRDTSENQFFLRLSSVTTEDTATYYCARR | | SGTDYSLTISNLEQEDIATYFCQQGNTLPWTFG | |
| | KGWLYAMDSWGQGTSVTVSS | | GGTKLEIK | |
| SCT-Da018 | DVQLQESGPGLVKPSQSLSLTCSVTGYSITSGY | 16 | DIQMTQTTSSLSASLGDRVTISCRASHDIHNYL | 261 |
| | FWNWFRQFPGNKLEWMGFINYDGSDNYNPSLKN | | NWYQQKPDGTVKLLIYYTSRLHSGVPSRFSGSG | |
| | RISITRDTSENQFFLRLSSVTTEDTATYYCARR | | SGTDYSLTISNLEQEDIATYFCQQGNTLPWTFG | |
| | KGWLYAMDSWGQGTSVTVSS | | GGTKLEIK | |
| SCT-Da019 | DVQLQESGPGLVKPSQSLSLTCSVTGYSITSGY | 17 | DIQMTQTTSSLSASLGDRVTISCRASQDISNYL | 257 |
| | FWNWFRQFPGNKLEWMGFINYDGSNNYNPSLKN | | NWYQQKPDGIVKLLIYYTSRLNSGVPSRFSGSG | |
| | RISITRDTSKNQFFLRLSSVTSEDTATYYCARR | | SGTDYSLTISNLEQEDIATYFCQQGNTLPWTFG | |
| | KGWLYAMDYWGQGTSVTVSS | | GGTKLEIK | |
| SCT-Da020 | DVQLQESGPGLVKPSQSLSLTCSVTGYSITSGY | 18 | EIVLTQSPTTMAASPGEKITITCSASSSISSNY | 262 |
| | FWNWFRQFPGNKLEWMGFINYDGSSNYNPSLKN | | LHWYQQKPGFSPKLLIYRTSNLASGVPARFSGS | |
| | RISITRDTSENQFFLRLSSVTTEDTATYYCARR | | GSGTSYSLTIGTMEAEDVATYYCQQGSSIPRTF | |
| | KGWLYAMDYWGQGTSVTVSS | | GSGTKLEIK | |
| SCT-Da021 | DVQLQESGPGLVKPSQSLSLTCSVTGYSITSGY | 19 | DIQMTQSTSSLSASLGDRVTISCRASQDISNYL | 250 |
| | FWNWFRQFPGNKLEWMGFLNYDGSNNYNPSLKN | | NWYQQKPDGTVKLLIYYTSRLHSGVPSRFSGSG | |
| | RISITRDTSENQFFLRFNSVTTEDTATYYCARR | | SGTDYSLTISSLEQEDIATYFCQQGNALPWTFG | |
| | KGWLYAMDYWGQGTSVTVSS | | GGTKLEIK | |
| SCT-Da022 | DVQLQESGPGLVKPSQSLSLTCSVTGYSITSGY | 19 | DIQMTQSTSSLSASLGDRVTISCRASQDISNYL | 250 |
| | FWNWFRQFPGNKLEWMGFLNYDGSNNYNPSLKN | | NWYQQKPDGTVKLLIYYTSRLHSGVPSRFSGSG | |
| | RISITRDTSENQFFLRFNSVTTEDTATYYCARR | | SGTDYSLTISSLEQEDIATYFCQQGNALPWTFG | |
| | KGWLYAMDYWGQGTSVTVSS | | GGTKLEIK | |
| SCT-Da023 | DVQLQESGPGLVKPSQSLSLTCSVTGYSITSGY | 21 | DIQMTQSTSSLSASLGDRVTISCRASQDISNYL | 263 |
| | FWNWFRQFPGNKLEWMGFLNYDGSNNYNPSLKN | | NWYQQKPDGTVKLLIYYTSRLHSGVPSRFSGSG | |
| | RISITRDTSENQFFLRLSSVTTEDTATYFCARR | | SGTDYSLTISSLEQEDIATYFCQQGIALPWTFG | |
| | KGWLYAMDYWGQGTSVTVSS | | GGTKLEIK | |
| SCT-Da024 | DVQLQESGPGLVKPSQSLSLTCSVTGYSITSGY | 22 | DIQMTQSTSSLSASLGDRVTISCRASQDISNYL | 263 |
| | FWNWFRQFPGNKLEWMGFLNYDGSNNYNPSLKN | | NWYQQKPDGTVKLLIYYTSRLHSGVPSRFSGSG | |
| | RISITRDTSENQFFLRLSSVTTEDTATYYCARR | | SGTDYSLTISSLEQEDIATYFCQQGIALPWTFG | |
| | KGWLYAMDYWGQGTSVTVSS | | GGTKLEIK | |
| SCT-Da025 | DVQLQESGPGLVKPSQSLSLTCSVTGYSITSGY | 22 | DIQMTQSTSSLSASLGDRVTISCRASQDISNYL | 250 |
| | FWNWFRQFPGNKLEWMGFLNYDGSNNYNPSLKN | | NWYQQKPDGTVKLLIYYTSRLHSGVPSRFSGSG | |
| | RISITRDTSENQFFLRLSSVTTEDTATYYCARR | | SGTDYSLTISSLEQEDIATYFCQQGNALPWTFG | |
| | KGWLYAMDYWGQGTSVTVSS | | GGTKLEIK | |
| SCT-Da026 | DVQLQESGPGLVKPSQSLSLTCSVTGYSITSGY | 22 | DIVLTQSPASLAVSLGQRATISCRASESVDSYG | 264 |
| | FWNWFRQFPGNKLEWMGFLNYDGSNNYNPSLKN | | NSFMHWYQQKPGQPPKLLIYIASNLESGVPARF | |
| | RISITRDTSENQFFLRLSSVTTEDTATYYCARR | | SGSGSRTDFTLTIDPVEADDAATYYCQQSNEDP | |
| | KGWLYAMDYWGQGTSVTVSS | | RTFGGGTKLEIK | |
| SCT-Da027 | DVQLQESGPGLVKPSQSLSLTCSVTGYSITSGY | 22 | SIVMTQTPKFLLVSAGDRVTITCRASQSVTNDV | 265 |
| | FWNWFRQFPGNKLEWMGFLNYDGSNNYNPSLKN | | AWYQQKPGQSPKLLIYYASNRYTGVPDRFTGSG | |
| | RISITRDTSENQFFLRLSSVTTEDTATYYCARR | | YGTDFTFTISTVQAEDLAVYFCQQDYNSPYTFG | |
| | KGWLYAMDYWGQGTSVTVSS | | GGTKLEIK | |
| SCT-Da028 | DVQLQESGPGLVKPSQSLSLTCSVTGYSITSGY | 23 | DIQMTQTTSSLSASLGDRVTISCRASQDISNYL | 266 |
| | FWNWFRQFPGNKLEWMGFLNYDGSNNYNPSLKN | | NWYQQKPDGIVKLLIYYTSRLNSGVPSRFSGSG | |
| | RISITRDTSKNQFFLRLSSVTTEDTATYYCARR | | SGTDYSLTISSLEQEDIATYFCQQGNTLPWTFG | |
| | KGWLYAMDYWGQGTSVTVSS | | GGTKLESK | |
| SCT-Da029 | DVQLQESGPGLVKPSQSLSLTCSVTGYSITSGY | 24 | DIQMTQSTSSLSASLGDRVSISCRASQDISNYL | 267 |
| | FWNWFRQFPGNKLEWMGFLNYDGSSNYNPSLKN | | NWYQQKPDGTVKLLIYYTSRLHSGVPSRFSGSG | |
| | RISITRDTSENQFFLRLSSVTTEDTATYYCARR | | SGTDYSLTISSLEQEDVATYFCQQGNALPWTFG | |
| | KGWLYAMDYWGQGTSVTVSS | | GGTKLEIK | |
| SCT-Da030 | DVQLQESGPGLVKPSQSLSLTCSVTGYSITSGY | 25 | DIQMTQTTSSLSASLGDRVTISCRTSQDISNYL | 268 |
| | FWNWIRQFPGNKLEWMGFINYDGSNNYNPSLKN | | NWYQQKPDGWKLLIYYTSRLNSGVPSRFSGSG | |
| | RISITRDTSKNQFFLKLNSVTTEDTATYYCARR | | SGTDYSLTISNLEQEDIATYFCQQGNTLPWTFG | |
| | KGWLYAMDYWGQGTSVTVSS | | GGTKLEIK | |
| SCT-Da031 | DVQLQESGPGLVKPSQSLSLTCSVTGYSITSGY | 26 | DIVMTQSHKFMSTSVGDRVSITCKASQDVGTAV | 269 |
| | YWNWIRQFPGNKLEWMGYISYDGSNNYNPSLKN | | AWYQQKPGQSPKLLIYWASTRHTGVPDRFTGSG | |
| | RISITRDTSKNQFFLKLNSVTTEDTATYYCARE | | SGTDFTLTISNVQSEDLADYFCQQYSSYPYTFG | |
| | GDYGSYWGQGTLVTVSA | | GGTKLEIK | |
| SCT-Da032 | DVQLQESGPGLVKPSQSLSLTCSVTGYSITSGY | 27 | DIQMTQTTSSLSASLGDRVTISCRASQDISNYL | 270 |
| | YWNWIRQFPGNKLEWMGYISYDGSNNYNPSLKN | | NWYQQKPDGTVKLLIYYTSRLHSGVPSRFSGSG | |
| | RISITRDTSKNQFFLKLTSVTTEDTATYYCTRR | | SGTDYSLTISNLEQEDIATYFCQQGNALPWTFG | |
| | KGWLYAMDYWGQGTSVTVSS | | GGTKLEIK | |
| SCT-Da033 | DVQLQESGPGLVKPSQSLSLTCTVTGYSITSDY | 28 | DIVMTQSHKFMSTSVGDRVSITCKASQDVNTAV | 271 |
| | AWNWIRQFPGNKLEWMGYISYSGSTNYNPSLKS | | AWYQQRPGQSPKLLIYSASYRFIGVPDRFTGSG | |
| | RISITRDTSKNQFFLQLNSVTTEDTATYYCASG | | SGTDFTFTISTVQAEDLAVYYCQQHYSTPFTFG | |
| | YYGIHYYAMDYWGQGTSVTVSS | | SGTKLEIK | |
| SCT-Da034 | DVQLQESGPGLVKSSQSLSLTCSVTGYSITSGY | 29 | DIQMTQTTSSLSASLGDRVTISCRASQDISNYL | 266 |
| | FWNWIRQFPGNKLEWMGFINYDGSSNYNPSLKN | | NWYQQKPDGIVKLLIYYTSRLNSGVPSRFSGSG | |
| | RISITRDTSKNQFFLKLNSVTTEDTATYYCARR | | SGTDYSLTISSLEQEDIATYFCQQGNTLPWTFG | |
| | KGWLYAMDYWGQGTSVTVSS | | GGTKLESK | |
| SCT-Da035 | EIHLQQTGPELVKPGASVKISCKASGYSFTAYI | 30 | DVLMTQTPLSLPVSLGDHASISCRSSQSIVHTN | 272 |
| | MLWVKQSHGKSLEWIGNINPYYGSTSYNLKFKG | | GNTYLEWYLQKPGQSPKLLIYKVSNRFSGVPDR | |
| | KATLTVDQSSSTAYMQLNSLTSDDSAVYYCARG | | FSGSGSGTDFTLKISRVEAEDLGLYYCFQGSHV | |
| | PPYYYGSSRDYALDYWGQGTSVTVSS | | PYTFGGGTKLEIK | |
| SCT-Da036 | EIQLQQTGPELVKPGASVKISCKASGYSFTDYI | 31 | DVVMTQTPLSLPVSLGDQASISCRSSQSLVHSN | 273 |
| | MLWVKQSHGKSLEWIGNINPYYDTTAYNLKFKG | | GNTYLHWYLQKPGQSPNLLIYKVSNRFSGVPDR | |
| | KATLTVDKSSSTAYMLLNSLTSEDSAVYYCATY | | FSGSGSGTDFTLKISRVEAEDLGVYFCSQSTHG | |
| | DGFAYWGQGTLVTVSA | | PWTFGGGTKLEIK | |
| SCT-Da037 | EIQLQQTGPELVKPGASVKISCKASGYSFTDYI | 32 | DVVMTQTPLSLPVSLGDQASISCRSSQSLVHSY | 274 |
| | MLWVKQSHGKSLEWIGNINPYYGTTSYNLKFKG | | GNTYLHWHLQKPGQSPKLLIYKVSNRFSGVPDR | |
| | KATLTVDKSSSTAYMQLNSLTSEDSAVYYCATY | | FSGSGSGTDFTLKISRVEAEDLGVYFCSQSTHG | |
| | DGFAYWGQGTLVTVSA | | PWTFGGGTKLEIK | |
| SCT-Da038 | EIQLQQTGPELVKPGASVKISCKASGYSFTDYI | 33 | DVVMTQTPLSLPVSLGDQASISCRSSQSLVHSN | 275 |
| | MLWVKQSHGKSLEWIGNINPYYYTTSYNLKFKG | | GNTYLHWHLQKPGQSPKLLIYKVSNRFSGVPDR | |
| | KATLTVDKSSSTAYMQLNSLTSEDSAVYYCATY | | FSGSGSGTDFTLKISRVEAEDLGVYFCSQSTHG | |
| | DGFAYWGQGTLVTVSA | | PWTFGGGTKLEIK | |
| SCT-Da039 | EVHLQQSGAELVRPGALVKLSCKASGFNIKDYY | 34 | EIVLTQSPTPMAASPGEKITITCSASSSISSNY | 276 |
| | MHWVKQRPEQGLEWIGWIDPENGNTIYDPKFQG | | LHWYRQKPGFSPKLLIYRTSNLASGVPPRFSGS | |
| | KASITADTSSNTAYLQLSSLTSEDTAVYYCTIG | | GSGTSYSLTIGTMEAEDVATYYCQQGSSLPRTF | |
| | VRRDYFDYWGQGTTLTVSS | | GSGTKLEIK | |
| SCT-Da040 | EVKLEESGGGLVQPGGSMRLSCVASGFTFSNYW | 35 | QIVLTQSPAIMSASPGEKVTITCSASSSVNYMH | 277 |
| | MNWVRQSPEKGLEWVAEIRLKFNNYTTHYAESV | | WFQQKPGTSPKLWIYSTSNLASGVPARFSGSGS | |
| | KGRFTISRDDSKSSVYLQMNNLRTEDTGIYYCI | | GTSYSLTISRMEAEDAATYYCQQRSTYPRTFGG | |
| | KFGGMDYWGPGTSVTVSS | | GTKLEIK | |
| SCT-Da041 | EVKLVESGGGLVKPGGSLKLSCAASGFAFNSYD | 36 | DILMTQSPSSMSVSLGDTVSITCHASQGISSNI | 278 |
| | MSWVRQTPEKRLEWVATISDGGSFTYYPDSVKG | | GWLQRKPGKSFKGLIYHGTNLEDGLPSRFSGSG | |
| | RFTISRDNARNTLYLHMSSLRSEDTALYYCARH | | SGADYSLTISSLESEDFADYYCVQYAQFPYTFG | |
| | QITTLRMDYWGQGTSVTVSS | | GGTKLELK | |
| SCT-Da042 | EVKLVESGGGLVKPGGSLKLSCAASGFAFSSYD | 37 | DILMTQSPSSMSVSLGDTVSITCHASQGISSNI | 279 |
| | MSWVRQTPEKRLEWVAAISDGGSYTYYPDSVKG | | GWLQQKPGKSFKGLIYHGTNLEDGLPSRFSGSG | |
| | RFTISRDNAGNTLYLQMSSLRSEDTALYYCARH | | SGADYSLTISSLESEDFADYYCVQYAQFPYTFG | |
| | RVTTVRMDYWGQGTSVTVSS | | GGTKLEIK | |
| SCT-Da043 | EVKLVESGGGLVKPGGSLKLSCAASGFAFSSYD | 38 | DILMTQSPSSMSVSLGDTVSITCHASQGISSNI | 280 |
| | MSWVRQTPEKRLEWVATISDGGSYTYYPDSVKG | | GWLQQKPGKSFKGLIYHGTNLEDGLPSRFSGSG | |
| | RFTISRDNAGKTLYLQMSSLRPEDTALYYCARH | | SGADYSLTISSLESEDFADYYCVQYAQFPYTFG | |
| | RVTTVRMDYWGQGTSVTVSS | | GGTKLEIL | |
| SCT-Da044 | EVKLVESGGGLVKPGGTLKLSCAASGFAFSDYD | 39 | DILMTQSPSSMSVSLGDSVSITCHASQGISSNI | 281 |
| | MSWVRQTPEKRLEWVATISDGGSYTYYPDSVKG | | GWLQQKAGKSFKGLIYHGTNLEDGVPSRFSGSG | |
| | RFTISRDNARNTLYLQMSSLRSEDTALYYCARH | | SGADYSLTISSLESEDFADYYCVQYAQFPYTFG | |
| | LITTLRMDYWGQGASVTVSS | | GGTKVEIK | |
| SCT-Da045 | EVKLVESGGGLVKPGGTLKLSCAASGFAFSDYD | 39 | DILMTQSPSSMSVSLGDTVTITCHASQGISSNI | 282 |
| | MSWVRQTPEKRLEWVATISDGGSYTYYPDSVKG | | GWLQQKPGKSFKGLIYHGTNLEDGVPSKFGGSG | |
| | RFTISRDNARNTLYLQMSSLRSEDTALYYCARH | | SGADYSLTISSLESEDFADYYCVQYAQFPYTFG | |
| | LITTLRMDYWGQGASVTVSS | | GGTKLEIK | |
| SCT-Da046 | EVKLVESGGGLVKPGGTLKLSCAASGFAFSDYD | 40 | DILMTQSPSSMSVSLGDSVSITCHASQGISSNI | 283 |
| | MSWVRQTPEKRLEWVATISDGGSYTYYPDSVKG | | GWLQQKAGKSFKGLIYHGTNLEDGVPSRFSGSG | |
| | RFTVSRDNARNTLYLQMSSLRSEDTALYYCARH | | SGADYSLTISSLESEDFADYYCVQYAQFPYTFG | |
| | LITTLRMDYWGQGASVTVSS | | GGTTLEIK | |
| SCT-Da047 | EVKLVESGGGLVKPGGTLKLSCAASGFAFSDYD | 41 | DILMTQSPSSMSVSLGDSVSITCHASQGISSNM | 284 |
| | MSWVRQTPEKRLEWVATISNDGSYTYYPDSVKG | | GWLQQKAGKSFKGLIYHGTNLEDGVPSRFSGSG | |
| | RFTISRDNARNTLYLQMSSLRSEDTALYYCARH | | SGADYSLTISSLESEDFADYYCVQYAQFPYTFG | |
| | LITTLRMDYWGQGASVTVSS | | GGTKLEIK | |
| SCT-Da048 | EVKLVESGGGLVKPGGTLKLSCAASGFAFSDYD | 41 | DILMTQSPSSMSVSLGDSVSITCHASQGISSNM | 285 |
| | MSWVRQTPEKRLEWVATISNDGSYTYYPDSVKG | | GWLQQKTGKSFKGLIYHGTNLEDGVPSRFSGSG | |
| | RFTISRDNARNTLYLQMSSLRSEDTALYYCARH | | SGADYSLTISSLESEDFADYYCVQYAQFPYTFG | |
| | LITTLRMDYWGQGASVTVSS | | GGTKLEIK | |
| SCT-Da049 | EVMLVASGGGLVKPGGSLKLSCAASGFTFSSYT | 42 | DIVLTQSPASLTVSLGQRATISCRASKSVSTSA | 286 |
| | VSWVRQTPEKRLEWVASIISGGGDIYYPDSVRG | | YSYMHWYQQKPGQPPKLLIYLASNLESGVPARF | |
| | RFTISRDNARNNLYLQMSSLRSEDTALYYCARW | | SGSGSGTDFTLNIHPVEEEDAATYYCQHSRELL | |
| | YSAGYFDVWGAGTTVTVSS | | TFGSGTKLEIK | |
| SCT-Da050 | EVMLVASGGGLVKPGGSLKLSCAASGFTFSSYT | 43 | DIVLTQSPPSLAVSLGQRATISCRASKSVSTSA | 287 |
| | VSWVRQTPEKRLEWVATISGGGVTYYPDSVRGR | | YSYMHWYQQKPGQPPKLLIYLASNLESGVPARF | |
| | FTISRDNAKNNLYLQMRSLRSEDTALYYCARWY | | SGSGSGTDFTLNIHPVEEEDAATYYCQHSRELL | |
| | SVGYFDVWGAGTTVTVSS | | TFGSGTKLEIK | |
| SCT-Da051 | EVMLVASGGGLVKPGGSLKLSCAASGFTFSSYT | 44 | DIVLTQSPASLTVSLGQRATISCRASKSVSTSA | 288 |
| | VSWVRQTPEKRLEWVATITGGGDTYYPDSVRGR | | YSYMHWYQQKLGQPPKLLIYLASNLESGVPARF | |
| | FTISRDNAKNNLYLQMSSLRSEDTALYYCARWY | | SGSGSGTDFTLNIHPVEEEDAATYYCQHSRELL | |
| | SVGYFDVWGAGTTVTVSS | | TFGSGTKLEIK | |
| SCT-Da052 | EVMLVASGGGLVKPGGSLKLSCAASGFTFSSYT | 45 | DIVLTQSPASLAVSLGQRATISCRASKSVSTSA | 289 |
| | VSWVRQTPEKRLEWVATITSGGGDTYYPDSVRG | | YSYMHWYQQKPGQPPKLLIYLASNLESGVPARF | |
| | RFTISRDNAKNNLYLEMSSLRSEDTALYYCARW | | SGSGSGTDFTLNIHPVEEEDAATYYCQHSRELL | |
| | YSAGYFDVWGAGTTVTVSS | | TFGSGTKLEIK | |
| SCT-Da053 | EVMLVASGGGLVKPGGSLNLSCAASGFTFSSYT | 46 | DIVLTQSPPSLAVSLGQRATISCRASKSVSTSA | 287 |
| | VSWVRQTPEKRLEWVATISGGGVTYYPDTVRGR | | YSYMHWYQQKPGQPPKLLIYLASNLESGVPARF | |
| | FTISRDNAKNNLYLQMRSLRSEDTALYYCARWY | | SGSGSGTDFTLNIHPVEEEDAATYYCQHSRELL | |
| | SVGYFDVWGAGTTVTVSS | | TFGSGTKLEIK | |
| SCT-Da054 | EVMLVESGGGLVKPGGSLKLSCAASGFTFSSYA | 47 | DIVLTQSPASLAVSLGQRATISCRASKSVSTSN | 290 |
| | MSWVRQTPEKRLEWVATIFFGSSVTYFPASVKG | | YSYMHWYQQKPGQPPKLLLYLASNLESGVPARF | |
| | RFTISRDNANNTLFLQLSSLRSEDTAMYYCARQ | | SGSGSGTDFTLNIHPVEEEDAATYYCQHSRELP | |
| | GTSPWFAYWGQGTLVTVSA | | TFGGGTKLEIK | |
| SCT-Da055 | EVMLVESGGGLVKPGGSLKLSCAASGFTFSSYT | 48 | DIVLTQSPASLAVSLGQRATISCRASKSVSTSG | 291 |
| | MSWVRQTPEKRLEWVATIISGGGNTYYPDSVKG | | YSYMHWYQQKPGQPPKLLIYLASNLESGVPARF | |
| | RFTISRDNAKNNLYLQMSSLRSEDTALYYCARW | | SGSGSGTDFTLNIHPVEEEDAATYYCQHSRELL | |
| | YSAGYFDVWGAGTTVTVSS | | TFGSGTKLEIK | |
| SCT-Da056 | EVMLVESGGGLVKPGGSLKLSCAASGFTFSSYT | 49 | DIVLTQSPASLAVSLGQRATISCRASKSVSTSG | 292 |
| | MSWVRQTPEKRLEWVATISGGVGDTYYPDSVKG | | YSYMHWYQQKPGQPPKLLIYLASNLESGVPARF | |
| | RFTISRDNAKNNLYLQMSSLRSEDTALYYCARQ | | SGSGSGTDFTLNIHPVEEEDAATYYCQHSRELP | |
| | GGFPWFAYWGQGTLVTVSA | | YTFGGGTKLEIK | |
| SCT-Da057 | EVQLQPSGAELVKPGASVKLSCTASGFNIEDTY | 50 | DIVMTQFQKFMSTSVGDRVSVTCKASQNVDTNV | 293 |
| | IHWMKQRPEQGLEWIGRIDPANGNTISDPKFQG | | AWYQQKPGQSPKLLIYSGSYRFSGVPDRFTGSG | |
| | KATITADTSSNTAYLQLSSLTSEDTAVYYCARD | | SGTDFTLTISNVQSEDLAEYFCQQYNTYPYTFG | |
| | GYYAPGYWGQGTTLTVSS | | GGTKLEIK | |
| SCT-Da058 | EVQLQPSGAELVKPGASVKLSCTASGFNIEDTY | 51 | DIVMTQSQKFMSTSVGDRVSVTCKASQNVDTNV | 294 |
| | IHWMKQRPEQGLEWIGRIDPANGNTISDPKFRG | | AWYQQKPGQSPKLLIYSASYRFSGVPDRFTGSG | |
| | KATITADTSSNTAYLQLSSLTSEDTAVYYCARD | | SGTDFTLTISNVQSEDLAEYFCQQYNSYPYTFG | |
| | GYYAPGYWGQGTTLTVSS | | GGTKLEIK | |
| SCT-Da059 | EVQLQPSGSELVKPGASVKLSCTASGFNIEDTY | 52 | DIVMTQSQKFMSTSVGDRVSVTCKASQNVDTNV | 294 |
| | IHWMKQRPEQGLEWIGRIDPANGYTTYDPKFQD | | AWYQQKPGQSPKLLIYSASYRFSGVPDRFTGSG | |
| | KATITADTSSNTAYLQLSSLTSEDTAVYYCARD | | SGTDFTLTISNVQSEDLAEYFCQQYNSYPYTFG | |
| | GYYAPGYWGQGTTLTVSS | | GGTKLEIK | |
| SCT-Da060 | EVQLQQFGAELVKPGASMKISCKASGYTFTDYN | 53 | DIVLTQSPVSLVVSLGQRATISCRANETVDSYG | 295 |
| | MAWVKQSHGKSLEWIGDINPNYDSTSYNQKFKG | | SSFMHWYQQKPGHPPKLLIYRASNLESGIPARF | |
| | KATLTVDKSSSTAYMELRSLTSEDAALYYCARS | | SGSGSRTDFTLTINPVEADDVATYFCQQSNEDP | |
| | GFDGYYYAMDYWGQGTSVTVSS | | FTFGSGTKLEIK | |
| SCT-Da061 | EVQLQQFGAELVKPGASVKISCKASDYTFTDYN | 54 | DIQMTQTTSSLSASLGDRVTISCSASQGISNYL | 296 |
| | MDWVKQSHGKSLEWIGDINPNNGGTIYNLKFKG | | NWYQQKPDGTVKLLIYYTSSLHSGVPSRFSGSG | |
| | KATLTVDKSSSTAYMELRSLTSEDTAVYYCARR | | SGTDYSLTISNLEPEDIATYYCQQYSKLPLTFG | |
| | HYGNYFYAMDYWGQGTSVTVSS | | AGTKLELK | |
| SCT-Da062 | EVQLQQFGAELVKPGASVKISCKASGYTFTDYN | 55 | DIVLTQSPASLAVSLGQRATISCRASESVDSYG | 297 |
| | IDWVKQSHGKSLEWIGDINPNGDFTTYNLKFKG | | NSFMHWYQQKPGQPPKLLVYIASNLESGVPARF | |
| | KATLTVDKSSSTAYMELRSLTSEDTAVYYCARR | | SGSGSRTDFTLTIDPVEADDAAAYYCQQSNEDP | |
| | HYGNYFYAMDYWGQGTSVTVSS | | RTFGGGTKLEIK | |
| SCT-Da063 | EVQLQQFGAELVKPGASVKISCKASGYTFTDYN | 56 | DIVLTQSPASLAVSLGQRATISCRASESVDSYG | 264 |
| | MDWVKQNHGKSLEWIGDINPNNGGTIYNLKFKG | | NSFMHWYQQKPGQPPKLLIYIASNLESGVPARF | |
| | KATLTVDKSSSTAYMELRSLTSEDTAVYYCARR | | SGSGSRTDFTLTIDPVEADDAATYYCQQSNEDP | |
| | HYGNYFYAMDYWGQGTSVTVSS | | RTFGGGTKLEIK | |
| SCT-Da064 | EVQLQQFGAELVKPGASVKISCKASGYTFTDYN | 57 | DIQMTQSSSYLSVSLGGRVTITCKASDHINNWL | 298 |
| | MDWVKQSHGKSLEWIGDINPNDDSTKYNLKFKG | | AWYQQKPGNAPRLLISGATSLETGVPSRFSGSG | |
| | KATLTVDKSSSTAYMELRSLTSEDTAVYYCARR | | SGKDYTLSITSLQTEDVATYYCQQYWSTPTFGG | |
| | HYGNYFYAMDYWGQGTSVTVSS | | GTKLEIK | |
| SCT-Da065 | EVQLQQFGAELVKPGASVKISCKASGYTFTDYN | 57 | DIVLTQSPASLAVSLGQRATISCRASESVDSFG | 299 |
| | MDWVKQSHGKSLEWIGDINPNDDSTKYNLKFKG | | HSFMHWYQQKPGQPPKLLIYLASNLESGVPARF | |
| | KATLTVDKSSSTAYMELRSLTSEDTAVYYCARR | | SGSGSRTDFTLTIDPVEADDGATYYCQQSNEDP | |
| | HYGNYFYAMDYWGQGTSVTVSS | | RTFGGGTKLEIK | |
| SCT-Da066 | EVQLQQFGAELVKPGASVKISCKASGYTFTDYN | 58 | DIVLTQSPASLAVSLGQRATISCRASESVDSFG | 300 |
| | MDWVKQSHGKSLEWIGDINPNDDSTSYNLKFKG | | NSFMHWYQQKPGQPPKLLIYIASNLESGVPARF | |
| | KATLTVDKSSSTAYMELRSLTSEDTAVYYCARR | | SGSGSRTDFTLTIGPVEADDAATYYCQQSNEDP | |
| | HYGNYFYAMDYWGQGTSVTVSS | | RTFGGGTKLEII | |
| SCT-Da067 | EVQLQQFGAELVKPGASVKISCKASGYTFTDYN | 59 | DIVLTQSPASLAVSLGQRATISCRASESVDNFG | 301 |
| | MDWVKQSHGKSLEWIGDINPNNGGTIYNLKFKG | | NSFMHWYQQKPGQPPKLLIYLASNLESGVPARF | |
| | KATLTVDKSSSTAYMELRSLTSEDTAVYYCARR | | SGSGSRTDFTLTIDPVEADDAATYYCQQSNEDP | |
| | HYGNYFYAMDYWGQGTSVTVSS | | RTFGGGTKLEIK | |
| SCT-Da068 | EVQLQQFGAELVKPGASVKISCKASGYTFTDYN | 59 | DIVLTQSPASLAVSLGQRATISCRASESVDSYG | 264 |
| | MDWVKQSHGKSLEWIGDINPNNGGTIYNLKFKG | | NSFMHWYQQKPGQPPKLLIYIASNLESGVPARF | |
| | KATLTVDKSSSTAYMELRSLTSEDTAVYYCARR | | SGSGSRTDFTLTIDPVEADDAATYYCQQSNEDP | |
| | HYGNYFYAMDYWGQGTSVTVSS | | RTFGGGTKLEIK | |
| SCT-Da069 | EVQLQQFGAELVKPGASVKISCKASGYTFTDYN | 60 | DIVLTQSPASLAVSLGQRATISCRASESVDSFG | 302 |
| | MDWVKQSHGKSLEWIGDINPNNGGTIYNQKFKG | | NSFMHWYQQKPGQPPKLLIYLASNLESGVPARF | |
| | KATLTVDKSSSTAYMELRSLTSEDTAVYYCARR | | SGSGSRTDFTLTIDPVEADDAATYYCQQSNEDP | |
| | HYGNYFYAMDYWGQGTSVTVSS | | RTFGGGTKLEIK | |
| SCT-Da070 | EVQLQQFGAELVKPGASVKISCKASGYTFTDYN | 60 | DIVLTQSPASLAVSLGQRATISCRASESVDSYG | 303 |
| | MDWVKQSHGKSLEWIGDINPNNGGTIYNQKFKG | | NSFMHWYQQKPGQPPKLLIYLASNLESGVPARF | |
| | KATLTVDKSSSTAYMELRSLTSEDTAVYYCARR | | SGSGSRTDFTLTIDPVEADDAATYYCQQSNEDP | |
| | HYGNYFYAMDYWGQGTSVTVSS | | RTFGGGTKLEIK | |
| SCT-Da071 | EVQLQQFGAELVKPGASVKISCKASGYTFTDYN | 60 | DIVLTQSPASLAVSLGQRVTISCKASQSVDYDG | 249 |
| | MDWVKQSHGKSLEWIGDINPNNGGTIYNQKFKG | | DSYMNWYQQKPGQPPKVLIYAASNLESGIPARF | |
| | KATLTVDKSSSTAYMELRSLTSEDTAVYYCARR | | SGSGSGTDFTLNIHPVEEEDAATYYCQQSNEDP | |
| | HYGNYFYAMDYWGQGTSVTVSS | | RTFGGGTKLEIK | |
| SCT-Da072 | EVQLQQFGAELVKPGASVKISCKASGYTFTDYN | 60 | DIVMTQSQKFMSTSVGDRVSVTCKASQNVGTDV | 304 |
| | MDWVKQSHGKSLEWIGDINPNNGGTIYNQKFKG | | AWYQQKPGQSPKLLIYSASYRFSGVPDRFTGSG | |
| | KATLTVDKSSSTAYMELRSLTSEDTAVYYCARR | | SGTDFSLAISNVQSEDLAEYFCQQYNSYPYTFG | |
| | HYGNYFYAMDYWGQGTSVTVSS | | GGAKLEIK | |
| SCT-Da073 | EVQLQQFGAELVKPGASVKISCKASGYTFTDYN | 61 | DIVLTQSPASLAVSLGQRVTISCKASQSVDYDG | 249 |
| | MDWVKQSHGKSLEWIGDINPNNGGTLYNLKFKG | | DSYMNWYQQKPGQPPKVLIYAASNLESGIPARF | |
| | KATLTVDKSSSTAYMELRSLTSEDTAVYYCARR | | SGSGSGTDFTLNIHPVEEEDAATYYCQQSNEDP | |
| | HYGNYFYAMDYWGQGTSVTVSS | | RTFGGGTKLEIK | |
| SCT-Da074 | EVQLQQFGAELVKPGASVKISCKASGYTFTDYN | 62 | DIQMTQSSSYLSVSLGGRVTITCKASDHINNWL | 305 |
| | MDWVKQSHGKSLEWIGDINPNYDSTNYNLKFKG | | GWYQQKPGNAPRLLISGATSLETGVPSRFSGSG | |
| | KATLTVDKSSSTAYMELRSLTSEDTAVYYCARR | | SGKDYTLSITSLQTEDVATYYCQQYWSIPYTFG | |
| | HYGNYFYAMDYWGQGTSVTVSS | | GGTKLEIK | |
| SCT-Da075 | EVQLQQFGAELVKPGASVKISCKASGYTFTDYN | 62 | DIVLTQSPASLAVSLGQRATISCRASESVDSFG | 306 |
| | MDWVKQSHGKSLEWIGDINPNYDSTNYNLKFKG | | NSFMHWYQQKPGQPPKLLIYIASNLESGVPARF | |
| | KATLTVDKSSSTAYMELRSLTSEDTAVYYCARR | | SGSGSRTDFTLTIDPVEADDAATYYCQQSNEDP | |
| | HYGNYFYAMDYWGQGTSVTVSS | | RTFGGGTRLEIK | |
| SCT-Da076 | EVQLQQFGAELVKPGASVKISCKASGYTFTDYN | 62 | DIVLTQSPASLAVSLGQRVTISCKASQSVDYDG | 249 |
| | MDWVKQSHGKSLEWIGDINPNYDSTNYNLKFKG | | DSYMNWYQQKPGQPPKVLIYAASNLESGIPARF | |
| | KATLTVDKSSSTAYMELRSLTSEDTAVYYCARR | | SGSGSGTDFTLNIHPVEEEDAATYYCQQSNEDP | |
| | HYGNYFYAMDYWGQGTSVTVSS | | RTFGGGTKLEIK | |
| SCT-Da077 | EVQLQQFGAELVKPGASVKISCKASGYTFTDYN | 63 | DIVLTQSPASLAVSLGQRATISCRASESVDSYG | 264 |
| | MDWVKQSHGKSLEWIGDISPNYESTNYSLKFKG | | NSFMHWYQQKPGQPPKLLIYIASNLESGVPARF | |
| | KATLTVDKSSSTAYMELRSLTSEDTAVYYCARR | | SGSGSRTDFTLTIDPVEADDAATYYCQQSNEDP | |
| | HYGNYFYAMDYWGQGTSVTVSS | | RTFGGGTKLEIK | |
| SCT-Da078 | EVQLQQFGAELVKPGASVKISCKASGYTFTDYN | 63 | DIVLTQSPDSLAVSLGQRATISCRASESVDSFG | 307 |
| | MDWVKQSHGKSLEWIGDISPNYESTNYSLKFKG | | HSFMHWYQQKPGQPPKLLIYLASNLESGVPARF | |
| | KATLTVDKSSSTAYMELRSLTSEDTAVYYCARR | | SGSGSRTDFTLTIDPVEADDAATYYCQQSNEDP | |
| | HYGNYFYAMDYWGQGTSVTVSS | | RTFGGGTKLEIK | |
| SCT-Da079 | EVQLQQFGAELVKPGASVKISCKASGYTFTDYN | 64 | DIVLTQSPASLAVSLGQRATISCRAGESVDSFG | 308 |
| | VDWVKQSHGKSLEWIGDINPNDDSTSYNLKFKG | | NSFMHWYQQKPGQPPKLLIYLASNLESGVPARF | |
| | KATLTVDKSSSTAYMELRSLTSEDTAVYYCARR | | SGSGSRTDFTLTIDPVEADDAAAYYCQQSNEDP | |
| | HYGNYFYAMDYWGQGTSVTVSS | | RTFGGGTKLEIK | |
| SCT-Da080 | EVQLQQFGTELVKPGASVKISCKASGYTFTDYN | 65 | DIQMTQTTSSLSASLGDRVTISCRASQDISNYL | 257 |
| | INWVKQSHGRSLEWIGDINPNYDSTSYNQNFKG | | NWYQQKPDGIVKLLIYYTSRLNSGVPSRFSGSG | |
| | KATLTVDKSSSTSYMELRSLTSEDTAVYYCARG | | SGTDYSLTISNLEQEDIATYFCQQGNTLPWTFG | |
| | GFYGNFFDYWGQGTTLTVSS | | GGTKLEIK | |
| SCT-Da081 | EVQLQQFGTELVKPGASVKISCKASGYTFTDYN | 66 | DIVLTQSPASLAVSLGQRATISCRASESVDSSG | 309 |
| | INWVKQSHGRSLEWIGDINPNYDSTSYNQNFKG | | NSFMHWYQQKPGQPPKLLIYRASNLESGIPARF | |
| | KATLTVDKSSSTSYMELRSLTSEDTAVYYGARG | | SGSGSRTDFTLTINPVEADDVATYYCQQSNEDP | |
| | GFYGNFFDYWGQGTTLTVSS | | FTFGSGTKLEIK | |
| SCT-Da082 | EVQLQQSGADLVRPGALVKLSCKTSGFNIKDYY | 67 | EIVLTQSPTTMAASPGEKITITCSASSSISSNY | 310 |
| | MHWVKQRPEQGLEWIGWIDPENGNTIYDPKFQG | | LNWYQQKPEFSPKLLIYRTSNLASGVPARFSGS | |
| | KASITADTSSSTAYLQLSSLTSEDTAVYYCSIG | | GSGTSYSLTIGTMEAEDVATYYCQQGSSLPRTF | |
| | VRRDYFAYWGQGTTLTVSS | | GSGTKLEIK | |
| SCT-Da083 | EVQLQQSGAEFVRPGALVKLSCKTSGFNIKDYY | 68 | DILMTQSPSSMSVSLGDSVSITCHASQGISSNI | 283 |
| | MHWVKQRPEQGLEWIGWIDPENGNTIYDPKFQG | | GWLQQKAGKSFKGLIYHGTNLEDGVPSRFSGSG | |
| | KASITADTSSNTAYLQLSSLTSEDTAVYYCTIG | | SGADYSLTISSLESEDFADYYCVQYAQFPYTFG | |
| | VRRDYFDYWGQGTTLTVSS | | GGTTLEIK | |
| SCT-Da084 | EVQLQQSGAEFVRPGALVKLSCKTSGFNIKDYY | 68 | EIVLTQSPTTMAASPGEKITITCSASSSISSNY | 311 |
| | MHWVKQRPEQGLEWIGWIDPENGNTIYDPKFQG | | LHWYQQKPGFSPKLFIYRTSNLASGVPARFSGS | |
| | KASITADTSSNTAYLQLSSLTSEDTAVYYCTIG | | GSGTSYSLTIGTMEAEDVATYYCQQGSSIPRTF | |
| | VRRDYFDYWGQGTTLTVSS | | GSGTKLEIK | |
| SCT-Da085 | EVQLQQSGAELVKPGASVKLSCTASGFNIEDTY | 69 | EIVLTQSPTTMAASPGEKITITCSASSSISSNY | 312 |
| | IHWMKQRPEQGLEWIGRIDPANGNPIYDPKFQG | | LNWYQQKPEFSPKLLIYRTSNLASGVPARFSGS | |
| | KATITADSSSNTAYLQLSSLTSEDTAVYYCARD | | GSGTSYSLTIGTMEAEDVATYYCQQGSSIPRTF | |
| | GYYAPGYWGQGTTLTVSS | | GSGTKLEIK | |
| SCT-Da086 | EVQLQQSGAELVKPGASVKLSCTASGFNIEDTY | 70 | DIVMTQFQKFMSTSVGDRVSVTCKASQNVDTNV | 293 |
| | IHWMKQRPEQGLEWIGRIDPANGNPKSDPRFQG | | AWYQQKPGQSPKLLIYSGSYRFSGVPDRFTGSG | |
| | KATITADSSSNTAYLQFSSLTSEDTAVYYCARD | | SGTDFTLTISNVQSEDLAEYFCQQYNTYPYTFG | |
| | GYYAPGYWGQGTTLTVSS | | GGTKLEIK | |
| SCT-Da087 | EVQLQQSGAELVKPGASVKLSCTASGFNIEDTY | 71 | DIVMTQSQKFMSTSVGDRVSVTCKASQNVGTNV | 313 |
| | IHWMKQRPEQGLEWIGRIDPANGNRKYDPRFQG | | AWYQQKPGQSPKLLIYSASYRYSGVPDRFTGSG | |
| | KATITADSSSNTAYLQLSSLTSEDTAVYYCARD | | SGTDFTLAISNVQSEDLAEYFCQQYNSYPYTFG | |
| | GYYAPGYWGQGTTLTVSS | | GGTKLEIK | |
| SCT-Da088 | EVQLQQSGAELVKPGASVKLSCTASGFNIEDTY | 72 | DIQMTQSPSSLSASLGGKVTITCKASQDINKYI | 314 |
| | IHWMKQRPEQGLEWIGRIDPANGNTISDPRFRG | | AWYQHKPVKGPRLLIHYTSTLQPGIPSRFSGSG | |
| | KATITADTSSNTAYLQFSSLTSEDTAVYYCARD | | SGRDYSFSISNLEPEDIATYYCLQYDNLRTFGG | |
| | GYYAPGYWGQGTTLTVSS | | GTKLEIK | |
| SCT-Da089 | EVQLQQSGAELVKPGASVKLSCTASGFNIEDTY | 72 | DIQMTQSSSYLSVSLGGRVTITCKASDHINNWL | 305 |
| | IHWMKQRPEQGLEWIGRIDPANGNTISDPRFRG | | GWYQQKPGNAPRLLISGATSLETGVPSRFSGSG | |
| | KATITADTSSNTAYLQFSSLTSEDTAVYYCARD | | SGKDYTLSITSLQTEDVATYYCQQYWSIPYTFG | |
| | GYYAPGYWGQGTTLTVSS | | GGTKLEIK | |
| SCT-Da090 | EVQLQQSGAELVKPGASVKLSCTASGFNIEDTY | 72 | DIVMTQFQKFMSTSVGDRVSVTCKASQNVDTNV | 293 |
| | IHWMKQRPEQGLEWIGRIDPANGNTISDPRFRG | | AWYQQKPGQSPKLLIYSGSYRFSGVPDRFTGSG | |
| | KATITADTSSNTAYLQFSSLTSEDTAVYYCARD | | SGTDFTLTISNVQSEDLAEYFCQQYNTYPYTFG | |
| | GYYAPGYWGQGTTLTVSS | | GGTKLEIK | |
| SCT-Da091 | EVQLQQSGAELVKPGASVKLSCTASGFNIEDTY | 73 | DIVMTQAAPSIPVTPGESVSISCRSSKSLLHSN | 315 |
| | MHWVKERPEQGLEWIGRNDPANGNTEYDPKFQG | | GNTFLYWFLLRPGQSPHLLIYRMSNLASGVPDR | |
| | KATITSDTSSNTAYLQLSTLTSEDTAVYYCASL | | FSGSGSGTAFTLRISRVEAEDVGVYYCMQHLEF | |
| | WVDYWGQGTSVTVSS | | PYTFGGGTKLEIK | |
| SCT-Da092 | EVQLQQSGAELVKPGASVKLSCTASGFNIKDTY | 74 | DIVMTQSQKFMSTSVGDRVSVTCKASQNVGTNV | 316 |
| | IHWMKQRPEQGLEWIGRIDPANANTQYDPKFQG | | AWYQQRPGQSPKLLIYSASYRYSGVPDRFTGSG | |
| | KATITADTSSNTAYLQLSSLTSEDTAVYYCAHD | | SGTDFTLTISNVQSEDLAEYFCQQYNNYPYTFG | |
| | GYYAPGYWGQGTTLTVSS | | GGTKLEIK | |
| SCT-Da093 | EVQLQQSGAELVKPGASVKLSCTASGFNIKDTY | 75 | DIVMTQSQKFMSTSVGDRVSVTCKASQNVGTDV | 317 |
| | IHWMKQRPEQGLEWIGRIDPANGNTIYDPKFQG | | AWYQQKPGQSPKLLIYSASYRFSGVPDRFTGSG | |
| | KATITADTSSNTAYLQLSSLTSEDTAVYYCAHD | | SGTDFTLTISNVQSEDLAEYFCQQYNIYPYTFG | |
| | GYYAPGYWGQGTTLTVSS | | GGTKLEIK | |
| SCT-Da094 | EVQLQQSGAELVKPGASVKLSCTASGFNIKDTY | 75 | DIVMTQSQKFMSTSVGDRVSVTCKASQNVGTDV | 318 |
| | IHWMKQRPEQGLEWIGRIDPANGNTIYDPKFQG | | AWYQQKPGQSPKLLIYSASYRFSGVPDRFTGSG | |
| | KATITADTSSNTAYLQLSSLTSEDTAVYYCAHD | | SGTDFTLTISNVQSEDLAEYFCQQYNSYPYTFG | |
| | GYYAPGYWGQGTTLTVSS | | GGTKLEIK | |
| SCT-Da095 | EVQLQQSGAELVKPGASVKLSCTASGFNIKDTY | 75 | ETTVTQSPASLSMAIGEKVTIRCITSTDIDDDM | 319 |
| | IHWMKQRPEQGLEWIGRIDPANGNTIYDPKFQG | | NWYQQKPGEPPKLLISEGNTLRPGVPSRFSSSG | |
| | KATITADTSSNTAYLQLSSLTSEDTAVYYCAHD | | YGTDFVFTIENMLSEDVADYYCLQSDNLPFTFG | |
| | GYYAPGYWGQGTTLTVSS | | SGTKLEIK | |
| SCT-Da096 | EVQLQQSGAELVKPGASVKLSCTASGFNIKDTY | 76 | DIVMTQSQKFMSTSVGDRVSVTCKASQNVGTNV | 320 |
| | IHWMKQRPEQGLEWIGRIDPANGNTKYDPKFQG | | AWYQQKPGQSPKLLIYSASYRFSGVPDRFTGSG | |
| | KATITADTSSNTAYLQLSSLTSEDTAVYYCAHD | | SGTDFTLTISNVQSEDLAEYFCQQYNSYPYTFG | |
| | GYYAPGYWGQGTTLTVSS | | GGTKLEIK | |
| SCT-Da097 | EVQLQQSGAELVKPGASVKLSCTASGFNIKDTY | 77 | DIVMTQSQKFMSTSVGDRVSVTCKASQNVGTDV | 321 |
| | IHWMKQRPIQGLEWIGRIDPANGNSHYDPKFQG | | AWYQQKPGQSPKLLIYSASYRFSGVPDRFTGSG | |
| | KATITADTSSNTAYLQLSSLTSEDTAVYYCAHD | | SGTDFTLTISNVQSEDLAEYSCQQYNSYPYTFG | |
| | GYYAPGYWGQGTTLTVSS | | GGTKLEIK | |
| SCT-Da098 | EVQLQQSGAELVKPGASVKLSCTASGFNIKDTY | 77 | DIVMTQSQKFMSTSVGDRVSVTCKASQNVGTNV | 322 |
| | IHWMKQRPIQGLEWIGRIDPANGNSHYDPKFQG | | AWYQQKPGQSPKLLIYSASYRYSGVPDRFTGSG | |
| | KATITADTSSNTAYLQLSSLTSEDTAVYYCAHD | | SGTDFTLTISNVQSEDLAEYFCQQYNSYPYTFG | |
| | GYYAPGYWGQGTTLTVSS | | GGTKLEIK | |
| SCT-Da099 | EVQLQQSGAELVKPGASVKLSCTASGFNIKDTY | 77 | DIVMTQSQKFMSTSVGDRVSVTCKASQNVGTNV | 323 |
| | IHWMKQRPIQGLEWIGRIDPANGNSHYDPKFQG | | AWYQQKSGQSPKLLIYSASYRFSGVPDRFTGSG | |
| | KATITADTSSNTAYLQLSSLTSEDTAVYYCAHD | | SGTDFTLTISNVQSEDLAEYFCQQYNSYPYTFG | |
| | GYYAPGYWGQGTTLTVSS | | GGTKLEIK | |
| SCT-Da100 | EVQLQQSGAELVKPGASVKLSCTASGFNIKDTY | 77 | DNVMTQSQKFMSTSVGDRVSVTCKASQNVGTNV | 324 |
| | IHWMKQRPIQGLEWIGRIDPANGMSHYDPKFQG | | AWYQQKSGQSPKLLIYSASYRFSGVPDRFTGSG | |
| | KATITADTSSNTAYLQLSSLTSEDTAVYYCAHD | | SGTDFTLTISNVQSEDLAEYFCQQYNSYPYTFG | |
| | GYYAPGYWGQGTTLTVSS | | GGTKLEIK | |
| SCT-Da101 | EVQLQQSGAELVKPGASVKLSCTASGFNIKDTY | 78 | DIVMTQSQKFMSTSVGDRVSVTCKASQNVGTDV | 318 |
| | IHWMMQRPEQGLEWIGRIDPANGNTIYDPKFQG | | AWYQQKPGQSPKLLIYSASYRFSGVPDRFTGSG | |
| | KATITADTSSNTAYLHLSSLTSEDTAVYYCAHD | | SGTDFTLTISNVQSEDLAEYFCQQYNSYPYTFG | |
| | GYYAPGYWGQGTTLTVSS | | GGTKLEIK | |
| SCT-Da102 | EVQLQQSGAELVKPGASVKLSCTASGFNIKDTY | 79 | DIVMTQSQKFMSTSVGDRVSVTCKASQNVGTDV | 321 |
| | IHWMMQRPEQGLEWIGRIDPANGNTIYDPKFQG | | AWYQQKPGQSPKLLIYSASYRFSGVPDRFTGSG | |
| | KATITADTSSNTAYLQLSSLTSEDTAVYYCAHD | | SGTDFTLTISNVQSEDLAEYSCQQYNSYPYTFG | |
| | GYYAPGNWGQGTTLTVSS | | GGTKLEIK | |
| SCT-Da103 | EVQLQQSGAELVKPGASVKLSCTASGFNIKDTY | 80 | DIQMTQTTSSLSASLGDRVTISCSASQGISNYL | 325 |
| | IHWMMQRPEQGLEWIGRIDPANGNTIYDPKFQG | | NWYQQKPDGTVKLLIYYTSSLHSGVPSRFSGSG | |
| | KATITADTSSNTAYLQLSSLTSEDTAVYYCAHD | | SGTDYSLTISNLEPEDIATYYCQQYSKLPYTFG | |
| | GYYAPGYWGQGTTLTVSS | | GGTKLEIK | |
| SCT-Da104 | EVQLQQSGAELVKPGASVKLSCTASGFNIKDTY | 80 | DIVMTQSQKFMSTSVGDRVSVTCKASQNVGTDV | 326 |
| | IHWMMQRPEQGLEWIGRIDPANGNTIYDPKFQG | | AWYQQKPGQSPKLLIYSASYRFSGVPDRFTGSG | |
| | KATITADTSSNTAYLQLSSLTSEDTAVYYCAHD | | SGTDFTLAISNVQSEDLAEYFCQQYNSYPYTFG | |
| | GYYAPGYWGQGTTLTVSS | | GGTKLEIK | |
| SCT-Da105 | EVQLQQSGAELVKPGASVKLSCTASGFNIKDTY | 80 | DIVMTQSQKFMSTSVGDRVSVTCKASQNVGTDV | 318 |
| | IHWMMQRPEQGLEWIGRIDPANGNTIYDPKFQG | | AWYQQKPGQSPKLLIYSASYRFSGVPDRFTGSG | |
| | KATITADTSSNTAYLQLSSLTSEDTAVYYCAHD | | SGTDFTLTISNVQSEDLAEYFCQQYNSYPYTFG | |
| | GYYAPGYWGQGTTLTVSS | | GGTKLEIK | |
| SCT-Da106 | EVQLQQSGAELVKPGASVKLSCTASGFNIKDTY | 81 | DIVMTQSQKFMSTSVGDRVSVTCKASQNVGTDV | 318 |
| | IHWMMQRPEQGLEWIGRIDPANGNTNYDPKFQG | | AWYQQKPGQSPKLLIYSASYRFSGVPDRFTGSG | |
| | KATITADTSSNTAYLQLSSLTSEDTAVYYCAHD | | SGTDFTLTISNVQSEDLAEYFCQQYNSYPYTFG | |
| | GYYAPGYWGQGTTLTVSS | | GGTKLEIK | |
| SCT-Da107 | EVQLQQSGAELVKPGASVKLSCTASGFNIKDTY | 82 | DILMTQSPSSMSVSLGDTVSITCHASQGISSNI | 327 |
| | IHWVKQRPEQGLEWIGRIDPANGNRIYDPKFQG | | GWLQRKPGKSFKGLIYHGTNLEDGLPSRFSGSG | |
| | KATITADSSSNTAYLQLSSLTSEDTAVYFCARD | | SGADYSLTISSLESEDFADYYCVQYAQFPYTFG | |
| | GYYAPGYWGQGTTLTVSS | | GGTKLEIK | |
| SCT-Da108 | EVQLQQSGAELVKPGASVKLSCTASGFNIKDTY | 82 | DIVLTQSPASLAVSLGQRATISCKASQSVDYDG | 328 |
| | IHWVKQRPEQGLEWIGRIDPANGNRIYDPKFQG | | DNYMNWYQQKPGQPPKLLIYAASNLESGIPARF | |
| | KATITADSSSNTAYLQLSSLTSEDTAVYFCARD | | SGSGSGTDFTLNIHPVEEEDAATYYCQQSYEDP | |
| | GYYAPGYWGQGTTLTVSS | | WTFGGGTKLEIK | |
| SCT-Da109 | EVQLQQSGAELVKPGASVKLSCTASGFNIKDTY | 82 | DIVMTQSQKFMSTSVGDGVSVTCKASQNVDSNV | 329 |
| | IHWVKQRPEQGLEWIGRIDPANGNRIYDPKFQG | | AWYHQKPGQSPKLLIYSASYRFSGVPDRFTGSG | |
| | KATITADSSSNTAYLQLSSLTSEDTAVYFCARD | | SGTDFTLTISNVQSEDLAEYFCQQYNSYPYTFG | |
| | GYYAPGYWGQGTTLTVSS | | GGTKLEVK | |
| SCT-Da110 | EVQLQQSGAELVKPGASVKLSCTASGFNIKDTY | 82 | DIVMTQSQKFMSTSVGDRVSVTCKASQNVGTNI | 330 |
| | IHWVKQRPEQGLEWIGRIDPANGNRIYDPKFQG | | AWYQQKPGQSPKLLIYSASYRYSGVPDRFSGSG | |
| | KATITADSSSNTAYLQLSSLTSEDTAVYFCARD | | SGTDFTLAISNVQSEDLAEYFCQQYNTYPYTFG | |
| | GYYAPGYWGQGTTLTVSS | | GGTKLEIK | |
| SCT-Da111 | EVQLQQSGAELVKPGASVKLSCTASGFNIKDTY | 83 | DIVMTQSPATLSVTPGDRVSLSCRASQSISDYL | 331 |
| | IHWVKQRPEQGLEWIGRIDPANGNRIYDPKFQG | | HWYQQKSHESPRLLIKYVSQSISGIPSRFSGSG | |
| | KATITADSSSNTAYLQLSSLTSEDTAVYYCARD | | SGSDFTLSINSVEPEDVGVYYCQNGHSFPFTFG | |
| | GYYAPGYWGQGTTLTVSS | | SGTKLEIK | |
| SCT-Da112 | EVQLQQSGAELVKPGASVKLSCTASGFNIKDTY | 83 | DIVMTQSQKFMSTSVGDRVSVTCKASQNVGTDV | 318 |
| | IHWVKQRPEQGLEWIGRIDPANGNRIYDPKFQG | | AWYQQKPGQSPKLLIYSASYRFSGVPDRFTGSG | |
| | KATITADSSSNTAYLQLSSLTSEDTAVYYCARD | | SGTDFTLTISNVQSEDLAEYFCQQYNSYPYTFG | |
| | GYYAPGYWGQGTTLTVSS | | GGTKLEIK | |
| SCT-Da113 | EVQLQQSGAELVKPGASVKLSCTASGFNIKDTY | 83 | DIVMTQSQKFMSTSVGDRVSVTCKASQNVGTDV | 332 |
| | IHWVKQRPEQGLEWIGRIDPANGNRIYDPKFQG | | AWYQQKPGQSPKLLIYSASYRYSGVPDRFTGSG | |
| | KATITADSSSNTAYLQLSSLTSEDTAVYYCARD | | SGTDFTLAISNVQSEDLAEYFCQQYNSYPYTFG | |
| | GYYAPGYWGQGTTLTVSS | | GGTKLEIK | |
| SCT-Da114 | EVQLQQSGAELVKPGASVKLSCTASGFNIKDTY | 83 | QIVLTQSPALMSASPGEKVTMTCSASSSVSYMY | 333 |
| | IHWVKQRPEQGLEWIGRIDPANGNRIYDPKFQG | | WYQQKPRSSPKPWIYLTSNLASGVPARFSGSGS | |
| | KATITADSSSNTAYLQLSSLTSEDTAVYYCARD | | GTSYSLTISSMEAEDAATYYCQQWSSNPLTFGA | |
| | GYYAPGYWGQGTTLTVSS | | GTKLELK | |
| SCT-Da115 | EVQLQQSGAELVKPGASVKLSCTASGFNIKDTY | 84 | DIVMTQSQKFMSTSVGDRVSVTCKASQNVGTNV | 334 |
| | IHWVKQRPEQGLEWIGRIDPANGNRKYDPKFQA | | AWYQQKPGQSPKLLIYSASYRFSGVPDRFAGSG | |
| | KATITADSSSNTAYLQLSSLTSEDTAVYYCARD | | SGTDFTLAISNVQSEDLADYFCQQYNSYPYTFG | |
| | GYYAPGYWGQGTTLTVSS | | GGTKLEMK | |
| SCT-Da116 | EVQLQQSGAELVKPGASVKLSCTASGFNIKDTY | 85 | DIVMTQSQKFMSTSVGDRVSVTCKASQNVGTDV | 304 |
| | IHWVKQRPEQGLEWIGRIDPANGNRKYDPKFQG | | AWYQQKPGQSPKLLIYSASYRFSGVPDRFTGSG | |
| | KATITADSLSNTAYLQLSSLTSEDTAVYYCARD | | SGTDFSLAISNVQSEDLAEYFCQQYNSYPYTFG | |
| | GYYAPGHWGQGTTLTVSS | | GGAKLEIK | |
| SCT-Da117 | EVQLQQSGAELVKPGASVKLSCTASGFNIKDTY | 85 | DIVMTQSQKFMSTSVGDRVSVTCKASQNVGTDV | 326 |
| | IHWVKQRPEQGLEWIGRIDPANGNRKYDPKFQG | | AWYQQKPGQSPKLLIYSASYRFSGVPDRFTGSG | |
| | KATITADSLSNTAYLQLSSLTSEDTAVYYCARD | | SGTDFTLAISNVQSEDLAEYFCQQYNSYPYTFG | |
| | GYYAPGHWGQGTTLTVSS | | GGTKLEIK | |
| SCT-Da118 | EVQLQQSGAELVKPGASVKLSCTASGFNIKDTY | 85 | DIVMTQSQKFMSTSVGDRVSVTCKASQNVGTDV | 318 |
| | IHWVKQRPEQGLEWIGRIDPANGNRKYDPKFQG | | AWYQQKPGQSPKLLIYSASYRFSGVPDRFTGSG | |
| | KATITADSLSNTAYLQLSSLTSEDTAVYYCARD | | SGTDFTLTISNVQSEDLAEYFCQQYNSYPYTFG | |
| | GYYAPGHWGQGTTLTVSS | | GGTKLEIK | |
| SCT-Da119 | EVQLQQSGAELVKPGASVKLSCTASGFNIKDTY | 85 | DIVMTQSQKFMSTSVGDRVSVTCKASQNVGTDV | 332 |
| | IHWVKQRPEQGLEWIGRIDPANGNRKYDPKFQG | | AWYQQKPGQSPKLLIYSASYRYSGVPDRFTGSG | |
| | KATITADSLSNTAYLQLSSLTSEDTAVYYCARD | | SGTDFTLAISNVQSEDLAEYFCQQYNSYPYTFG | |
| | GYYAPGHWGQGTTLTVSS | | GGTKLEIK | |
| SCT-Da120 | EVQLQQSGAELVKPGASVKLSCTASGFNIKDTY | 86 | DIVMTQSQKFMSTSVGDRVSVTCKASQNVGTDV | 335 |
| | IHWVKQRPEQGLEWIGRIDPANGNRKYDPKFQG | | AWYQQKPGQSPKLLIYSASYWFSGVPDRFTGSG | |
| | KATITADSSSNTAYLQLSSLASEDTAVYYCARD | | SGTDFTLAISNVQSEDLAEYFCQQYNSYPYTFG | |
| | GYSAPGYWGQGTTLTVSS | | GGTKLEIK | |
| SCT-Da121 | EVQLQQSGAELVKPGASVKLSCTASGFNIKDTY | 87 | DIVLTQSPASLAVSLGQRATISCRASESVESNG | 336 |
| | IHWVKQRPEQGLEWIGRIDPANGNRKYDPKFQG | | NSFMHWYQQKPGQSPKLLIYRASNLESGIPARF | |
| | KATITADSSSNTAYLQLSSLTSEDTAVYYCARD | | SGSGSRTDFTLTIYPVEADDVATYYCQQTDEDP | |
| | GYYAPGYWGQGTTLTVSS | | FTFGSGTKLGIK | |
| SCT-Da122 | EVQLQQSGAELVKPGASVKLSCTASGFNIKDTY | 87 | DIVMTQSPATLSVTPGDRVSLSCRASQRISDYL | 337 |
| | IHWVKQRPEQGLEWIGRIDPANGNRKYDPKFQG | | HWYQQKSHESPRLLIKYASQSISGIPSRFSGSG | |
| | KATITADSSSNTAYLQLSSLTSEDTAVYYCARD | | SGSDFTLSINSVEPEDVGMYYCQNGHSFPFTFG | |
| | GYYAPGYWGQGTTLTVSS | | SGTKLEIK | |
| SCT-Da123 | EVQLQQSGAELVKPGASVKLSCTASGFNIKDTY | 87 | DIVMTQSQKFMSTSVGDRVSVTCKASQNVGTDV | 326 |
| | IHWVKQRPEQGLEWIGRIDPANGNRKYDPKFQG | | AWYQQKPGQSPKLLIYSASYRFSGVPDRFTGSG | |
| | KATITADSSSNTAYLQLSSLTSEDTAVYYCARD | | SGTDFTLAISNVQSEDLAEYFCQQYNSYPYTFG | |
| | GYYAPGYWGQGTTLTVSS | | GGTKLEIK | |
| SCT-Da124 | EVQLQQSGAELVKPGASVKLSCTASGFNIKDTY | 87 | DIVMTQSQKFMSTSVGDRVSVTCKASQNVGTDV | 338 |
| | IHWVKQRPEQGLEWIGRIDPANGNRKYDPKFQG | | AWYQQKPGQSPKLLIYSASYRFSGVPDRFTGSG | |
| | KATITADSSSNTAYLQLSSLTSEDTAVYYCARD | | SGTDFTLAISNVQSEDLAEYFCQQYNSYPYTFG | |
| | GYYAPGYWGQGTTLTVSS | | GGTKVEIK | |
| SCT-Da125 | EVQLQQSGAELVKPGASVKLSCTASGFNIKDTY | 87 | DIVMTQSQKFMSTSVGDRVSVTCKASQNVGTDV | 339 |
| | IHWVKQRPEQGLEWIGRIDPANGNRKYDPKFQG | | AWYQQKPGQSPKLLIYSASYRYNGVPDRFTGSG | |
| | KATITADSSSNTAYLQLSSLTSEDTAVYYCARD | | SGTDFTLAISNVQSEDLAEYFCQQYNSYPYTFG | |
| | GYYAPGYWGQGTTLTVSS | | GGTKLEIK | |
| SCT-Da126 | EVQLQQSGAELVKPGASVKLSCTASGFNIKDTY | 87 | DIVMTQSQKFMSTSVGDRVSVTCKASQNVGTDV | 340 |
| | IHWVKQRPEQGLEWIGRIDPANGNRKYDPKFQG | | AWYQQKPGQSPKLLIYSASYRYNGVPDRFTGSG | |
| | KATITADSSSNTAYLQLSSLTSEDTAVYYCARD | | SGTDFTLAISNVQSEDLAEYFCQQYNTYPYTFG | |
| | GYYAPGYWGQGTTLTVSS | | GGTKLEIK | |
| SCT-Da127 | EVQLQQSGAELVKPGASVKLSCTASGFNIKDTY | 87 | DVQITQSPSYLAASPGETITINCRASKSISKYL | 341 |
| | IHWVKQRPEQGLEWIGRIDPANGNRKYDPKFQG | | AWYQEKPGKTNKLLIYSGSTLQSGIPSRFSGSG | |
| | KATITADSSSNTAYLQLSSLTSEDTAVYYCARD | | SGTDFTLTISSLEPEDFAMYYCQQHNEYPLTFG | |
| | GYYAPGYWGQGTTLTVSS | | AGTKLELK | |
| SCT-Da128 | EVQLQQSGAELVKPGASVKLSCTASGFNIKDTY | 87 | QIVLTQSPALMSASPGEKVTMTCSASSSVSYMY | 333 |
| | IHWVKQRPEQGLEWIGRIDPANGNRKYDPKFQG | | WYQQKPRSSPKPWIYLTSNLASGVPARFSGSGS | |
| | KATITADSSSNTAYLQLSSLTSEDTAVYYCARD | | GTSYSLTISSMEAEDAATYYCQQWSSNPLTFGA | |
| | GYYAPGYWGQGTTLTVSS | | GTKLELK | |
| SCT-Da129 | EVQLQQSGAELVKPGASVKLSCTASGFNIKDTY | 88 | DIVMTQSQKFMSTSVGDRVSVTCKASQNVGTDV | 318 |
| | IHWVKQRPEQGLEWIGRIDPANGNRKYDPKFQG | | AWYQQKPGQSPKLLIYSASYRFSGVPDRFTGSG | |
| | TATITADSSSNTAYLQLSSLTSEDTAVYYCARD | | SGTDFTLTISNVQSEDLAEYFCQQYNSYPYTFG | |
| | GYYAPGYWGQGTTLTVSS | | GGTKLEIK | |
| SCT-Da130 | EVQLQQSGAELVKPGASVKLSCTASGFNIKDTY | 89 | DIVMTQSQKFMSTSVGDRVSVTCKASQNVGTNV | 342 |
| | MHWVKQRPEQGLEWIGRIDPANGNRKYDPKFQG | | AWYQQKPGQSPKLLIYSASYRFSGVPDRFTGSG | |
| | KATITADSSSNTAYLQLSSLTSEDTAVYYCARD | | SGTDFTLAISNVQSEDLAEYFCQQYNSYPYTFG | |
| | GYYAPGYWGQGTTLTVSS | | GGTQLEIK | |
| SCT-Da131 | EVQLQQSGAELVKPGASVKLSCTASGFNIKDTY | 89 | DIVMTQSQKFMSTSVGDRVSVTCKASQNVGTNV | 313 |
| | MHWVKQRPEQGLEWIGRIDPANGNRKYDPKFQG | | AWYQQKPGQSPKLLIYSASYRYSGVPDRFTGSG | |
| | KATITADSSSNTAYLQLSSLTSEDTAVYYCARD | | SGTDFTLAISNVQSEDLAEYFCQQYNSYPYTFG | |
| | GYYAPGYWGQGTTLTVSS | | GGTKLEIK | |
| SCT-Da132 | EVQLQQSGAELVKPGASVKLSCTASGFNIKDTY | 90 | DIVMTQSQKFMSTSVGDRVSVTCKASQNVGTNV | 342 |
| | MHWVKQRPEQGLEWIGRIDPANGNRKYDPKFQG | | AWYQQKPGQSPKLLIYSASYRFSGVPDRFTGSG | |
| | KATITADSSSNTAYLQLSSLTSEDTAVYYWARD | | SGTDFTLAISNVQSEDLAEYFCQQYNSYPYTFG | |
| | GYYAPGYWGQGTTLTVSS | | GGTQLEIK | |
| SCT-Da133 | EVQLQQSGAELVKPGASVKLSCTASGFNIKDTY | 91 | DIVMTQSQKFMSTSVGDGVSVTCKASQNVDSNV | 329 |
| | MHWVKQRPEQGLEWIGRIDPANGNTGYDPKFQG | | AWYHQKPGQSPKLLIYSASYRFSGVPDRFTGSG | |
| | KATITADTSSNTAYLQLSSLTSEDTAVYYCAHD | | SGTDFTLTISNVQSEDLAEYFCQQYNSYPYTFG | |
| | GYYAPGYWGQGTTLTVSS | | GGTKLEVK | |
| SCT-Da134 | EVQLQQSGAELVKPGASVKLSCTASGFNIKDTY | 91 | DIVMTQSQKFMSTSVGDRVSVTCKASQNVDTNV | 294 |
| | MHWVKQRPEQGLEWIGRIDPANGNTGYDPKFQG | | AWYQQKPGQSPKLLIYSASYRFSGVPDRFTGSG | |
| | KATITADTSSNTAYLQLSSLTSEDTAVYYCAHD | | SGTDFTLTISNVQSEDLAEYFCQQYNSYPYTFG | |
| | GYYAPGYWGQGTTLTVSS | | GGTKLEIK | |
| SCT-Da135 | EVQLQQSGAELVKPGASVKLSCTASGFNIKDTY | 91 | QIVLTQSPALMSASPGEKVTMTCSASSSVSYMY | 333 |
| | MHWVKQRPEQGLEWIGRIDPANGNTGYDPKFQG | | WYQQKPRSSPKPWIYLTSNLASGVPARFSGSGS | |
| | KATITADTSSNTAYLQLSSLTSEDTAVYYCAHD | | GTSYSLTISSMEAEDAATYYCQQWSSNPLTFGA | |
| | GYYAPGYWGQGTTLTVSS | | GTKLELK | |
| SCT-Da136 | EVQLQQSGAELVKPGASVKLSCTASGFNIKDTY | 91 | SIVMTQTPKFLLVSAGDRVTITCKASQSVSNDV | 343 |
| | MHWVKQRPEQGLEWIGRIDPANGNTGYDPKFQG | | AWYQQKPGQSPKLLIYYASNRYTGVPDRFTGSG | |
| | KATITADTSSNTAYLQLSSLTSEDTAVYYCAHD | | YGTDFTFTISTVQAEDLAVYFCQQDYSSPWTFG | |
| | GYYAPGYWGQGTTLTVSS | | GGTKLEIK | |
| SCT-Da137 | EVQLQQSGAELVKPGASVKLSCTASGFNIKDTY | 92 | DIVMTQSQKFMSTSVGDRVSVTCKASQNVDTNV | 344 |
| | MHWVKQRPEQGLEWIGRIDPANGNTHYDPKFQV | | AWYQQKPGQSPKLLIYSASYRFSGVPDRFTGSG | |
| | KATITADTSSNTAYLQLSSLTSEDTAVYYCTHD | | SGTDFTLTISNVQSEDLAEYFCQQYNIYPYTFG | |
| | GYYAPGYWGQGTTLTVSS | | GGTKLEIK | |
| SCT-Da138 | EVQLQQSGAELVKPGASVKLSCTASGFNIKDTY | 92 | DIVMTQSQKFMSTSVGDRVSVTCKASQNVDTNV | 345 |
| | MHWVKQRPEQGLEWIGRIDPANGNTHYDPKFQV | | AWYQQKPGQSPKLLIYSASYRYSGVPDRFTGSG | |
| | KATITADTSSNTAYLQLSSLTSEDTAVYYCTHD | | SGTDFTLTISNVQSEDLAEYFCQQYNSYPYTFG | |
| | GYYAPGYWGQGTTLTVSS | | GGTKLEIK | |
| SCT-Da139 | EVQLQQSGAELVKPGASVKLSCTASGFNIKDTY | 92 | DIVMTQTPLSLPVSLGDQASISCRSSQSLVHSN | 346 |
| | MHWVKQRPEQGLEWIGRIDPANGNTHYDPKFQV | | GNTYLHWHLQKPGQSPKLLIYKVSNRFSGVPDR | |
| | KATITADTSSNTAYLQLSSLTSEDTAVYYCTHD | | FSGSGSGTDFTLKISRVEAEDLGVYFCSQSTHA | |
| | GYYAPGYWGQGTTLTVSS | | PWTFGGGTKLEIK | |
| SCT-Da140 | EVQLQQSGAELVKPGASVKLSCTASGFNIKDTY | 93 | DIVMTQSQKFMSTSVGDRVSVTCKASQNVGTNV | 316 |
| | MHWVKQRPEQGLEWIGRIDPANGNTIYDPKFQA | | AWYQQRPGQSPKLLIYSASYRYSGVPDRFTGSG | |
| | KATITADTSSNTAYLQLSSLTSEDTAVYYCAHD | | SGTDFTLTISNVQSEDLAEYFCQQYNNYPYTFG | |
| | GYYAPGYWGQGTTLTVSS | | GGTKLEIK | |
| SCT-Da141 | EVQLQQSGAELVKPGASVKLSCTASGFNIKDTY | 94 | DIVMTQSQEFMSTSVGDRVSVTCKASQNVGTNV | 347 |
| | MHWVKQRPEQGLEWIGRIDPANGNTIYDPNFQG | | AWYQQKPGQSPKLLIYSASYRFSGVPDRFTGSG | |
| | KATITADTSSNTAYLQLSSLTSEDTAVYYCAHD | | SGTDFTLTISNVQSEDLAEYFCQQYNNYPYTFG | |
| | GYYAPGYWGQGTTLTVSS | | GGTKLEIK | |
| SCT-Da142 | EVQLQQSGAELVKPGASVKLSCTASGFNIKDTY | 95 | DIVMTQSQKFMSTSVGDRVSVTCKASQNVGTNV | 348 |
| | MHWVKQRPEQGLEWIGRIDPANGNTNYDPKFQG | | AWYQQKPGQSPELLIYSASYRYSGVPDRFTGSG | |
| | KATITTDTSSNTAYLQLSSLTSEDTAVYYCAHD | | SGTDFTLTISNVQSEDLAEYFCQQYNSYPFTFG | |
| | GYYAPGFWGQGTTLTVSS | | GGTKLEIK | |
| SCT-Da143 | EVQLQQSGAELVKPGASVKLSCTASGFNIKDTY | 96 | EIVLTQSPTTMATSPGEKITITCSASSSIISNY | 349 |
| | MNWVKQRPEQGLEWIGRIDPANGNTKYDPKFQG | | LHWYQQKPGFSPKLLIYRTSNLASGVPARFSGS | |
| | KATITADTSSNTAYLQLSSLTSEDTAVYYCAPF | | GSGTSYSLTIGTMETEDVATYYCQQGSSLPRTF | |
| | SYGTLAYWGQGTTLTVSS | | GSGTKLEIK | |
| SCT-Da144 | EVQLQQSGAELVKPGASVKLSCTASGLNIKDTY | 97 | DIVMTQSQKFMSTSVGDRVSVTCKASQNVGTNV | 342 |
| | IHWVKQRPEQGLEWIGRIDPANGNRKYDPKFQG | | AWYQQKPGQSPKLLIYSASYRFSGVPDRFTGSG | |
| | KATITADSSSNTAYLQLSSLTSEDTAVYYCARD | | SGTDFTLAISNVQSEDLAEYFCQQYNSYPYTFG | |
| | GYYAPGYWGQGTTLTVSS | | GGTQLEIK | |
| SCT-Da145 | EVQLQQSGAELVKPGASVKLSCTASGVNIKDTY | 98 | DIVMTQSQKFMSTSVGDRVSVTCKASQNVGTDV | 304 |
| | IHWVKQRPEQGLEWIGRIDPANGNRKSDPKFQG | | AWYQQKPGQSPKLLIYSASYRFSGVPDRFTGSG | |
| | KATITADSSSNTAYLQLSSLTSEDTAVYYCARD | | SGTDFSLAISNVQSEDLAEYFCQQYNSYPYTFG | |
| | GYYAPGYWGQGTTLTVSS | | GGAKLEIK | |
| SCT-Da146 | EVQLQQSGAELVKPGASVKLSCTASGVNIKDTY | 99 | DILMTQSPSSMSVSLGDSVSITCHASQGISSNM | 284 |
| | IHWVKQRPEQGLEWIGRIDPANGNRKYDPKFQG | | GWLQQKAGKSFKGLIYHGTNLEDGVPSRFSGSG | |
| | KATITADSSSNTAYLQLSSLTSEDTAVYYCARD | | SGADYSLTISSLESEDFADYYCVQYAQFPYTFG | |
| | GYYAPGYWGQGTTLTVSS | | GGTKLEIK | |
| SCT-Da147 | EVQLQQSGAELVKPGASVKLSCTASGVNIKDTY | 99 | DIQMTQTTSSLSASLGDRVTISCSASQGISNYL | 296 |
| | IHWVKQRPEQGLEWIGRIDPANGNRKYDPKFQG | | NWYQQKPDGTVKLLIYYTSSLHSGVPSRFSGSG | |
| | KATITADSSSNTAYLQLSSLTSEDTAVYYCARD | | SGTDYSLTISNLEPEDIATYYCQQYSKLPLTFG | |
| | GYYAPGYWGQGTTLTVSS | | AGTKLELK | |
| SCT-Da148 | EVQLQQSGAELVKPGASVKLSCTASGVNIKDTY | 99 | DIVMTQSQKFMSTSVGDRVSVTCKASQNVGTDV | 304 |
| | IHWVKQRPEQGLEWIGRIDPANGNRKYDPKFQG | | AWYQQKPGQSPKLLIYSASYRFSGVPDRFTGSG | |
| | KATITADSSSNTAYLQLSSLTSEDTAVYYCARD | | SGTDFSLAISNVQSEDLAEYFCQQYNSYPYTFG | |
| | GYYAPGYWGQGTTLTVSS | | GGAKLEIK | |
| SCT-Da149 | EVQLQQSGAELVKPGASVKLSCTASGVNIKDTY | 99 | DIVMTQSQKFMSTSVGDRVSVTCKASQNVGTDV | 350 |
| | IHWVKQRPEQGLEWIGRIDPANGNRKYDPKFQG | | AWYQQKPGQSPKLLIYSASYRFSGVPDRFTGSG | |
| | KATITADSSSNTAYLQLSSLTSEDTAVYYCARD | | SGTDFSLAISNVQSEDLAEYFCQQYNSYPYTFG | |
| | GYYAPGYWGQGTTLTVSS | | GGTKLEIK | |
| SCT-Da150 | EVQLQQSGAELVKPGASVKLSCTASGVNIKDTY | 99 | DIVMTQSQKFMSTSVGDRVSVTCKASQNVGTDV | 351 |
| | IHWVKQRPEQGLEWIGRIDPANGNRKYDPKFQG | | AWYQQKPGQSPKLLIYSASYRFSGVPDRFTGSG | |
| | KATITADSSSNTAYLQLSSLTSEDTAVYYCARD | | SGTDFTLAISNVQSEDLAEYFCQQYNSYPYTFG | |
| | GYYAPGYWGQGTTLTVSS | | GGAKLEIK | |
| SCT-Da151 | EVQLQQSGAELVKPGASVKLSCTASGVNIKDTY | 99 | DIVMTQSQKFMSTSVGDRVSVTCKASQNVGTDV | 318 |
| | IHWVKQRPEQGLEWIGRIDPANGNRKYDPKFQG | | AWYQQKPGQSPKLLIYSASYRFSGVPDRFTGSG | |
| | KATITADSSSNTAYLQLSSLTSEDTAVYYCARD | | SGTDFTLTISNVQSEDLAEYFCQQYNSYPYTFG | |
| | GYYAPGYWGQGTTLTVSS | | GGTKLEIK | |
| SCT-Da152 | EVQLQQSGAELVKPGASVKLSCTASGVNIKDTY | 99 | DIVMTQSQKFMSTSVGDRVSVTCKASQNVGTNV | 352 |
| | IHWVKQRPEQGLEWIGRIDPANGNRKYDPKFQG | | AWYQQKPGQSPKLLIYSASYRFSGVPDRFTGSG | |
| | KATITADSSSNTAYLQLSSLTSEDTAVYYCARD | | SGTDFSLAISNVQSEDLAEYFCQQYNSYPYTFG | |
| | GYYAPGYWGQGTTLTVSS | | GGTKLEIK | |
| SCT-Da153 | EVQLQQSGAELVKPGASVKLSCTASGVNIKDTY | 99 | DIVMTQSQKFMSTSVGDRVSVTCKASQNVGTNV | 353 |
| | IHWVKQRPEQGLEWIGRIDPANGNRKYDPKFQG | | AWYQQKPGQSPKLLIYSASYRFSGVPDRFTGSG | |
| | KATITADSSSNTAYLQLSSLTSEDTAVYYCARD | | SGTDFTLAISNVQSEDLAEYFCQQYNSYPYTFG | |
| | GYYAPGYWGQGTTLTVSS | | GGTKLEIK | |
| SCT-Da154 | EVQLQQSGAELVKPGASVKLSCTASGVNIKDTY | 99 | SIVMTQTPKFLLVSAGDRFTITCKASQNVDNYV | 354 |
| | IHWVKQRPEQGLEWIGRIDPANGNRKYDPKFQG | | AWYQQKPGQSPKLLIYYASNRYTGVPDRFTGSG | |
| | KATITADSSSNTAYLQLSSLTSEDTAVYYCARD | | YGTDFTFTITTVQAEDLAVYFCQQDYSSPYTFG | |
| | GYYAPGYWGQGTTLTVSS | | GGTKLEIK | |
| SCT-Da155 | EVQLQQSGAELVKPGASVKLSCTVSGFNIKDTY | 100 | DIVMTQAAPSVPVTPGESVSISCRSNKSLLHSN | 355 |
| | MHWVKQRPEQGLEWIGMIDPANFNTKYDPKFQG | | GNTFLYWFLQRPGQSPQVLIYRHSNLASGVPDR | |
| | KATVTADTSSNTAYLQLSSLTSEDTAVYYCASL | | FSGSGSGTAFTLRISRVESEDVGVYYCMQHLEY | |
| | WVDYWGQGTSVTVSS | | PYTFGGGTKLEIK | |
| SCT-Da156 | EVQLQQSGAELVKPGASVKVSCTASGFNIKDTY | 101 | DIQMTQSPSSLSASLGGKVTITCKASQDINKYI | 314 |
| | IHWVKQRPEQGLEWIGRIDPANGNRKYDPKFQG | | AWYQHKPVKGPRLLIHYTSTLQPGIPSRFSGSG | |
| | KATITADSLSNTAYLQLSSLTSEDTAVYYCARD | | SGRDYSFSISNLEPEDIATYYCLQYDNLRTFGG | |
| | GYYAPGHWGQGTTLSVSS | | GTKLEIK | |
| SCT-Da157 | EVQLQQSGAELVKPGASVKVSCTASGFNIKDTY | 101 | DIVMTQSQKFMSTSVGDRVSVTCKASQNVGTDV | 304 |
| | IHWVKQRPEQGLEWIGRIDPANGNRKYDPKFQG | | AWYQQKPGQSPKLLIYSASYRFSGVPDRFTGSG | |
| | KATITADSLSNTAYLQLSSLTSEDTAVYYCARD | | SGTDFSLAISNVQSEDLAEYFCQQYNSYPYTFG | |
| | GYYAPGHWGQGTTLSVSS | | GGAKLEIK | |
| SCT-Da158 | EVQLQQSGAELVKPGASVKVSCTASGFNIKDTY | 101 | DIVMTQSQKFMSTSVGDRVSVTCKASQNVGTDV | 332 |
| | IHWVKQRPEQGLEWIGRIDPANGNRKYDPKFQG | | AWYQQKPGQSPKLLIYSASYRYSGVPDRFTGSG | |
| | KATITADSLSNTAYLQLSSLTSEDTAVYYCARD | | SGTDFTLAISNVQSEDLAEYFCQQYNSYPYTFG | |
| | GYYAPGHWGQGTTLSVSS | | GGTKLEIK | |
| SCT-Da159 | EVQLQQSGAELVKPGASVKVSCTASGFNIKDTY | 101 | DIVMTQSQKFMSTSVGDRVSVTCKASQNVGTNV | 322 |
| | IHWVKQRPEQGLEWIGRIDPANGNRKYDPKFQG | | AWYQQKPGQSPKLLIYSASYRYSGVPDRFTGSG | |
| | KATITADSLSNTAYLQLSSLTSEDTAVYYCARD | | SGTDFTLTISNVQSEDLAEYFCQQYNSYPYTFG | |
| | GYYAPGHWGQGTTLSVSS | | GGTKLEIK | |
| SCT-Da160 | EVQLQQSGAELVRPGALVKLSCKASGCNIKDYY | 102 | EIVLTQSPTTMAASPGEKITITCSASSSISSNY | 356 |
| | MHWVKQRPEQGLEWIGWIDPENGNTIYDPKFQA | | LNWYQQKPEFSPKLLIYRTSYLASGVPARFSGS | |
| | KASITADTSSNTAYLQFSSLTSEDTAVYYCTIG | | GSGTSYSLTIGTMEAEDVATYYCQQGSSIPRTF | |
| | VRRDYFAYWGQGTTLTVSS | | GSGTKLEIK | |
| SCT-Da161 | EVQLQQSGAELVRPGALVKLSCKASGCNIKDYY | 103 | EIVLTQSPTTMAASPGEKITITCSASSSISSNY | 356 |
| | MHWVKQRPEQGLEWIGWIDPENGNTIYDSKFQA | | LNWYQQKPEFSPKLLIYRTSYLASGVPARFSGS | |
| | KASITADTSSNTAYLQFSSLTSEDTAVYYCAIG | | GSGTSYSLTIGTMEAEDVATYYCQQGSSIPRTF | |
| | VRRDYFAYWGQGTTLTVSS | | GSGTKLEIK | |
| SCT-Da162 | EVQLQQSGAELVRPGALVKLSCKASGFNIKDFY | 104 | EIVLTQSPTTMAASPGEKITITCSATSIISSNY | 357 |
| | MHWVKQRPEQGLEWIGWIDPENGNTIYDPKFQG | | LHWYQQKPGFSPKLLIYRTSNLASGVPARFSGS | |
| | KASITADTSSNTAYLQLSSLTSEDTAVYYCAIG | | GSGTSYSLTIGTMEAEDVATYYCQQGSSIPRTF | |
| | VRRDYFGYWGQGTTLTVSS | | GSGTKLDIK | |
| SCT-Da163 | EVQLQQSGAELVRPGALVKLSCKASGFNIKDYY | 105 | DIQMTQSTSSLSASLGDRVSISCRASQDISNYL | 267 |
| | MHWVKQRPEQGLEWIGWIDPENGNTIYDPKFQG | | NWYQQKPDGTVKLLIYYTSRLHSGVPSRFSGSG | |
| | KARITADTSSNTAYLQLSSLTSEDTAVYYCAIG | | SGTDYSLTISSLEQEDVATYFCQQGNALPWTFG | |
| | VRRDYFAYWGQGTTLTVSS | | GGTKLEIK | |
| SCT-Da164 | EVQLQQSGAELVRPGALVKLSCKASGFNIKDYY | 105 | DIQMTQTTSSLSASLGDRVTISCRASHDIHNYL | 261 |
| | MHWVKQRPEQGLEWIGWIDPENGNTIYDPKFQG | | NWYQQKPDGTVKLLIYYTSRLHSGVPSRFSGSG | |
| | KARITADTSSNTAYLQLSSLTSEDTAVYYCAIG | | SGTDYSLTISNLEQEDIATYFCQQGNTLPWTFG | |
| | VRRDYFAYWGQGTTLTVSS | | GGTKLEIK | |
| SCT-Da165 | EVQLQQSGAELVRPGALVKLSCKASGFNIKDYY | 105 | EIVLTQSPTTMAASPGEKITIICNASSSISSNY | 358 |
| | MHWVKQRPEQGLEWIGWIDPENGNTIYDPKFQG | | FNWYQQKPEFSPKLLIYRTSNLASGVPARFSGS | |
| | KARITADTSSNTAYLQLSSLTSEDTAVYYCAIG | | GSGTSYSLTIGTMEAEDVATYYCQQGSSLPRTF | |
| | VRRDYFAYWGQGTTLTVSS | | GSGTKLEIK | |
| SCT-Da166 | EVQLQQSGAELVRPGALVKLSCKASGFNIKDYY | 105 | EIVLTQSPTTMAASPGEKITITCSANSNISSNY | 359 |
| | MHWVKQRPEQGLEWIGWIDPENGNTIYDPKFQG | | LNWYQQKPVFSPKLLIYRTSNLASGVPTRFSGS | |
| | KARITADTSSNTAYLQLSSLTSEDTAVYYCAIG | | GSGTSYSLTIGTMEAEDVATYYCQQGSSIPRTF | |
| | VRRDYFAYWGQGTTLTVSS | | GSGTKLEIK | |
| SCT-Da167 | EVQLQQSGAELVRPGALVKLSCKASGFNIKDYY | 106 | DIKMTQSPSSMYASLGERVTITCKASQDIKSYL | 360 |
| | MHWVKQRPEQGLEWIGWIDPENGNTIYDPKFQG | | SWYQQKPWKSPKTLIYYATSLADGVPSRFSGSG | |
| | KASITADTSSNTAYLQLSSLTSEDTAVYYCAIG | | SGQDYSLTISSLESDDTATYYCLQHGESPFTFG | |
| | VRRDYFAYWGQGTTLTVSS | | SGTKLEIK | |
| SCT-Da168 | EVQLQQSGAELVRPGALVKLSCKASGFNIKDYY | 106 | DIVLTQSPASLAVSLGQRATISCRASKSVSSSG | 361 |
| | MHWVKQRPEQGLEWIGWIDPENGNTIYDPKFQG | | YNYLHWYQQKPGQPPKLLIYLASNLESGVPARF | |
| | KASITADTSSNTAYLQLSSLTSEDTAVYYCAIG | | SGSGSGTDFTLNIHPVEEEDAATYYCQHSGELP | |
| | VRRDYFAYWGQGTTLTVSS | | LTFGAGTKLELK | |
| SCT-Da169 | EVQLQQSGAELVRPGALVKLSCKASGFNIKDYY | 106 | DIVMTQSPATLSVTPGDRVSLSCRASQSISDYL | 362 |
| | MHWVKQRPEQGLEWIGWIDPENGNTIYDPKFQG | | HWYQQKSHESPRLLIKYASQSISGIPSRFSGSG | |
| | KASITADTSSNTAYLQLSSLTSEDTAVYYCAIG | | SGSDFTLSINSVEPEDVGVYYCQNGHSFPPFTF | |
| | VRRDYFAYWGQGTTLTVSS | | GSGTKLEIK | |
| SCT-Da170 | EVQLQQSGAELVRPGALVKLSCKASGFNIKDYY | 106 | DIVMTQTPLSLPVSLGDQASISCRSSQSLVHSD | 363 |
| | MHWVKQRPEQGLEWIGWIDPENGNTIYDPKFQG | | GNTYLHWYLQKPGQSPKLLIYKVSNRFSGVPDR | |
| | KASITADTSSNTAYLQLSSLTSEDTAVYYCAIG | | FSGSGSGTDFTLMISRVEAEDLGVYFCSQSTHV | |
| | VRRDYFAYWGQGTTLTVSS | | PWTFGGGTKLEIK | |
| SCT-Da171 | EVQLQQSGAELVRPGALVKLSCKASGFNIKDYY | 106 | EIVLTQSPTTMAASPGEKITITCSASSSINSNY | 364 |
| | MHWVKQRPEQGLEWIGWIDPENGNTIYDPKFQG | | LNWYQQKPEFSPKLLIYRTSNLASGVPTRFSGS | |
| | KASITADTSSNTAYLQLSSLTSEDTAVYYCAIG | | GSGTSYSLTIGTMEAEDVATYYCQQGSSIPRTF | |
| | VRRDYFAYWGQGTTLTVSS | | GSGTKLEIK | |
| SCT-Da172 | EVQLQQSGAELVRPGALVKLSCKASGFNIKDYY | 106 | EIVLTQSPTTMAASPGEKITITCSASSSISSNY | 312 |
| | MHWVKQRPEQGLEWIGWIDPENGNTIYDPKFQG | | LNWYQQKPEFSPKLLIYRTSNLASGVPARFSGS | |
| | KASITADTSSNTAYLQLSSLTSEDTAVYYCAIG | | GSGTSYSLTIGTMEAEDVATYYCQQGSSIPRTF | |
| | VRRDYFAYWGQGTTLTVSS | | GSGTKLEIK | |
| SCT-Da173 | EVQLQQSGAELVRPGALVKLSCKASGFNIKDYY | 106 | EIVLTQSPTTMAASPGEKITITCSASSSISSNY | 365 |
| | MHWVKQRPEQGLEWIGWIDPENGNTIYDPKFQG | | LNWYQQKPEFSPKLLIYRTSNLASGVPGRFSGS | |
| | KASITADTSSNTAYLQLSSLTSEDTAVYYCAIG | | GSGTSYSLTIGTMEAEDVATYYCQQGSSIPRTF | |
| | VRRDYFAYWGQGTTLTVSS | | GSGTKLEIK | |
| SCT-Da174 | EVQLQQSGAELVRPGALVKLSCKASGFNIKDYY | 106 | QIVLTQSPALMAASPGEKVTITCSVSSSISYMY | 366 |
| | MHWVKQRPEQGLEWIGWIDPENGNTIYDPKFQG | | WYQQKSETSPKPWIYGTSNLASGVPVRFSGSGS | |
| | KASITADTSSNTAYLQLSSLTSEDTAVYYCAIG | | GTSYSLTISSMEAEDAATYYCQQWSSYPLTFGA | |
| | VRRDYFAYWGQGTTLTVSS | | GTKLELK | |
| SCT-Da175 | EVQLQQSGAELVRPGALVKLSCKASGFNIKDYY | 107 | EIVLTQSPTTMAASPGEKITITCSASSSISSNY | 262 |
| | MHWVKQRPEQGLEWIGWIDPENGNTIYDPKFQG | | LHWYQQKPGFSPKLLIYRTSNLASGVPARFSGS | |
| | KASITADTSSNTAYLQLSSLTSEDTAVYYCAIG | | GSGTSYSLTIGTMEAEDVATYYCQQGSSIPRTF | |
| | VRRDYFDYWGQGTTLTVSS | | GSGTKLEIK | |
| SCT-Da176 | EVQLQQSGAELVRPGALVKLSCKTSGFNIKDYY | 108 | DILMTQSPSSMSVSLGDSVSITCHASQGISSNI | 283 |
| | MHWVKQRPEQGLEWIGWIDPENGNTIYDPKFQD | | GWLQQKAGKSFKGLIYHGTNLEDGVPSRFSGSG | |
| | KAGITADTSSNTAYLQLSSLTSEDSAVYYCAIG | | SGADYSLTISSLESEDFADYYCVQYAQFPYTFG | |
| | VRRDYFAYWGQGTTLTVSS | | GGTTLEIK | |
| SCT-Da177 | EVQLQQSGAELVRPGALVKLSCKTSGFNIKDYY | 108 | EIVLTQSPTTMAASPGEKITITCSANSNISSNY | 359 |
| | MHWVKQRPEQGLEWIGWIDPENGNTIYDPKFQD | | LNWYQQKPVFSPKLLIYRTSNLASGVPTRFSGS | |
| | KAGITADTSSNTAYLQLSSLTSEDSAVYYCAIG | | GSGTSYSLTIGTMEAEDVATYYCQQGSSIPRTF | |
| | VRRDYFAYWGQGTTLTVSS | | GSGTKLEIK | |
| SCT-Da178 | EVQLQQSGAELVRPGALVKLSCKTSGFNIKDYY | 108 | EIVLTQSPTTMAASPGEKITITCSASSSISSNY | 367 |
| | MHWVKQRPEQGLEWIGWIDPENGNTIYDPKFQD | | LNWYQQKPVFSPKLLIYRTSNLASGVPTRFSGS | |
| | KAGITADTSSNTAYLQLSSLTSEDSAVYYCAIG | | GSGTSYSLTIGTMEAEDVATYYCQQGSSIPRTF | |
| | VRRDYFAYWGQGTTLTVSS | | GSGTKLEIK | |
| SCT-Da179 | EVQLQQSGAELVRPGALVKLSCKTSGFNIKDYY | 109 | EIVLTQSPTTVAASPGEKITITCSASSSISSNH | 368 |
| | MHWVKQRPEQGLEWIGWIDPENGNTIYDPKFQG | | LNWYQQKPGFSPKLLIYRTSNLASGVPARFSGS | |
| | KASITADTSSNTAYLQLNSLTSEDTAVYYCAIG | | GSGTSYSLTIGTMEAEDVATYYCQQGSSIPRTF | |
| | VRRDYFDYWGQGTTLTVSS | | GSGTKLEIK | |
| SCT-Da180 | EVQLQQSGAELVRPGALVKLSCKTSGFNIKDYY | 110 | EIVLTQSPTTVAASPGEKITITCSASSSISSNH | 368 |
| | MHWVKQRPEQGLEWIGWIDPENGNTIYDPKFQG | | LNWYQQKPGFSPKLLIYRTSNLASGVPARFSGS | |
| | KASITADTSSNTAYLQLSSLTSEDTAVYYCAIG | | GSGTSYSLTIGTMEAEDVATYYCQQGSSIPRTF | |
| | VRRDYFDYWGQGTTLTVSS | | GSGTKLEIK | |
| SCT-Da181 | EVQLQQSGPELLKPGASVKISCKTSGYTFTEYT | 111 | QIVLTQSPALMSASPGEKVTMTCSASSSVSYMY | 333 |
| | LHWVKQSHGKSLEWIGVINPNSGGTTYNQKFKD | | WYQQKPRSSPKPWIYLTSNLASGVPARFSGSGS | |
| | KATLTVDKSSSSAYMELRSLTSEDSAVYYCTRP | | GTSYSLTISSMEAEDAATYYCQQWSSNPLTFGA | |
| | HSLLRLRREFDYWGQGTTLTVSS | | GTKLELK | |
| SCT-Da182 | EVQLQQSGPELVKPGASMKISCKVSGFSFTGYT | 112 | DIVLTQSPASLAVSLGQRATISCKASQSVDYNG | 369 |
| | MNWVKQSHGKNLEWIGLINTYNGGTFYNQKFKA | | YSYLNWYQQKPGQPPKLLIYPASNLESGIPARF | |
| | KATLTVDKSSSTAHMELLSLTSEDSAVYYCARS | | SGSGSGTDFTLNIHPVEEEDAATYYCQQSKEDP | |
| | PGRYSMDYWGQGTSVTVSS | | YTFGGGTKLEIK | |
| SCT-Da183 | EVQLQQSGPELVKPGASMQISCKTSGYTFTEYT | 113 | QIVLTQSPALMSASPGEKVTMTCSASSSVSYMY | 333 |
| | VHWVKQSHGKSLEWIGVINPSTGGTTYNQKFKG | | WYQQKPRSSPKPWIYLTSNLASGVPARFSGSGS | |
| | KATLTVDQSSSTAYMELRSLTSEDSAVYYCARP | | GTSYSLTISSMEAEDAATYYCQQWSSNPLTFGA | |
| | HSLLRLRREFDYWGQGTTLTVSS | | GTKLELK | |
| SCT-Da184 | EVQLQQSGPELVKPGASVKISCKTSGYTFAEYT | 114 | QIVLTQSPALMSASPGEKVTMTCSASSSVSYMY | 370 |
| | MHWVKQSHGKSLEWIGVINPNNGGTTYNQQFKG | | WYQQKPRSSPKPWIYLTSNLASGVPARFSGSGS | |
| | KATLTVDKSSSTAYMELRSLTSEDSAVYYCTRP | | GTSYSLTISSMEAEDAATYYCQQWSSNPLTFGT | |
| | HSLLRLRREFDYWGQGTTLTVSS | | GTKLELK | |
| SCT-Da185 | EVQLQQSGPELVKPGASVKISCKTSGYTFTEYP | 115 | QIVLTQSPAIMSASPGEKVTMTCSASSSVSYMH | 371 |
| | MHWVKQSHGKSLEWIGGINPNNGGTTYNQKFKG | | WYQQKSGTSPKRWIYDTSKLASGVPARFSGSGS | |
| | KATLTVDKSSSTAYMELRSLTSEDSAVYYCALY | | GTSYSLTISSMEAEDAATYYCQQWSSNPLTFGA | |
| | GNYWYFDVWGAGTTVTVSS | | GTKLELK | |
| SCT-Da186 | EVQLQQSGPELVKPGASVKISCKTSGYTFTEYT | 116 | QIVLTQSPALMSASPGEKVTMTCSASSSVSYMY | 372 |
| | IHWVKQSHGKSLEWIGVINPNSGGTTYNQKFKG | | WYQQKPRSSPKPWIYLTSNLASGVPARFSGSGS | |
| | KATLTVDHSSSTAYMELRSLTSEDSAVYYCARP | | GTSYSLTISTMEAEDAATYYCQQWSSNPLTFGA | |
| | HSLLRLRREFDYWGQGTTLTVSS | | GTKLELK | |
| SCT-Da187 | EVQLQQSGPELVKPGASVKISCKTSGYTFTEYT | 117 | QIVLTQSPALMSASPGEKVTMTCSATSSVSYMY | 373 |
| | IHWVKQSHGKSLEWIGVINPNSGGTTYNQKFKG | | WYQQKPRSSPKPWIYLTSNLASGVPARFSGSGS | |
| | KATLTVDQSSSTAYMELRSLTSEDSAVYYCARP | | GTSYSLTISSMEAEDAATYYCQQWSSNPLTFGA | |
| | HSLLRLRREFDYWGQGTTLTVSS | | GTKLELK | |
| SCT-Da188 | EVQLQQSGPELVKPGASVKISCKTSGYTFTEYT | 118 | QIVLTQSPALMSASPGEKVTMTCSASSSVSYMY | 333 |
| | MHLVKQSHGKSLEWIGVINPNNGGTTYNQKFKG | | WYQQKPRSSPKPWIYLTSNLASGVPARFSGSGS | |
| | KATLTVDKSSSTAYMELRSLTSEDSAVYYCARP | | GTSYSLTISSMEAEDAATYYCQQWSSNPLTFGA | |
| | HSLLRLRREFDYWGQGTTLTVSS | | GTKLELK | |
| SCT-Da189 | EVQLQQSGPELVKPGASVKISCKTSGYTFTEYT | 119 | QIVLTQSPALMSASPGEKVTMTCSASSSVSYMY | 333 |
| | MHWVKQSHGKSLEWIGAVNPNTGGTTYNQKFKD | | WYQQKPRSSPKPWIYLTSNLASGVPARFSGSGS | |
| | KATLTIDKSSSTAYMELRSLTSEDSAVYYCARP | | GTSYSLTISSMEAEDAATYYCQQWSSNPLTFGA | |
| | HSLLRLRREFDYWGQGTTLTVSS | | GTKLELK | |
| SCT-Da190 | EVQLQQSGPELVKPGASVKISCKTSGYTFTEYT | 120 | QIVLTQSPALMSASPGEKVTMTCSASSSVSYMY | 374 |
| | MHWVKQSHGKSLEWIGGINPNNGGTSYNQKFKG | | WYQQKPRSSPKPWIYLTSNLASGVPARFSGSGS | |
| | KATLTVDKSSSTAYMELRSLTSEDSAVYYCARP | | GTSYSLTISSMEAEDAATYYCHQWSSNPLTFGA | |
| | HSLLRLRREFDYWGQGTTLTVSS | | GTKLELK | |
| SCT-Da191 | EVQLQQSGPELVKPGASVKISCKTSGYTFTEYT | 121 | DIVMTQSQKFMSTSVGDGVSVTCKASQNVDSNV | 329 |
| | MHWVKQSHGKSLEWIGVINPNNGGTTYNQKFKG | | AWYHQKPGQSPKLLIYSASYRFSGVPDRFTGSG | |
| | KATLTVDKSSSTAYMELRSLTSEDSAVYYCARP | | SGTDFTLTISNVQSEDLAEYFCQQYNSYPYTFG | |
| | HSLLRLRREFDYWGQGTTLTVSS | | GGTKLEVK | |
| SCT-Da192 | EVQLQQSGPELVKPGASVKISCKTSGYTFTEYT | 121 | QIVLTQSPALMSASPGEKVTMTCSASSSVSYMY | 333 |
| | MHWVKQSHGKSLEWIGVINPNNGGTTYNQKFKG | | WYQQKPRSSPKPWIYLTSNLASGVPARFSGSGS | |
| | KATLTVDKSSSTAYMELRSLTSEDSAVYYCARP | | GTSYSLTISSMEAEDAATYYCQQWSSNPLTFGA | |
| | HSLLRLRREFDYWGQGTTLTVSS | | GTKLELK | |
| SCT-Da193 | EVQLQQSGPELVKPGASVKISCKTSGYTFTEYT | 122 | QIVLTQSPALMSASPGEKVTMTCSASSSVSYMY | 375 |
| | MHWVKQSHGKSLEWIGVINPNNGGTTYNQKFKG | | WYQQKPRSSPKPWIYLTSNLASGVPARFNGSGS | |
| | KATLTVDKSSSTAYMELRSLTSEDSAVYYCTRP | | GTSYSLTISSMEAEDAATYYCQQWSSNPLTFGA | |
| | HSLLRLRREFDYWGQGTTLTVSS | | GTKLELK | |
| SCT-Da194 | EVQLQQSGPELVKPGASVKISCKTSGYTFTEYT | 123 | QIVLTQSPALMSVSPGEKVTMTCSASSSVSYMY | 376 |
| | MHWVKQSHGKSLEWIGVINPNSGGTTYNQKFKD | | WYQQKPRSSPKPWIYLTSNLASGVPARFSGSGS | |
| | KATLTVDKSSSTAYMELRSLTSEDSAVYYCTRP | | GTSYSLTISSMEAEDAATYYCQQWSSNPLTFGA | |
| | HSLLRLRREFDYWGQGTTLTVSS | | GTKLELK | |
| SCT-Da195 | EVQLQQSGPELVKPGASVKISCKTSGYTFTEYT | 124 | QIVLTQSPALMSASPGEKVTMTCSASSSVSYIY | 377 |
| | MHWVKQSHGKSLEWIGVINPNSGGTTYNQKFKG | | WYQQKPRSSPKPWIYLTSNLASGVPARFSGSGS | |
| | KATLTVDKSSSTAYMELRSLTSEDSAVYYCARP | | GTSYSLTISSMEAEDAATYYCQQWSSNPLTFGA | |
| | HSLLRLRREFDYWGQGTTLTVSS | | GTKLELK | |
| SCT-Da196 | EVQLQQSGPELVKPGASVKISCKTSGYTFTEYT | 124 | QIVLTQSPALMSASPGEKVTMTCSASSSVSYMY | 378 |
| | MHWVKQSHGKSLEWIGVINPNSGGTTYNQKFKG | | WYQQKPRSSPKPWIYLTSSLASGVPARFSGSGS | |
| | KATLTVDKSSSTAYMELRSLTSEDSAVYYCARP | | GTSFSLTISNMEAEDAATYYCHQWSSNPLTFGA | |
| | HSLLRLRREFDYWGQGTTLTVSS | | GTKLELK | |
| SCT-Da197 | EVQLQQSGPELVKPGASVKISCKTSGYTFTEYT | 125 | QIVLTQSPALMSASPGEKVTMTCSASSSVSYMY | 379 |
| | MHWVKQSHGKSLEWIGVINPNSGGTTYNQKFKG | | WYQQKPRSSPKPWIYLTSNLASGVPARFSGSGS | |
| | KATLTVDQSSSTAYMELLSLTSEDSAVYYCARP | | GTSYSLTISSMEAEDAATYYCQQWSSNPLTFGA | |
| | HSLLRLRREFDYWGQGTTLTVSS | | GTKLELN | |
| SCT-Da198 | EVQLQQSGPELVKPGASVKISCKTSGYTFTEYT | 126 | DIVMTQSQKFMSTSVGDRVSVTCKASQNVDTNV | 294 |
| | MHWVKQSHGKSLEWIGVINPNSGGTTYNQKFKG | | AWYQQKPGQSPKLLIYSASYRFSGVPDRFTGSG | |
| | KATLTVDQSSSTAYMELRSLTSEDSAVYYCARP | | SGTDFTLTISNVQSEDLAEYFCQQYNSYPYTFG | |
| | HSLLRLRREFDYWGQGTTLTVSS | | GGTKLEIK | |
| SCT-Da199 | EVQLQQSGPELVKPGASVKISCKTSGYTFTEYT | 126 | QIVLTQSPALMSASPGEKVTMTCSASSSVSYIY | 377 |
| | MHWVKQSHGKSLEWIGVINPNSGGTTYNQKFKG | | WYQQKPRSSPKPWIYLTSNLASGVPARFSGSGS | |
| | KATLTVDQSSSTAYMELRSLTSEDSAVYYCARP | | GTSYSLTISSMEAEDAATYYCQQWSSNPLTFGA | |
| | HSLLRLRREFDYWGQGTTLTVSS | | GTKLELK | |
| SCT-Da200 | EVQLQQSGPELVKPGASVKISCKTSGYTFTEYT | 126 | QIVLTQSPALMSASPGEKVTMTCSASSSVSYMY | 333 |
| | MHWVKQSHGKSLEWIGVINPNSGGTTYNQKFKG | | WYQQKPRSSPKPWIYLTSNLASGVPARFSGSGS | |
| | KATLTVDQSSSTAYMELRSLTSEDSAVYYCARP | | GTSYSLTISSMEAEDAATYYCQQWSSNPLTFGA | |
| | HSLLRLRREFDYWGQGTTLTVSS | | GTKLELK | |
| SCT-Da201 | EVQLQQSGPELVKPGASVKISCKTSGYTFTEYT | 127 | QIVLTQSPALMSASPGEKVTMTCSASSSVSYMY | 333 |
| | MHWVKQSHGKSLEWIGVINPNSGGTTYNQKFKG | | WYQQKPRSSPKPWIYLTSNLASGVPARFSGSGS | |
| | KATLTVDQSSSTAYMELRSLTYEDSAVYYCARP | | GTSYSLTISSMEAEDAATYYCQQWSSNPLTFGA | |
| | HSLLRLRREFDYWGQGTTLTVSS | | GTKLELK | |
| SCT-Da202 | EVQLQQSGPELVKPGASVKISCKTSGYTFTEYT | 128 | DIVMTQSQKFMSTSVGDRVSVTCKASQNVDTNV | 294 |
| | MHWVKQSHGKSLEWIGVINPNSGGTTYNQKFKG | | AWYQQKPGQSPKLLIYSASYRFSGVPDRFTGSG | |
| | KATLTVDQSSTTAYMELRSLTSEDSAVYYCARP | | SGTDFTLTISNVQSEDLAEYFCQQYNSYPYTFG | |
| | HSLLRLRREFDYWGQGTTLTVSS | | GGTKLEIK | |
| SCT-Da203 | EVQLQQSGPELVKPGASVKISCKTSGYTFTEYT | 128 | EIVLTQSPTTMAASPGEKITITCSANSNISSNY | 359 |
| | MHWVKQSHGKSLEWIGVINPNSGGTTYNQKFKG | | LNWYQQKPVFSPKLLIYRTSNLASGVPTRFSGS | |
| | KATLTVDQSSTTAYMELRSLTSEDSAVYYCARP | | GSGTSYSLTIGTMEAEDVATYYCQQGSSIPRTF | |
| | HSLLRLRREFDYWGQGTTLTVSS | | GSGTKLEIK | |
| SCT-Da204 | EVQLQQSGPELVKPGASVKISCKTSGYTFTEYT | 128 | QIVLSQSPAILSASPGEKVTMTCRASSSVSYMH | 380 |
| | MHWVKQSHGKSLEWIGVINPNSGGTTYNQKFKG | | WYQQKPGSSPKPWIYATSNLASGVPARFSGSGS | |
| | KATLTVDQSSTTAYMELRSLTSEDSAVYYCARP | | GTSYSLTISRVEAEDAATYYCQQWSSNPLTFGA | |
| | HSLLRLRREFDYWGQGTTLTVSS | | GTKLELK | |
| SCT-Da205 | EVQLQQSGPELVKPGASVKISCKTSGYTFTEYT | 128 | QIVLTQSPALMSASPGEKVTMTCSASSSVSYMY | 333 |
| | MHWVKQSHGKSLEWIGVINPNSGGTTYNQKFKG | | WYQQKPRSSPKPWIYLTSNLASGVPARFSGSGS | |
| | KATLTVDQSSTTAYMELRSLTSEDSAVYYCARP | | GTSYSLTISSMEAEDAATYYCQQWSSNPLTFGA | |
| | HSLLRLRREFDYWGQGTTLTVSS | | GTKLELK | |
| SCT-Da206 | EVQLQQSGPELVKPGASVKISCKTSGYTFTEYT | 129 | QIVLTQSPALMSASPGEKVTMTCSASSSVSYMY | 333 |
| | MHWVKQSHGKSLEWIGVINPNTGGTTYNQKFKG | | WYQQKPRSSPKPWIYLTSNLASGVPARFSGSGS | |
| | KATLTVDKSSTTAYMELRSLTSEDSAVYYCARP | | GTSYSLTISSMEAEDAATYYCQQWSSNPLTFGA | |
| | HSLLRLRREFDYWGQGTTLTVSS | | GTKLELK | |
| SCT-Da207 | EVQLQQSGPELVKPGASVKISCKTSGYTFTEYT | 130 | QIVLTQSPALMSASPGEKVTMTCSASSSVSYMY | 333 |
| | MHWVKQSHGKSLEWIGVINPNTGGTTYNQKFKG | | WYQQKPRSSPKPWIYLTSNLASGVPARFSGSGS | |
| | KATLTVDQSSTTAYMELRSLTSEDSAVYYCARP | | GTSYSLTISSMEAEDAATYYCQQWSSNPLTFGA | |
| | HSLLRLRREFDYWGQGTTLTVSS | | GTKLELK | |
| SCT-Da208 | EVQLQQSGPELVKPGASVKISCKTSGYTFTEYT | 131 | QIVLTQSPALMSASPGEKVTMTCSASSSVSYIY | 381 |
| | MHWVKQSQGKSLEWIGAVNLNTGGTTYNQKFKD | | WYQQKPGSSPKPWIYLTSNLASGVPPRFSGSGS | |
| | KATLTIDKSSSTAYMELRSLTSEDSAVYYCARP | | GTSYSLTISSMEAEDAASYYCQQWSSNPLTFGA | |
| | HSLLRLRREFDYWGQGTTLTVSS | | GTKLELK | |
| SCT-Da209 | EVQLQQSGPELVKPGASVKMSCKASGYTFTSYV | 132 | DIVMTQSQKFMSTSVGDRVSVTCKASQNVGTDV | 317 |
| | IHWVKQKPGQGLEWIGYINPYHDGTKYNEKFKG | | AWYQQKPGQSPKLLIYSASYRFSGVPDRFTGSG | |
| | KATLTSDKSSSTAYMELSSLTSEDSAVYYCARS | | SGTDFTLTISNVQSEDLAEYFCQQYNIYPYTFG | |
| | RTVIGFDYWGQGTTLTVSS | | GGTKLEIK | |
| SCT-Da210 | EVQLQQSGPELVKPGASVKMSCKASGYTFTSYV | 133 | DIVMTQSPATLSVTPGDRVSLSCRASQSISDYL | 382 |
| | MHWVKQKPGQGLEWIGYINPYHDGTKYNEKFIG | | HWYQQKSHESPRLLIKYASQSISGIPSRFSGSG | |
| | KATLTSDKSSSTAYMELSSLTSEDSAVYYCARS | | SGTDFTLSINSVEPEDVGVYYCQSGHSFPLTFG | |
| | RTVIGFDYWGQGTTLTVSS | | AGTKLEVK | |
| SCT-Da211 | EVQLQQSGPELVKPGASVKMSCKASGYTFTSYV | 134 | DIVMTQSQKFMSTSVGDRVSVTCKASQNVGTDV | 317 |
| | MHWVKQKPGQGLEWIGYINPYHDGTKYNEKFKG | | AWYQQKPGQSPKLLIYSASYRFSGVPDRFTGSG | |
| | KATLTSDKSSSTAYMELSSLTSEDSAVYYCARS | | SGTDFTLTISNVQSEDLAEYFCQQYNIYPYTFG | |
| | RTVIGFDYWGQGTTLTVSS | | GGTKLEIK | |
| SCT-Da212 | EVQLQQSGPELVKPGASVKMSCKASGYTFTSYV | 135 | DIQMTQSPASLSASVGETVTITCRASENIYSYL | 383 |
| | MHWVKQKPGQGLEWIGYINPYNDATKNNEKFKV | | AWHKQKQGKSPQFLVYNAKTLAEGVPSRFSGSG | |
| | KATLTSDKSSNTAYMELSSLTSEDSAVYYCARG | | SGTQFSLKINGLQPEDFGSYYCQHHYVIPPTFG | |
| | VRLLDYWGQGTTLTVSS | | GGTKLEIK | |
| SCT-Da213 | EVQLQQSGPELVKPGASVKMSCKASGYTFTSYV | 136 | DIVMTQSPATLSVTPGDRVSLSCRASQNISDYL | 384 |
| | MHWVKQKPGQGLEWIGYINPYTDGTKYNEKFKG | | HWYQQRSHESPRLLIKYASLSISGIPSRFSGSG | |
| | KATLTSDKSSSTAYMELSSLTSEDSAVYYCAKS | | SGSDFTLSINSVEPEDVGLYYCQNGHSFPLTFG | |
| | RTVIGFDYWGQGTTLTVSS | | AGTKLELK | |
| SCT-Da214 | EVQLQQSGPELVKPGASVKMSCKASGYTFTSYV | 137 | DIVMTQSPATLSVTPGDRVSLSCRASQNISDYL | 385 |
| | MHWVRQKPGQGLEWIGYINPYNDGTKYNEKFKG | | HWYQQRSHESPRLLIKYASLSISGIPSRFSGSG | |
| | KATLTSDKSSSTANMELSSLTSEDSAVYYCARS | | SGSDFTLSINSVEPEDVGVYYCQNAHSFPLTFG | |
| | RTVVGFDYWGQGTTLTVSS | | AGTKLELK | |
| SCT-Da215 | EVQLQQSGPELVKPGASVKMSCKASGYTFTTYV | 138 | QNVLTQSPALMSASPGEKVTMTCSASSSVSYMY | 386 |
| | MHWVKQKPGQGLEWICYINPYNDGSNYNEKFKG | | WYQQKPRSSPKPWIYLTSNLASGVPARFSGSGS | |
| | KATLTSDKSSTTAYMELSSLTSEDSAVYYCARW | | GTSYSLTISSMEAEDAATYYCQQWSSNPLTFGA | |
| | VYYDFGGSMDYWGQGTSVTVSS | | GTKLELK | |
| SCT-Da216 | EVQLQQSGPELVKPGASVKMSCKASGYTFTTYV | 139 | QIVLTQSPALMSASPGEKVTMTCSASSSVSYMY | 333 |
| | MHWVKQKPGQGLEWIGYINPYNDGSNYNEKFKG | | WYQQKPRSSPKPWIYLTSNLASGVPARFSGSGS | |
| | KATLTSDKSSTTAYMELSSLTSEDSAVYYCARW | | GTSYSLTISSMEAEDAATYYCQQWSSNPLTFGA | |
| | VYYDFGGSMDYWGQGTSVTVSS | | GTKLELK | |
| SCT-Da217 | EVQLQQSGPELVKPGASVKMSCKASGYTFTTYV | 139 | QNVLTQSPALMSASPGEKVTMTCSASSSVSYMY | 386 |
| | MHWVKQKPGQGLEWIGYINPYNDGSNYNEKFKG | | WYQQKPRSSPKPWIYLTSNLASGVPARFSGSGS | |
| | KATLTSDKSSTTAYMELSSLTSEDSAVYYCARW | | GTSYSLTISSMEAEDAATYYCQQWSSNPLTFGA | |
| | VYYDFGGSMDYWGQGTSVTVSS | | GTKLELK | |
| SCT-Da218 | EVQLQQSGPELVKPGTSVKISCKTSGYTFTEYT | 140 | QIVLTQSPALMSASPGEKVTMTCSASSSVSYIY | 387 |
| | MHWVKQSHGKSLEWIGAVNPNTGGTTYNQKFKD | | WYQQKPRSSPKPLIYLTSNLASGVPARFSGSGS | |
| | KATLTIDKSSSTAYMQLRSLTSEDSAVYYCARP | | GTSYSLTISSMEAEDAASYYCQQWSSNPLTFGA | |
| | HSLLRLRREFDYWGQGTTLTVSS | | GTKLELK | |
| SCT-Da219 | EVQLQQSGPELVKPGTSVKMSCKASGYTFTSYV | 141 | DIVMTQSPATLSVTPGDRVSLSCRASQSISDYL | 388 |
| | MHWVKQKPGQGLEWIGYINPYHDGTQYNEKFKG | | HWYQQKSHESPRLLIKYASQSISGIPSRFSGSG | |
| | KATLTSDKSSSTAYMELSSLTSEDSAVYYCARS | | SGSDFTLSINSVEPEDVGVYYCQNGHSFPLTFG | |
| | RTVIGFDYWGQGTTLTVSS | | AGTKLELK | |
| SCT-Da220 | EVQLQQSGPELVKSGASVKISCKTSGYTFTEYT | 142 | QIVLTQSPALMSASPGEKVTMTCSASSSVSYMY | 333 |
| | MHWVKQSHGKSLEWIGVINPNSGGTTYNQKFKG | | WYQQKPRSSPKPWIYLTSNLASGVPARFSGSGS | |
| | KATLTVDQSSSTAYMELRSLTSEESAVYYCTRP | | GTSYSLTISSMEAEDAATYYCQQWSSNPLTFGA | |
| | HSLLRLRREFDYWGQGTTLTVSS | | GTKLELK | |
| SCT-Da221 | EVQLQQSGPELVTPGASVNISCKTSGYTFTEYT | 143 | QIVLTQSPALMSASPGEKVTMTCSASSSVSYMY | 389 |
| | MHWVRQSHGKSLEWIGVINPNSGGTTYNQKFKD | | WYQQKPRSSPKPWIYLTSNLASGVPARFSGSGS | |
| | KATLTVDKSSTTAFMELRSLTSEDSAVYYCARP | | GTSYSLTISNMEAEDAATYYCQQWSSNPLTFGA | |
| | HSLLRLRREFDYWGQGTTLTVSS | | GTKLELK | |
| SCT-Da222 | EVQLQQYGPELVKPGASVKISCKTSGYTFTEYT | 144 | DIVMTQSQKFMSTSVGDGVSVTCKASQNVDSNV | 329 |
| | MHWVRQSHGKSLEWIGVINPNNGGTTYNQKFKG | | AWYHQKPGQSPKLLIYSASYRFSGVPDRFTGSG | |
| | KATLTVDKSSTTAFMELRSLTSEDSAVYYCARP | | SGTDFTLTISNVQSEDLAEYFCQQYNSYPYTFG | |
| | HSLLRLRREFDYWGQGTTLTVSS | | GGTKLEVK | |
| SCT-Da223 | EVQLQQYGPELVKPGASVKISCKTSGYTFTEYT | 144 | QIVLTQSPALMSASPGEKVTMTCSASSSVSYMY | 333 |
| | MHWVRQSHGKSLEWIGVINPNNGGTTYNQKFKG | | WYQQKPRSSPKPWIYLTSNLASGVPARFSGSGS | |
| | KATLTVDKSSTTAFMELRSLTSEDSAVYYCARP | | GTSYSLTISSMEAEDAATYYCQQWSSNPLTFGA | |
| | HSLLRLRREFDYWGQGTTLTVSS | | GTKLELK | |
| SCT-Da224 | EVQLVESGGDLAKPGGSLKLSCAASGFTLSRYG | 145 | DIQMTQSPAFLSASVGESVTITCRASENIYSYL | 390 |
| | MSWVRQTPDKRLEWVATSSGGGIYTYYPDSVKG | | AWYQQKQGKSPQLLVYNAKTLAEGVPSRFSGSG | |
| | RFTISRDNAKNTLYLQMSSLKSEDTAMYYCARH | | SGTHFSLKINSLQPEDFGGYYCQHHYGIPPWTF | |
| | DGVPMDYWGQGTSVTVSS | | GGGTKLEIK | |
| SCT-Da225 | EVQLVESGGDLVKPGGSLKLSCAASGFTFSSYG | 146 | DIVLTQSPASLAVSLGQRATFSCRASKSVSTSG | 391 |
| | MSWVRQTPDKRLEWVATIFSGDSNTYYLDSVKG | | YSYMHWYQQKPGQPPKLLIYLASNLESGVPARF | |
| | RFIISRDNAKNTLYLQMSSLKSEDTAMYYCSRQ | | SGSGSGTDFTLNIHPVEEEDAAAYYCQHSRELP | |
| | GHRSWFAYWGQGTLVTVSA | | YTFGGGTKLEIK | |
| SCT-Da226 | EVQLVESGGDLVKPGGSLKLSCAASGFTFSTYG | 147 | EIVLTQSPTTMATSPGEKITITCSASSSIISNY | 349 |
| | MSWVRQTPDKRLEWVATISGGGIYTYYPDSVKG | | LHWYQQKPGFSPKLLIYRTSNLASGVPARFSGS | |
| | RFTISSDNAKNTLYLQMSSLKSEDTAMYYCVRM | | GSGTSYSLTIGTMETEDVATYYCQQGSSLPRTF | |
| | ITTYFDHWGQGTTLTVSS | | GSGTKLEIK | |
| SCT-Da227 | EVQLVESGGDLVKPGGSLKVSCAASGFTFSSYG | 148 | DIVLTQSPASLVVSLGQRATISCRASKSVSTSG | 392 |
| | MSWVRQTPDKRLEWVATIFSGDSYTYYSDSVKG | | YSYMHWYQQKPGQPPKLLIYLASNLESGVPARF | |
| | RFTISRDNAKNTLYLQMSSLKSEDTAMYYCARQ | | SGSGSGTDFTLNIHPVEEEDAATYYCQHSRELP | |
| | GHRSWFAYWGQGTLVTVSA | | STFGGGTKLEIK | |
| SCT-Da228 | EVQLVESGGGLVKPGGSLKLSCAASGFTFSSYA | 149 | DIQMTQSPASLSASVGETVTITCRASENIYSYL | 393 |
| | MSWVRQTPEKRLEWVATISSGGSYTYYPDSVKG | | VWYQQKQGKSPQLLVYYAKTLAEGVPSRFSGSG | |
| | RFTISRDIAENTLFLQMSSLRSEDTAMYYCARH | | SGTQFSLKINSMQPEDFGSYYCQHHYGTPLTFG | |
| | EDTTYYYAMDYWGQGTSVTVSS | | VGTKVELK | |
| SCT-Da229 | EVQLVESGGGLVLPKGSLKLSCAASGFTFNTYA | 150 | QIVLTQSPAIMSASPGEKVTLTCSASSSVSSSH | 394 |
| | MNWVRQAPGKGLEWVARIRIKSNNYATSYADSV | | MYWYQQKPGSSPKLWIYSTSNLASGVPARFSGS | |
| | KDRFTISRDDSQSMLYLQMNNLKTEDTAVYYCV | | GSGTSYSLTISSMEAEDAASYFCHQWSSYPYTF | |
| | RQAYGNSYFFAMDYWGQGTSVTVSS | | GGGTKLEIK | |
| SCT-Da230 | EVQLVETGGGLVQPKGSLKLSCAASGFTFNTNA | 151 | DVVMTQTPLSLPVSLGDQASISCSSSQSLVYSN | 395 |
| | MNWVRQAPGKGLEWVARIRSKSNNYATNYVDSV | | GNTYLHWYLQKPGQSPKLLIYKVSNRFPGVPDR | |
| | KDRFTISRDDSQSILYLQMNNLKTEDTAMYYCT | | FSGSGSGTDFTLKISRVEAEDLGVYFCSQSTHV | |
| | AMDYWGQGTSVTVSS | | PYTFGGGTKLEIK | |
| SCT-Da231 | HVQLQQSGPELVRPGASVKLSCKASGYIFITYW | 152 | QIVLTQSPAIMSASPGEKVTISCSASSSVSYMY | 396 |
| | MTWVKQRPGQGLEWIGQIFPASGSTTYNEMFEG | | WYQQKPGSSPKPWIYRTSNLASGVPARFSGSGS | |
| | KATLTVDTSSSTAYMQLSSLTSEDSAVYYCASY | | GTSYSLTISSMEAEDAATYYCQQYHSFPRTFGG | |
| | DGYYSWFAYWGQGTLVTVSA | | GTKLEIK | |
| SCT-Da232 | QGQMQQSGAELVKPGASVKLSCKTSGFTFSHSY | 153 | DIQMTQSSSYLSVSLGGRVTITCKASDHINNWL | 397 |
| | ISWLKQKPGQSLEWIAWIYAGTGGTTYNQKFTG | | AWYQQKPGNAPRLLISGATSLETGVPSRFSGSG | |
| | KAQLTVDTSSSTAYMQFSSLTTEDSAIYYCARH | | SGKDYTLSITSLQIEDVATYSCQQYWSIPYTFG | |
| | ETYGSSPDYWGQGTTLTVSS | | GGTKLEIK | |
| SCT-Da233 | QGQMQQSGAELVKPGASVKLSCKTSGFTFSSFY | 154 | DIVMTQSHKFMSTSVGDRVNITCKASQDVNTAV | 398 |
| | ISWLKQKPGQSLEWIAWIDAGTGGTSYNQKFTG | | AWYQQKPGQSPKLLFYSTSYRYTGVPDRFTGSG | |
| | KAQLTVDTFSSTAYMQFSSLTTEDSAIYYCARH | | SGTDFTFTISSVQAEDLAVYYCQQHYSSPWTFG | |
| | VGRLRGYFDVWGAGTTVTVSS | | GGTKLEIK | |
| SCT-Da234 | QGQMQQSGAELVKPGASVKLSCKTSGFTFSSNY | 155 | DIVLTQSPAALSVTPGDSVSLSCRASQSISSTL | 399 |
| | ISWLKQKPGQSLEWIAWIYAGTGSTGYNQKFTG | | HWYQQKSHESPRLLIRYASQSISGIPSRFSGSG | |
| | KAQLTVDTFSSTAYMQFSSLTTEDSAIYYCASH | | SGTDFTLSINSVETEDFGMYFCHQSYSWPHTFG | |
| | DGHDAMDYWGQGTSVTVSS | | AGTKLELK | |
| SCT-Da235 | QGQMQQSGAELVKPGASVKLSCKTSGFTFSSNY | 156 | DIVMTQSHKFMSTSVGDRVSITCRASQDVSTAV | 400 |
| | ISWLNQKPGQSLEWIAWIDAGTGGSSYNQKFTG | | AWYQQKPGQSPKLLIYSASYRYTGVPDRFTVSG | |
| | KAQLTVDTSSSTAYMQFSSLTTEDSAIYYCARH | | SGTDFTFTISSVQAEDLAVYYCQQHYSTPWTFG | |
| | VGRLRGYFDVWGAGTRVTVSS | | GGTKLEIK | |
| SCT-Da236 | QGQMQQSGAELVKPGASVKLSCKTSGFTFSSRY | 157 | DIVLTQSPAALSVTPGDSVSLSCRASQSISNTL | 401 |
| | ISWLKQKPGQSLEWIAWIYAGTGGTGYNQKFTG | | HWYQQKSHESPRLLIRYASQSISGIPSRFSGSG | |
| | KAQLTVDTSSSTAYMQFSSLTTEDSAMYYCASH | | SGTDFTLSINSVETEDFGMYFCQQSNDWPHTFG | |
| | DGHDAMDYWGQGTSVTVSS | | AGTKLELK | |
| SCT-Da237 | QGQMQQSGAELVKPGASVKLSCKTSGFTFSSRY | 157 | DIVLTQSPAALSVTPGDSVSLSCRASQSISNTL | 402 |
| | ISWLKQKPGQSLEWIAWIYAGTGGTGYNQKFTG | | HWYQQKSHESPRLLIRYASQSISGIPSRFSGSG | |
| | KAQLTVDTSSSTAYMQFSSLTTEDSAMYYCASH | | SGTDFTLSINSVETEDFGMYFCQQSYDWPHTFG | |
| | DGHDAMDYWGQGTSVTVSS | | AGTKLELK | |
| SCT-Da238 | QGQMQQSGAELVKPGASVKLSCKTSGFTFSSRY | 158 | DIVLTQSPAALSVTPGDSVSLSCRASQSISNTL | 403 |
| | ISWLKQYPGQSLEWIAWIYAGTGGSGYNQKFTG | | HWYQQKSHESPRLLIRYASQSISGIPSRFSGSG | |
| | KAQLTVDTSSSTAYMQFSSLTTEDSAMYYCASH | | SGTDFTLSINSVETEDFGMYFCQQSYNWPHTFG | |
| | DGHDAMDYWGQGTSVTVSS | | AGTKLELK | |
| SCT-Da239 | QGQMQQSGAELVKPGASVKLSCKTSGFTFSSSY | 159 | DIQMTQSPASLSGSVGETVTITCRASENIYSFL | 404 |
| | ISWLKQKPGQSLEWIAWIYAGTGDTSYNQKFTG | | AWYQQKQGKSPQLLIYNARTLAGGVPSRFSGSR | |
| | KAQLTVDTSSSTAYMQFSSLTTEDSAIYYCASP | | SGTQFSLRINSLQPEDFGTYFCQHHYGPPFTFG | |
| | TGINWYFDVWGAGTTVTVSS | | GGTNLEIK | |
| SCT-Da240 | QGQMQQSGAELVKPGASVKLSCKTSGFTFSSSY | 160 | DIQMTQSPASLSASVGETVTITCRASENIYSFL | 405 |
| | ISWLKQKPGQSLEWIAWIYAGTGDTSYNQKFTG | | AWYQQKQGKSPQLLIYNARALAEGVTSRFSGSG | |
| | KAQLTVDTSSSTAYMQFSSLTTEDSAIYYCASP | | SGTQFSLRINSLQPEDFGTYFCQHHYGPPFTFG | |
| | TGVNWYFDVWGAGTTVTVSS | | GGTNLEIK | |
| SCT-Da241 | QGQMQQSGAELVKPGASVKLSCKTSGFTFSSSY | 161 | DIQMTQSSSYLSVSLGGRVTITCKASDHISNWL | 406 |
| | ISWLKQKPGQSLEWIAWIYAGTGGTSYNQKFTG | | AWYQQKPGNAPRLLISGATSLETGVPSRFSGSG | |
| | KAQLTVDTSSSTAYMQFSSLTTEDSAIYYCARP | | SGKDYTLSITSLQTEDVATYYCQQYWSLPYTFG | |
| | NGYGYGMDYWGQGTSVTVSS | | GGTKLEIK | |
| SCT-Da242 | QGQMQQSGAELVKPGASVKLSCKTSGFTFSSSY | 162 | DIQMTQSSSYLSVSLGGRVTITCKASDHINNWL | 407 |
| | ISWLKQKPGQSLEWIAWIYAGTGGTSYNQKFTG | | AWYQQKPGNAPRLLISGATSLETGVPSRFSGSG | |
| | KAQLTVDTSSSTAYMQFSSLTTEDSAIYYCARQ | | SGKDYTLSITSLQTEDVATYFCQQYWTIPYTFG | |
| | DSYGRGPDCWGQGTTLTVSS | | GGTKLEIK | |
| SCT-Da243 | QGQMQQSGAELVKPGASVKLSCKTSGFTFSSSY | 163 | DIQMTQSSSYLSVSLGGRVTITCKASDHINNWL | 408 |
| | ISWLKQKPGQSLEWIAWIYAGTGGTSYNQKFTG | | AWYQQKPGNAPRLLISGATSLETGVPSRFSGSG | |
| | KAQLTVDTSSSTAYMQFSSLTTEDSAIYYCARQ | | SGKDYTLSITSLQTEDVATYYCQQYWSIPYTFG | |
| | DSYGRSPDYWGQGTTLTVSS | | GGTKLEIK | |
| SCT-Da244 | QGQMQQSGAELVKPGASVKLSCKTSGFTFSSSY | 164 | DIQMTQSSSYLSVSLGGRVTITCKASDHINNWL | 397 |
| | ISWLKQKPGQSLEWIAWIYAGTGGTTYNQKFTG | | AWYQQKPGNAPRLLISGATSLETGVPSRFSGSG | |
| | KAQLTVDTSSSTAYMQFSSLTTEDSAIYYCARH | | SGKDYTLSITSLQIEDVATYSCQQYWSIPYTFG | |
| | ETYGSGPDYWGQGTTLTVSS | | GGTKLEIK | |
| SCT-Da245 | QGQMQQSGAELVKPGASVKLSCKTSGFTFSSSY | 165 | DIQMTQSPASLSASVGETVTITCRASGNIHNYL | 409 |
| | ISWLKQKPGQSLEWIAWIYAGTGGTTYNQKFTG | | AWYQQKQGKSPQLLVYNAKTLADGVPSRFSGSG | |
| | KAQLTVDTSSSTAYMQFSSLTTEDSAIYYCARH | | SGTQYSLKINSLQPEDFGSYYCQHFWSTPWTFG | |
| | ETYGSSPDYWGQGTTLTVSS | | GGTKLEIK | |
| SCT-Da246 | QGQMQQSGAELVKPGASVKLSCKTSGFTFSSSY | 165 | DIQMTQSSSYLSVSLGGRVTITCKASDHINNWL | 397 |
| | ISWLKQKPGQSLEWIAWIYAGTGGTTYNQKFTG | | AWYQQKPGNAPRLLISGATSLETGVPSRFSGSG | |
| | KAQLTVDTSSSTAYMQFSSLTTEDSAIYYCARH | | SGKDYTLSITSLQIEDVATYSCQQYWSIPYTFG | |
| | ETYGSSPDYWGQGTTLTVSS | | GGTKLEIK | |
| SCT-Da247 | QGQMQQSGAELVKPGASVKLSCKTSGFTFSSSY | 166 | DIQMTQSSSYLSVSLGGRVTITCKASDHINNWL | 305 |
| | ISWLKQKPGQSLEWIAWIYAGTGGTTYNQKFTG | | GWYQQKPGNAPRLLISGATSLETGVPSRFSGSG | |
| | KAQLTVDTSSSTAYMQFSSLTTEDSAIYYCARQ | | SGKDYTLSITSLQTEDVATYYCQQYWSIPYTFG | |
| | DSYGRSPDYWGQGTTLTVSS | | GGTKLEIK | |
| SCT-Da248 | QGQMQQSGAELVKPGASVKLSCKTSGFTFSSSY | 167 | DIQMTQSSSYLSVSLGGRVTITCKASDHINNWL | 397 |
| | ISWLKQKPGQSLEWIAWIYAGTGGTTYNQKFTG | | AWYQQKPGNAPRLLISGATSLETGVPSRFSGSG | |
| | RAQLTVDTSSSTAYMQFSSLTTEDSAIYYCARQ | | SGKDYTLSITSLQIEDVATYSCQQYWSIPYTFG | |
| | DSYGRSPDYWGQGTTLTVSS | | GGTKLEIK | |
| SCT-Da249 | QGQMQQSGAELVKPGASVKLSCRTSGFTFSSRY | 168 | DIVLTQSPATLSVTPGDRVSLSCRASQSINNYL | 410 |
| | ISWLKQKPGQSLEWIAWIYAGTGGTSYNQKFTG | | HWYQQKSHESPRLLIKYASHSISGIPSRFSGSG | |
| | KAQLTVDTSSSTAYMQFSSLTTEDSAIYYCASH | | SGTDFTLSINSVETEDFGVYFCQQSNSWPLTFG | |
| | DGLDAMDYWGQGTSVTVSS | | AGTKLELK | |
| SCT-Da250 | QGQMQQSGAELVKPGASVRLSCKTSGFTFSSFY | 169 | DIVMTQSHKFMSTSVGDRVSITCKASQDVSTAV | 411 |
| | ISWLKQKPGQSLEWIAWIYAGPGYTSYNQKFTG | | AWYQQKPGQSPKLLIYSASYRYTGVPDHFTVSG | |
| | KAQLTVDTSSSTAYMQFSSLTTEDSAIYYCARH | | SGTDFTFTISSVQAEDLAVYYCQQHYSTPWTFG | |
| | VGRQRGYFDVWGAGTTVTVSS | | GGTKLEIK | |
| SCT-Da251 | QGQMQQSGAELVKSGASVKLSCKTSGFTFSSSY | 170 | DIQMTQSSSYLSVSLGGRVTITCKASDHINNWL | 412 |
| | ISWLKQKPGQSLEWIAWIYAGTGGTTYNEKFTG | | AWYQQKPGNAPRLLISGATSLEPGVPSRFSGSG | |
| | KAQLTVDTSSSTAYMQFSSLTTEDSAIYYCGRH | | SGKDYTLTITSLQTEDVATYYCQQYWSLPYMFG | |
| | NGYGIGMDYWGQGTSVTVSS | | GGTKLEIK | |
| SCT-Da252 | QGQMQQSGGELVKPGASVKLSCKTSGLTFSSSY | 171 | DIVMTQSHKFMSTSVGDRVSITCKASQDVTTAV | 413 |
| | ISWLKQKPGQSLEWIAWIDAGTGGTSYNQKFTG | | AWYQQKPGQSPKLLIYSASYRYTGVPDRFTVSG | |
| | KAQLTVDTSSSTAYMQFSSLTTEDSAIYYCARH | | SGTDFTLTISSVQAEDLAVYYCQQHYSAPWTFG | |
| | VGRLRGYFDVWGAGTTVTVSS | | GGTKLKIT | |
| SCT-Da253 | QGQMQQSGPELVKPGASVKLSCKTSGFTFSSNY | 172 | DIVLTQSPAALSVTPGDSVSLSCRASQSISNTL | 414 |
| | ISWLKQKPGQSLEWIAWIYAGTGGTGYNQKFTG | | HWYQQKSHESPRLLIRYASQSISGIPSRFSGSG | |
| | KAQLTVDTSSSTAYMQFSSLTTEDSAIYYCASH | | SGTDFTLSINSVETEDFGMYFCQQSNSWPHTFG | |
| | DGHDAMDYWGQGTSVTVSS | | AGTKLELK | |
| SCT-Da254 | QIQLQQSGAELVRPGVSVKISCKGSGYTFTDYP | 173 | DIVLTQSPASLAVSLGQRATISCRASKSVSTSG | 415 |
| | IHWVKQSHTTSLEWIGVIITYYGDATYNQKFKG | | YSYMHWYQQKPGQAPKLLIYLASNLESGVPARF | |
| | KATMTVDKSSSTAYMELARLTSDDSAIYYCASH | | SGSGSGTDFTLNIQPVEEEDAATYYCQHSRELY | |
| | RGKGYFDVWGAGTTVTVSS | | TFGGGTKLEIK | |
| SCT-Da255 | QIQLQQSGAELVRPGVSVKISCKGSGYTFTDYP | 173 | QIVLTQSPALMSASPGEKVTMTCSASSSVSYMY | 333 |
| | IHWVKQSHTTSLEWIGVIITYYGDATYNQKFKG | | WYQQKPRSSPKPWIYLTSNLASGVPARFSGSGS | |
| | KATMTVDKSSSTAYMELARLTSDDSAIYYCASH | | GTSYSLTISSMEAEDAATYYCQQWSSNPLTFGA | |
| | RGKGYFDVWGAGTTVTVSS | | GTKLELK | |
| SCT-Da256 | QIQLQQSGPELVKPGASVKISCKASGYTFTDYF | 174 | DIVLTQSPATLSVTPGDRVSLSCRASQSISNYL | 416 |
| | INWVKQKPGQGLEWIGWIYPGNINTNYNENFKG | | HWYQQKSRESPRLLIKYASQSISGIPSRFSGSG | |
| | KATLTVDTSSSTAYMQLSSLTSEDTAVYFCAGG | | SGTDFTLSINSVETEDFGMYFCQQSNSWPLTFG | |
| | NYALDYWGQGTSVTVSS | | AGTKLELK | |
| SCT-Da257 | QIQLQQSGPELVKPGASVKISCKASGYTFTDYY | 175 | DIVLTQSPATLSVTPGDRVSLSCRASQSISNYL | 417 |
| | INWVKQKPGQGLEWIGCIYPGNDNSNYNEKFKG | | HWYQQKSHESPRLLIKYASQSISGIPSRFSGSG | |
| | KATLTVDTSSSTAYMQLSSLTSEDTAVYFCAGG | | SGTDFTLSINSVETEDFGMYFCQQSNSWPLTFG | |
| | NYAMDYWGQGTSVTVSS | | AGTKLELK | |
| SCT-Da258 | QIQLQQSGPELVNPGASVMISCKASGYTFTAYY | 176 | DIVLTQSPATLSVTPGDRVSLSCRASQSISNYL | 418 |
| | MNWVKQKPGQGLEWIGWIYPGNDNTNYNEKFKG | | HWYQQKSHESPRLLIKYASQSISGIPSRFSGSG | |
| | KATLTVDTSSSTAFMQLSSLTSEDTAVYFCAGG | | SGTDFTLSISSVETEDFGMYFCQQSNSWPLTFG | |
| | NYAMDYWGLGTSVTVSS | | AGTKLELK | |
| SCT-Da259 | QIQLVQSGPELKKPGETVKISCKASGYTFTNSG | 177 | QIVLSQSPAILSASPGEKVTMTCRASSSVSYMH | 419 |
| | MNWVKQAPGKGLKWMGWINTYTGEPTYVDDFKG | | WYQQKPGSSPKPWIFATSNLAYGVPDRFSGSGS | |
| | RFAFSLETSASTAYLQINNLKNEDTATYFCAPT | | GTSYSLTISRMEAEDAATYYCQQWSSTPPTFGG | |
| | IQVRRLFDYWGQGTTLTVSS | | GTKLEIK | |
| SCT-Da260 | QIQLVQSGPELKKPGETVKISCKASGYTFTNSG | 178 | QIVLSQSPAILSASPGEKVTMTCRASSSVSYMH | 419 |
| | MNWVKQAPGKGLKWMGWINTYTGEPTYVDDFKG | | WYQQKPGSSPKPWIFATSNLAYGVPDRFSGSGS | |
| | RFAFSLETSASTAYLQINNLKNEDTATYFCAPT | | GTSYSLTISRMEAEDAATYYCQQWSSTPPTFGG | |
| | IQVRRLVDYWGQGTTLTVSS | | GTKLEIK | |
| SCT-Da261 | QIQLVQSGPELKKPGETVKISCKASGYTFTNYG | 179 | DIVLTQSPASLAVSLGQRATISCRASESVDSYG | 420 |
| | MNWVKQAPGKGLKWMGWINTNTGEPTYAEEFKG | | NSFMHWYQQKPGQPPKLLIYRASNLESGIPARF | |
| | RFAFSLETSASTAYLQINNLKNEDTATYFCARR | | SGSGSRTDFTLTINPVEADDVATYYCQQSNEDP | |
| | GYYGRGDYWGQGTTLTVSS | | YTFGGGTKLEIK | |
| SCT-Da262 | QVQLKESGPGLVAPSQSLSITCTVSGFSLTSYD | 180 | SIVMTQTPKFLLVSAGDRVTITCKASQSVSNDV | 421 |
| | ISWIRQPPGKGLEWLGVIWTGGGTNYNSAFMSR | | AWYQQKPGQSPKLLIYYASNRYTGVPDRFTGSG | |
| | LSISKDNSKSQVFLKMNSLQTDDTAIYYCVRWG | | YGTDFTFTISTVQAEDLAVYFCQQDYSSPPTFG | |
| | DDYDWFAYWGQGTLVTVSA | | GGTKLEIK | |
| SCT-Da263 | QVQLKQSGPSLVQPAQSLSITCTVSGFSLTTYG | 181 | DIVMSQSPSSLAVSVGEKVTMSCKSSLSLLYSS | 422 |
| | VHWLRQSPGKGLEWLGVIWRGGGTDYNAPFMSR | | NQKNYLAWYQQKPGQSPKLLIYWASTRESGVPD | |
| | LSITKDDSKSQLFFKMNSLHADDTAIYYCAIYS | | RFTGSGSGTGFTLTISSVKAEDLAVYYCQQYYS | |
| | DHDTYALDYWGQGTSVTVSS | | YPLTFGAGTKLELK | |
| SCT-Da264 | QVQLQHSGAELVRPGVSVKISCKGSGYTFTDYA | 182 | DIVMTQSHKFMSTSVGDRVSITCKASQDVGTAV | 423 |
| | MHWVKQSHAKSLEWIGVISNYNGDVTYNQKFKG | | AWYQQKPGQSPKLLIYRASTRHTGVPDRFTGSG | |
| | KATMTVDKSSSTAYMELARLTSEDSAIYYCARR | | SGTDFILTISNVQSEDLADYFCQQYSTYYTFGG | |
| | HGNRAMDYWGQGTSVTVSS | | GTKLEIK | |
| SCT-Da265 | QVQLQQPGAELVKPGASVKLSCKASGYSFTSYY | 183 | DIVMTQSHKFMSTSVGDRVSITCKASQDVSTAV | 424 |
| | MYWVKQRPGHGLEWIGEINPSNGGTYFNVKFQS | | AWYQQKPGQSPKLLIYSASYRYTGVPDRFTGSG | |
| | KATLTVDKSSTTAYMQLSSLTSEDSAVYFCTRE | | SGTDFTFTISSVQAEDLAVYYCQQHYSTPWTFG | |
| | GNYGYSWYAYWGQGTLVTVSA | | GGTKLEIK | |
| SCT-Da266 | QVQLQQPGAELVKPGASVKLSCKASGYTFTSYW | 184 | DVVMTQTPLTLSVTIGQPASISCKSSQSLLDSD | 425 |
| | MHWVKQRPGQGLEWIGEIDPSDSYTNYNQKFKG | | GKTYLNWLLQRPGQSPKRLIYLVSKLDSGVPDR | |
| | KATLTVDKSSSTAYMQLSSLTSEDSAVYYCAGG | | FTGSGSGTDFTLKISRVEAEDLGVYYCWQGTHF | |
| | IHFDYWGQGTTLTVSS | | PFTFGSGTKLEIK | |
| SCT-Da267 | QVQLQQPGAELVKPGASVKMSCKASGYTFTSYN | 185 | DIVLTQSPASLAVSQGQRATISCRASESVDSYG | 426 |
| | LHWLKLTPGQGLEWIGIIYPGKDDTSYNQKFKG | | YSFMHWYQQKPGQPPKLLIYLASNLESGVPARF | |
| | KATLTADTSSSTAYMQLSSLTSEDSAVYYCARV | | SGSGSRTDFTLTIDPVEADDAATYYCQQNNEDP | |
| | YYGNYGGFDYWGQGTLVTVSA | | FTFGSGTKLEIK | |
| SCT-Da268 | QVQLQQPGSELVRPGASVKLSCKASGYTFTSYW | 186 | DIVMTQTPLSLPVSLGDQASISCRSSQSLVHSN | 427 |
| | MYWVKQRPGQGLEWIGNIYPGSGSVNYDEKFKS | | GNTYLHWYLQKPGQSPKLLIYKVSNRFSGVPDR | |
| | KATLTVDTSSSTAYMQLSSLTSEDSAVYYCTRG | | FSGSGSGTDFTLKISRVEAEDLGVYFCSQSTHV | |
| | GIGVRSPFDVWGAGTTVTVSS | | WTFGGGTKLEIK | |
| SCT-Da269 | QVQLQQPGSVLVRPGASVKLSCKASGYTFTSSW | 187 | DIQMTQSSSYLSVSLGGRVTITCKASDHINNWL | 428 |
| | MHWAKQRPGQGLEWIGEIHPNSGNTNYNEKFKG | | AWYQQKPGNAPRLLISGATSLETGIPSRFSGSG | |
| | KATLTIDTSSSTAYVDLSSLASEDSAVYYCVRL | | SGKDYSLSITSLQIEDVATYSCQQYWSIPYTFG | |
| | GLFDFWGQGTTLTVSS | | GGTKLEIK | |
| SCT-Da270 | QVQLQQSGAELAKPGASVKMSCKASGYTFTNYW | 188 | DIQMTQSPASLAASVGETVTITCRTSENIYYSL | 429 |
| | MHWIKQRPGQGLEWIGYISPRTGNTEYNQKFKD | | AWFQQKQGKSPQLLIYNANSLEDGVPSRFSASG | |
| | KATLTADKSSSIVYMQLSSLTSEDSAVYYCTRG | | SGTQFSMKIKSMQPEDTATYFCKQSYDVPYTFG | |
| | EAYWGQGTLVTVSA | | GGTKLEIK | |
| SCT-Da271 | QVQLQQSGAELAKPGASVKMSCKASGYTFTNYW | 188 | DIQMTQSPASLAASVGETVTITCRTSENIYYSL | 430 |
| | MHWIKQRPGQGLEWIGYISPRTGNTEYNQKFKD | | AWFQQKQGKSPQLLIYNANSLEDGVPSRFSGSG | |
| | KATLTADKSSSIVYMQLSSLTSEDSAVYYCTRG | | SGTQFSMKIKSMQPEDTATYFCKQSYDVPYTFG | |
| | EAYWGQGTLVTVSA | | GGTKLEIK | |
| SCT-Da272 | QVQLQQSGAELAKPGASVKMSCKASGYTFTNYW | 189 | DIQMTQSPASLAASVGETVTITCRTSENIYYSL | 431 |
| | MHWVKQRPGQGLEWIGYISPRTGNTEYNQKFKD | | AWFQLKQGKSPQLLIYNANTLEDGVPSRFSASG | |
| | KATLTADKSSSIVYIQLSSLTSEDSAVYYCTRG | | SGTQFSMKIKSMQPEDTATYFCKQSYDVPYTFG | |
| | EAYWGQGTLVTVSA | | GGTKLEIK | |
| SCT-Da273 | QVQLQQSGAELAKPGASVKMSCKASGYTFTNYW | 190 | DIQMTQSPASLAASVGETVTITCRTSENIYYSL | 429 |
| | MHWVKQRPGQGLEWIGYISPRTGNTEYNQKFKD | | AWFQQKQGKSPQLLIYNANSLEDGVPSRFSASG | |
| | KATLTADKSSSIVYMQLSSLTSEDSALYYCTRG | | SGTQFSMKIKSMQPEDTATYFCKQSYDVPYTFG | |
| | EAYWGQGTLVTVSA | | GGTKLEIK | |
| SCT-Da274 | QVQLQQSGAELAKPGASVKMSCKASGYTFTNYW | 190 | QIVLTQSPALMSASPGEKVTMTCSASSSVSYIY | 387 |
| | MHWVKQRPGQGLEWIGYISPRTGNTEYNQKFKD | | WYQQKPRSSPKPLIYLTSNLASGVPARFSGSGS | |
| | KATLTADKSSSIVYMQLSSLTSEDSALYYCTRG | | GTSYSLTISSMEAEDAASYYCQQWSSNPLTFGA | |
| | EAYWGQGTLVTVSA | | GTKLELK | |
| SCT-Da275 | QVQLQQSGAELAKPGASVKMSCKASGYTFTNYW | 191 | DIQMTQSPASLAASVGETVTITCRTSENIYYSL | 430 |
| | MHWVKQRPGQGLEWIGYISPRTGNTEYNQKFKD | | AWFQQKQGKSPQLLIYNANSLEDGVPSRFSGSG | |
| | KATLTADKSSSIVYMQLSSLTSEDSAVYYCARG | | SGTQFSMKIKSMQPEDTATYFCKQSYDVPYTFG | |
| | EAYWGQGTLVTVSA | | GGTKLEIK | |
| SCT-Da276 | QVQLQQSGAELAKPGASVKMSCKASGYTFTNYW | 192 | DIQMTQSPASLAASVGETVTITCRTSENIYYSL | 432 |
| | MHWVKQRPGQGLEWIGYISPRTGNTEYNQKFKD | | AWFQLKQGKSPQLLIYNANSLEDGVPSRFSGSG | |
| | KATLTADKSSSIVYMQLSSLTSEDSAVYYCTRG | | SGTQFSMKIKSMQPEDSATYFCKQSYDVPYTFG | |
| | EAYWGQGTLVTVSA | | GGTKLEIK | |
| SCT-Da277 | QVQLQQSGAELAKPGASVKMSCKASGYTFTNYW | 193 | DIQMTQSPASLAASVGETVTITCRTSENIYYSL | 433 |
| | MHWVKQRPGQGLEWIGYISPRTGNTEYNQKFKD | | AWFQQKQGKSPQLLIYNAKSLEDGVPSRFSASG | |
| | KATLTADKSSSIVYMQLSSLTSEDSAVYYCTRG | | SGTQFSMKIKSMQPEDTATYFCKQSYDVPYTFG | |
| | EAYWGQGTLVTVSA | | GGTKLEIK | |
| SCT-Da278 | QVQLQQSGAELAKPGASVKMSCKASGYTFTNYW | 194 | DIQMTQSPASLAASVGETVTITCRTSENIYYSL | 434 |
| | MHWVKQRPGQGLEWIGYISPRTGNTEYNQKFKG | | AWFQQKQGKSPQLLIYNANSLEDGVPSRFSGSG | |
| | KATLTADKSSSIVYMHLSSLTSEDSAVYYCARG | | SGTQFSMKIKSMQPEDTATYFCKQAYDVPYTFG | |
| | EAYWGQGTLVTVSA | | GGTKLEIK | |
| SCT-Da279 | QVQLQQSGAELAKPGASVKMSCKASGYTFTNYW | 194 | DIQMTQSPASLAASVGETVTITCRTSENIYYSL | 434 |
| | MHWVKQRPGQGLEWIGYISPRTGNTEYNQKFKG | | AWFQQKQGKSPQLLIYNANSLEDGVPSRFSGSG | |
| | KATLTADKSSSIVYMHLSSLTSEDSAVYYCARG | | SGTQFSMKIKSMQPEDTATYFCKQAYDVPYTFG | |
| | EAYWGQGTLVTVSA | | GGTKLEIK | |
| SCT-Da280 | QVQLQQSGAELAKPGASVKMSCKASGYTFTNYW | 196 | DIQMTQSPASLAASVGETVTITCRTSENIYYSL | 429 |
| | MHWVKQRPGQGLEWIGYISPRTGNTEYNQKFRD | | AWFQQKQGKSPQLLIYNANSLEDGVPSRFSASG | |
| | KATLTADKSSSIVYMQLSSLTSEDSAVYYCTRG | | SGTQFSMKIKSMQPEDTATYFCKQSYDVPYTFG | |
| | EAYWGQGTLVTVSA | | GGTKLEIK | |
| SCT-Da281 | QVQLQQSGAELAKPGASVKMSCKASGYTFTNYW | 197 | DIQMTQSPASLAASVGETVTITCRTSENIYYSL | 434 |
| | MHWVKQRPGQGLEWIGYISPRTGNTNYNQRFKD | | AWFQQKQGKSPQLLIYNANSLEDGVPSRFSGSG | |
| | KATLTADKSSSIVYMQLSSLTSEDSAVYYCARG | | SGTQFSMKIKSMQPEDTATYFCKQAYDVPYTFG | |
| | EAYWGQGTLVTVSA | | GGTKLEIK | |
| SCT-Da282 | QVQLQQSGAELAKPGASVKMSCKASGYTFTNYW | 198 | DIQMTQSPASLAASVGETVTITCRASENIYYTL | 435 |
| | MHWVKQRPGQGLEWIGYISPRTTYTEYNQKFKD | | AWFQQKQGNSPQLLIYNANSLEDGVPSRFSGSG | |
| | KATLTADKSSSTAFLQLSSLTSEDSAVYYCTRG | | SGTQYSMKINSMQPEDTATYFCKQAYDVPYTFG | |
| | EAYWGQGTLVTVSA | | GGTKLEIK | |
| SCT-Da283 | QVQLQQSGAELAKPGASVKMSCKASGYTFTTYW | 199 | DIQMTQPTSSLSASLGDRVTISCRASQDISNYL | 252 |
| | MHWVKQRPGQGLEWIGYINPSTGYTYYNQKFKD | | NWYQQKPDGTVKLLIYYTSRLHSGVPSRFSGSG | |
| | KATLTADKSSSTAYMQLSSLTSEDSAVYFCASY | | SGTDYSLTISNLDQEDIATYFCQQGNTLPWTFG | |
| | YGTSQAWFAYWGQGTLVTVSA | | GGTKLEIK | |
| SCT-Da284 | QVQLQQSGAELAKPGASVKMSCKASGYTFTTYW | 199 | DIVLTQSPASLAVSLGQRATISCRASESVDNFG | 301 |
| | MHWVKQRPGQGLEWIGYINPSTGYTYYNQKFKD | | NSFMHWYQQKPGQPPKLLIYLASNLESGVPARF | |
| | KATLTADKSSSTAYMQLSSLTSEDSAVYFCASY | | SGSGSRTDFTLTIDPVEADDAATYYCQQSNEDP | |
| | YGTSQAWFAYWGQGTLVTVSA | | RTFGGGTKLEIK | |
| SCT-Da285 | QVQLQQSGAELAKPGASVKMSCKASGYTFTTYW | 199 | DIVLTQSPASLAVSLGQRATISCRASESVDSYG | 264 |
| | MHWVKQRPGQGLEWIGYINPSTGYTYYNQKFKD | | NSFMHWYQQKPGQPPKLLIYIASNLESGVPARF | |
| | KATLTADKSSSTAYMQLSSLTSEDSAVYFCASY | | SGSGSRTDFTLTIDPVEADDAATYYCQQSNEDP | |
| | YGTSQAWFAYWGQGTLVTVSA | | RTFGGGTKLEIK | |
| SCT-Da286 | QVQLQQSGAELAKPGASVKMSCKASGYTFTTYW | 199 | DIVMTQFQKFMSTSVGDRVSVTCKASQNVDTNV | 293 |
| | MHWVKQRPGQGLEWIGYINPSTGYTYYNQKFKD | | AWYQQKPGQSPKLLIYSGSYRFSGVPDRFTGSG | |
| | KATLTADKSSSTAYMQLSSLTSEDSAVYFCASY | | SGTDFTLTISNVQSEDLAEYFCQQYNTYPYTFG | |
| | YGTSQAWFAYWGQGTLVTVSA | | GGTKLEIK | |
| SCT-Da287 | QVQLQQSGAELAKPGASVKMSCKASGYTFTTYW | 199 | DIVMTQSPATLSVTPGDRVSLSCRASQRISDYL | 337 |
| | MHWVKQRPGQGLEWIGYINPSTGYTYYNQKFKD | | HWYQQKSHESPRLLIKYASQSISGIPSRFSGSG | |
| | KATLTADKSSSTAYMQLSSLTSEDSAVYFCASY | | SGSDFTLSINSVEPEDVGMYYCQNGHSFPFTFG | |
| | YGTSQAWFAYWGQGTLVTVSA | | SGTKLEIK | |
| SCT-Da288 | QVQLQQSGAELAKPGASVKMSCKASGYTFTTYW | 199 | DIVMTQSQKFMSTSVGDRVSVTCKASQNVDTNV | 294 |
| | MHWVKQRPGQGLEWIGYINPSTGYTYYNQKFKD | | AWYQQKPGQSPKLLIYSASYRFSGVPDRFTGSG | |
| | KATLTADKSSSTAYMQLSSLTSEDSAVYFCASY | | SGTDFTLTISNVQSEDLAEYFCQQYNSYPYTFG | |
| | YGTSQAWFAYWGQGTLVTVSA | | GGTKLEIK | |
| SCT-Da289 | QVQLQQSGAELAKPGASVKMSCKASGYTFTTYW | 199 | DIVMTQSQKFMSTSVGDRVSVTCKASQNVGTDV | 318 |
| | MHWVKQRPGQGLEWIGYINPSTGYTYYNQKFKD | | AWYQQKPGQSPKLLIYSASYRFSGVPDRFTGSG | |
| | KATLTADKSSSTAYMQLSSLTSEDSAVYFCASY | | SGTDFTLTISNVQSEDLAEYFCQQYNSYPYTFG | |
| | YGTSQAWFAYWGQGTLVTVSA | | GGTKLEIK | |
| SCT-Da290 | QVQLQQSGAELAKPGASVKMSCKASGYTFTTYW | 199 | EIVLTQSPTTMAASPGEKITIICNASSSISSNY | 358 |
| | MHWVKQRPGQGLEWIGYINPSTGYTYYNQKFKD | | FNWYQQKPEFSPKLLIYRTSNLASGVPARFSGS | |
| | KATLTADKSSSTAYMQLSSLTSEDSAVYFCASY | | GSGTSYSLTIGTMEAEDVATYYCQQGSSLPRTF | |
| | YGTSQAWFAYWGQGTLVTVSA | | GSGTKLEIK | |
| SCT-Da291 | QVQLQQSGAELAKPGASVKMSCKASGYTFTTYW | 199 | EIVLTQSPTTMAASPGEKITITCSASSSISSNY | 312 |
| | MHWVKQRPGQGLEWIGYINPSTGYTYYNQKFKD | | LNWYQQKPEFSPKLLIYRTSNLASGVPARFSGS | |
| | KATLTADKSSSTAYMQLSSLTSEDSAVYFCASY | | GSGTSYSLTIGTMEAEDVATYYCQQGSSIPRTF | |
| | YGTSQAWFAYWGQGTLVTVSA | | GSGTKLEIK | |
| SCT-Da292 | QVQLQQSGAELAKPGASVKMSCKASGYTFTTYW | 199 | QIVLTQSPAIMSASPGEKVTMTCSASSSVSYMH | 371 |
| | MHWVKQRPGQGLEWIGYINPSTGYTYYNQKFKD | | WYQQKSGTSPKRWIYDTSKLASGVPARFSGSGS | |
| | KATLTADKSSSTAYMQLSSLTSEDSAVYFCASY | | GTSYSLTISSMEAEDAATYYCQQWSSNPLTFGA | |
| | YGTSQAWFAYWGQGTLVTVSA | | GTKLELK | |
| SCT-Da293 | QVQLQQSGAELAKPGASVKMSCKASGYTFTTYW | 199 | QIVLTQSPALMAASPGEKVTITCSVSSSISYMY | 366 |
| | MHWVKQRPGQGLEWIGYINPSTGYTYYNQKFKD | | WYQQKSETSPKPWIYGTSNLASGVPVRFSGSGS | |
| | KATLTADKSSSTAYMQLSSLTSEDSAVYFCASY | | GTSYSLTISSMEAEDAATYYCQQWSSYPLTFGA | |
| | YGTSQAWFAYWGQGTLVTVSA | | GTKLELK | |
| SCT-Da294 | QVQLQQSGAELARPGASVKLSCKASGYTFTSYW | 200 | DIQMTQSPASLAASVGETVTITCRTSENIYYSL | 430 |
| | MQWIKQRPGQGLEWIGAIYPGDGDTRYTQKFKG | | AWFQQKQGKSPQLLIYNANSLEDGVPSRFSGSG | |
| | KATLTADKSSSTAYMQLSSLASEDSAVYYCARV | | SGTQFSMKIKSMQPEDTATYFCKQSYDVPYTFG | |
| | EADDLWYFDVWGAGTTVTVSS | | GGTKLEIK | |
| SCT-Da295 | QVQLQQSGAELARPGASVKLSCKASGYTFTSYW | 200 | DIVLTQSPASLAVSLGQRATISCKASQSVDYDG | 328 |
| | MQWIKQRPGQGLEWIGAIYPGDGDTRYTQKFKG | | DNYMNWYQQKPGQPPKLLIYAASNLESGIPARF | |
| | KATLTADKSSSTAYMQLSSLASEDSAVYYCARV | | SGSGSGTDFTLNIHPVEEEDAATYYCQQSYEDP | |
| | EADDLWYFDVWGAGTTVTVSS | | WTFGGGTKLEIK | |
| SCT-Da296 | QVQLQQSGAELARPGASVKLSCKASGYTFTSYW | 200 | DIVLTQSPASLAVSLGQRATISCRASESVDSYG | 264 |
| | MQWIKQRPGQGLEWIGAIYPGDGDTRYTQKFKG | | NSFMHWYQQKPGQPPKLLIYIASNLESGVPARF | |
| | KATLTADKSSSTAYMQLSSLASEDSAVYYCARV | | SGSGSRTDFTLTIDPVEADDAATYYCQQSNEDP | |
| | EADDLWYFDVWGAGTTVTVSS | | RTFGGGTKLEIK | |
| SCT-Da297 | QVQLQQSGAELARPGASVKLSCKASGYTFTSYW | 200 | DIVMTQSHKFMSTSVGDRVSITCKASQDVSTAV | 411 |
| | MQWIKQRPGQGLEWIGAIYPGDGDTRYTQKFKG | | AWYQQKPGQSPKLLIYSASYRYTGVPDHFTVSG | |
| | KATLTADKSSSTAYMQLSSLASEDSAVYYCARV | | SGTDFTFTISSVQAEDLAVYYCQQHYSTPWTFG | |
| | EADDLWYFDVWGAGTTVTVSS | | GGTKLEIK | |
| SCT-Da298 | QVQLQQSGAELARPGASVKLSCKASGYTFTSYW | 200 | DIVMTQSHKFMSTSVGDRVSITCKASQDVTTAV | 413 |
| | MQWIKQRPGQGLEWIGAIYPGDGDTRYTQKFKG | | AWYQQKPGQSPKLLIYSASYRYTGVPDRFTVSG | |
| | KATLTADKSSSTAYMQLSSLASEDSAVYYCARV | | SGTDFTLTISSVQAEDLAVYYCQQHYSAPWTFG | |
| | EADDLWYFDVWGAGTTVTVSS | | GGTKLKIT | |
| SCT-Da299 | QVQLQQSGAELARPGASVKLSCKASGYTFTSYW | 200 | DIVMTQSQKFMSTSVGDRVSVTCKASQNVGTDV | 304 |
| | MQWIKQRPGQGLEWIGAIYPGDGDTRYTQKFKG | | AWYQQKPGQSPKLLIYSASYRFSGVPDRFTGSG | |
| | KATLTADKSSSTAYMQLSSLASEDSAVYYCARV | | SGTDFSLAISNVQSEDLAEYFCQQYNSYPYTFG | |
| | EADDLWYFDVWGAGTTVTVSS | | GGAKLEIK | |
| SCT-Da300 | QVQLQQSGAELARPGASVKLSCKASGYTFTSYW | 200 | DIVMTQSQKFMSTSVGDRVSVTCKASQNVGTDV | 318 |
| | MQWIKQRPGQGLEWIGAIYPGDGDTRYTQKFKG | | AWYQQKPGQSPKLLIYSASYRFSGVPDRFTGSG | |
| | KATLTADKSSSTAYMQLSSLASEDSAVYYCARV | | SGTDFTLTISNVQSEDLAEYFCQQYNSYPYTFG | |
| | EADDLWYFDVWGAGTTVTVSS | | GGTKLEIK | |
| SCT-Da301 | QVQLQQSGAELARPGASVKLSCKASGYTFTSYW | 200 | DNVMTQSQKFMSTSVGDRVSVTCKASQNVGTNV | 324 |
| | MQWIKQRPGQGLEWIGAIYPGDGDTRYTQKFKG | | AWYQQKSGQSPKLLIYSASYRFSGVPDRFTGSG | |
| | KATLTADKSSSTAYMQLSSLASEDSAVYYCARV | | SGTDFTLTISNVQSEDLAEYFCQQYNSYPYTFG | |
| | EADDLWYFDVWGAGTTVTVSS | | GGTKLEIK | |
| SCT-Da302 | QVQLQQSGAELARPGASVKLSCKASGYTFTSYW | 200 | QIVLTQSPALMSASPGEKVTMTCSASSSVSYMY | 333 |
| | MQWIKQRPGQGLEWIGAIYPGDGDTRYTQKFKG | | WYQQKPRSSPKPWIYLTSNLASGVPARFSGSGS | |
| | KATLTADKSSSTAYMQLSSLASEDSAVYYCARV | | GTSYSLTISSMEAEDAATYYCQQWSSNPLTFGA | |
| | EADDLWYFDVWGAGTTVTVSS | | GTKLELK | |
| SCT-Da303 | QVQLQQSGAELARPGASVKLSCKASGYTFTSYW | 200 | SIVMTQTPKFLLVSAGDRVTITCRASQSVTNDV | 265 |
| | MQWIKQRPGQGLEWIGAIYPGDGDTRYTQKFKG | | AWYQQKPGQSPKLLIYYASNRYTGVPDRFTGSG | |
| | KATLTADKSSSTAYMQLSSLASEDSAVYYCARV | | YGTDFTFTISTVQAEDLAVYFCQQDYNSPYTFG | |
| | EADDLWYFDVWGAGTTVTVSS | | GGTKLEIK | |
| SCT-Da304 | QVQLQQSGAELSKPGASVKMSCKASGYTFTTYW | 201 | DIQMTQSPASLAASVGETVTITCRTSENIYYSL | 429 |
| | MHWVKQRPGQGLEWIGYINPSTGYTYYNQKFKD | | AWFQQKQGKSPQLLIYNANSLEDGVPSRFSASG | |
| | KATLTADKSSSTAYMQLSSLTSEDSAVYFCASY | | SGTQFSMKIKSMQPEDTATYFCKQSYDVPYTFG | |
| | YGTSQAWFAYWGQGTLVTVSA | | GGTKLEIK | |
| SCT-Da305 | QVQLQQSGAELVRPGTSVKVSCKASGYAFTNYL | 202 | DIQMTQSPSSLSASLGERVSLTCRASQEISGYL | 436 |
| | IEWVKQRPGQGLEWIGVINPGSGGPNYNEKFKG | | TWLQQKPDGTIKRLIYAASTLDSGVPKRFSGSR | |
| | KATLTADKSSSTAYMQLSSLTSDDSAVYFCASD | | SESDYSLTISSLESEDFADYYCLQSASYPPTFG | |
| | NYGNYDVLDYWGQGTSVTVSS | | GGTKLEIK | |
| SCT-Da306 | QVQLQQSGAELVRPGTSVKVSCKASGYAFTNYL | 203 | DIVMTQAAPSVPVTPGESVSISCRSSKSLLHSN | 437 |
| | IEWVKQRPGQGLEWIGVINPGSGGTNYNEKFKG | | GNTYLYWFLQRPGQSPQLLIYRMSNLASGVPDR | |
| | KATLTADKSSSTAYMQLSSLTSDDSAVYFCDRE | | FSGSGSGTAFTLRISRVEAEDVGVYYCMQHLEY | |
| | DAYWGQGTLVTVSA | | PYTFGGGTKLEIK | |
| SCT-Da307 | QVQLQQSGPDLVRPGVSVKISCKGSGYTFTDYA | 204 | DIVMTQSPATLSVTPGDRVSLSCRASQSISDYL | 438 |
| | LHWVKQSHAKSLEWIGVINTYSSNTYYNQNFEG | | HWYQQKSHESPRLLIKYASQSISGIPSRFSGSG | |
| | KATMTVDKSSNTAYMELARLTSEDSAIYYCARR | | SGSDFTLSINSVEPEDVGVYYCQNGHSFPFTFG | |
| | RGSHRDAYFDYWGQGTTLTVSS | | SGTKLEIK | |
| SCT-Da308 | QVQLQQSGPDLVRPGVSVKISCKGSGYTFTDYA | 204 | DIVMTQSPATLSVTPGDRVSLSCRASQSISDYL | 439 |
| | LHWVKQSHAKSLEWIGVINTYSSNTYYNQNFEG | | HWYQQKSHESPRLLIKYASQSISGIPSRFSGSG | |
| | KATMTVDKSSNTAYMELARLTSEDSAIYYCARR | | SGSDFTLSINSVEPEDVGVYYCQNGHSFPFTFG | |
| | RGSHRDAYFDYWGQGTTLTVSS | | SGTRLEIK | |
| SCT-Da309 | QVQLQQSGPDLVRPGVSVKISCKGSGYTFTDYA | 205 | DIVMTQSPATLSVTLGDRVSLSCRASQSISDYL | 440 |
| | LHWVKQSHAKSLEWIGVINTYSSNTYYNQNFEG | | HWYQQKSHESPRLLIKYASQSISGIPSRFSGSG | |
| | KATMTVDKSSSTAYMELARLTSEDSAIYYCARR | | SGSDFTLSINSVEPEDVGVYYCQNGHSFPFTFG | |
| | RGSHRDAYFDYWGQGTTLTVSS | | SGTKLEIK | |
| SCT-Da310 | QVQLQQSGPDLVRPGVSVKISCKGSGYTFTDYA | 205 | DIVMTQSPATLSVTPGDRVSLSCRASQSISDYL | 438 |
| | LHWVKQSHAKSLEWIGVINTYSSNTYYNQNFEG | | HWYQQKSHESPRLLIKYASQSISGIPSRFSGSG | |
| | KATMTVDKSSSTAYMELARLTSEDSAIYYCARR | | SGSDFTLSINSVEPEDVGVYYCQNGHSFPFTFG | |
| | RGSHRDAYFDYWGQGTTLTVSS | | SGTKLEIK | |
| SCT-Da311 | QVQLQQSGPELVKPGASVKMSCKASGYTFTDYV | 206 | DVVMTQTPLSLPVSLGDQASISCRSRQSLVHSN | 441 |
| | MSWVKQRTGQGLEWIGEIYPGGNRTYYSEKFKG | | GITYLHWYLQKPGQSPKLLIDKVFNRFSGVPDR | |
| | KATLTADKSSNTAYMQVSSLTAEDSAVYFCARF | | FSGSGSGTDFTLKISRVEAEDLGVYFCSQSTHV | |
| | NDWAMDHWGQGTSVTVSS | | PWTFGGGTKLEIK | |
| SCT-Da312 | QVQLQQSGPELVKPGASVKMSCKASGYTFTDYV | 207 | DVVMTQTPLSLPVSLGDQASISCRSRQSLVHSN | 442 |
| | MSWVKQRTGQGLEWIGEIYPGGNSTYYNEKFKG | | GITYLHWYLQKPGQSPKLLIYKVSNRFSGVPDR | |
| | KATLTADKSSNTAYMQLSSLTSEDSAVYFCARF | | FSGSGSGTDFTLKISRVEAEDLGVYFCSQSTHV | |
| | NDWSMDYWGQGTSVTVSS | | PWTFGGGTKLEIK | |
| SCT-Da313 | QVQLQQSGPELVKPGASVKMSCKASGYTFTDYV | 208 | DVVMTQTPLSLPVSLGDQASISCRSRQSLVHSN | 443 |
| | MSWVTQRTGQGLEWIGEIYPGGSSIHYNEKFKD | | GITYLHWYLQKPGQSPKLLIYKVSNRFSGVPDR | |
| | KATLTADKSSNTAYMQLSSLTSEDSAVYFCARF | | FSGSGSGTDFTLKISRVEAEDLGVYFCSQSTHF | |
| | NDWAMDYWGQGTSVTVSS | | PWTFGGGTKLEIK | |
| SCT-Da314 | QVQLQQSGPELVKPGASVKMSCKASGYTFTDYV | 209 | DVVMTQTPLSLPVSLGDQASISCRSRQSLVHSN | 443 |
| | MSWVTQRTGQGLEWIGEIYPGGSSIYYNEKFKD | | GITYLHWYLQKPGQSPKLLIYKVSNRFSGVPDR | |
| | KATLTADKSSNTAYMQLSSLTSEDSAVYFCARF | | FSGSGSGTDFTLKISRVEAEDLGVYFCSQSTHF | |
| | NDWAMDYWGQGTSVTVSS | | PWTFGGGTKLEIK | |
| SCT-Da315 | QVQLQQSGPELVKPGASVRISCKASGYTFTSYF | 210 | DIVLTQSPATLSVTPGDRVSLSCRASQSINNYL | 444 |
| | IHWVKQRPGQGLEWIGWIYPGNVNTNYNERFKG | | HWYQQKSHESPRLLIKYASHSISGIPSRFSGSG | |
| | KATLTADKSSSTAYMQLSSLTSEDSAVYFCARD | | SGTDFTLSINSVETEDFGMYFCQQSSTWPLTFG | |
| | YAMDYWGQGTSVTVSS | | AGTKLELK | |
| SCT-Da316 | QVQLQQSGPELVKPGASVRISCKASGYTFTTYF | 211 | DIVLIQSPATLSVTPGDRVSLSCRASQGINNYL | 445 |
| | IHWVKQRPGQGLEWIGWIYPGNVNVNYNEKFKG | | HWYQQKSHESPRLLIKYASHSISGIPSRFSGSG | |
| | KATLTADKSSSTAYMHLSSLTSEDSAVYFCSRD | | SGTDFTLSINSVETEDFGMYFCQQSNSWPLTFG | |
| | YAMDYWGQGTSVTVSS | | AGTKLELK | |
| SCT-Da317 | QVQLQQSGPELVKPGASVRISCKASGYTFTTYY | 212 | DIVLTQSPATLSVTPGDRVSLSCRASQSINNYL | 446 |
| | IHWVKQRPGQGLEWIGWIYPGNVNTNYNEKFKG | | HWYQQKSHESPRLLIKYASHSISGIPSRFSGSG | |
| | KATLTADKSSSTAYMQLSSLTSEDSAVYFCTRD | | SGTDFTLSINSVESEDFGKYFCQQSNSWPLTFG | |
| | YAMDYWGQGTSVTVSS | | AGTQLELK | |
| SCT-Da318 | QVQLQQSGPELVKPGASVRISCKASGYTFTTYY | 213 | DIVLIQSPATLSVTPGDRVSLSCRASQSINNYL | 447 |
| | IHWVKQRPGQGLEWIGWIYPGNVNVNYNEKFKG | | HWYQQKSHESPRLLIKYASHSISGIPSRFSGSG | |
| | KATLTADKSSSTAYMQLSSLTSEDSAVYFCTRD | | SGTDFTLSINSVETEDFGMYFCQQSNSWPLTFG | |
| | YAMDYWGQGTSVTVSS | | AGTKLELK | |
| SCT-Da319 | QVQLQQSGPELVKPGASVRISCKASGYTFTTYY | 214 | DIVLTQSPATLSVTPGDRVSLSCRASQSINNYL | 410 |
| | IHWVKQRPGQGLEWIGWIYPGNVNVNYNERFKG | | HWYQQKSHESPRLLIKYASHSISGIPSRFSGSG | |
| | KATLTADKSSSTAYMQLSSLTSEDSAVYFCTRD | | SGTDFTLSINSVETEDFGVYFCQQSNSWPLTFG | |
| | YAMDYWGQGTSVTVSS | | AGTKLELK | |
| SCT-Da320 | QVQLQQSGPELVKPGASVRISCTASGYTFTTYY | 215 | DIVLTQSPATLSVTPGDRVSLSCRASQSINNYL | 448 |
| | IHWVKQRPGQGLEWIGWIYPGNVNTNYNEKFKG | | HWYQQKSHESPRLLIKYASHSISGIPSRFSGSG | |
| | KATLTADKSSSTAYMQLSSLTSEDSAVYFCARD | | SGTDFTLSINSVETEDFGMYFCQQSNSWPLTFG | |
| | YAMDYWGQGTSVTVSS | | AGTKLELK | |
| SCT-Da321 | QVQLQQSGPELVKPGASVRISCTASGYTFTTYY | 215 | QIVLTQSPALMSASPGEKVTMTCSASSSVSYMY | 333 |
| | IHWVKQRPGQGLEWIGWIYPGNVNTNYNEKFKG | | WYQQKPRSSPKPWIYLTSNLASGVPARFSGSGS | |
| | KATLTADKSSSTAYMQLSSLTSEDSAVYFCARD | | GTSYSLTISSMEAEDAATYYCQQWSSNPLTFGA | |
| | YAMDYWGQGTSVTVSS | | GTKLELK | |
| SCT-Da322 | QVQLQQSGPELVKPWASVRISCKASGYTFTTYY | 216 | DIVLTQSPATLSVTPGDRVSLSCRASQRINSYL | 449 |
| | IHWVKQRPGQGLEWIGWIYPGNVNVNYNERFKG | | HWYQQKSHESPRLLIKYASHSISGIPSRFSGSG | |
| | KATLTADKPSSTAYMQLSSLTSEDSAVYFCSRD | | SGTDFTLSINSVETEDFGMYFCQQSNSWPLTFG | |
| | YAMDYWGQGTSVTVSS | | AGTKLELK | |
| SCT-Da323 | QVQLQQSGPELVRPGVSVKISCKGFGYTFTDYA | 217 | DIVMTQSPATLSVTPGDRVSLSCRASQSISDYL | 450 |
| | MHWVKQSHAKSLEWIGVINTYSGNTYYNQKFKD | | HWYQQKSHESPRLLIKSASQSISGIPSRFSGSG | |
| | KATMTVDKSSSTAYMELARLTSEDSAIYYCARR | | SGSDFTLSINSVEPEDVGVYYCQNGHSFPFTFG | |
| | RGSHRDAYFDYWGQGTPLTVSS | | SGTKLEIE | |
| SCT-Da324 | QVQLQQSGPELVRPGVSVKISCKGSGYTFTDYA | 218 | DVVMAPTPLTLSVTIGQPASISCKSSQSLLDSD | 451 |
| | IHWVKQSHAKSLEWIGVINTHSDNTNYFQRFKG | | GETYLNWLLQRPGQSPKRLIYLVSKLDSGVPDR | |
| | KATMTVDKSSSTAYMELARLTSEDSAIYYCARP | | FTGSGSGTDFTLKISRVEAEDLGVYYCWEGTHF | |
| | NYDGHWYFDVWGAGTTVTVSS | | WPFGGGTKLEIK | |
| SCT-Da325 | QVQLQQSGPELVRPGVSVKISCKGSGYTFTDYA | 219 | DIVMTQSPATLSVTPGDRVSLSCRASQSISDYL | 452 |
| | IHWVKQSHAKSLEWIGVINTYSGNTNYNQNFKV | | YWYQQKSHESPRLLIKYASQSISGIPSRFSGSG | |
| | KATMTVDKSSNTAYMELARLTSEDSAIYYCARR | | SGSDFTLSISSVEPEDVGVYYCQNGHSFPFTFG | |
| | RGIHRDAYFDYWGQGTTLTVSS | | SGTKLEIK | |
| SCT-Da326 | QVQLQQSGPELVRPGVSVKISCKGSGYTFTDYA | 220 | ENVLTQSPVIMSASPGEKVTMTCRASSSVSSSY | 453 |
| | MHWVKQSHAKSLEWIGIINSYSGNTNYNQKFKG | | LHWYQQKSGASPKLWIYSTSNLASGVPARFSGS | |
| | KATMTVDKSSSTAYMELARLTSEDSAIYYCARW | | GSGTSYSLTISSVEAEDAATYYCQQYSGYPLTF | |
| | AITTGVYYAMDYWGQGTSVTVSS | | GSGTKLEIK | |
| SCT-Da327 | QVQLQQSGPELVRPGVSVKISCKGSGYTFTDYA | 221 | DIVMTQSPATLSVTPGDRVSLSCRASQSISDYL | 331 |
| | MHWVKQSHAKSLEWIGVINTYSANTNYNQKFKG | | HWYQQKSHESPRLLIKYVSQSISGIPSRFSGSG | |
| | KATMTVDKSSSTAYLELARLTSEDSAIYYCARR | | SGSDFTLSINSVEPEDVGVYYCQNGHSFPFTFG | |
| | RGSHRDAYFDYWGQGTTLTVSS | | SGTKLEIK | |
| SCT-Da328 | QVQLQQSGPELVRPGVSVKISCKGSGYTFTDYA | 222 | DIVLTQSPASLAVSLGQRATISCRASESVDNYG | 454 |
| | MHWVKQSHAKSLEWIGVINTYSGNTNYNQNFKV | | ISFMNWFQQKPGQPPKLLIYAASNQGSGVPARF | |
| | KATMTVDKSSSTAYMELARLTSEDSAIYYCARR | | SGSGSGTDFSLNIHPMEEDDTAMYFCQQSKEVP | |
| | RGSHRDAYFDYWGQGTTLTVSS | | TFGGGTKLEIK | |
| SCT-Da329 | QVQLQQSGPELVRPGVSVKISCKGSGYTFTDYA | 222 | DIVMTQSPATLSVTPGDRVSLSCRASQSISDYL | 455 |
| | MHWVKQSHAKSLEWIGVINTYSGNTNYNQNFKV | | HWYQQKSHESPRLLIKYASQSISGIPSRFSGSG | |
| | KATMTVDKSSSTAYMELARLTSEDSAIYYCARR | | SGSDFTLSISSVEPEDVGVYYCQNGHSFPFTFG | |
| | RGSHRDAYFDYWGQGTTLTVSS | | SGTKLEIK | |
| SCT-Da330 | QVQLQQSGPELVRPGVSVKISCKGSGYTFTDYA | 222 | QIVLTQSPALMAASPGEKVTITCSVSSSISYMY | 366 |
| | MHWVKQSHAKSLEWIGVTNTYSGNTNYNQNFKV | | WYQQKSETSPKPWIYGTSNLASGVPVRFSGSGS | |
| | KATMTVDKSSSTAYMELARLTSEDSAIYYCARR | | GTSYSLTISSMEAEDAATYYCQQWSSYPLTFGA | |
| | RGSHRDAYFDYWGQGTTLTVSS | | GTKLELK | |
| SCT-Da331 | QVQLQQSGPELVRPGVSVKISCKGSGYTFTDYA | 223 | DIVMTQSPATLSVTPGDRVSLSCRASQSISDYL | 438 |
| | MHWVKQSHVKSLEWIGVINTYSGNTNYNQKFKG | | HWYQQKSHESPRLLIKYASQSISGIPSRFSGSG | |
| | KATMTVDKSSSTAYMELARLTSEDSAIYYCARR | | SGSDFTLSINSVEPEDVGVYYCQNGHSFPFTFG | |
| | RGSHQDAYFDYWGQGTTLTVSS | | SGTKLEIK | |
| SCT-Da332 | QVQLQQSGPELVRPGVSVKISCKGSGYTFTDYA | 224 | DIVMTQSPATLSVTPGDRVSLSCRASQSISDYL | 456 |
| | VHWMKQSHAKSLEWIGVINTYSGNTYYNLHFKD | | HWYQQKSHESPRLLIKYASQSISGIPSRFSGSG | |
| | KATMTVDKSSSTAYMELATLTSEDSAIYYCARR | | SGSDFTLSIMMVEPEDVGVYYCQNSHSFPFTFG | |
| | RGSHRDAYFDYWGQGTTLTVSS | | SGTKLEIK | |
| SCT-Da333 | QVQLQQSGPELVRPGVSVKISCKGSGYTFTDYA | 225 | DIVMTQSHKFMSTSVGDRVSITCRASQDVSTAV | 400 |
| | VHWVKQSHAKSLEWIGVINTYSGNTYYNLHFKG | | AWYQQKPGQSPKLLIYSASYRYTGVPDRFTVSG | |
| | KATMTVDKSSSTAYMELARLTSEDSAIYYCARR | | SGTDFTFTISSVQAEDLAVYYCQQHYSTPWTFG | |
| | RGSHRDAYFDYWGQGTTLTVSS | | GGTKLEIK | |
| SCT-Da334 | QVQLQQSGPELVRPGVSVKISCKGSGYTFTDYA | 225 | DIVMTQSPATLSVTPGDRVSLSCRASQSISDYL | 457 |
| | VHWVKQSHAKSLEWIGVINTYSGNTYYMLHFKG | | HWYQQKSHESPRLLIKYASQSISGIPSRFSGSG | |
| | KATMTVDKSSSTAYMELARLTSEDSAIYYCARR | | SGSDFTLSINSVDPEDVGMYYCQNSHSFPFTFG | |
| | RGSHRDAYFDYWGQGTTLTVSS | | SGTKLEIK | |
| SCT-Da335 | QVQLQQSGPELVRPGVSVKISCKGSGYTFTDYA | 225 | DIVMTQSPATLSVTPGDSVSLSCRASQTISDYL | 458 |
| | VHWVKQSHAKSLEWIGVINTYSGNTYYNLHFKG | | HWYQQKSHESPRLLIKYASQSISGIPSRFSGSG | |
| | KATMTVDKSSSTAYMELARLTSEDSAIYYCARR | | SGSDFTLSINSVEPEDVGVYYCQNSHSFPFTFG | |
| | RGSHRDAYFDYWGQGTTLTVSS | | SGTKLEIK | |
| SCT-Da336 | QVQLQQSGPELVRPGVSVKISCKGSGYTFTDYA | 226 | DIVMTQSPATLSVTPGDRVSLSCRASQSISDYL | 456 |
| | VHWVKQSHAKSLEWIGVINTYSGNTYYNLHFKG | | HWYQQKSHESPRLLIKYASQSISGIPSRFSGSG | |
| | KATMTVDKSSSTAYMELATLTSEDSAIYYCARR | | SGSDFTLSINNVEPEDVGVYYCQNSHSFPFTFG | |
| | RGSHRDAYFDYWGQGTTLTVSS | | SGTKLEIK | |
| SCT-Da337 | QVQLQQSGPELVRPGVSVKISCKGSGYTFTDYA | 227 | DIVMTQSPATLSVTPGDRVSLSCRASQSISDYL | 459 |
| | VHWVKQSHAKSLEWIGVINTYSGNTYYNLNFTG | | HWYQQKSHESPRLLIKYASQSISGIPSRFSGSG | |
| | KATMTVDKSSSTAYMELARLTSEDSAIYYCARR | | SGSDFTLTINSVEPEDIGLYYCQNGHSFPFTFG | |
| | RGSHRDAYFDYWGQGTTLTVSS | | SGTKLEIK | |
| SCT-Da338 | QVQLQQSGPELVSPGVSVKISCKGSGYTFTDYA | 228 | DIVMTQSPATLSVTPGDRVSLSCRASQSISDYL | 457 |
| | VHWVKQSHAKSLEWIGVINTYSGNTYFNLHFKG | | HWYQQKSHESPRLLIKYASQSISGIPSRFSGSG | |
| | KATMTVDKSSSTAYMELASLTSEDSAIYYCARR | | SGSDFTLSINSVDPEDVGMYYCQNSHSFPFTFG | |
| | RGSHRDAYFDYWGQGTTLTVSS | | SGTKLEIK | |
| SCT-Da339 | QVQLQQSGPEVVKPGASVKMSCKASGYTFTDYV | 229 | DVVMTQTPLSLPVSLGDQASISCRSRQSLVHSN | 460 |
| | MTWVKQRTGQGLEWIGEIYPGGNRTYYKEKFKG | | GITYLHWYLQKPGQSPKLLIFKVSNRFSGVPDR | |
| | KATLTADKSSNTAYMQLSSLTSEDSAVYFGARF | | FSGSGSGTDFTLKISRVEAEDLGVYFCSQSTHV | |
| | NDWAMDYWGQGTSVTVSS | | PWTFGGGTKLEIK | |
| SCT-Da340 | QVQLQQSGTELVRPGASVTLSCKTSGYTFTDYE | 230 | SIVVTQTPKFLLVSAGDRVTITCKASQSVSNDV | 461 |
| | MHWVKQTPVHGLEWIGGIDPETGATAYNQKFKG | | AWYQQKPGQSPKLLIYYASNRYTGVPDRFTGSG | |
| | KATLTADKSSSTAYMELRSLTSEDSAVYYCTAR | | YGTDFTFTISTVQAEDLAVYFCQQDYTSPPTFG | |
| | QLGLPFAYWGQGTLVTVSA | | GGTKLEIK | |
| SCT-Da341 | QVTLKESGPGILQPSQTLSLTCSFSGFSLNTSN | 231 | SIVMTQTPKFLLVSAGDRVTITCKANQFVSNFV | 462 |
| | LGVGWIRQPSGKGLEWLAHIWWDDDKRYNPALK | | AWYQQKPGQSPKLLIYYASNRYTGVPDRFTGSG | |
| | SRLTISKDSSSNQVFLKIASVDTADTATYYCAR | | YGTDFTFTITAVQAEDLAVYFCQQDYSSPYTFG | |
| | KNHYYGHWYFDVWGAGTTVTVSS | | GGTKLEIK | |
| SCT-Da342 | QVTLKESGPGILQPSQTLSLTCSFSGFSLNTSN | 232 | SIVMTQTPKFLLVSAGDRVTITCKASQFVSNFV | 463 |
| | MGVGWIRQPSGKGLEWLAHIWWDDDKRYNPALK | | AWYQQKPGQSPKLLIYYASNRYTGVPDRFTGSG | |
| | SRLTISKDSSSNQVFLKIASVDTADTATYYCAR | | YGTDFTFTITTVQAEDLAVYFCQQDYSSPYTFG | |
| | KNHYYGHWYFDVWGAGTTVTVSS | | GGTKLEIK | |
| SCT-Da343 | QVTLKESGPGILQPSQTLSLTCSFSGFSLSTPG | 233 | SIVMTQTPKFLLVSAGDRVTITCKASQSVNNDV | 464 |
| | LGVGWIRQPSGKGLEWLAHIWWDDDKRYNPALK | | AWYQQKPGQSPKLLIYYASNRYTGVPDRFTGSG | |
| | SRLTISKDTSSNQVFLKIASVDTADTATYYCAR | | YGTDFTFTISTVQAEDLAVYFCQQDYNSPYAFG | |
| | KNHYYGHWYFDVWGAGTTVTVSS | | GGTKLEIK | |
| SCT-Da344 | QVTLKESGPGILQPSQTLSLTCSFSGFSLSTPN | 234 | SIVMTQTPKFLLVSAGDRVTITCKASQSVINDV | 465 |
| | MGVGWIRQPSGKGLEWLAHIWWDDDKRQNAALK | | AWYQQKPGQSPKLLIYYASNRYTGVPDRFTGSG | |
| | SRLTISKETSSNQVFLKIASVDTADTATYYCAR | | YGTDFTFTISSVQAEDLAVYFCQQDYSSPYTFG | |
| | KIHYYGHWYFDVWGAGTTVTVSS | | GGTKLEIK | |
| SCT-Da345 | QVTLKESGPGILQPSQTLSLTCSFSGFSLSTPS | 235 | SIVMTQTPKFLLVSAGDRVTITCKASQSVINDV | 466 |
| | MGVGWIRQPSGKGLEWLAHIWWDDDKRSDPALK | | AWFQQKPGQSPNLLIYYASNRYTGVPARFTGSG | |
| | SRLTISKDTSSNQVFLKIASVDTADTGTYYCAR | | YGTDFTFTISTVQAEDLAVYFCQQDYSSPYTFG | |
| | KNHYSGHWFFDVWGAGTTVTVSS | | GGTKLEIK | |
| SCT-Da346 | QVTLKESGPGILQPSQTLSLTCSFSGFSLSTSG | 236 | SIVMTQTPKFLLVSAGDRVTITCKASQSVSNYV | 467 |
| | MGVGWIRQPSGKGLEWLAHIWWDDDKRYNPALK | | AWYQQKPGQSPKLLIYYASNRYTGVPDRFTGSG | |
| | SRLTISKDSSSNQVFLKIASVDTADTATYYCAR | | YGTDFTFTITTVQAEDLAVYFCQQDYSSPYTFG | |
| | KNHYYGHWYFDVWGAGTTVTVSS | | GGTKLEIK | |
| SCT-Da347 | QVTLKESGPGILQPSQTLSLTCSFSGFSLSTSN | 237 | SIVMTQTPKFLLVSAGDRVTITCKASQSVINDV | 468 |
| | LGVGWIRQPSGKGLEWLAHIWWDDDKRYNPDLE | | AWYQQKPGQSPKLLIYYASNRYTGVPDRFTGSG | |
| | SRLTISKDTSSNQVFLKIASVDTADTATYYCAR | | YGTDFTFTISTVQAEDLAVYFCQQDYSSPYTFG | |
| | KSHYYGHWYFDVWGAGTTVTVSS | | GGTKLEIK | |
| SCT-Da348 | QVTLKESGPGILQPSQTLSLTCSFSGFSLSTSN | 238 | SVVMTQTPKFLLVSAGDRITITCKASQSVINDV | 469 |
| | MGVGWIRQPSGKGLEWLAHIWWDDDKRYHPALK | | AWYQQKPGQSPKLLIYYASNRYTGVPDRFTGSG | |
| | SRLTISKDTSSNQVFLKIASVDTADTATYYCAR | | YGTDFTFTISTVQAEDLAVYFCQQDYNSPFTFG | |
| | KSHYYGHWYFDFWGAGTTVTVSS | | GGTKLEIK | |
| SCT-Da349 | QVTLKESGPGILQPSQTLSLTCSFSGFSLSTSN | 239 | DIVMTQSQKFMSTSVGDRVSVTCKASQNVGTDV | 304 |
| | MGVGWIRQPSGKGLEWLAHIWWDDDKRYNPALK | | AWYQQKPGQSPKLLIYSASYRFSGVPDRFTGSG | |
| | SRLTISKDSSNNQVFLKIASVDTSDTAIYYCGR | | SGTDFSLAISNVQSEDLAEYFCQQYNSYPYTFG | |
| | KSHYYGHWYFDVWGAGTTVTVSS | | GGAKLEIK | |
| SCT-Da350 | QVTLKESGPGILQPSQTLSLTCSFSGFSLSTSN | 239 | SIVMTQTPKFLLVSAGDRFTITCKASQNVDNYV | 354 |
| | MGVGWIRQPSGKGLEWLAHIWWDDDKRYNPALK | | AWYQQKPGQSPKLLIYYASNRYTGVPDRFTGSG | |
| | SRLTISKDSSNNQVFLKIASVDTSDTAIYYCGR | | YGTDFTFTITTVQAEDLAVYFCQQDYSSPYTFG | |
| | KSHYYGHWYFDVWGAGTTVTVSS | | GGTKLEIK | |
| SCT-Da351 | QVTLKESGPGILQPSQTLSLTCSFSGFSLSTSN | 239 | SIVMTQTPKFLLVSTGDRFTITCKASQNVDNYV | 470 |
| | MGVGWIRQPSGKGLEWLAHIWWDDDKRYNPALK | | AWYQQKPGQSPKLLIYYASNRYTGVPDRFTGSG | |
| | SRLTISKDSSNNQVFLKIASVDTSDTAIYYCGR | | YGTDFTFTITTVQAEDLAVYFCQQDYSSPYTFG | |
| | KSHYYGHWYFDVWGAGTTVTVSS | | GGTKLEIK | |
| SCT-Da352 | QVTLKESGPGILQPSQTLSLTCSFSGFSLSTSN | 240 | SLVMTQTPKFLLVSPGDRITITCKASQSVINDV | 471 |
| | MGVGWIRQPSGKGLEWLAHIWWDDDKRYNPALK | | AWYQQKPGQSPKLLIYYASNRNTGVPDRFTGSG | |
| | SRLTISKDTSSNQVFLKIASVDTADSATYYCAR | | YGTDFTLTISTVQAEDLAVYFCQQDYNSPYTFG | |
| | KNHYYGHWYFDFWGAGTTVTVSS | | GGTKLEIK | |
| SCT-Da353 | QVTLKESGPGILQPSQTLSLTCSFSGFSLSTSN | 241 | SIVMTQTPKFLLVSAGDRITITCKASQSVINDV | 472 |
| | MGVGWIRQPSGKGLEWLAHIWWDDDKRYNPALK | | AWYQQKPGQSPKLLMYYASNRNTGVPDRFTGSG | |
| | SRLTISKDTSSNQVFLKIASVDTADTATYYCAR | | YGTDFTLTISTVQAEDLAVYFCQQDYNSPYTFG | |
| | KNHYYGHWYFDFWGAGTTVTVSS | | GGTKLEIK | |
| SCT-Da354 | QVTLKESGPGILQPSQTLSLTCSFSGFSLSTSN | 242 | DIQMTQSPASLSVSVGETVTITCRASENIYSNL | 473 |
| | MGVGWIRQPSGKGLEWLAHIWWDDDKRYNPDLK | | AWYQQKQGKSPQLLVYAATNLADGVPSRFSGSG | |
| | SRLTISKDTSSNQVFLKIASVDTADTATYYCAR | | SGTQYSLKINSLQSEDFGSYYCQHFWGTPFTFG | |
| | KNHYYGHWYFDVWGAGTTVTVSS | | SGTKLEIK | |
| SCT-Da355 | QVTLKESGPGILQPSQTLSLTCSFSGFSLSTSN | 242 | DIQMTQSPSSLSASLGGKVTITCKASQDINKYI | 474 |
| | MGVGWIRQPSGKGLEWLAHIWWDDDKRYNPDLK | | SWYQHKPGKGPRLLIHYTSTLQPGIPSRFSGSG | |
| | SRLTISKDTSSNQVFLKIASVDTADTATYYCAR | | SGRDYSFSISNLEPEDIATYYCLQYDNLWTFGG | |
| | KNHYYGHWYFDVWGAGTTVTVSS | | GTKLEIK | |
| SCT-Da356 | QVTLKESGPGILQPSQTLSLTCSFSGFSLSTSN | 242 | SIVMTQTPKFLLVSAGDRVTITCKASQSVINDV | 475 |
| | MGVGWIRQPSGKGLEWLAHIWWDDDKRYNPDLK | | AWFQQQPGQSPKLLIYYASNRYTGVPDRFTGSG | |
| | SRLTISKDTSSNQVFLKIASVDTADTATYYCAR | | YGTDFTFTISTVQAEDLAVYFCQQDYSSPYTFG | |
| | KNHYYGHWYFDVWGAGTTVTVSS | | GGTKLEIK | |
| SCT-Da357 | QVTLKESGPGILQPSQTLSLTCSFSGFSLSTSN | 242 | SIVMTQTPKFLLVSAGDRVTITCRASQSVTNDV | 265 |
| | MGVGWIRQPSGKGLEWLAHIWWDDDKRYNPDLK | | AWYQQKPGQSPKLLIYYASNRYTGVPDRFTGSG | |
| | SRLTISKDTSSNQVFLKIASVDTADTATYYCAR | | YGTDFTFTISTVQAEDLAVYFCQQDYNSPYTFG | |
| | KNHYYGHWYFDVWGAGTTVTVSS | | GGTKLEIK | |
| SCT-Da358 | QVTLKESGPGILQPSQTLSLTCSFSGFSLSTSN | 243 | SIVMTQTPKFLLVSAGDRVTITCKASQSVSNDV | 476 |
| | MGVGWIRQPSGKGLEWLAHIWWDDDKRYNPDLK | | AWYQQKPGQSPKLLIYYASNRYTGVPDRFTGSG | |
| | SRLTISKDTSSNQVFLKIASVDTADTATYYCAR | | YGTDFTFTISTVQAEDLAVYFCQQDYNSPYTFG | |
| | KSHYYGHWYFDVWGAGTTVTVSS | | GGTKLEIK | |
| SCT-Da359 | QVTLKESGPGILQPSQTLSLTCSFSGFSLSTSN | 244 | SIVMTQTPKFLLVSAGDRVTITCKASQSVINDV | 468 |
| | MGVGWIRQPSGKGLEWLAHIWWDDDKRYNPDLK | | AWYQQKPGQSPKLLIYYASNRYTGVPDRFTGSG | |
| | SRLTISKDTSSNQVFLKIASVDTADTATYYCGR | | YGTDFTFTISTVQAEDLAVYFCQQDYSSPYTFG | |
| | KSHYYGHWYFDVWGAGTTVTVSS | | GGTKLEIK | |
| SCT-Da360 | QVTLKESGPGILQPSQTLSLTCSFSGFSLSTSN | 245 | NIVMTQTPRFLLVSAGDRVTITCKASQTVTNDV | 477 |
| | MGVGWIRQPSGKGLEWLAHIWWDDDTRYKPALK | | AWYQQKPGQSPKLLIYYASNRYTGVPDRFTGSG | |
| | SRLTISKDTSSNQVFLKIASVDTADTATYFCAR | | YGTDFTFTISTVQAEDLAVYFCQQDYISPYTFG | |
| | KNHYYGHWYFDVWGAGTTVTVSS | | GGTKLEIK | |
| SCT-Da361 | QVTLKESGPGILQPSQTLSLTCSFSGFSLSTYG | 246 | DIQMTQTTSSLSASLGDRVTINCSASQGIINYL | 478 |
| | IGVGWIRQPSGKGLEWLAHIWWNDNKYYNTALK | | NWYQQKPDGTVKLLIYYTSSLHSGVPSRFSGSG | |
| | SRLTISKDTSNNQVFLKIASVDTADTATYYCAR | | SGTDYSLTISNLEPEDIASYYCQQYSKLPYTFG | |
| | IDYWFAYWGQGTLVTVSA | | GGTKVEIK | |
| SCT-Da362 | QVTLKESGPGILQPSQTLSLTCSFSGFSLSTYG | 246 | QIVLTQSPALMSASPGEKVTMTCSASSSVSYMY | 333 |
| | IGVGWIRQPSGKGLEWLAHIWWNDNKYYNTALK | | WYQQKPRSSPKPWIYLTSNLASGVPARFSGSGS | |
| | SRLTISKDTSNNQVFLKIASVDTADTATYYCAR | | GTSYSLTISSMEAEDAATYYCQQWSSNPLTFGA | |
| | IDYWFAYWGQGTLVTVSA | | GTKLELK | |
| SCT-Da363 | EVQLVESGGDLVQPGGSLRLSCKTSGFTFSSFY | 973 | DIQMTQSPSSLSASVGDRVTITCRASQDVSTAV | 974 |
| | ISWVRQAPGKGLEWIAWIYAGPGYTSYADSVKG | | AWYQQKPGKAPKLLIYSASYRYSGVPSRFSGSG | |
| | RFTISRDNSRNTLYLQMNSLRAEDTAVYYCARH | | SGTDFTLTISSLQPEDFATYYCQQHYSTPWTFG | |
| | VGRQRGYFDVWGQGTLVTVSS | | QGTKVEIK | |
| SCT-Da364 | EVQLVESGGDLVQPGGSLRLSCKTSGFTFSSFY | 973 | DIVMTQSQRFMSTTVGDRVSITCKASQDVSTAV | 975 |
| | ISWVRQAPGKGLEWIAWIYAGPGYTSYADSVKG | | AWYQQKPGQSPKLLIYSASYRYTGVPDRFTGSG | |
| | RFTISRDNSRNTLYLQMNSLRAEDTAVYYCARH | | SGTDFTLTISNMQSEDLADFFCQQHYSTPWTFG | |
| | VGRQRGYFDVWGQGTLVTVSS | | GGTKLEIK | |
| SCT-Da365 | EVQLQQSGPELEKPGASVKLSCKASGYTFTEYT | 976 | DIQLTQSPSSLSASVGDRVTMTCRASSSVSYIH | 977 |
| | MHWVKQSHGKSLEWIGVINPNNGGTTYNQKFRG | | WFQQKPGKAPKPWIYLTSNLASGVPVRFSGSGS | |
| | KATLTVDKSSSTAYMQLKSLTSEDSAVYYCARP | | GTDYTFTISSLQPEDIATYYCQQWSSNPLTFGG | |
| | HSLLRLRREFDYWGAGTTVTVSS | | GTKLEIK | |
| SCT-Da366 | EVQLQQSGPELEKPGASVKLSCKASGYTFTEYT | 976 | QIVLIQSPAIMSASPGEKVTMTCSASSSVSYMY | 978 |
| | MHWVKQSHGKSLEWIGVINPNNGGTTYNQKFRG | | WYQQKPGSSPRLLIYLTSNLASGVPVRFSGSGS | |
| | KATLTVDKSSSTAYMQLKSLTSEDSAVYYCARP | | GTSYSLTISRMEAEDAATYYCQQWSSNPLTFGG | |
| | HSLLRLRREFDYWGAGTTVTVSS | | GTKLEIK | |
| SCT-Da367 | EVQLQQSGPELEKPGASVKLSCKASGYTFTEYT | 976 | QIVSTQSPAIMSASPGEKVTMTCSASSSVSYMQ | 979 |
| | MHWVKQSHGKSLEWIGVINPNNGGTTYNQKFRG | | WYQQKPGTSPKRWIYLTSKLASGVPARFSGSGS | |
| | KATLTVDKSSSTAYMQLKSLTSEDSAVYYCARP | | GTSYSLTISSMEAEDAATYYCQQWSSNPLTFGG | |
| | HSLLRLRREFDYWGAGTTVTVSS | | GTKLEIK | |
| SCT-Da368 | EVQLVQSGAEVKKPGASVKVSCKTSGYTFTEYT | 980 | DIQLTQSPSSLSASVGDRVTMTCRASSSVSYIH | 977 |
| | MHWVKQAPGQGLEWIGVINPNNGGTTYNQKFQG | | WFQQKPGKAPKPWIYLTSNLASGVPVRFSGSGS | |
| | RVTMTRDTSISTAYMELGSLRSDDTAVYYCARP | | GTDYTFTISSLQPEDIATYYCQQWSSNPLTFGG | |
| | HSLLRLRREFDYWGQGTMVTVSS | | GTKLEIK | |
| SCT-Da369 | EVQLVQSGAEVKKPGASVKVSCKTSGYTFTEYT | 980 | QIVLIQSPAIMSASPGEKVTMTCSASSSVSYMY | 978 |
| | MHWVKQAPGQGLEWIGVINPNNGGTTYNQKFQG | | WYQQKPGSSPRLLIYLTSNLASGVPVRFSGSGS | |
| | RVTMTRDTSISTAYMELGSLRSDDTAVYYCARP | | GTSYSLTISRMEAEDAATYYCQQWSSNPLTFGG | |
| | HSLLRLRREFDYWGQGTMVTVSS | | GTKLEIK | |
| SCT-Da370 | EVQLVQSGAEVKKPGASVKVSCKTSGYTFTEYT | 980 | QIVSTQSPAIMSASPGEKVTMTCSASSSVSYMQ | 979 |
| | MHWVKQAPGQGLEWIGVINPNNGGTTYNQKFQG | | WYQQKPGTSPKRWIYLTSKLASGVPARFSGSGS | |
| | RVTMTRDTSISTAYMELGSLRSDDTAVYYCARP | | GTSYSLTISSMEAEDAATYYCQQWSSNPLTFGG | |
| | HSLLRLRREFDYWGQGTMVTVSS | | GTKLEIK | |
| SCT-Da371 | QVQLQQPGAELVKPGASVKMSCKASGVNIKDTY | 981 | DIQMTQSPSSLSASVGDRVTITCKASQNVGTDV | 982 |
| | IHWVKQTPGRGLEWIGRIDPANGNRKYNQKFKG | | AWYQQKPGKAPKALIYSASYRFSGVPYRFSGSG | |
| | KATLTADKSSSTAYMQLSSLTSEDSAVYYCARD | | SGTDFTLTISSLQPEDFATYFCQQYNSYPYTFG | |
| | GYYAPGYWGAGTTVTVSA | | QGTKVEIK | |
| SCT-Da372 | QVQLQQPGAELVKPGASVKMSCKASGVNIKDTY | 981 | DIVMTQSQRFMSTTVGDRVSITCKASQNVGTDV | 983 |
| | IHWVKQTPGRGLEWIGRIDPANGNRKYNQKFKG | | AWYQQKPGQSPKLLIYSASYRYTGVPDRFTGSG | |
| | KATLTADKSSSTAYMQLSSLTSEDSAVYYCARD | | SGTDFTLTISNMQSEDLADFFCQQYNSYPYTFG | |
| | GYYAPGYWGAGTTVTVSA | | GGTKLEIK | |
| SCT-Da373 | EVQLVQSGAEVKKPGASVKVSCKASGVNIKDTY | 984 | DIQMTQSPSSLSASVGDRVTITCKASQNVGTDV | 982 |
| | IHWVRQAPGQRLEWIGRIDPANGNRKYDPKFQG | | AWYQQKPGKAPKALIYSASYRFSGVPYRFSGSG | |
| | RVTITADTSASTAYMELSSLRSEDTAVYYCARD | | SGTDFTLTISSLQPEDFATYFCQQYNSYPYTFG | |
| | GYYAPGYWGQGTLVTVSS | | QGTKVEIK | |
| SCT-Da374 | EVQLVQSGAEVKKPGASVKVSCKASGVNIKDTY | 984 | DIVMTQSQRFMSTTVGDRVSITCKASQNVGTDV | 983 |
| | IHWVRQAPGQRLEWIGRIDPANGNRKYDPKFQG | | AWYQQKPGQSPKLLIYSASYRYTGVPDRFTGSG | |
| | RVTITADTSASTAYMELSSLRSEDTAVYYCARD | | SGTDFTLTISNMQSEDLADFFCQQYNSYPYTFG | |
| | GYYAPGYWGQGTLVTVSS | | GGTKLEIK | |

The preceding merely illustrates the principles of the disclosure. It will be appreciated that those skilled in the art will be able to devise various arrangements which, although not explicitly described or shown herein, embody the principles of the disclosure and are included within its spirit and scope. Furthermore, all examples and conditional language recited herein are principally intended to aid the reader in understanding the principles of the disclosure and the concepts contributed by the inventors to furthering the art and are to be construed as being without limitation to such specifically recited examples and conditions. Moreover, all statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the appended claims.

### REFERENCES CITED

### Other References

Castriconi, R., Dondero, A., Augugliaro, R., Cantoni, C., Carnemolla, B., Sementa, A. R., Negri, F., Conte, R., Corrias, M. V., Moretta, L., et al. (2004). Identification of 4Ig-B7-H3 as a neuroblastoma-associated molecule that exerts a protective role from an NK cell-mediated lysis. Proc. Natl. Acad. Sci. U.S.A 101, 12640-12645.
Chapoval, A. I., Ni, J., Lau, J. S., Wilcox, R. A., Flies, D. B., Liu, D., Dong, H., Sica, G. L., Zhu, G., Tamada, K., et al. (2001). B7-H3: a costimulatory molecule for T cell activation and IFN-gamma production. Nat. Immunol. 2, 269-274.
Hashiguchi, M., Kobori, H., Ritprajak, P., Kamimura, Y., Kozono, H., and Azuma, M. (2008). Triggering receptor expressed on myeloid cell-like transcript 2 (TLT-2) is a counter-receptor for B7-H3 and enhances T cell responses. Proc. Natl. Acad. Sci. U.S.A 105, 10495-10500.
Hofmeyer, K. A., Ray, A., and Zang, X. (2008). The contrasting role of B7-H3. Proc. Natl. Acad. Sci. U.S.A 105, 10277-10278.
Loos, M., Hedderich, D. M., Ottenhausen, M., Giese, N. A., Laschinger, M., Esposito, I., Kleeff, J., and Friess, H. (2009). Expression of the costimulatory molecule B7-H3 is associated with prolonged survival in human pancreatic cancer. BMC Cancer 9, 463.
Nygren, M. K., Tekle, C., Ingebrigtsen, V. A., and Fodstad, O. (2011). B7-H3 and its relevance in cancer; immunological and non-immunological perspectives. Front. Biosci. Elite Ed. 3, 989-993.
Prasad, D. V. R., Nguyen, T., Li, Z., Yang, Y., Duong, J., Wang, Y., and Dong, C. (2004). Murine B7-H3 is a negative regulator of T cells. J. Immunol. Baltim. Md. 1950 173, 2500-2506.
Roth, T. J., Sheinin, Y., Lohse, C. M., Kuntz, S. M., Frigola, X., Inman, B. A., Krambeck, A. E., McKenney, M. E., Karnes, R. J., Blute, M. L., et al. (2007). B7-H3 ligand expression by prostate cancer: a novel marker of prognosis and potential target for therapy. Cancer Res. 67, 7893-7900.
Saatian, B., Yu, X.-Y., Yu, X., Lane, A. P., Doyle, T., Casolaro, V., and Spannhake, E. W. (2004). Expression of genes for B7-H3 and other T cell ligands by nasal epithelial cells during differentiation and activation. Am. J. Physiol. Lung Cell. Mol. Physiol. 287, L217-225.
Steinberger, P., Majdic, O., Derdak, S. V., Pfistershammer, K., Kirchberger, S., Klauser, C., Zlabinger, G., Pickl, W. F., Stockl, J., and Knapp, W. (2004). Molecular characterization of human 4Ig-B7-H3, a member of the B7 family with four Ig-like domains. J. Immunol. Baltim. Md. 1950 172, 2352-2359.
Suh, W.-K., Gajewska, B. U., Okada, H., Gronski, M. A., Bertram, E. M., Dawicki, W., Duncan, G. S., Bukczynski, J., Plyte, S., Elia, A., et al. (2003). The B7 family member B7-H3 preferentially down-regulates T helper type 1-mediated immune responses. Nat. Immunol. 4, 899-906.
Sun, M., Richards, S., Prasad, D. V. R., Mai, X. M., Rudensky, A., and Dong, C. (2002). Characterization of mouse and human B7-H3 genes. J. Immunol. Baltim. Md. 1950 168, 6294-6297.
Sun, Y., Wang, Y., Zhao, J., Gu, M., Giscombe, R., Lefvert, A. K., and Wang, X. (2006). B7-H3 and B7-H4 expression in non-small-cell lung cancer. Lung Cancer Amst. Neth. 53, 143-151.
Wang, L., Fraser, C. C., Kikly, K., Wells, A. D., Han, R., Coyle, A. J., Chen, L., and Hancock, W. W. (2005). B7-H3 promotes acute and chronic allograft rejection. Eur. J. Immunol. 35, 428-438.
Wu, C.-P., Jiang, J.-T., Tan, M., Zhu, Y.-B., Ji, M., Xu, K.-F., Zhao, J.-M., Zhang, G.-B., and Zhang, X.-G. (2006). Relationship between co-stimulatory molecule B7-H3 expression and gastric carcinoma histology and prognosis. World J. Gastroenterol. 12, 457-459.
Yamato, I., Sho, M., Nomi, T., Akahori, T., Shimada, K., Hotta, K., Kanehiro, H., Konishi, N., Yagita, H., and Nakajima, Y. (2009). Clinical importance of B7-H3 expression in human pancreatic cancer. Br. J. Cancer 101, 1709-1716.

1. An antibody that binds human B7-H3 (SEQ ID NO:1) comprising:
a) a HCDR1 having the amino acid sequence of SEQ ID NO:574, a HCDR2 having the amino acid sequence of SEQ ID NO:560, a HCDR3 having the amino acid sequence of SEQ ID NO:546, a LCDR1 having the amino acid sequence of SEQ ID NO:904, a LCDR2 having the amino acid sequence of SEQ ID NO:882, and a LCDR3 having the amino acid sequence of SEQ ID NO:902; or
b) a HCDR1 having the amino acid sequence of SEQ ID NO:585, a HCDR2 having the amino acid sequence of SEQ ID NO:586, a HCDR3 having the amino acid sequence of SEQ ID NO:587, a LCDR1 having the amino acid sequence of SEQ ID NO:833, a LCDR2 having the amino acid sequence of SEQ ID NO:834, and a LCDR3 having the amino acid sequence of SEQ ID NO:835; or
c) a HCDR1 having the amino acid sequence of SEQ ID NO:582, a HCDR2 having the amino acid sequence of SEQ ID NO:552, a HCDR3 having the amino acid sequence of SEQ ID NO:589, a LCDR1 having the amino acid sequence of SEQ ID NO:944, a LCDR2 having the amino acid sequence of SEQ ID NO:957, and a LCDR3 having the amino acid sequence of SEQ ID NO:947; or
d) a HCDR1 having the amino acid sequence of SEQ ID NO:634, a HCDR2 having the amino acid sequence of SEQ ID NO:652, a HCDR3 having the amino acid sequence of SEQ ID NO:653, a LCDR1 having the amino acid sequence of SEQ ID NO:884, a LCDR2 having the amino acid sequence of SEQ ID NO:882, and a LCDR3 having the amino acid sequence of SEQ ID NO:885; or
e) a HCDR1 having the amino acid sequence of SEQ ID NO:634, a HCDR2 having the amino acid sequence of SEQ ID NO:652, a HCDR3 having the amino acid sequence of SEQ ID NO:653, a LCDR1 having the amino acid sequence of SEQ ID NO:884, a LCDR2 having the amino acid sequence of SEQ ID NO:882, and a LCDR3 having the amino acid sequence of SEQ ID NO:885; or
f) a HCDR1 having the amino acid sequence of SEQ ID NO:582, a HCDR2 having the amino acid sequence of SEQ ID NO:552, a HCDR3 having the amino acid sequence of SEQ ID NO:589, a LCDR1 having the amino acid sequence of SEQ ID NO:944, a LCDR2 having the amino acid sequence of SEQ ID NO:957, and a LCDR3 having the amino acid sequence of SEQ ID NO:947.

2. The antibody of claim 1, wherein the HCDR1 has the amino acid sequence of SEQ ID NO:574, the HCDR2 has the amino acid sequence of SEQ ID NO:560, the HCDR3 has the amino acid sequence of SEQ ID NO:546, the LCDR1 has the amino acid sequence of SEQ ID NO:904, the LCDR2 has the amino acid sequence of SEQ ID NO:882, and the LCDR3 has the amino acid sequence of SEQ ID NO:902.

3. The antibody of claim 1, wherein the HCDR1 has the amino acid sequence of SEQ ID NO:585, the HCDR2 has the amino acid sequence of SEQ ID NO:586, the HCDR3 has the amino acid sequence of SEQ ID NO:587, the LCDR1 has the amino acid sequence of SEQ ID NO:833, the LCDR2 has the amino acid sequence of SEQ ID NO:834, and the LCDR3 has the amino acid sequence of SEQ ID NO:835.

4. The antibody of claim 1, wherein the HCDR1 has the amino acid sequence of SEQ ID NO:582, the HCDR2 has the amino acid sequence of SEQ ID NO:552, the HCDR3 has the amino acid sequence of SEQ ID NO:589, the LCDR1 has the amino acid sequence of SEQ ID NO:944, the LCDR2 has the amino acid sequence of SEQ ID NO:957, and the LCDR3 has the amino acid sequence of SEQ ID NO:947.

5. The antibody of claim 1, wherein the HCDR1 has the amino acid sequence of SEQ ID NO:634, the HCDR2 has the amino acid sequence of SEQ ID NO:652, the HCDR3 has the amino acid sequence of SEQ ID NO:653, the LCDR1 has the amino acid sequence of SEQ ID NO:884, the LCDR2 has the amino acid sequence of SEQ ID NO:882, and the LCDR3 has the amino acid sequence of SEQ ID NO:885.

6. The antibody of claim 1, wherein the HCDR1 has the amino acid sequence of SEQ ID NO:634, the HCDR2 has the amino acid sequence of SEQ ID NO:652, the HCDR3 has the amino acid sequence of SEQ ID NO:653, the LCDR1 has the amino acid sequence of SEQ ID NO:884, the LCDR2 has the amino acid sequence of SEQ ID NO:882, and the LCDR3 has the amino acid sequence of SEQ ID NO:885.

7. The antibody of claim 1, wherein the HCDR1 has the amino acid sequence of SEQ ID NO:582, the HCDR2 has the amino acid sequence of SEQ ID NO:552, the HCDR3 has the amino acid sequence of SEQ ID NO:589, the LCDR1 has the amino acid sequence of SEQ ID NO:944, the LCDR2 has the amino acid sequence of SEQ ID NO:957, and the LCDR3 has the amino acid sequence of SEQ ID NO:947.

8. An antibody that binds human B7-H3 (SEQ ID NO:1) comprising:
a) a VH having the amino acid sequence of SEQ ID NO:99, and a VL having the amino acid sequence of SEQ ID NO:304; or
b) a VH having the amino acid sequence of SEQ ID NO:112, and a VL having the amino acid sequence of SEQ ID NO:369; or
c) a VH having the amino acid sequence of SEQ ID NO:121, and a VL having the amino acid sequence of SEQ ID NO:333; or
d) a VH having the amino acid sequence of SEQ ID NO:169, and a VL having the amino acid sequence of SEQ ID NO:411; or
e) a VH having the amino acid sequence of SEQ ID NO:973, and a VL having the amino acid sequence of SEQ ID NO:975; or
f) a VH having the amino acid sequence of SEQ ID NO:980, and a VL having the amino acid sequence of SEQ ID NO:978.

9. The antibody of claim 8, wherein the VH has the amino acid sequence of SEQ ID NO:99, and the VL has the amino acid sequence of SEQ ID NO:304.

10. The antibody of claim 8, wherein the VH has the amino acid sequence of SEQ ID NO:112, and the VL has the amino acid sequence of SEQ ID NO:369.

11. The antibody of claim 8, wherein the VH has the amino acid sequence of SEQ ID NO:121, and the VL has the amino acid sequence of SEQ ID NO:333.

12. The antibody of claim 8, wherein the VH has the amino acid sequence of SEQ ID NO:169, and the VL has the amino acid sequence of SEQ ID NO:411.

13. The antibody of claim 8, wherein the VH has the amino acid sequence of SEQ ID NO:973, and the VL has the amino acid sequence of SEQ ID NO:975.

14. The antibody of claim 8, wherein the VH has the amino acid sequence of SEQ ID NO:980, and the VL has the amino acid sequence of SEQ ID NO:978.

15. An antibody that binds human B7-H3 (SEQ ID NO:1) comprising:
a) a heavy-chain comprising the amino acid sequence of SEQ ID NO:99 and a light-chain comprising the amino acid sequence of SEQ ID NO:304; or
b) a heavy-chain comprising the amino acid sequence of SEQ ID NO: 112 and a light-chain comprising the amino acid sequence of SEQ ID NO:369; or
c) a heavy-chain comprising the amino acid sequence of SEQ ID NO:121 and a light-chain comprising the amino acid sequence of SEQ ID NO:333; or
d) a heavy-chain comprising the amino acid sequence of SEQ ID NO:169 and a light-chain comprising the amino acid sequence of SEQ ID NO:411; or
e) a heavy-chain comprising the amino acid sequence of SEQ ID NO:973 and a light-chain comprising the amino acid sequence of SEQ ID NO:975; or
f) a heavy-chain comprising the amino acid sequence of SEQ ID NO:980 and a light-chain comprising the amino acid sequence of SEQ ID NO:978.

16. The antibody of claim 15, wherein the heavy-chain comprises the amino acid sequence of SEQ ID NO:99 and the light-chain comprises the amino acid sequence of SEQ ID NO:304.

17. The antibody of claim 15, wherein the heavy-chain comprises the amino acid sequence of SEQ ID NO:112 and the light-chain comprises the amino acid sequence of SEQ ID NO:369.

18. The antibody of claim 15, wherein the heavy-chain comprises the amino acid sequence of SEQ ID NO:121 and the light-chain comprises the amino acid sequence of SEQ ID NO:333; or wherein the heavy-chain comprises the amino acid sequence of SEQ ID NO:169 and the light-chain comprises the amino acid sequence of SEQ ID NO:411; or wherein the heavy-chain comprises the amino acid sequence of SEQ ID NO:973 and the light-chain comprises the amino acid sequence of SEQ ID NO:975; or wherein the heavy-chain comprises the amino acid sequence of SEQ ID NO:980 and the light-chain comprises the amino acid sequence of SEQ ID NO:978.

19.-21. (canceled)

22. A pharmaceutical composition comprising the antibody of claim 1.

23. A kit comprising the antibody of claim 1.
